SEQUENCE LISTING (1067 sequences)

SEQ ID NO: 1 - protein, 539 aa, Human metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 2 - protein, 8 aa, Artificial Sequence
  Description of Artificial Sequence Synthetic peptide
GGTFTRLT

SEQ ID NO: 3 - protein, 8 aa, Artificial Sequence
  Description of Artificial Sequence Synthetic peptide
IIPAVDIT

SEQ ID NO: 4 - protein, 12 aa, Artificial Sequence
  Description of Artificial Sequence Synthetic peptide
ATVLDWSNALDI

SEQ ID NO: 5 - protein, 9 aa, Artificial Sequence
  Description of Artificial Sequence Synthetic peptide
GGSVNSDHW

SEQ ID NO: 6 - protein, 7 aa, Artificial Sequence
  Description of Artificial Sequence Synthetic peptide
LNHIGTF

SEQ ID NO: 7 - protein, 18 aa, Artificial Sequence
  Description of Artificial Sequence Synthetic peptide
ARRPPPLPEYFYYYFMDV

SEQ ID NO: 8 - protein, 8 aa, Artificial Sequence
  Description of Artificial Sequence Synthetic peptide
GGTFSNYI

SEQ ID NO: 9 - protein, 8 aa, Artificial Sequence
  Description of Artificial Sequence Synthetic peptide
IIPIVGME

SEQ ID NO: 10 - protein, 15 aa, Artificial Sequence
  Description of Artificial Sequence Synthetic peptide
ATEGLESGYPPYLEH

SEQ ID NO: 11 - protein, 8 aa, Artificial Sequence
  Description of Artificial Sequence Synthetic peptide
GGTFSNDI

SEQ ID NO: 12 - protein, 8 aa, Artificial Sequence
  Description of Artificial Sequence Synthetic peptide
IIPLVNMT

SEQ ID NO: 13 - protein, 15 aa, Artificial Sequence
  Description of Artificial Sequence Synthetic peptide
VRDLPSAGFPAPFDI

SEQ ID NO: 14 - protein, 8 aa, Artificial Sequence
  Description of Artificial Sequence Synthetic peptide
GYNFANYW

SEQ ID NO: 15 - protein, 8 aa, Artificial Sequence
  Description of Artificial Sequence Synthetic peptide
IYPGDSDT

SEQ ID NO: 16 - protein, 11 aa, Artificial Sequence
  Description of Artificial Sequence Synthetic peptide
ARPLAVSAFDI

SEQ ID NO: 17 - protein, 8 aa, Artificial Sequence
  Description of Artificial Sequence Synthetic peptide
GFPFDSFA

SEQ ID NO: 18 - protein, 8 aa, Artificial Sequence
  Description of Artificial Sequence Synthetic peptide
VWYDGGDK

SEQ ID NO: 19 - protein, 9 aa, Artificial Sequence
  Description of Artificial Sequence Synthetic peptide
ARGMITFDY

SEQ ID NO: 20 - protein, 8 aa, Artificial Sequence
  Description of Artificial Sequence Synthetic peptide
GFMFTSYA

SEQ ID NO: 21 - protein, 8 aa, Artificial Sequence
  Description of Artificial Sequence Synthetic peptide
ITGGGNFV

SEQ ID NO: 22 - protein, 16 aa, Artificial Sequence
  Description of Artificial Sequence Synthetic peptide
AREDYNSGANLHWFDP

SEQ ID NO: 23 - protein, 8 aa, Artificial Sequence
  Description of Artificial Sequence Synthetic peptide
GGIFDTNV

SEQ ID NO: 24 - protein, 8 aa, Artificial Sequence
  Description of Artificial Sequence Synthetic peptide
IIPPFGTA

SEQ ID NO: 25 - protein, 15 aa, Artificial Sequence
  Description of Artificial Sequence Synthetic peptide
ARDRTVIKYADGMDV

SEQ ID NO: 26 - protein, 8 aa, Artificial Sequence
  Description of Artificial Sequence Synthetic peptide
GGTLSNYA

SEQ ID NO: 27 - protein, 8 aa, Artificial Sequence
  Description of Artificial Sequence Synthetic peptide
IIPIFGTA

SEQ ID NO: 28 - protein, 15 aa, Artificial Sequence
  Description of Artificial Sequence Synthetic peptide
AQDRTVTKYAHGLDV

SEQ ID NO: 29 - protein, 8 aa, Artificial Sequence
  Description of Artificial Sequence Synthetic peptide
GASIGGYY

SEQ ID NO: 30 - protein, 7 aa, Artificial Sequence
  Description of Artificial Sequence Synthetic peptide
IYYSGRT

SEQ ID NO: 31 - protein, 12 aa, Artificial Sequence
  Description of Artificial Sequence Synthetic peptide
ASLTITPGWFDS

SEQ ID NO: 32 - protein, 8 aa, Artificial Sequence
  Description of Artificial Sequence Synthetic peptide
AFSISGYN

SEQ ID NO: 33 - protein, 8 aa, Artificial Sequence
  Description of Artificial Sequence Synthetic peptide
ITGGSNYI

SEQ ID NO: 34 - protein, 13 aa, Artificial Sequence
  Description of Artificial Sequence Synthetic peptide
ARETQPSPGSFDS

SEQ ID NO: 35 - protein, 8 aa, Artificial Sequence
  Description of Artificial Sequence Synthetic peptide
GFTLTSYS

SEQ ID NO: 36 - protein, 8 aa, Artificial Sequence
  Description of Artificial Sequence Synthetic peptide
ISSSSSYI

SEQ ID NO: 37 - protein, 12 aa, Artificial Sequence
  Description of Artificial Sequence Synthetic peptide
ARVARELDPFDM

SEQ ID NO: 38 - protein, 8 aa, Artificial Sequence
  Description of Artificial Sequence Synthetic peptide
GFSITSYG

SEQ ID NO: 39 - protein, 8 aa, Artificial Sequence
  Description of Artificial Sequence Synthetic peptide
ISSSSSYI

SEQ ID NO: 40 - protein, 13 aa, Artificial Sequence
  Description of Artificial Sequence Synthetic peptide
ARDAGLVPPWMDS

SEQ ID NO: 41 - protein, 8 aa, Artificial Sequence
  Description of Artificial Sequence Synthetic peptide
EFTLSGYA

SEQ ID NO: 42 - protein, 8 aa, Artificial Sequence
  Description of Artificial Sequence Synthetic peptide
ISSSSSYI

SEQ ID NO: 43 - protein, 14 aa, Artificial Sequence
  Description of Artificial Sequence Synthetic peptide
VRERPTAILPYLDL

SEQ ID NO: 44 - protein, 8 aa, Artificial Sequence
  Description of Artificial Sequence Synthetic peptide
GFTFSSYS

SEQ ID NO: 45 - protein, 8 aa, Artificial Sequence
  Description of Artificial Sequence Synthetic peptide
ISASSSYI

SEQ ID NO: 46 - protein, 18 aa, Artificial Sequence
  Description of Artificial Sequence Synthetic peptide
ARANYGSGNYYNPKWFDP

SEQ ID NO: 47 - protein, 8 aa, Artificial Sequence
  Description of Artificial Sequence Synthetic peptide
GGTINSYA

SEQ ID NO: 48 - protein, 8 aa, Artificial Sequence
  Description of Artificial Sequence Synthetic peptide
IIPIFGTA

SEQ ID NO: 49 - protein, 24 aa, Artificial Sequence
  Description of Artificial Sequence Synthetic peptide
ARGTRYIAVMVPSMQDYYFFGMDV

SEQ ID NO: 50 - protein, 8 aa, Artificial Sequence
  Description of Artificial Sequence Synthetic peptide
GFSLSGYG

SEQ ID NO: 51 - protein, 8 aa, Artificial Sequence
  Description of Artificial Sequence Synthetic peptide
ITASSSYI

SEQ ID NO: 52 - protein, 12 aa, Artificial Sequence
  Description of Artificial Sequence Synthetic peptide
ATNGKLPPYFDH

SEQ ID NO: 53 - protein, 8 aa, Artificial Sequence
  Description of Artificial Sequence Synthetic peptide
GGSFGGYH

SEQ ID NO: 54 - protein, 7 aa, Artificial Sequence
  Description of Artificial Sequence Synthetic peptide
IDHGGNT

SEQ ID NO: 55 - protein, 14 aa, Artificial Sequence
  Description of Artificial Sequence Synthetic peptide
ARGFGVVINNWFDP

SEQ ID NO: 56 - protein, 9 aa, Artificial Sequence
  Description of Artificial Sequence Synthetic peptide
GASITSSHW

SEQ ID NO: 57 - protein, 7 aa, Artificial Sequence
  Description of Artificial Sequence Synthetic peptide
IFHSGST

SEQ ID NO: 58 - protein, 15 aa, Artificial Sequence
  Description of Artificial Sequence Synthetic peptide
ARDDQIVVMPAGFDR

SEQ ID NO: 59 - protein, 10 aa, Artificial Sequence
  Description of Artificial Sequence Synthetic peptide
GDSVTSGAYS

SEQ ID NO: 60 - protein, 7 aa, Artificial Sequence
  Description of Artificial Sequence Synthetic peptide
INYSGRT

SEQ ID NO: 61 - protein, 15 aa, Artificial Sequence
  Description of Artificial Sequence Synthetic peptide
ARANVVGYSGFYFDY

SEQ ID NO: 62 - protein, 10 aa, Artificial Sequence
  Description of Artificial Sequence Synthetic peptide
GDSVTSGAHS

SEQ ID NO: 63 - protein, 7 aa, Artificial Sequence
  Description of Artificial Sequence Synthetic peptide
INYSGRT

SEQ ID NO: 64 - protein, 15 aa, Artificial Sequence
  Description of Artificial Sequence Synthetic peptide
ARANVVGYSGYYFDY

SEQ ID NO: 65 - protein, 8 aa, Artificial Sequence
  Description of Artificial Sequence Synthetic peptide
GGSFSGYH

SEQ ID NO: 66 - protein, 7 aa, Artificial Sequence
  Description of Artificial Sequence Synthetic peptide
VDHGGNT

SEQ ID NO: 67 - protein, 14 aa, Artificial Sequence
  Description of Artificial Sequence Synthetic peptide
ARGFGVVINNWFDP

SEQ ID NO: 68 - protein, 8 aa, Artificial Sequence
  Description of Artificial Sequence Synthetic peptide
GGSLSSYC

SEQ ID NO: 69 - protein, 7 aa, Artificial Sequence
  Description of Artificial Sequence Synthetic peptide
IYYSGGT

SEQ ID NO: 70 - protein, 15 aa, Artificial Sequence
  Description of Artificial Sequence Synthetic peptide
ARGEASGYLNYYFDY

SEQ ID NO: 71 - protein, 8 aa, Artificial Sequence
  Description of Artificial Sequence Synthetic peptide
GYTFSGYY

SEQ ID NO: 72 - protein, 8 aa, Artificial Sequence
  Description of Artificial Sequence Synthetic peptide
INPNSGGT

SEQ ID NO: 73 - protein, 16 aa, Artificial Sequence
  Description of Artificial Sequence Synthetic peptide
VRGQQLTRQYYFGMDV

SEQ ID NO: 74 - protein, 8 aa, Artificial Sequence
  Description of Artificial Sequence Synthetic peptide
GGSVSTYY

SEQ ID NO: 75 - protein, 7 aa, Artificial Sequence
  Description of Artificial Sequence Synthetic peptide
IYHNGNI

SEQ ID NO: 76 - protein, 15 aa, Artificial Sequence
  Description of Artificial Sequence Synthetic peptide
ATVSFIGYSLYYFDR

SEQ ID NO: 77 - protein, 8 aa, Artificial Sequence
  Description of Artificial Sequence Synthetic peptide
GFSLTTDE

SEQ ID NO: 78 - protein, 8 aa, Artificial Sequence
  Description of Artificial Sequence Synthetic peptide
ISASGHTI

SEQ ID NO: 79 - protein, 20 aa, Artificial Sequence
  Description of Artificial Sequence Synthetic peptide
ARVGRLGGVGYYYALGPFDL

SEQ ID NO: 80 - protein, 10 aa, Artificial Sequence
  Description of Artificial Sequence Synthetic peptide
GDSVTRNAYY

SEQ ID NO: 81 - protein, 7 aa, Artificial Sequence
  Description of Artificial Sequence Synthetic peptide
MYYSGST

SEQ ID NO: 82 - protein, 13 aa, Artificial Sequence
  Description of Artificial Sequence Synthetic peptide
GEATLTTYDAFDV

SEQ ID NO: 83 - protein, 10 aa, Artificial Sequence
  Description of Artificial Sequence Synthetic peptide
GGSISSSSLY

SEQ ID NO: 84 - protein, 7 aa, Artificial Sequence
  Description of Artificial Sequence Synthetic peptide
IYYGGSS

SEQ ID NO: 85 - protein, 15 aa, Artificial Sequence
  Description of Artificial Sequence Synthetic peptide
ARVGSPVTTTVWFDP

SEQ ID NO: 86 - protein, 8 aa, Artificial Sequence
  Description of Artificial Sequence Synthetic peptide
GFNVRNYY

SEQ ID NO: 87 - protein, 7 aa, Artificial Sequence
  Description of Artificial Sequence Synthetic peptide
IYGGGTT

SEQ ID NO: 88 - protein, 18 aa, Artificial Sequence
  Description of Artificial Sequence Synthetic peptide
ARDPGTVMSTENLQGMDV

SEQ ID NO: 89 - protein, 8 aa, Artificial Sequence
  Description of Artificial Sequence Synthetic peptide
GGSFSSYP

SEQ ID NO: 90 - protein, 8 aa, Artificial Sequence
  Description of Artificial Sequence Synthetic peptide
IIPLSGTT

SEQ ID NO: 91 - protein, 22 aa, Artificial Sequence
  Description of Artificial Sequence Synthetic peptide
ARSGAPTYYYDSSGFHGGAFHF

SEQ ID NO: 92 - protein, 8 aa, Artificial Sequence
  Description of Artificial Sequence Synthetic peptide
GGSITSHY

SEQ ID NO: 93 - protein, 7 aa, Artificial Sequence
  Description of Artificial Sequence Synthetic peptide
IYYSGST

SEQ ID NO: 94 - protein, 12 aa, Artificial Sequence
  Description of Artificial Sequence Synthetic peptide
ARCMPLYHVMHV

SEQ ID NO: 95 - protein, 8 aa, Artificial Sequence
  Description of Artificial Sequence Synthetic peptide
GFTFSSHA

SEQ ID NO: 96 - protein, 8 aa, Artificial Sequence
  Description of Artificial Sequence Synthetic peptide
ISYDGSNR

SEQ ID NO: 97 - protein, 18 aa, Artificial Sequence
  Description of Artificial Sequence Synthetic peptide
ARDFQPSPMVYGVGYFDY

SEQ ID NO: 98 - protein, 8 aa, Artificial Sequence
  Description of Artificial Sequence Synthetic peptide
GFTFSSSA

SEQ ID NO: 99 - protein, 8 aa, Artificial Sequence
  Description of Artificial Sequence Synthetic peptide
ISYHGTNK

SEQ ID NO: 100 - protein, 12 aa, Artificial Sequence
  Description of Artificial Sequence Synthetic peptide
AKEAAVSGNFDS

SEQ ID NO: 101 - protein, 8 aa, Artificial Sequence
  Description of Artificial Sequence Synthetic peptide
GFTFSNYA

SEQ ID NO: 102 - protein, 8 aa, Artificial Sequence
  Description of Artificial Sequence Synthetic peptide
INDRGETT

SEQ ID NO: 103 - protein, 17 aa, Artificial Sequence
  Description of Artificial Sequence Synthetic peptide
TRAYVVLSVYAPKGFDY

SEQ ID NO: 104 - protein, 8 aa, Artificial Sequence
  Description of Artificial Sequence Synthetic peptide
RGAITDYH

SEQ ID NO: 105 - protein, 7 aa, Artificial Sequence
  Description of Artificial Sequence Synthetic peptide
ISSSGST

SEQ ID NO: 106 - protein, 14 aa, Artificial Sequence
  Description of Artificial Sequence Synthetic peptide
ASSSGRYWGGALDL

SEQ ID NO: 107 - protein, 8 aa, Artificial Sequence
  Description of Artificial Sequence Synthetic peptide
GYNFDNYY

SEQ ID NO: 108 - protein, 8 aa, Artificial Sequence
  Description of Artificial Sequence Synthetic peptide
IDPSGGRI

SEQ ID NO: 109 - protein, 10 aa, Artificial Sequence
  Description of Artificial Sequence Synthetic peptide
ARLEATHEGP

SEQ ID NO: 110 - protein, 8 aa, Artificial Sequence
  Description of Artificial Sequence Synthetic peptide
GFGFRSYW

SEQ ID NO: 111 - protein, 8 aa, Artificial Sequence
  Description of Artificial Sequence Synthetic peptide
INNDGSSA

SEQ ID NO: 112 - protein, 15 aa, Artificial Sequence
  Description of Artificial Sequence Synthetic peptide
AREFGVLDYHYGMDV

SEQ ID NO: 113 - protein, 8 aa, Artificial Sequence
  Description of Artificial Sequence Synthetic peptide
GFSFGSYY

SEQ ID NO: 114 - protein, 8 aa, Artificial Sequence
  Description of Artificial Sequence Synthetic peptide
TSSDGSTT

SEQ ID NO: 115 - protein, 15 aa, Artificial Sequence
  Description of Artificial Sequence Synthetic peptide
ARELGILDYYYGMDV

SEQ ID NO: 116 - protein, 8 aa, Artificial Sequence
  Description of Artificial Sequence Synthetic peptide
GFIFSNYA

SEQ ID NO: 117 - protein, 8 aa, Artificial Sequence
  Description of Artificial Sequence Synthetic peptide
ISYDGTNK

SEQ ID NO: 118 - protein, 15 aa, Artificial Sequence
  Description of Artificial Sequence Synthetic peptide
ARDPERLADLGRMDF

SEQ ID NO: 119 - protein, 10 aa, Artificial Sequence
  Description of Artificial Sequence Synthetic peptide
GASVSSEYYY

SEQ ID NO: 120 - protein, 7 aa, Artificial Sequence
  Description of Artificial Sequence Synthetic peptide
IYHNGDT

SEQ ID NO: 121 - protein, 13 aa, Artificial Sequence
  Description of Artificial Sequence Synthetic peptide
ARLAVAGVTGFHY

SEQ ID NO: 122 - protein, 8 aa, Artificial Sequence
  Description of Artificial Sequence Synthetic peptide
GFTFSRYA

SEQ ID NO: 123 - protein, 8 aa, Artificial Sequence
  Description of Artificial Sequence Synthetic peptide
MSHDGSNR

SEQ ID NO: 124 - protein, 18 aa, Artificial Sequence
  Description of Artificial Sequence Synthetic peptide
AKDPGGLGYSYGLGYFDY

SEQ ID NO: 125 - protein, 8 aa, Artificial Sequence
  Description of Artificial Sequence Synthetic peptide
GFNFNTYA

SEQ ID NO: 126 - protein, 8 aa, Artificial Sequence
  Description of Artificial Sequence Synthetic peptide
ISGSGGVK

SEQ ID NO: 127 - protein, 16 aa, Artificial Sequence
  Description of Artificial Sequence Synthetic peptide
AKSQNPQLVFFDSFDM

SEQ ID NO: 128 - protein, 8 aa, Artificial Sequence
  Description of Artificial Sequence Synthetic peptide
GFRFNAFS

SEQ ID NO: 129 - protein, 8 aa, Artificial Sequence
  Description of Artificial Sequence Synthetic peptide
IDSRSNII

SEQ ID NO: 130 - protein, 22 aa, Artificial Sequence
  Description of Artificial Sequence Synthetic peptide
AKALCVVNYDFWSGLLGCAMDV

SEQ ID NO: 131 - protein, 10 aa, Artificial Sequence
  Description of Artificial Sequence Synthetic peptide
GFSLNTYGMS

SEQ ID NO: 132 - protein, 7 aa, Artificial Sequence
  Description of Artificial Sequence Synthetic peptide
IDWDGDK

SEQ ID NO: 133 - protein, 17 aa, Artificial Sequence
  Description of Artificial Sequence Synthetic peptide
AQSVIVPAQSFVSWFDP

SEQ ID NO: 134 - protein, 8 aa, Artificial Sequence
  Description of Artificial Sequence Synthetic peptide
GFKFNAFS

SEQ ID NO: 135 - protein, 8 aa, Artificial Sequence
  Description of Artificial Sequence Synthetic peptide
IDSRSNII

SEQ ID NO: 136 - protein, 22 aa, Artificial Sequence
  Description of Artificial Sequence Synthetic peptide
AKALCVVNYDFWSGLLGCAMDV

SEQ ID NO: 137 - protein, 8 aa, Artificial Sequence
  Description of Artificial Sequence Synthetic peptide
GDIFSSHA

SEQ ID NO: 138 - protein, 8 aa, Artificial Sequence
  Description of Artificial Sequence Synthetic peptide
IIPLVGIR

SEQ ID NO: 139 - protein, 13 aa, Artificial Sequence
  Description of Artificial Sequence Synthetic peptide
ARDRTDWSGGLDI

SEQ ID NO: 140 - protein, 10 aa, Artificial Sequence
  Description of Artificial Sequence Synthetic peptide
GFSLSANSVG

SEQ ID NO: 141 - protein, 7 aa, Artificial Sequence
  Description of Artificial Sequence Synthetic peptide
IYWDDDK

SEQ ID NO: 142 - protein, 9 aa, Artificial Sequence
  Description of Artificial Sequence Synthetic peptide
VRKHYDLLQ

SEQ ID NO: 143 - protein, 8 aa, Artificial Sequence
  Description of Artificial Sequence Synthetic peptide
GYSFSNYY

SEQ ID NO: 144 - protein, 8 aa, Artificial Sequence
  Description of Artificial Sequence Synthetic peptide
INPSGGST

SEQ ID NO: 145 - protein, 11 aa, Artificial Sequence
  Description of Artificial Sequence Synthetic peptide
VRSLANGSGSP

SEQ ID NO: 146 - protein, 8 aa, Artificial Sequence
  Description of Artificial Sequence Synthetic peptide
GFTFSNYP

SEQ ID NO: 147 - protein, 8 aa, Artificial Sequence
  Description of Artificial Sequence Synthetic peptide
ISYDGRTK

SEQ ID NO: 148 - protein, 10 aa, Artificial Sequence
  Description of Artificial Sequence Synthetic peptide
AREGLEYLNY

SEQ ID NO: 149 - protein, 8 aa, Artificial Sequence
  Description of Artificial Sequence Synthetic peptide
GFTFTSFS

SEQ ID NO: 150 - protein, 8 aa, Artificial Sequence
  Description of Artificial Sequence Synthetic peptide
ISHDGIIK

SEQ ID NO: 151 - protein, 17 aa, Artificial Sequence
  Description of Artificial Sequence Synthetic peptide
ARDFPGSRGFFRYYFDH

SEQ ID NO: 152 - protein, 8 aa, Artificial Sequence
  Description of Artificial Sequence Synthetic peptide
GRAFLTYD

SEQ ID NO: 153 - protein, 8 aa, Artificial Sequence
  Description of Artificial Sequence Synthetic peptide
MNPGSGNA

SEQ ID NO: 154 - protein, 14 aa, Artificial Sequence
  Description of Artificial Sequence Synthetic peptide
ARGEVSMRWRWLDP

SEQ ID NO: 155 - protein, 8 aa, Artificial Sequence
  Description of Artificial Sequence Synthetic peptide
EDTFTNYY

SEQ ID NO: 156 - protein, 8 aa, Artificial Sequence
  Description of Artificial Sequence Synthetic peptide
INPSGGST

SEQ ID NO: 157 - protein, 11 aa, Artificial Sequence
  Description of Artificial Sequence Synthetic peptide
ARQGRTAACDY

SEQ ID NO: 158 - protein, 8 aa, Artificial Sequence
  Description of Artificial Sequence Synthetic peptide
GFTFRSYT

SEQ ID NO: 159 - protein, 8 aa, Artificial Sequence
  Description of Artificial Sequence Synthetic peptide
ILHDGTNR

SEQ ID NO: 160 - protein, 10 aa, Artificial Sequence
  Description of Artificial Sequence Synthetic peptide
ARESLEWMLD

SEQ ID NO: 161 - protein, 8 aa, Artificial Sequence
  Description of Artificial Sequence Synthetic peptide
GYSFISYY

SEQ ID NO: 162 - protein, 8 aa, Artificial Sequence
  Description of Artificial Sequence Synthetic peptide
INPSGGST

SEQ ID NO: 163 - protein, 11 aa, Artificial Sequence
  Description of Artificial Sequence Synthetic peptide
AREQAVGAYDI

SEQ ID NO: 164 - protein, 8 aa, Artificial Sequence
  Description of Artificial Sequence Synthetic peptide
GYIFTGYF

SEQ ID NO: 165 - protein, 8 aa, Artificial Sequence
  Description of Artificial Sequence Synthetic peptide
VNPYSGAT

SEQ ID NO: 166 - protein, 14 aa, Artificial Sequence
  Description of Artificial Sequence Synthetic peptide
AKDTSNSGPDLLEH

SEQ ID NO: 167 - protein, 8 aa, Artificial Sequence
  Description of Artificial Sequence Synthetic peptide
EYTFTNHY

SEQ ID NO: 168 - protein, 8 aa, Artificial Sequence
  Description of Artificial Sequence Synthetic peptide
INPTGGST

SEQ ID NO: 169 - protein, 11 aa, Artificial Sequence
  Description of Artificial Sequence Synthetic peptide
ARDRGMRYFDY

SEQ ID NO: 170 - protein, 10 aa, Artificial Sequence
  Description of Artificial Sequence Synthetic peptide
GGSISSSSNS

SEQ ID NO: 171 - protein, 7 aa, Artificial Sequence
  Description of Artificial Sequence Synthetic peptide
IFYFGST

SEQ ID NO: 172 - protein, 16 aa, Artificial Sequence
  Description of Artificial Sequence Synthetic peptide
ARHILSSSGVSGWLDP

SEQ ID NO: 173 - protein, 8 aa, Artificial Sequence
  Description of Artificial Sequence Synthetic peptide
GYTFTRHA

SEQ ID NO: 174 - protein, 8 aa, Artificial Sequence
  Description of Artificial Sequence Synthetic peptide
VHGGNENT

SEQ ID NO: 175 - protein, 10 aa, Artificial Sequence
  Description of Artificial Sequence Synthetic peptide
ATAMPGAFNM

SEQ ID NO: 176 - protein, 8 aa, Artificial Sequence
  Description of Artificial Sequence Synthetic peptide
GFSFSTFW

SEQ ID NO: 177 - protein, 8 aa, Artificial Sequence
  Description of Artificial Sequence Synthetic peptide
IKPDGSEK

SEQ ID NO: 178 - protein, 20 aa, Artificial Sequence
  Description of Artificial Sequence Synthetic peptide
ARDRGYCRGGSCYRRNAFDL

SEQ ID NO: 179 - protein, 8 aa, Artificial Sequence
  Description of Artificial Sequence Synthetic peptide
GYIFSNYR

SEQ ID NO: 180 - protein, 8 aa, Artificial Sequence
  Description of Artificial Sequence Synthetic peptide
ITAGSGNT

SEQ ID NO: 181 - protein, 12 aa, Artificial Sequence
  Description of Artificial Sequence Synthetic peptide
AREYNWNDAFPY

SEQ ID NO: 182 - protein, 8 aa, Artificial Sequence
  Description of Artificial Sequence Synthetic peptide
GFTFSNYA

SEQ ID NO: 183 - protein, 8 aa, Artificial Sequence
  Description of Artificial Sequence Synthetic peptide
IGGSVGTT

SEQ ID NO: 184 - protein, 15 aa, Artificial Sequence
  Description of Artificial Sequence Synthetic peptide
AKVPSFFVFEDGFDM

SEQ ID NO: 185 - protein, 8 aa, Artificial Sequence
  Description of Artificial Sequence Synthetic peptide
GFTFSLYT

SEQ ID NO: 186 - protein, 8 aa, Artificial Sequence
  Description of Artificial Sequence Synthetic peptide
ISADNSNT

SEQ ID NO: 187 - protein, 17 aa, Artificial Sequence
  Description of Artificial Sequence Synthetic peptide
TRGPAYYFDSSGFAFDI

SEQ ID NO: 188 - protein, 8 aa, Artificial Sequence
  Description of Artificial Sequence Synthetic peptide
GFTFESAW

SEQ ID NO: 189 - protein, 10 aa, Artificial Sequence
  Description of Artificial Sequence Synthetic peptide
IKSKLDHGTT

SEQ ID NO: 190 - protein, 22 aa, Artificial Sequence
  Description of Artificial Sequence Synthetic peptide
ATDRSDVRFLEWLGNYQFGMDV

SEQ ID NO: 191 - protein, 8 aa, Artificial Sequence
  Description of Artificial Sequence Synthetic peptide
GFTFSSYA

SEQ ID NO: 192 - protein, 8 aa, Artificial Sequence
  Description of Artificial Sequence Synthetic peptide
ITYDGSGK

SEQ ID NO: 193 - protein, 14 aa, Artificial Sequence
  Description of Artificial Sequence Synthetic peptide
VKSGSFSSAFWLED

SEQ ID NO: 194 - protein, 8 aa, Artificial Sequence
  Description of Artificial Sequence Synthetic peptide
GYTFTSHD

SEQ ID NO: 195 - protein, 8 aa, Artificial Sequence
  Description of Artificial Sequence Synthetic peptide
MNPNSATT

SEQ ID NO: 196 - protein, 19 aa, Artificial Sequence
  Description of Artificial Sequence Synthetic peptide
ARNRHYYYADGPTRDAFDI

SEQ ID NO: 197 - protein, 8 aa, Artificial Sequence
  Description of Artificial Sequence Synthetic peptide
GYTFTSHD

SEQ ID NO: 198 - protein, 8 aa, Artificial Sequence
  Description of Artificial Sequence Synthetic peptide
MNPNSDTT

SEQ ID NO: 199 - protein, 19 aa, Artificial Sequence
  Description of Artificial Sequence Synthetic peptide
ARGRHNYYTNGPTRDGFDI

SEQ ID NO: 200 - protein, 8 aa, Artificial Sequence
  Description of Artificial Sequence Synthetic peptide
GFVFRFSG

SEQ ID NO: 201 - protein, 8 aa, Artificial Sequence
  Description of Artificial Sequence Synthetic peptide
ISDDGSQK

SEQ ID NO: 202 - protein, 13 aa, Artificial Sequence
  Description of Artificial Sequence Synthetic peptide
ARGDYGVSYFLAH

SEQ ID NO: 203 - protein, 8 aa, Artificial Sequence
  Description of Artificial Sequence Synthetic peptide
GGTLSNYA

SEQ ID NO: 204 - protein, 8 aa, Artificial Sequence
  Description of Artificial Sequence Synthetic peptide
IIPVMNVA

SEQ ID NO: 205 - protein, 17 aa, Artificial Sequence
  Description of Artificial Sequence Synthetic peptide
ATARGLGSLEGGDAFDV

SEQ ID NO: 206 - protein, 8 aa, Artificial Sequence
  Description of Artificial Sequence Synthetic peptide
GYTFNGHY

SEQ ID NO: 207 - protein, 8 aa, Artificial Sequence
  Description of Artificial Sequence Synthetic peptide
INPNSGGT

SEQ ID NO: 208 - protein, 20 aa, Artificial Sequence
  Description of Artificial Sequence Synthetic peptide
ARDNFGFWSASSGRHNWLDS

SEQ ID NO: 209 - protein, 8 aa, Artificial Sequence
  Description of Artificial Sequence Synthetic peptide
GVTLTDYY

SEQ ID NO: 210 - protein, 8 aa, Artificial Sequence
  Description of Artificial Sequence Synthetic peptide
INPARGGT

SEQ ID NO: 211 - protein, 18 aa, Artificial Sequence
  Description of Artificial Sequence Synthetic peptide
ARSGSTHNIYSGFYAFDH

SEQ ID NO: 212 - protein, 8 aa, Artificial Sequence
  Description of Artificial Sequence Synthetic peptide
GFTFNNYW

SEQ ID NO: 213 - protein, 8 aa, Artificial Sequence
  Description of Artificial Sequence Synthetic peptide
VKSDGSIT

SEQ ID NO: 214 - protein, 20 aa, Artificial Sequence
  Description of Artificial Sequence Synthetic peptide
ARESRPYDPLTGSHPDAFDI

SEQ ID NO: 215 - protein, 8 aa, Artificial Sequence
  Description of Artificial Sequence Synthetic peptide
GYTFTSYG

SEQ ID NO: 216 - protein, 8 aa, Artificial Sequence
  Description of Artificial Sequence Synthetic peptide
ISVHTDDT

SEQ ID NO: 217 - protein, 17 aa, Artificial Sequence
  Description of Artificial Sequence Synthetic peptide
ARDRTDISGRVRDAFDL

SEQ ID NO: 218 - protein, 9 aa, Artificial Sequence
  Description of Artificial Sequence Synthetic peptide
RFSINSGYY

SEQ ID NO: 219 - protein, 7 aa, Artificial Sequence
  Description of Artificial Sequence Synthetic peptide
ISHAGSI

SEQ ID NO: 220 - protein, 12 aa, Artificial Sequence
  Description of Artificial Sequence Synthetic peptide
AASSNWHNALEY

SEQ ID NO: 221 - protein, 9 aa, Artificial Sequence
  Description of Artificial Sequence Synthetic peptide
RYSINSGYY

SEQ ID NO: 222 - protein, 7 aa, Artificial Sequence
  Description of Artificial Sequence Synthetic peptide
VSHAGVT

SEQ ID NO: 223 - protein, 12 aa, Artificial Sequence
  Description of Artificial Sequence Synthetic peptide
AASSNWHNALEY

SEQ ID NO: 224 - protein, 8 aa, Artificial Sequence
  Description of Artificial Sequence Synthetic peptide
AFTFSDYA

SEQ ID NO: 225 - protein, 8 aa, Artificial Sequence
  Description of Artificial Sequence Synthetic peptide
INTSGGRT

SEQ ID NO: 226 - protein, 9 aa, Artificial Sequence
  Description of Artificial Sequence Synthetic peptide
ARFFYVMDV

SEQ ID NO: 227 - protein, 8 aa, Artificial Sequence
  Description of Artificial Sequence Synthetic peptide
GFNFRTHW

SEQ ID NO: 228 - protein, 8 aa, Artificial Sequence
  Description of Artificial Sequence Synthetic peptide
TNGDGSVT

SEQ ID NO: 229 - protein, 20 aa, Artificial Sequence
  Description of Artificial Sequence Synthetic peptide
ARERLDYSSTRGHFPGDFDS

SEQ ID NO: 230 - protein, 8 aa, Artificial Sequence
  Description of Artificial Sequence Synthetic peptide
GGTFTNFA

SEQ ID NO: 231 - protein, 8 aa, Artificial Sequence
  Description of Artificial Sequence Synthetic peptide
IIPFVGPA

SEQ ID NO: 232 - protein, 19 aa, Artificial Sequence
  Description of Artificial Sequence Synthetic peptide
ARVRGFGETAYFYHYYMDV

SEQ ID NO: 233 - protein, 10 aa, Artificial Sequence
  Description of Artificial Sequence Synthetic peptide
GDSISSSSFY

SEQ ID NO: 234 - protein, 7 aa, Artificial Sequence
  Description of Artificial Sequence Synthetic peptide
IYYTGST

SEQ ID NO: 235 - protein, 14 aa, Artificial Sequence
  Description of Artificial Sequence Synthetic peptide
ARQEGSSWSNWFDP

SEQ ID NO: 236 - protein, 8 aa, Artificial Sequence
  Description of Artificial Sequence Synthetic peptide
GFTFNNYA

SEQ ID NO: 237 - protein, 8 aa, Artificial Sequence
  Description of Artificial Sequence Synthetic peptide
ISDDGSKK

SEQ ID NO: 238 - protein, 14 aa, Artificial Sequence
  Description of Artificial Sequence Synthetic peptide
ARDPTPLGEYYFDY

SEQ ID NO: 239 - protein, 10 aa, Artificial Sequence
  Description of Artificial Sequence Synthetic peptide
GGSISSSTYY

SEQ ID NO: 240 - protein, 7 aa, Artificial Sequence
  Description of Artificial Sequence Synthetic peptide
IYYSGST

SEQ ID NO: 241 - protein, 12 aa, Artificial Sequence
  Description of Artificial Sequence Synthetic peptide
ARLVSPYDQFDY

SEQ ID NO: 242 - protein, 8 aa, Artificial Sequence
  Description of Artificial Sequence Synthetic peptide
GFMFSSYA

SEQ ID NO: 243 - protein, 8 aa, Artificial Sequence
  Description of Artificial Sequence Synthetic peptide
ISGSGGNT

SEQ ID NO: 244 - protein, 14 aa, Artificial Sequence
  Description of Artificial Sequence Synthetic peptide
AKDLGNDYFYYIDV

SEQ ID NO: 245 - protein, 8 aa, Artificial Sequence
  Description of Artificial Sequence Synthetic peptide
GLTFSTYV

SEQ ID NO: 246 - protein, 8 aa, Artificial Sequence
  Description of Artificial Sequence Synthetic peptide
ISYDGTNE

SEQ ID NO: 247 - protein, 20 aa, Artificial Sequence
  Description of Artificial Sequence Synthetic peptide
ARGTADYDVWSGSAWGGFDY

SEQ ID NO: 248 - protein, 10 aa, Artificial Sequence
  Description of Artificial Sequence Synthetic peptide
GDSISSSDYC

SEQ ID NO: 249 - protein, 7 aa, Artificial Sequence
  Description of Artificial Sequence Synthetic peptide
SVYSGDT

SEQ ID NO: 250 - protein, 13 aa, Artificial Sequence
  Description of Artificial Sequence Synthetic peptide
ARGNDFWSGSFDY

SEQ ID NO: 251 - protein, 10 aa, Artificial Sequence
  Description of Artificial Sequence Synthetic peptide
GGSISRSTHY

SEQ ID NO: 252 - protein, 7 aa, Artificial Sequence
  Description of Artificial Sequence Synthetic peptide
IYYTGNT

SEQ ID NO: 253 - protein, 13 aa, Artificial Sequence
  Description of Artificial Sequence Synthetic peptide
ARHETYDYGVFNI

SEQ ID NO: 254 - protein, 8 aa, Artificial Sequence
  Description of Artificial Sequence Synthetic peptide
GYTFTSYG

SEQ ID NO: 255 - protein, 8 aa, Artificial Sequence
  Description of Artificial Sequence Synthetic peptide
ISAYNGNT

SEQ ID NO: 256 - protein, 16 aa, Artificial Sequence
  Description of Artificial Sequence Synthetic peptide
ARDAPFGRDDGYGMDV

SEQ ID NO: 257 - protein, 8 aa, Artificial Sequence
  Description of Artificial Sequence Synthetic peptide
GFTFTTYT

SEQ ID NO: 258 - protein, 8 aa, Artificial Sequence
  Description of Artificial Sequence Synthetic peptide
ISSDGNNK

SEQ ID NO: 259 - protein, 13 aa, Artificial Sequence
  Description of Artificial Sequence Synthetic peptide
ARGYYSGGDAFDV

SEQ ID NO: 260 - protein, 8 aa, Artificial Sequence
  Description of Artificial Sequence Synthetic peptide
AYTFTSYG

SEQ ID NO: 261 - protein, 8 aa, Artificial Sequence
  Description of Artificial Sequence Synthetic peptide
ISAYRGNT

SEQ ID NO: 262 - protein, 15 aa, Artificial Sequence
  Description of Artificial Sequence Synthetic peptide
ARTSYYDSSYYPIDY

SEQ ID NO: 263 - protein, 10 aa, Artificial Sequence
  Description of Artificial Sequence Synthetic peptide
GFSLNTGGVS

SEQ ID NO: 264 - protein, 7 aa, Artificial Sequence
  Description of Artificial Sequence Synthetic peptide
IYWDGDK

SEQ ID NO: 265 - protein, 11 aa, Artificial Sequence
  Description of Artificial Sequence Synthetic peptide
AHRENSAYIDF

SEQ ID NO: 266 - protein, 8 aa, Artificial Sequence
  Description of Artificial Sequence Synthetic peptide
GFTFSSYA

SEQ ID NO: 267 - protein, 8 aa, Artificial Sequence
  Description of Artificial Sequence Synthetic peptide
ISYDGSNK

SEQ ID NO: 268 - protein, 12 aa, Artificial Sequence
  Description of Artificial Sequence Synthetic peptide
ARDVQQQLALDY

SEQ ID NO: 269 - protein, 8 aa, Artificial Sequence
  Description of Artificial Sequence Synthetic peptide
GFTFSDYS

SEQ ID NO: 270 - protein, 8 aa, Artificial Sequence
  Description of Artificial Sequence Synthetic peptide
ISSTGSTI

SEQ ID NO: 271 - protein, 13 aa, Artificial Sequence
  Description of Artificial Sequence Synthetic peptide
ARGKTTVTIWFDP

SEQ ID NO: 272 - protein, 9 aa, Artificial Sequence
  Description of Artificial Sequence Synthetic peptide
GGSISSNNW

SEQ ID NO: 273 - protein, 7 aa, Artificial Sequence
  Description of Artificial Sequence Synthetic peptide
IYHSGST

SEQ ID NO: 274 - protein, 20 aa, Artificial Sequence
  Description of Artificial Sequence Synthetic peptide
ARRPNDYGDYSYYYYYYMDV

SEQ ID NO: 275 - protein, 8 aa, Artificial Sequence
  Description of Artificial Sequence Synthetic peptide
GYNFPAYG

SEQ ID NO: 276 - protein, 8 aa, Artificial Sequence
  Description of Artificial Sequence Synthetic peptide
ISAYNVNT

SEQ ID NO: 277 - protein, 17 aa, Artificial Sequence
  Description of Artificial Sequence Synthetic peptide
ARVPRPPLSGTYSTADF

SEQ ID NO: 278 - protein, 8 aa, Artificial Sequence
  Description of Artificial Sequence Synthetic peptide
GGSFSGYY

SEQ ID NO: 279 - protein, 7 aa, Artificial Sequence
  Description of Artificial Sequence Synthetic peptide
INHSGST

SEQ ID NO: 280 - protein, 18 aa, Artificial Sequence
  Description of Artificial Sequence Synthetic peptide
ARGPIRYSSYYYYYGLDV

SEQ ID NO: 281 - protein, 8 aa, Artificial Sequence
  Description of Artificial Sequence Synthetic peptide
GFTFSSYG

SEQ ID NO: 282 - protein, 8 aa, Artificial Sequence
  Description of Artificial Sequence Synthetic peptide
IRYDGSNK

SEQ ID NO: 283 - protein, 15 aa, Artificial Sequence
  Description of Artificial Sequence Synthetic peptide
AKDSGSYYYYYYMDV

SEQ ID NO: 284 - protein, 10 aa, Artificial Sequence
  Description of Artificial Sequence Synthetic peptide
GGSISSSSYF

SEQ ID NO: 285 - protein, 7 aa, Artificial Sequence
  Description of Artificial Sequence Synthetic peptide
VFYSGST

SEQ ID NO: 286 - protein, 10 aa, Artificial Sequence
  Description of Artificial Sequence Synthetic peptide
ARHSWDRWVY

SEQ ID NO: 287 - protein, 8 aa, Artificial Sequence
  Description of Artificial Sequence Synthetic peptide
GFPFNFYW

SEQ ID NO: 288 - protein, 8 aa, Artificial Sequence
  Description of Artificial Sequence Synthetic peptide
INNDGSSP

SEQ ID NO: 289 - protein, 12 aa, Artificial Sequence
  Description of Artificial Sequence Synthetic peptide
ARAARFGDYVDQ

SEQ ID NO: 290 - protein, 8 aa, Artificial Sequence
  Description of Artificial Sequence Synthetic peptide
GVSISSHY

SEQ ID NO: 291 - protein, 7 aa, Artificial Sequence
  Description of Artificial Sequence Synthetic peptide
LYTSGTT

SEQ ID NO: 292 - protein, 18 aa, Artificial Sequence
  Description of Artificial Sequence Synthetic peptide
ARDVRLTVFGALHNWFDP

SEQ ID NO: 293 - protein, 8 aa, Artificial Sequence
  Description of Artificial Sequence Synthetic peptide
GGSISSYY

SEQ ID NO: 294 - protein, 7 aa, Artificial Sequence
  Description of Artificial Sequence Synthetic peptide
IYTSGST

SEQ ID NO: 295 - protein, 16 aa, Artificial Sequence
  Description of Artificial Sequence Synthetic peptide
ARDASGLSWYDWYFDL

SEQ ID NO: 296 - protein, 8 aa, Artificial Sequence
  Description of Artificial Sequence Synthetic peptide
GFSVRTNY

SEQ ID NO: 297 - protein, 7 aa, Artificial Sequence
  Description of Artificial Sequence Synthetic peptide
IYSDGRT

SEQ ID NO: 298 - protein, 16 aa, Artificial Sequence
  Description of Artificial Sequence Synthetic peptide
ARDPTFYSSSWWYLDH

SEQ ID NO: 299 - protein, 8 aa, Artificial Sequence
  Description of Artificial Sequence Synthetic peptide
GFTFSSYW

SEQ ID NO: 300 - protein, 8 aa, Artificial Sequence
  Description of Artificial Sequence Synthetic peptide
IKQDGSEK

SEQ ID NO: 301 - protein, 20 aa, Artificial Sequence
  Description of Artificial Sequence Synthetic peptide
ARYYYDSSGYYYYYYYYMDV

SEQ ID NO: 302 - protein, 8 aa, Artificial Sequence
  Description of Artificial Sequence Synthetic peptide
GFSFSSYV

SEQ ID NO: 303 - protein, 8 aa, Artificial Sequence
  Description of Artificial Sequence Synthetic peptide
VSAGGGST

SEQ ID NO: 304 - protein, 14 aa, Artificial Sequence
  Description of Artificial Sequence Synthetic peptide
AKDKYPHYYYGMDV

SEQ ID NO: 305 - protein, 7 aa, Artificial Sequence
  Description of Artificial Sequence Synthetic peptide
GGTFDNA

SEQ ID NO: 306 - protein, 8 aa, Artificial Sequence
  Description of Artificial Sequence Synthetic peptide
IIPLVDIV

SEQ ID NO: 307 - protein, 11 aa, Artificial Sequence
  Description of Artificial Sequence Synthetic peptide
ASAFQDGWFDP

SEQ ID NO: 308 - protein, 8 aa, Artificial Sequence
  Description of Artificial Sequence Synthetic peptide
GDIMSSHS

SEQ ID NO: 309 - protein, 8 aa, Artificial Sequence
  Description of Artificial Sequence Synthetic peptide
IIPLFGMK

SEQ ID NO: 310 - protein, 11 aa, Artificial Sequence
  Description of Artificial Sequence Synthetic peptide
AREENLSWLDY

SEQ ID NO: 311 - protein, 8 aa, Artificial Sequence
  Description of Artificial Sequence Synthetic peptide
GYSITTYG

SEQ ID NO: 312 - protein, 8 aa, Artificial Sequence
  Description of Artificial Sequence Synthetic peptide
SSAHTGNT

SEQ ID NO: 313 - protein, 14 aa, Artificial Sequence
  Description of Artificial Sequence Synthetic peptide
AMGAYTSSRNWFDP

SEQ ID NO: 314 - protein, 8 aa, Artificial Sequence
  Description of Artificial Sequence Synthetic peptide
GFTFSNAW

SEQ ID NO: 315 - protein, 10 aa, Artificial Sequence
  Description of Artificial Sequence Synthetic peptide
IKPHTDGATT

SEQ ID NO: 316 - protein, 9 aa, Artificial Sequence
  Description of Artificial Sequence Synthetic peptide
TTGPRLSLM

SEQ ID NO: 317 - protein, 8 aa, Artificial Sequence
  Description of Artificial Sequence Synthetic peptide
GGTLTRYT

SEQ ID NO: 318 - protein, 8 aa, Artificial Sequence
  Description of Artificial Sequence Synthetic peptide
IIPFLDIA

SEQ ID NO: 319 - protein, 11 aa, Artificial Sequence
  Description of Artificial Sequence Synthetic peptide
ARDHSGNYFDL

SEQ ID NO: 320 - protein, 8 aa, Artificial Sequence
  Description of Artificial Sequence Synthetic peptide
GYTFSSYA

SEQ ID NO: 321 - protein, 8 aa, Artificial Sequence
  Description of Artificial Sequence Synthetic peptide
ISGSGANT

SEQ ID NO: 322 - protein, 10 aa, Artificial Sequence
  Description of Artificial Sequence Synthetic peptide
ARGLYTTGDY

SEQ ID NO: 323 - protein, 8 aa, Artificial Sequence
  Description of Artificial Sequence Synthetic peptide
GFTFSSYA

SEQ ID NO: 324 - protein, 8 aa, Artificial Sequence
  Description of Artificial Sequence Synthetic peptide
ITESGDDA

SEQ ID NO: 325 - protein, 23 aa, Artificial Sequence
  Description of Artificial Sequence Synthetic peptide
AKDMGLYKSSWYGNFFYYYYMDV

SEQ ID NO: 326 - protein, 8 aa, Artificial Sequence
  Description of Artificial Sequence Synthetic peptide
GFTVSSKY

SEQ ID NO: 327 - protein, 7 aa, Artificial Sequence
  Description of Artificial Sequence Synthetic peptide
IYDGGST

SEQ ID NO: 328 - protein, 18 aa, Artificial Sequence
  Description of Artificial Sequence Synthetic peptide
ARDRGTYSGYDWYSGMDV

SEQ ID NO: 329 - protein, 8 aa, Artificial Sequence
  Description of Artificial Sequence Synthetic peptide
GFRFDDYA

SEQ ID NO: 330 - protein, 8 aa, Artificial Sequence
  Description of Artificial Sequence Synthetic peptide
INWIGGTT

SEQ ID NO: 331 - protein, 13 aa, Artificial Sequence
  Description of Artificial Sequence Synthetic peptide
ARGVGYYYYYMDV

SEQ ID NO: 332 - protein, 10 aa, Artificial Sequence
  Description of Artificial Sequence Synthetic peptide
GGSISSGDYY

SEQ ID NO: 333 - protein, 7 aa, Artificial Sequence
  Description of Artificial Sequence Synthetic peptide
IYYSGST

SEQ ID NO: 334 - protein, 14 aa, Artificial Sequence
  Description of Artificial Sequence Synthetic peptide
ARITGEGLPQAFDY

SEQ ID NO: 335 - protein, 8 aa, Artificial Sequence
  Description of Artificial Sequence Synthetic peptide
GYTFSNYG

SEQ ID NO: 336 - protein, 8 aa, Artificial Sequence
  Description of Artificial Sequence Synthetic peptide
ISTHNQNV

SEQ ID NO: 337 - protein, 24 aa, Artificial Sequence
  Description of Artificial Sequence Synthetic peptide
ARMIPVGTTYTPYWTGWTPGGMDI

SEQ ID NO: 338 - protein, 10 aa, Artificial Sequence
  Description of Artificial Sequence Synthetic peptide
GGSISSSSYY

SEQ ID NO: 339 - protein, 7 aa, Artificial Sequence
  Description of Artificial Sequence Synthetic peptide
IYYSGST

SEQ ID NO: 340 - protein, 13 aa, Artificial Sequence
  Description of Artificial Sequence Synthetic peptide
ARLGVGLYEAFDI

SEQ ID NO: 341 - protein, 11 aa, Artificial Sequence
  Description of Artificial Sequence Synthetic peptide
ESLLNSDGKTY

SEQ ID NO: 342 - protein, 3 aa, Artificial Sequence
  Description of Artificial Sequence Synthetic peptide
DLS

SEQ ID NO: 343 - protein, 9 aa, Artificial Sequence
  Description of Artificial Sequence Synthetic peptide
MQGLHLPWT

SEQ ID NO: 344 - protein, 6 aa, Artificial Sequence
  Description of Artificial Sequence Synthetic peptide
QSIRGH

SEQ ID NO: 345 - protein, 3 aa, Artificial Sequence
  Description of Artificial Sequence Synthetic peptide
GAS

SEQ ID NO: 346 - protein, 9 aa, Artificial Sequence
  Description of Artificial Sequence Synthetic peptide
QQSYSNPYS

SEQ ID NO: 347 - protein, 6 aa, Artificial Sequence
  Description of Artificial Sequence Synthetic peptide
QTVGAH

SEQ ID NO: 348 - protein, 3 aa, Artificial Sequence
  Description of Artificial Sequence Synthetic peptide
GAS

SEQ ID NO: 349 - protein, 9 aa, Artificial Sequence
  Description of Artificial Sequence Synthetic peptide
QQYGASPPT

SEQ ID NO: 350 - protein, 7 aa, Artificial Sequence
  Description of Artificial Sequence Synthetic peptide
QSLNSAS

SEQ ID NO: 351 - protein, 3 aa, Artificial Sequence
  Description of Artificial Sequence Synthetic peptide
GAS

SEQ ID NO: 352 - protein, 9 aa, Artificial Sequence
  Description of Artificial Sequence Synthetic peptide
QQYAFSPPT

SEQ ID NO: 353 - protein, 6 aa, Artificial Sequence
  Description of Artificial Sequence Synthetic peptide
QSISNY

SEQ ID NO: 354 - protein, 3 aa, Artificial Sequence
  Description of Artificial Sequence Synthetic peptide
AAS

SEQ ID NO: 355 - protein, 9 aa, Artificial Sequence
  Description of Artificial Sequence Synthetic peptide
QQSASTPHT

SEQ ID NO: 356 - protein, 7 aa, Artificial Sequence
  Description of Artificial Sequence Synthetic peptide
QNVDSSY

SEQ ID NO: 357 - protein, 3 aa, Artificial Sequence
  Description of Artificial Sequence Synthetic peptide
GAS

SEQ ID NO: 358 - protein, 10 aa, Artificial Sequence
  Description of Artificial Sequence Synthetic peptide
QQYGTSPTWT

SEQ ID NO: 359 - protein, 9 aa, Artificial Sequence
  Description of Artificial Sequence Synthetic peptide
SSNIGAFFD

SEQ ID NO: 360 - protein, 3 aa, Artificial Sequence
  Description of Artificial Sequence Synthetic peptide
GDR

SEQ ID NO: 361 - protein, 11 aa, Artificial Sequence
  Description of Artificial Sequence Synthetic peptide
QSYDRGLSAWV

SEQ ID NO: 362 - protein, 6 aa, Artificial Sequence
  Description of Artificial Sequence Synthetic peptide
QTISPY

SEQ ID NO: 363 - protein, 3 aa, Artificial Sequence
  Description of Artificial Sequence Synthetic peptide
GTN

SEQ ID NO: 364 - protein, 9 aa, Artificial Sequence
  Description of Artificial Sequence Synthetic peptide
QQSHTTPLT

SEQ ID NO: 365 - protein, 6 aa, Artificial Sequence
  Description of Artificial Sequence Synthetic peptide
QSLLTY

SEQ ID NO: 366 - protein, 3 aa, Artificial Sequence
  Description of Artificial Sequence Synthetic peptide
SAS

SEQ ID NO: 367 - protein, 9 aa, Artificial Sequence
  Description of Artificial Sequence Synthetic peptide
QQSHTTPLT

SEQ ID NO: 368 - protein, 9 aa, Artificial Sequence
  Description of Artificial Sequence Synthetic peptide
SSNIGAGYD

SEQ ID NO: 369 - protein, 3 aa, Artificial Sequence
  Description of Artificial Sequence Synthetic peptide
GNT

SEQ ID NO: 370 - protein, 11 aa, Artificial Sequence
  Description of Artificial Sequence Synthetic peptide
QSYERNLDGAL

SEQ ID NO: 371 - protein, 9 aa, Artificial Sequence
  Description of Artificial Sequence Synthetic peptide
SSNIGAGYD

SEQ ID NO: 372 - protein, 3 aa, Artificial Sequence
  Description of Artificial Sequence Synthetic peptide
GNY

SEQ ID NO: 373 - protein, 11 aa, Artificial Sequence
  Description of Artificial Sequence Synthetic peptide
QSYDTSLSGWI

SEQ ID NO: 374 - protein, 9 aa, Artificial Sequence
  Description of Artificial Sequence Synthetic peptide
SSNIGAGYD

SEQ ID NO: 375 - protein, 3 aa, Artificial Sequence
  Description of Artificial Sequence Synthetic peptide
ANN

SEQ ID NO: 376 - protein, 11 aa, Artificial Sequence
  Description of Artificial Sequence Synthetic peptide
QSYDRSLSGSV

SEQ ID NO: 377 - protein, 9 aa, Artificial Sequence
  Description of Artificial Sequence Synthetic peptide
SSNLGTGYD

SEQ ID NO: 378 - protein, 3 aa, Artificial Sequence
  Description of Artificial Sequence Synthetic peptide
GNT

SEQ ID NO: 379 - protein, 11 aa, Artificial Sequence
  Description of Artificial Sequence Synthetic peptide
QSYDSSLGGLV

SEQ ID NO: 380 - protein, 9 aa, Artificial Sequence
  Description of Artificial Sequence Synthetic peptide
SSNIGAGYD

SEQ ID NO: 381 - protein, 3 aa, Artificial Sequence
  Description of Artificial Sequence Synthetic peptide
GNN

SEQ ID NO: 382 - protein, 11 aa, Artificial Sequence
  Description of Artificial Sequence Synthetic peptide
QSYDRSLSGWV

SEQ ID NO: 383 - protein, 9 aa, Artificial Sequence
  Description of Artificial Sequence Synthetic peptide
SSNIGAGYD

SEQ ID NO: 384 - protein, 3 aa, Artificial Sequence
  Description of Artificial Sequence Synthetic peptide
GNN

SEQ ID NO: 385 - protein, 11 aa, Artificial Sequence
  Description of Artificial Sequence Synthetic peptide
QSYDSSLSGSI

SEQ ID NO: 386 - protein, 6 aa, Artificial Sequence
  Description of Artificial Sequence Synthetic peptide
QGISSY

SEQ ID NO: 387 - protein, 3 aa, Artificial Sequence
  Description of Artificial Sequence Synthetic peptide
AAS

SEQ ID NO: 388 - protein, 11 aa, Artificial Sequence
  Description of Artificial Sequence Synthetic peptide
QQVTSYPFSIT

SEQ ID NO: 389 - protein, 9 aa, Artificial Sequence
  Description of Artificial Sequence Synthetic peptide
GSNIGAGYD

SEQ ID NO: 390 - protein, 3 aa, Artificial Sequence
  Description of Artificial Sequence Synthetic peptide
DNN

SEQ ID NO: 391 - protein, 11 aa, Artificial Sequence
  Description of Artificial Sequence Synthetic peptide
QSYDRNLEGVV

SEQ ID NO: 392 - protein, 6 aa, Artificial Sequence
  Description of Artificial Sequence Synthetic peptide
QTINSY

SEQ ID NO: 393 - protein, 3 aa, Artificial Sequence
  Description of Artificial Sequence Synthetic peptide
TVS

SEQ ID NO: 394 - protein, 9 aa, Artificial Sequence
  Description of Artificial Sequence Synthetic peptide
QQSYNSPYT

SEQ ID NO: 395 - protein, 6 aa, Artificial Sequence
  Description of Artificial Sequence Synthetic peptide
QTISSY

SEQ ID NO: 396 - protein, 3 aa, Artificial Sequence
  Description of Artificial Sequence Synthetic peptide
GAS

SEQ ID NO: 397 - protein, 9 aa, Artificial Sequence
  Description of Artificial Sequence Synthetic peptide
QQSKSIPYT

SEQ ID NO: 398 - protein, 6 aa, Artificial Sequence
  Description of Artificial Sequence Synthetic peptide
QSVGAY

SEQ ID NO: 399 - protein, 3 aa, Artificial Sequence
  Description of Artificial Sequence Synthetic peptide
AGS

SEQ ID NO: 400 - protein, 9 aa, Artificial Sequence
  Description of Artificial Sequence Synthetic peptide
QQSLGDFLT

SEQ ID NO: 401 - protein, 6 aa, Artificial Sequence
  Description of Artificial Sequence Synthetic peptide
QSVGAY

SEQ ID NO: 402 - protein, 3 aa, Artificial Sequence
  Description of Artificial Sequence Synthetic peptide
AGS

SEQ ID NO: 403 - protein, 9 aa, Artificial Sequence
  Description of Artificial Sequence Synthetic peptide
QQSLGDFLT

SEQ ID NO: 404 - protein, 6 aa, Artificial Sequence
  Description of Artificial Sequence Synthetic peptide
QTISSY

SEQ ID NO: 405 - protein, 3 aa, Artificial Sequence
  Description of Artificial Sequence Synthetic peptide
TVS

SEQ ID NO: 406 - protein, 9 aa, Artificial Sequence
  Description of Artificial Sequence Synthetic peptide
QQSYGAPYT

SEQ ID NO: 407 - protein, 6 aa, Artificial Sequence
  Description of Artificial Sequence Synthetic peptide
KLGDKY

SEQ ID NO: 408 - protein, 3 aa, Artificial Sequence
  Description of Artificial Sequence Synthetic peptide
QDN

SEQ ID NO: 409 - protein, 8 aa, Artificial Sequence
  Description of Artificial Sequence Synthetic peptide
QAWDSGTV

SEQ ID NO: 410 - protein, 8 aa, Artificial Sequence
  Description of Artificial Sequence Synthetic peptide
SSNIRSNA

SEQ ID NO: 411 - protein, 3 aa, Artificial Sequence
  Description of Artificial Sequence Synthetic peptide
YDG

SEQ ID NO: 412 - protein, 11 aa, Artificial Sequence
  Description of Artificial Sequence Synthetic peptide
AAWDDSLNGPV

SEQ ID NO: 413 - protein, 6 aa, Artificial Sequence
  Description of Artificial Sequence Synthetic peptide
QNIASF

SEQ ID NO: 414 - protein, 3 aa, Artificial Sequence
  Description of Artificial Sequence Synthetic peptide
AAS

SEQ ID NO: 415 - protein, 9 aa, Artificial Sequence
  Description of Artificial Sequence Synthetic peptide
QQSYNTPFT

SEQ ID NO: 416 - protein, 7 aa, Artificial Sequence
  Description of Artificial Sequence Synthetic peptide
QSITSNY

SEQ ID NO: 417 - protein, 3 aa, Artificial Sequence
  Description of Artificial Sequence Synthetic peptide
GAS

SEQ ID NO: 418 - protein, 9 aa, Artificial Sequence
  Description of Artificial Sequence Synthetic peptide
QQYGSSLYI

SEQ ID NO: 419 - protein, 8 aa, Artificial Sequence
  Description of Artificial Sequence Synthetic peptide
NSNIGKNY

SEQ ID NO: 420 - protein, 3 aa, Artificial Sequence
  Description of Artificial Sequence Synthetic peptide
ENN

SEQ ID NO: 421 - protein, 11 aa, Artificial Sequence
  Description of Artificial Sequence Synthetic peptide
GAWDSSLNGVL

SEQ ID NO: 422 - protein, 6 aa, Artificial Sequence
  Description of Artificial Sequence Synthetic peptide
QTISTY

SEQ ID NO: 423 - protein, 3 aa, Artificial Sequence
  Description of Artificial Sequence Synthetic peptide
AAS

SEQ ID NO: 424 - protein, 10 aa, Artificial Sequence
  Description of Artificial Sequence Synthetic peptide
QQTYSSLALT

SEQ ID NO: 425 - protein, 6 aa, Artificial Sequence
  Description of Artificial Sequence Synthetic peptide
QTIRSY

SEQ ID NO: 426 - protein, 3 aa, Artificial Sequence
  Description of Artificial Sequence Synthetic peptide
AAT

SEQ ID NO: 427 - protein, 9 aa, Artificial Sequence
  Description of Artificial Sequence Synthetic peptide
QQSFSTLYS

SEQ ID NO: 428 - protein, 12 aa, Artificial Sequence
  Description of Artificial Sequence Synthetic peptide
QSVLYSSNSKSY

SEQ ID NO: 429 - protein, 3 aa, Artificial Sequence
  Description of Artificial Sequence Synthetic peptide
WAS

SEQ ID NO: 430 - protein, 9 aa, Artificial Sequence
  Description of Artificial Sequence Synthetic peptide
QQFYTSPHT

SEQ ID NO: 431 - protein, 9 aa, Artificial Sequence
  Description of Artificial Sequence Synthetic peptide
SSDIGAGSD

SEQ ID NO: 432 - protein, 3 aa, Artificial Sequence
  Description of Artificial Sequence Synthetic peptide
GNT

SEQ ID NO: 433 - protein, 11 aa, Artificial Sequence
  Description of Artificial Sequence Synthetic peptide
QSYDSGLSGYV

SEQ ID NO: 434 - protein, 6 aa, Artificial Sequence
  Description of Artificial Sequence Synthetic peptide
QSVTSN

SEQ ID NO: 435 - protein, 3 aa, Artificial Sequence
  Description of Artificial Sequence Synthetic peptide
GAS

SEQ ID NO: 436 - protein, 10 aa, Artificial Sequence
  Description of Artificial Sequence Synthetic peptide
QQYNAWPPWT

SEQ ID NO: 437 - protein, 7 aa, Artificial Sequence
  Description of Artificial Sequence Synthetic peptide
SGPSNYA

SEQ ID NO: 438 - protein, 7 aa, Artificial Sequence
  Description of Artificial Sequence Synthetic peptide
VNSDGSH

SEQ ID NO: 439 - protein, 9 aa, Artificial Sequence
  Description of Artificial Sequence Synthetic peptide
QTWGSGIYV

SEQ ID NO: 440 - protein, 6 aa, Artificial Sequence
  Description of Artificial Sequence Synthetic peptide
QSVSNW

SEQ ID NO: 441 - protein, 3 aa, Artificial Sequence
  Description of Artificial Sequence Synthetic peptide
KAS

SEQ ID NO: 442 - protein, 9 aa, Artificial Sequence
  Description of Artificial Sequence Synthetic peptide
QHYNGYPYT

SEQ ID NO: 443 - protein, 12 aa, Artificial Sequence
  Description of Artificial Sequence Synthetic peptide
QSLLYTSNNKNS

SEQ ID NO: 444 - protein, 3 aa, Artificial Sequence
  Description of Artificial Sequence Synthetic peptide
WAS

SEQ ID NO: 445 - protein, 9 aa, Artificial Sequence
  Description of Artificial Sequence Synthetic peptide
QQYYTAPYT

SEQ ID NO: 446 - protein, 8 aa, Artificial Sequence
  Description of Artificial Sequence Synthetic peptide
NSNIGSST

SEQ ID NO: 447 - protein, 3 aa, Artificial Sequence
  Description of Artificial Sequence Synthetic peptide
SDN

SEQ ID NO: 448 - protein, 12 aa, Artificial Sequence
  Description of Artificial Sequence Synthetic peptide
GTWDDGLNGPYV

SEQ ID NO: 449 - protein, 8 aa, Artificial Sequence
  Description of Artificial Sequence Synthetic peptide
SSDIGSNS

SEQ ID NO: 450 - protein, 3 aa, Artificial Sequence
  Description of Artificial Sequence Synthetic peptide
SSI

SEQ ID NO: 451 - protein, 11 aa, Artificial Sequence
  Description of Artificial Sequence Synthetic peptide
ATWDDSLNGPV

SEQ ID NO: 452 - protein, 8 aa, Artificial Sequence
  Description of Artificial Sequence Synthetic peptide
SSNIGRNP

SEQ ID NO: 453 - protein, 3 aa, Artificial Sequence
  Description of Artificial Sequence Synthetic peptide
KNS

SEQ ID NO: 454 - protein, 11 aa, Artificial Sequence
  Description of Artificial Sequence Synthetic peptide
AAWDDSLNGPV

SEQ ID NO: 455 - protein, 12 aa, Artificial Sequence
  Description of Artificial Sequence Synthetic peptide
QSVLFSPNNKNY

SEQ ID NO: 456 - protein, 3 aa, Artificial Sequence
  Description of Artificial Sequence Synthetic peptide
WAS

SEQ ID NO: 457 - protein, 9 aa, Artificial Sequence
  Description of Artificial Sequence Synthetic peptide
QQYNSNPYT

SEQ ID NO: 458 - protein, 6 aa, Artificial Sequence
  Description of Artificial Sequence Synthetic peptide
RSVRNY

SEQ ID NO: 459 - protein, 3 aa, Artificial Sequence
  Description of Artificial Sequence Synthetic peptide
DAS

SEQ ID NO: 460 - protein, 9 aa, Artificial Sequence
  Description of Artificial Sequence Synthetic peptide
QQRSNWGLT

SEQ ID NO: 461 - protein, 6 aa, Artificial Sequence
  Description of Artificial Sequence Synthetic peptide
QSVTNN

SEQ ID NO: 462 - protein, 3 aa, Artificial Sequence
  Description of Artificial Sequence Synthetic peptide
GAS

SEQ ID NO: 463 - protein, 10 aa, Artificial Sequence
  Description of Artificial Sequence Synthetic peptide
QQYNGWPPWT

SEQ ID NO: 464 - protein, 8 aa, Artificial Sequence
  Description of Artificial Sequence Synthetic peptide
TSNIGNSY

SEQ ID NO: 465 - protein, 3 aa, Artificial Sequence
  Description of Artificial Sequence Synthetic peptide
EDN

SEQ ID NO: 466 - protein, 11 aa, Artificial Sequence
  Description of Artificial Sequence Synthetic peptide
GTWDNSLTEVV

SEQ ID NO: 467 - protein, 9 aa, Artificial Sequence
  Description of Artificial Sequence Synthetic peptide
TSNIGAGFD

SEQ ID NO: 468 - protein, 3 aa, Artificial Sequence
  Description of Artificial Sequence Synthetic peptide
ANT

SEQ ID NO: 469 - protein, 12 aa, Artificial Sequence
  Description of Artificial Sequence Synthetic peptide
QTYDTTLSGSTV

SEQ ID NO: 470 - protein, 6 aa, Artificial Sequence
  Description of Artificial Sequence Synthetic peptide
QSVRSN

SEQ ID NO: 471 - protein, 3 aa, Artificial Sequence
  Description of Artificial Sequence Synthetic peptide
GAS

SEQ ID NO: 472 - protein, 10 aa, Artificial Sequence
  Description of Artificial Sequence Synthetic peptide
QQYNNWPPGA

SEQ ID NO: 473 - protein, 9 aa, Artificial Sequence
  Description of Artificial Sequence Synthetic peptide
TSNIGAGFD

SEQ ID NO: 474 - protein, 3 aa, Artificial Sequence
  Description of Artificial Sequence Synthetic peptide
ANT

SEQ ID NO: 475 - protein, 12 aa, Artificial Sequence
  Description of Artificial Sequence Synthetic peptide
QTYDRSLSGSTV

SEQ ID NO: 476 - protein, 6 aa, Artificial Sequence
  Description of Artificial Sequence Synthetic peptide
QRIGSY

SEQ ID NO: 477 - protein, 3 aa, Artificial Sequence
  Description of Artificial Sequence Synthetic peptide
DAS

SEQ ID NO: 478 - protein, 8 aa, Artificial Sequence
  Description of Artificial Sequence Synthetic peptide
QQRTDWFT

SEQ ID NO: 479 - protein, 7 aa, Artificial Sequence
  Description of Artificial Sequence Synthetic peptide
RSVNSDY

SEQ ID NO: 480 - protein, 3 aa, Artificial Sequence
  Description of Artificial Sequence Synthetic peptide
AAS

SEQ ID NO: 481 - protein, 9 aa, Artificial Sequence
  Description of Artificial Sequence Synthetic peptide
QQYGSAVWT

SEQ ID NO: 482 - protein, 6 aa, Artificial Sequence
  Description of Artificial Sequence Synthetic peptide
QSISSR

SEQ ID NO: 483 - protein, 3 aa, Artificial Sequence
  Description of Artificial Sequence Synthetic peptide
DAS

SEQ ID NO: 484 - protein, 10 aa, Artificial Sequence
  Description of Artificial Sequence Synthetic peptide
QHYSIYSRWT

SEQ ID NO: 485 - protein, 7 aa, Artificial Sequence
  Description of Artificial Sequence Synthetic peptide
QSISSNS

SEQ ID NO: 486 - protein, 3 aa, Artificial Sequence
  Description of Artificial Sequence Synthetic peptide
GTS

SEQ ID NO: 487 - protein, 9 aa, Artificial Sequence
  Description of Artificial Sequence Synthetic peptide
QQYGSSPLT

SEQ ID NO: 488 - protein, 6 aa, Artificial Sequence
  Description of Artificial Sequence Synthetic peptide
QSISND

SEQ ID NO: 489 - protein, 3 aa, Artificial Sequence
  Description of Artificial Sequence Synthetic peptide
SAS

SEQ ID NO: 490 - protein, 9 aa, Artificial Sequence
  Description of Artificial Sequence Synthetic peptide
QQSYTTPPL

SEQ ID NO: 491 - protein, 6 aa, Artificial Sequence
  Description of Artificial Sequence Synthetic peptide
QTISNF

SEQ ID NO: 492 - protein, 3 aa, Artificial Sequence
  Description of Artificial Sequence Synthetic peptide
AAL

SEQ ID NO: 493 - protein, 9 aa, Artificial Sequence
  Description of Artificial Sequence Synthetic peptide
QQSYITPWT

SEQ ID NO: 494 - protein, 6 aa, Artificial Sequence
  Description of Artificial Sequence Synthetic peptide
QGIRSW

SEQ ID NO: 495 - protein, 3 aa, Artificial Sequence
  Description of Artificial Sequence Synthetic peptide
AAS

SEQ ID NO: 496 - protein, 10 aa, Artificial Sequence
  Description of Artificial Sequence Synthetic peptide
QQSNSFSLFT

SEQ ID NO: 497 - protein, 6 aa, Artificial Sequence
  Description of Artificial Sequence Synthetic peptide
QSLSGN

SEQ ID NO: 498 - protein, 3 aa, Artificial Sequence
  Description of Artificial Sequence Synthetic peptide
GTS

SEQ ID NO: 499 - protein, 11 aa, Artificial Sequence
  Description of Artificial Sequence Synthetic peptide
QQYNNWPPVFT

SEQ ID NO: 500 - protein, 7 aa, Artificial Sequence
  Description of Artificial Sequence Synthetic peptide
QLINGNY

SEQ ID NO: 501 - protein, 3 aa, Artificial Sequence
  Description of Artificial Sequence Synthetic peptide
DVS

SEQ ID NO: 502 - protein, 10 aa, Artificial Sequence
  Description of Artificial Sequence Synthetic peptide
QHYGRSPSPT

SEQ ID NO: 503 - protein, 6 aa, Artificial Sequence
  Description of Artificial Sequence Synthetic peptide
QSISNF

SEQ ID NO: 504 - protein, 3 aa, Artificial Sequence
  Description of Artificial Sequence Synthetic peptide
DVS

SEQ ID NO: 505 - protein, 11 aa, Artificial Sequence
  Description of Artificial Sequence Synthetic peptide
QQRSNWPPGYT

SEQ ID NO: 506 - protein, 9 aa, Artificial Sequence
  Description of Artificial Sequence Synthetic peptide
SSNIGATYD

SEQ ID NO: 507 - protein, 3 aa, Artificial Sequence
  Description of Artificial Sequence Synthetic peptide
GNN

SEQ ID NO: 508 - protein, 12 aa, Artificial Sequence
  Description of Artificial Sequence Synthetic peptide
QSYDSVLSGSVI

SEQ ID NO: 509 - protein, 6 aa, Artificial Sequence
  Description of Artificial Sequence Synthetic peptide
KLGSKY

SEQ ID NO: 510 - protein, 3 aa, Artificial Sequence
  Description of Artificial Sequence Synthetic peptide
QDT

SEQ ID NO: 511 - protein, 9 aa, Artificial Sequence
  Description of Artificial Sequence Synthetic peptide
QAWDSSSFV

SEQ ID NO: 512 - protein, 6 aa, Artificial Sequence
  Description of Artificial Sequence Synthetic peptide
QSISSW

SEQ ID NO: 513 - protein, 3 aa, Artificial Sequence
  Description of Artificial Sequence Synthetic peptide
KAF

SEQ ID NO: 514 - protein, 10 aa, Artificial Sequence
  Description of Artificial Sequence Synthetic peptide
QQYGSFSEFT

SEQ ID NO: 515 - protein, 9 aa, Artificial Sequence
  Description of Artificial Sequence Synthetic peptide
SGDVGGYTF

SEQ ID NO: 516 - protein, 3 aa, Artificial Sequence
  Description of Artificial Sequence Synthetic peptide
EVT

SEQ ID NO: 517 - protein, 11 aa, Artificial Sequence
  Description of Artificial Sequence Synthetic peptide
SSYSSTNNLRV

SEQ ID NO: 518 - protein, 6 aa, Artificial Sequence
  Description of Artificial Sequence Synthetic peptide
NIGSKS

SEQ ID NO: 519 - protein, 3 aa, Artificial Sequence
  Description of Artificial Sequence Synthetic peptide
DDS

SEQ ID NO: 520 - protein, 11 aa, Artificial Sequence
  Description of Artificial Sequence Synthetic peptide
HVSDDNSDHVF

SEQ ID NO: 521 - protein, 8 aa, Artificial Sequence
  Description of Artificial Sequence Synthetic peptide
GSNIGNNY

SEQ ID NO: 522 - protein, 3 aa, Artificial Sequence
  Description of Artificial Sequence Synthetic peptide
NTD

SEQ ID NO: 523 - protein, 11 aa, Artificial Sequence
  Description of Artificial Sequence Synthetic peptide
AAWDDTLNGYV

SEQ ID NO: 524 - protein, 7 aa, Artificial Sequence
  Description of Artificial Sequence Synthetic peptide
QSVSRTY

SEQ ID NO: 525 - protein, 3 aa, Artificial Sequence
  Description of Artificial Sequence Synthetic peptide
GTS

SEQ ID NO: 526 - protein, 8 aa, Artificial Sequence
  Description of Artificial Sequence Synthetic peptide
QHFGSSLT

SEQ ID NO: 527 - protein, 7 aa, Artificial Sequence
  Description of Artificial Sequence Synthetic peptide
HSVSSSY

SEQ ID NO: 528 - protein, 3 aa, Artificial Sequence
  Description of Artificial Sequence Synthetic peptide
GAS

SEQ ID NO: 529 - protein, 11 aa, Artificial Sequence
  Description of Artificial Sequence Synthetic peptide
QQYLTSPPGWT

SEQ ID NO: 530 - protein, 6 aa, Artificial Sequence
  Description of Artificial Sequence Synthetic peptide
QSISNF

SEQ ID NO: 531 - protein, 3 aa, Artificial Sequence
  Description of Artificial Sequence Synthetic peptide
DAS

SEQ ID NO: 532 - protein, 7 aa, Artificial Sequence
  Description of Artificial Sequence Synthetic peptide
QQRSTWT

SEQ ID NO: 533 - protein, 6 aa, Artificial Sequence
  Description of Artificial Sequence Synthetic peptide
ELGHKY

SEQ ID NO: 534 - protein, 3 aa, Artificial Sequence
  Description of Artificial Sequence Synthetic peptide
QDT

SEQ ID NO: 535 - protein, 9 aa, Artificial Sequence
  Description of Artificial Sequence Synthetic peptide
QAWDSGTVL

SEQ ID NO: 536 - protein, 6 aa, Artificial Sequence
  Description of Artificial Sequence Synthetic peptide
DLGNQY

SEQ ID NO: 537 - protein, 3 aa, Artificial Sequence
  Description of Artificial Sequence Synthetic peptide
QDN

SEQ ID NO: 538 - protein, 9 aa, Artificial Sequence
  Description of Artificial Sequence Synthetic peptide
QAWDSRTYV

SEQ ID NO: 539 - protein, 6 aa, Artificial Sequence
  Description of Artificial Sequence Synthetic peptide
QNIYNY

SEQ ID NO: 540 - protein, 3 aa, Artificial Sequence
  Description of Artificial Sequence Synthetic peptide
DAS

SEQ ID NO: 541 - protein, 7 aa, Artificial Sequence
  Description of Artificial Sequence Synthetic peptide
QQRSDWT

SEQ ID NO: 542 - protein, 12 aa, Artificial Sequence
  Description of Artificial Sequence Synthetic peptide
LSLLYSSNNKNY

SEQ ID NO: 543 - protein, 3 aa, Artificial Sequence
  Description of Artificial Sequence Synthetic peptide
WAS

SEQ ID NO: 544 - protein, 9 aa, Artificial Sequence
  Description of Artificial Sequence Synthetic peptide
QQYYSTPIT

SEQ ID NO: 545 - protein, 8 aa, Artificial Sequence
  Description of Artificial Sequence Synthetic peptide
TGNIASNY

SEQ ID NO: 546 - protein, 3 aa, Artificial Sequence
  Description of Artificial Sequence Synthetic peptide
EDN

SEQ ID NO: 547 - protein, 10 aa, Artificial Sequence
  Description of Artificial Sequence Synthetic peptide
QSYDSGHHVV

SEQ ID NO: 548 - protein, 12 aa, Artificial Sequence
  Description of Artificial Sequence Synthetic peptide
ESVLYNFNNKNY

SEQ ID NO: 549 - protein, 3 aa, Artificial Sequence
  Description of Artificial Sequence Synthetic peptide
WAS

SEQ ID NO: 550 - protein, 8 aa, Artificial Sequence
  Description of Artificial Sequence Synthetic peptide
HQFYSTHS

SEQ ID NO: 551 - protein, 12 aa, Artificial Sequence
  Description of Artificial Sequence Synthetic peptide
ESIFYYSNSKSY

SEQ ID NO: 552 - protein, 3 aa, Artificial Sequence
  Description of Artificial Sequence Synthetic peptide
WAS

SEQ ID NO: 553 - protein, 9 aa, Artificial Sequence
  Description of Artificial Sequence Synthetic peptide
QQYYTNPRT

SEQ ID NO: 554 - protein, 6 aa, Artificial Sequence
  Description of Artificial Sequence Synthetic peptide
NIGSKS

SEQ ID NO: 555 - protein, 3 aa, Artificial Sequence
  Description of Artificial Sequence Synthetic peptide
DDS

SEQ ID NO: 556 - protein, 12 aa, Artificial Sequence
  Description of Artificial Sequence Synthetic peptide
HVWDSYSDPNYV

SEQ ID NO: 557 - protein, 8 aa, Artificial Sequence
  Description of Artificial Sequence Synthetic peptide
RSNMGINW

SEQ ID NO: 558 - protein, 3 aa, Artificial Sequence
  Description of Artificial Sequence Synthetic peptide
RNS

SEQ ID NO: 559 - protein, 11 aa, Artificial Sequence
  Description of Artificial Sequence Synthetic peptide
AVWDDSLDGRL

SEQ ID NO: 560 - protein, 8 aa, Artificial Sequence
  Description of Artificial Sequence Synthetic peptide
SSNMGINW

SEQ ID NO: 561 - protein, 3 aa, Artificial Sequence
  Description of Artificial Sequence Synthetic peptide
RNS

SEQ ID NO: 562 - protein, 11 aa, Artificial Sequence
  Description of Artificial Sequence Synthetic peptide
AVWDDVVSGRL

SEQ ID NO: 563 - protein, 6 aa, Artificial Sequence
  Description of Artificial Sequence Synthetic peptide
QGVRND

SEQ ID NO: 564 - protein, 3 aa, Artificial Sequence
  Description of Artificial Sequence Synthetic peptide
AAS

SEQ ID NO: 565 - protein, 9 aa, Artificial Sequence
  Description of Artificial Sequence Synthetic peptide
LQVYSYPFT

SEQ ID NO: 566 - protein, 12 aa, Artificial Sequence
  Description of Artificial Sequence Synthetic peptide
ESVLYTSNSKNY

SEQ ID NO: 567 - protein, 3 aa, Artificial Sequence
  Description of Artificial Sequence Synthetic peptide
WAS

SEQ ID NO: 568 - protein, 9 aa, Artificial Sequence
  Description of Artificial Sequence Synthetic peptide
QQYFTIPRT

SEQ ID NO: 569 - protein, 9 aa, Artificial Sequence
  Description of Artificial Sequence Synthetic peptide
SGSVSTSHF

SEQ ID NO: 570 - protein, 3 aa, Artificial Sequence
  Description of Artificial Sequence Synthetic peptide
STI

SEQ ID NO: 571 - protein, 12 aa, Artificial Sequence
  Description of Artificial Sequence Synthetic peptide
VLYLGGGIPHWV

SEQ ID NO: 572 - protein, 6 aa, Artificial Sequence
  Description of Artificial Sequence Synthetic peptide
QSISTY

SEQ ID NO: 573 - protein, 3 aa, Artificial Sequence
  Description of Artificial Sequence Synthetic peptide
VAS

SEQ ID NO: 574 - protein, 9 aa, Artificial Sequence
  Description of Artificial Sequence Synthetic peptide
QQGYSIPWT

SEQ ID NO: 575 - protein, 6 aa, Artificial Sequence
  Description of Artificial Sequence Synthetic peptide
QTISGY

SEQ ID NO: 576 - protein, 3 aa, Artificial Sequence
  Description of Artificial Sequence Synthetic peptide
TAS

SEQ ID NO: 577 - protein, 9 aa, Artificial Sequence
  Description of Artificial Sequence Synthetic peptide
QQSYRMPLT

SEQ ID NO: 578 - protein, 6 aa, Artificial Sequence
  Description of Artificial Sequence Synthetic peptide
QTITTY

SEQ ID NO: 579 - protein, 3 aa, Artificial Sequence
  Description of Artificial Sequence Synthetic peptide
AAS

SEQ ID NO: 580 - protein, 9 aa, Artificial Sequence
  Description of Artificial Sequence Synthetic peptide
QQSYSTPYT

SEQ ID NO: 581 - protein, 9 aa, Artificial Sequence
  Description of Artificial Sequence Synthetic peptide
NFNIGAGYD

SEQ ID NO: 582 - protein, 3 aa, Artificial Sequence
  Description of Artificial Sequence Synthetic peptide
GNT

SEQ ID NO: 583 - protein, 10 aa, Artificial Sequence
  Description of Artificial Sequence Synthetic peptide
QSYDSSLSMV

SEQ ID NO: 584 - protein, 6 aa, Artificial Sequence
  Description of Artificial Sequence Synthetic peptide
QSISSY

SEQ ID NO: 585 - protein, 3 aa, Artificial Sequence
  Description of Artificial Sequence Synthetic peptide
TVS

SEQ ID NO: 586 - protein, 9 aa, Artificial Sequence
  Description of Artificial Sequence Synthetic peptide
QQSYNIPYT

SEQ ID NO: 587 - protein, 6 aa, Artificial Sequence
  Description of Artificial Sequence Synthetic peptide
QSIGTY

SEQ ID NO: 588 - protein, 3 aa, Artificial Sequence
  Description of Artificial Sequence Synthetic peptide
AAS

SEQ ID NO: 589 - protein, 9 aa, Artificial Sequence
  Description of Artificial Sequence Synthetic peptide
QQSYTKPYT

SEQ ID NO: 590 - protein, 7 aa, Artificial Sequence
  Description of Artificial Sequence Synthetic peptide
QTVSSDY

SEQ ID NO: 591 - protein, 3 aa, Artificial Sequence
  Description of Artificial Sequence Synthetic peptide
DAS

SEQ ID NO: 592 - protein, 8 aa, Artificial Sequence
  Description of Artificial Sequence Synthetic peptide
QQYGSPYT

SEQ ID NO: 593 - protein, 6 aa, Artificial Sequence
  Description of Artificial Sequence Synthetic peptide
QSISSY

SEQ ID NO: 594 - protein, 3 aa, Artificial Sequence
  Description of Artificial Sequence Synthetic peptide
AAS

SEQ ID NO: 595 - protein, 9 aa, Artificial Sequence
  Description of Artificial Sequence Synthetic peptide
QQSYSTPRT

SEQ ID NO: 596 - protein, 6 aa, Artificial Sequence
  Description of Artificial Sequence Synthetic peptide
QTITIW

SEQ ID NO: 597 - protein, 3 aa, Artificial Sequence
  Description of Artificial Sequence Synthetic peptide
DAV

SEQ ID NO: 598 - protein, 9 aa, Artificial Sequence
  Description of Artificial Sequence Synthetic peptide
QQYNNYPYT

SEQ ID NO: 599 - protein, 6 aa, Artificial Sequence
  Description of Artificial Sequence Synthetic peptide
NIGSKS

SEQ ID NO: 600 - protein, 3 aa, Artificial Sequence
  Description of Artificial Sequence Synthetic peptide
DDS

SEQ ID NO: 601 - protein, 11 aa, Artificial Sequence
  Description of Artificial Sequence Synthetic peptide
QVWDSSGDRVV

SEQ ID NO: 602 - protein, 8 aa, Artificial Sequence
  Description of Artificial Sequence Synthetic peptide
SSNIGNNY

SEQ ID NO: 603 - protein, 3 aa, Artificial Sequence
  Description of Artificial Sequence Synthetic peptide
DNN

SEQ ID NO: 604 - protein, 13 aa, Artificial Sequence
  Description of Artificial Sequence Synthetic peptide
GTWDSSLSVGFYV

SEQ ID NO: 605 - protein, 6 aa, Artificial Sequence
  Description of Artificial Sequence Synthetic peptide
QSVSSY

SEQ ID NO: 606 - protein, 3 aa, Artificial Sequence
  Description of Artificial Sequence Synthetic peptide
DAS

SEQ ID NO: 607 - protein, 11 aa, Artificial Sequence
  Description of Artificial Sequence Synthetic peptide
QQRSNWPPKWT

SEQ ID NO: 608 - protein, 6 aa, Artificial Sequence
  Description of Artificial Sequence Synthetic peptide
NIGSKS

SEQ ID NO: 609 - protein, 3 aa, Artificial Sequence
  Description of Artificial Sequence Synthetic peptide
DDS

SEQ ID NO: 610 - protein, 10 aa, Artificial Sequence
  Description of Artificial Sequence Synthetic peptide
QVWHSSSDWV

SEQ ID NO: 611 - protein, 6 aa, Artificial Sequence
  Description of Artificial Sequence Synthetic peptide
QSINRN

SEQ ID NO: 612 - protein, 3 aa, Artificial Sequence
  Description of Artificial Sequence Synthetic peptide
DAS

SEQ ID NO: 613 - protein, 8 aa, Artificial Sequence
  Description of Artificial Sequence Synthetic peptide
QQRSHWVT

SEQ ID NO: 614 - protein, 6 aa, Artificial Sequence
  Description of Artificial Sequence Synthetic peptide
QTINNW

SEQ ID NO: 615 - protein, 3 aa, Artificial Sequence
  Description of Artificial Sequence Synthetic peptide
KAS

SEQ ID NO: 616 - protein, 9 aa, Artificial Sequence
  Description of Artificial Sequence Synthetic peptide
QQYISYSRT

SEQ ID NO: 617 - protein, 7 aa, Artificial Sequence
  Description of Artificial Sequence Synthetic peptide
QSVSSSY

SEQ ID NO: 618 - protein, 3 aa, Artificial Sequence
  Description of Artificial Sequence Synthetic peptide
STF

SEQ ID NO: 619 - protein, 9 aa, Artificial Sequence
  Description of Artificial Sequence Synthetic peptide
QQYGSSPRT

SEQ ID NO: 620 - protein, 6 aa, Artificial Sequence
  Description of Artificial Sequence Synthetic peptide
QSVSSY

SEQ ID NO: 621 - protein, 3 aa, Artificial Sequence
  Description of Artificial Sequence Synthetic peptide
DAS

SEQ ID NO: 622 - protein, 10 aa, Artificial Sequence
  Description of Artificial Sequence Synthetic peptide
QQRSNWPPLT

SEQ ID NO: 623 - protein, 6 aa, Artificial Sequence
  Description of Artificial Sequence Synthetic peptide
QDISSW

SEQ ID NO: 624 - protein, 3 aa, Artificial Sequence
  Description of Artificial Sequence Synthetic peptide
AAS

SEQ ID NO: 625 - protein, 9 aa, Artificial Sequence
  Description of Artificial Sequence Synthetic peptide
QQANSFPLT

SEQ ID NO: 626 - protein, 6 aa, Artificial Sequence
  Description of Artificial Sequence Synthetic peptide
QGISSA

SEQ ID NO: 627 - protein, 3 aa, Artificial Sequence
  Description of Artificial Sequence Synthetic peptide
DAS

SEQ ID NO: 628 - protein, 9 aa, Artificial Sequence
  Description of Artificial Sequence Synthetic peptide
QQFNSYPLT

SEQ ID NO: 629 - protein, 6 aa, Artificial Sequence
  Description of Artificial Sequence Synthetic peptide
QSIGTH

SEQ ID NO: 630 - protein, 3 aa, Artificial Sequence
  Description of Artificial Sequence Synthetic peptide
AAS

SEQ ID NO: 631 - protein, 10 aa, Artificial Sequence
  Description of Artificial Sequence Synthetic peptide
QQSYSTPSIT

SEQ ID NO: 632 - protein, 7 aa, Artificial Sequence
  Description of Artificial Sequence Synthetic peptide
QSVSSSY

SEQ ID NO: 633 - protein, 3 aa, Artificial Sequence
  Description of Artificial Sequence Synthetic peptide
GAS

SEQ ID NO: 634 - protein, 8 aa, Artificial Sequence
  Description of Artificial Sequence Synthetic peptide
QQYGSSPT

SEQ ID NO: 635 - protein, 6 aa, Artificial Sequence
  Description of Artificial Sequence Synthetic peptide
NIGVRN

SEQ ID NO: 636 - protein, 3 aa, Artificial Sequence
  Description of Artificial Sequence Synthetic peptide
RDN

SEQ ID NO: 637 - protein, 11 aa, Artificial Sequence
  Description of Artificial Sequence Synthetic peptide
QVWDSNTAHVI

SEQ ID NO: 638 - protein, 6 aa, Artificial Sequence
  Description of Artificial Sequence Synthetic peptide
QSVSSN

SEQ ID NO: 639 - protein, 3 aa, Artificial Sequence
  Description of Artificial Sequence Synthetic peptide
GAS

SEQ ID NO: 640 - protein, 10 aa, Artificial Sequence
  Description of Artificial Sequence Synthetic peptide
QQYNNWPPYT

SEQ ID NO: 641 - protein, 8 aa, Artificial Sequence
  Description of Artificial Sequence Synthetic peptide
SSNIGRNY

SEQ ID NO: 642 - protein, 3 aa, Artificial Sequence
  Description of Artificial Sequence Synthetic peptide
RNN

SEQ ID NO: 643 - protein, 11 aa, Artificial Sequence
  Description of Artificial Sequence Synthetic peptide
AAWDDSLSGYV

SEQ ID NO: 644 - protein, 6 aa, Artificial Sequence
  Description of Artificial Sequence Synthetic peptide
QSLSRY

SEQ ID NO: 645 - protein, 3 aa, Artificial Sequence
  Description of Artificial Sequence Synthetic peptide
DAS

SEQ ID NO: 646 - protein, 8 aa, Artificial Sequence
  Description of Artificial Sequence Synthetic peptide
HQRHNWIT

SEQ ID NO: 647 - protein, 6 aa, Artificial Sequence
  Description of Artificial Sequence Synthetic peptide
LRLSTN

SEQ ID NO: 648 - protein, 3 aa, Artificial Sequence
  Description of Artificial Sequence Synthetic peptide
DAS

SEQ ID NO: 649 - protein, 8 aa, Artificial Sequence
  Description of Artificial Sequence Synthetic peptide
HQHSNWYT

SEQ ID NO: 650 - protein, 6 aa, Artificial Sequence
  Description of Artificial Sequence Synthetic peptide
QNINNF

SEQ ID NO: 651 - protein, 3 aa, Artificial Sequence
  Description of Artificial Sequence Synthetic peptide
DAS

SEQ ID NO: 652 - protein, 9 aa, Artificial Sequence
  Description of Artificial Sequence Synthetic peptide
QQRANWPLT

SEQ ID NO: 653 - protein, 12 aa, Artificial Sequence
  Description of Artificial Sequence Synthetic peptide
QSVLYTSNNRNF

SEQ ID NO: 654 - protein, 3 aa, Artificial Sequence
  Description of Artificial Sequence Synthetic peptide
WAS

SEQ ID NO: 655 - protein, 9 aa, Artificial Sequence
  Description of Artificial Sequence Synthetic peptide
QQYYSIPPM

SEQ ID NO: 656 - protein, 6 aa, Artificial Sequence
  Description of Artificial Sequence Synthetic peptide
QSVTSN

SEQ ID NO: 657 - protein, 3 aa, Artificial Sequence
  Description of Artificial Sequence Synthetic peptide
GAS

SEQ ID NO: 658 - protein, 9 aa, Artificial Sequence
  Description of Artificial Sequence Synthetic peptide
QQYDHWPRT

SEQ ID NO: 659 - protein, 6 aa, Artificial Sequence
  Description of Artificial Sequence Synthetic peptide
QSMSNS

SEQ ID NO: 660 - protein, 3 aa, Artificial Sequence
  Description of Artificial Sequence Synthetic peptide
AAS

SEQ ID NO: 661 - protein, 9 aa, Artificial Sequence
  Description of Artificial Sequence Synthetic peptide
QQSYSTPIT

SEQ ID NO: 662 - protein, 9 aa, Artificial Sequence
  Description of Artificial Sequence Synthetic peptide
SSNIGSFDL

SEQ ID NO: 663 - protein, 3 aa, Artificial Sequence
  Description of Artificial Sequence Synthetic peptide
EGD

SEQ ID NO: 664 - protein, 10 aa, Artificial Sequence
  Description of Artificial Sequence Synthetic peptide
CSYARSRTLV

SEQ ID NO: 665 - protein, 6 aa, Artificial Sequence
  Description of Artificial Sequence Synthetic peptide
QGISSY

SEQ ID NO: 666 - protein, 3 aa, Artificial Sequence
  Description of Artificial Sequence Synthetic peptide
AAS

SEQ ID NO: 667 - protein, 9 aa, Artificial Sequence
  Description of Artificial Sequence Synthetic peptide
QQLKSYPGT

SEQ ID NO: 668 - protein, 7 aa, Artificial Sequence
  Description of Artificial Sequence Synthetic peptide
QSVSSNY

SEQ ID NO: 669 - protein, 3 aa, Artificial Sequence
  Description of Artificial Sequence Synthetic peptide
DAS

SEQ ID NO: 670 - protein, 9 aa, Artificial Sequence
  Description of Artificial Sequence Synthetic peptide
QQYGSSPET

SEQ ID NO: 671 - protein, 6 aa, Artificial Sequence
  Description of Artificial Sequence Synthetic peptide
KLGDKY

SEQ ID NO: 672 - protein, 3 aa, Artificial Sequence
  Description of Artificial Sequence Synthetic peptide
QDS

SEQ ID NO: 673 - protein, 9 aa, Artificial Sequence
  Description of Artificial Sequence Synthetic peptide
QAWDSSTAV

SEQ ID NO: 674 - protein, 8 aa, Artificial Sequence
  Description of Artificial Sequence Synthetic peptide
TSNIGRNF

SEQ ID NO: 675 - protein, 3 aa, Artificial Sequence
  Description of Artificial Sequence Synthetic peptide
GND

SEQ ID NO: 676 - protein, 11 aa, Artificial Sequence
  Description of Artificial Sequence Synthetic peptide
ATWDDSVNGPV

SEQ ID NO: 677 - protein, 6 aa, Artificial Sequence
  Description of Artificial Sequence Synthetic peptide
QSISSY

SEQ ID NO: 678 - protein, 3 aa, Artificial Sequence
  Description of Artificial Sequence Synthetic peptide
AAS

SEQ ID NO: 679 - protein, 9 aa, Artificial Sequence
  Description of Artificial Sequence Synthetic peptide
QQSYSTPNT

SEQ ID NO: 680 - protein, 117 aa, Artificial Sequence
  Description of Artificial Sequence Synthetic polypeptide
QVQLVQSGAEVKKPGSSVKVSCWASGGTFDNAINWLRQAPGQGPEWMGRIIPLVDIVNRAPKFQDRVSFTADKSTNTVYMVLNSLTSDDTAVYYCASAFQDGWFDPWGQGTLVTVSS

SEQ ID NO: 681 - protein, 106 aa, Artificial Sequence
  Description of Artificial Sequence Synthetic polypeptide
EIVLTQSPATLSLSPGERATLSCRASQSLSRYLAWYQQKPGQAPRLLIYDASNRATGVPARFSGSGSGTDFTLTISSLEPEDFAVYYCHQRHNWITFGQGTRLEIK

SEQ ID NO: 682 - protein, 124 aa, Artificial Sequence
  Description of Artificial Sequence Synthetic polypeptide
EVQLVESGGGLVRPGGSLRLSCAASGFNVRNYYMSWVRQAPGKGLEWISVIYGGGTTFYAGSVKGRFTISRDDSKSTLFLQMNSLRADDTAVYYCARDPGTVMSTENLQGMDVWGQGTTVTVSS

SEQ ID NO: 683 - protein, 107 aa, Artificial Sequence
  Description of Artificial Sequence Synthetic polypeptide
DIQMTQSPSSLSASVGDRVTITCRASQTIRSYLNWYQQKPGKAPNLLIYAATSLQSGVPSRFSGSGSGTDFTLTISSLQPEDSATYYCQQSFSTLYSFGQGTRLEIK

SEQ ID NO: 684 - protein, 125 aa, Artificial Sequence
  Description of Artificial Sequence Synthetic polypeptide
QVQLVQSGAEVKKPGASVKVSCKASGVTLTDYYFHWIRQAPGQALEWMGRINPARGGTHYAEKFEDRVTMTTDTSTNTVYMDLSSLRSDDTAVYYCARSGSTHNIYSGFYAFDHWGQGTLVTVSS

SEQ ID NO: 685 - protein, 112 aa, Artificial Sequence
  Description of Artificial Sequence Synthetic polypeptide
DIVMTQSPDSLAVSLGERATINCKSSESVLYNFNNKNYLAWYQQKPGQPPKLLIYWASTRESGVPERFSGSGSGTDFTLTISSLQAEDVAVYYCHQFYSTHSFGGGTKVEIK

SEQ ID NO: 686 - protein, 122 aa, Artificial Sequence
  Description of Artificial Sequence Synthetic polypeptide
QLQLQESGPGLVKPSETLSLTCTVSGDSISSSSFYWGWIRQPPGKGLEWIATIYYTGSTEYNPSLKSRISISVDTSMNQFSLRLRSVTAADTAVYYCARQEGSSWSNWFDPWGQGTLVTVSS

SEQ ID NO: 687 - protein, 107 aa, Artificial Sequence
  Description of Artificial Sequence Synthetic polypeptide
DIQMTQSPSSLSASVGDRVTITCRASQSISTYLNWYQQKPGKAPKLLIYVASSLQSGVPSRFSGSGSGTDFTLTISSLQPEDFATYYCQQGYSIPWTFGHGTKVEIK

SEQ ID NO: 688 - protein, 118 aa, Artificial Sequence
  Description of Artificial Sequence Synthetic polypeptide
QVQLVQSGTEVKKPGSSVKVSCKDPGDIMSSHSIHWVRQAPGQGPEWMGRIIPLFGMKSYAPNFQGRVSISVDTSTNTAYLELSGLKSDDTAVYFCAREENLSWLDYWGQGTQVIVSS

SEQ ID NO: 689 - protein, 106 aa, Artificial Sequence
  Description of Artificial Sequence Synthetic polypeptide
EIVLTQSPATLSLSPGDRATLSCRASLRLSTNIAWYQQKPGQAPRLVIYDASKRATGIPARFSGSGSGTDFTLTISSLEPEDFAVYYCHQHSNWYTFGQGTKLEIK

SEQ ID NO: 690 - protein, 120 aa, Artificial Sequence
  Description of Artificial Sequence Synthetic polypeptide
QVQLVQSGAEVKRPGSSVKVSCKASGDIFSSHAISWVRQTPGQGLKWMGRIIPLVGIRNYAQDLQGRVTISADKSINTVYMELTNLRFDDTAVYYCARDRTDWSGGLDIWGQGTRVTVSS

SEQ ID NO: 691 - protein, 106 aa, Artificial Sequence
  Description of Artificial Sequence Synthetic polypeptide
EIVLTQSPATLSLSPGERATLSCRASQRIGSYLAWYQQKPGQAPRLLIYDASNRATGIPARFSGSGSGTDFTLTISRLEPEDSAIYYCQQRTDWFTFGGGTKVEIK

SEQ ID NO: 692 - protein, 124 aa, Artificial Sequence
  Description of Artificial Sequence Synthetic polypeptide
QLQLQESGPGLVKPSETLSLTCSVSGGSISSSSNSWTWIRQPPGKGLEWIATIFYFGSTYYNPSLRSRVAISVDTSENQFSLKLTSVTAADTAVYFCARHILSSSGVSGWLDPWGQGTLVTVSS

SEQ ID NO: 693 - protein, 106 aa, Artificial Sequence
  Description of Artificial Sequence Synthetic polypeptide
SYELTQPPSVSVSPGQTASITCSGEKLGSKYAHWYQQKPGRSPVLVIYQDTKRPSGIPERFSGSNSGSTATLTISGTQAMDEADYYCQAWDSSSFVFGTGTTVTVL

SEQ ID NO: 694 - protein, 117 aa, Artificial Sequence
  Description of Artificial Sequence Synthetic polypeptide
QITLKESGPTLVKPTQTLTLTCTVSGFSLSANSVGVAWFRQPPGKAPEWLGVIYWDDDKRFSPSLKTRLSITKDASTSQVVLRLTNVSPLDTATYYCVRKHYDLLQWGQGAPVTVSS

SEQ ID NO: 695 - protein, 108 aa, Artificial Sequence
  Description of Artificial Sequence Synthetic polypeptide
EIVLTQSPGTLSLSPGEGATLSCRASRSVNSDYIAWYQHKPGLAPRLLIYAASTRATDIPDRFRADVSGTDFTLTIDRLEPDDFAVYYCQQYGSAVWTFGQGTRVEVK

SEQ ID NO: 696 - protein, 118 aa, Artificial Sequence
  Description of Artificial Sequence Synthetic polypeptide
QVQLVQSGAEVKKPGASVKISCKASGYSFSNYYIQWVRQAPGQGLEWLGIINPSGGSTDYAQKFEGRVSMTRDTSTNTVYMEMSSLRSEDTASYYCVRSLANGSGSPWGQGTLVIVSS

SEQ ID NO: 697 - protein, 108 aa, Artificial Sequence
  Description of Artificial Sequence Synthetic polypeptide
DIQMTQSPSTLSASVGDRVTITCRASQSISSRLAWYQQKPGKAPNLLLYDASRSESGVPSRFSGSGSGTEFTLTISSLQPDDVATYYCQHYSIYSRWTFGQGTKVEIK

SEQ ID NO: 698 - protein, 122 aa, Artificial Sequence
  Description of Artificial Sequence Synthetic polypeptide
QVQLVQSGAGVRKPGSSVKVSCKASGGTFSNYIISWVRQAPGQGLEYIGRIIPIVGMEKYEEKFQGRVKITADESTSTVSMELTSLKSEDTATYYCATEGLESGYPPYLEHWGRGTLVIVSS

SEQ ID NO: 699 - protein, 107 aa, Artificial Sequence
  Description of Artificial Sequence Synthetic polypeptide
EIVLTQSPGTLSLSPGERATLSCRTSQTVGAHLAWYQQKPGQSPRLLIYGASSRATGIPDRFSGSGSGTVFTLTINRLEPEDLAVYYCQQYGASPPTFGQGTKVEIK

SEQ ID NO: 700 - protein, 118 aa, Artificial Sequence
  Description of Artificial Sequence Synthetic polypeptide
QVQLQESGPGLVKPSETLSLTCTVSGGSITSHYWSWVRQPPGKGLEWIAYIYYSGSTDYNPSLKSRVTISLDTSKNQVSLRLNSVTTADTAVYFCARCMPLYHVMHVWGHGTTVTVSS

SEQ ID NO: 701 - protein, 111 aa, Artificial Sequence
  Description of Artificial Sequence Synthetic polypeptide
QSVLTQPPSVSGAPGQRVTISCTGSSSDIGAGSDVHWYQQLPGTAPKLLIYGNTNRPSGVPDRFSGSKSGTSASLAITGLQAEDEADYYCQSYDSGLSGYVFGGGTKLTVL

SEQ ID NO: 702 - protein, 125 aa, Artificial Sequence
  Description of Artificial Sequence Synthetic polypeptide
QVQLVESGGGVVQPGKSLRLSCAASGFTFSSHAMDWVRQVPGKGLEWVAVISYDGSNRYYADSVKGRFTISRDTSKNTLYLQMNSLRPEDTAVYYCARDFQPSPMVYGVGYFDYWGQGTLVTVSP

SEQ ID NO: 703 - protein, 108 aa, Artificial Sequence
  Description of Artificial Sequence Synthetic polypeptide
EIVMTQSPATLSVSPGERATLSCRASQSVTSNLAWYQQKPGQAPRLLIYGASTRATGIPARFSGSGSGTEFTLTISSLQSEDFAVYYCQQYNAWPPWTFGQGTKVEIR

SEQ ID NO: 704 - protein, 121 aa, Artificial Sequence
  Description of Artificial Sequence Synthetic polypeptide
QVQLVQSGPEVKKPGASVKVSCKASGYSITTYGIDWVRQAPGQGLEWIGWSSAHTGNTNYAEKFQGRVTMTTDTSTYTTYMELRSLKSDDTAVYYCAMGAYTSSRNWFDPWGQGTLVIVSS

SEQ ID NO: 705 - protein, 107 aa, Artificial Sequence
  Description of Artificial Sequence Synthetic polypeptide
EIVLTQSPATLSLSPGERATLSCRASQNINNFLAWYQQKAGQAPRLLIYDASNRATGIPGRFSGSGSGTDFTLTISSLEPEDFAVYYCQQRANWPLTFGGGTKVEVK

SEQ ID NO: 706 - protein, 120 aa, Artificial Sequence
  Description of Artificial Sequence Synthetic polypeptide
QVQLQQWGAGLLRPSETLSLTCTVYGGSFGGYHWSWIRQSPGKGLEWIGEIDHGGNTNFNPSLKSRVTISVDRSKKQFSLKLTSVTAADTAVYYCARGFGVVINNWFDPWGQGTLVTVSS

SEQ ID NO: 707 - protein, 107 aa, Artificial Sequence
  Description of Artificial Sequence Synthetic polypeptide
DIQMTQSPSSLSASVGDRVTITCRASQTINSYLNWYQQKPGKAPTLLIYTVSSLQSGVPSRFSGSGSGTVFSLTISSLQPEDFATYYCQQSYNSPYTFGQGTKLEIK

SEQ ID NO: 708 - protein, 122 aa, Artificial Sequence
  Description of Artificial Sequence Synthetic polypeptide
QVQLQESGPRLVKPSGTLSLTCAVSGASITSSHWWTWVRQPPGKGLEWIGRIFHSGSTNYNPSLKSRVTISLDKSRNQFSLTLTSVTAADTAVYYCARDDQIVVMPAGFDRWGQGALVIVSS

SEQ ID NO: 709 - protein, 107 aa, Artificial Sequence
  Description of Artificial Sequence Synthetic polypeptide
DIQMTQSPSSLSASVGDRVTITCRASQTISSYLNWYQQTPGKAPNLLIYGASRLQGGVPSRFSGSGSGTDFTLTISSLQPEDFAIYFCQQSKSIPYTFGQGTRLEIK

SEQ ID NO: 710 - protein, 127 aa, Artificial Sequence
  Description of Artificial Sequence Synthetic polypeptide
EVQLVESGGGLVRPGGSLRLSCAASGFTFNNYWMHWVRQAPGKGLVWVSHVKSDGSITNYADSVKGRFTISRDNAKNTLYLEMSSLRVEDTAVYYCARESRPYDPLTGSHPDAFDIWGQGTMVTVSS

SEQ ID NO: 711 - protein, 113 aa, Artificial Sequence
  Description of Artificial Sequence Synthetic polypeptide
DIVMTQSPDSLAVSLGERATINCKSSESIFYYSNSKSYLAWYRQKPGQPPKLLIYWASTRESGVPDRFSGSGSGTDFTLTISSLQPEDVAVYYCQQYYTNPRTFGQGTRLEIK

SEQ ID NO: 712 - protein, 124 aa, Artificial Sequence
  Description of Artificial Sequence Synthetic polypeptide
QVQLVQSGPEVKGPGASVRVSCKANGYTFTSYGINWVRQTPGQGLQWMGWISVHTDDTKDVQQFQDRVTMTTDTSTSTVFMELRNLRFDDTAVYYCARDRTDISGRVRDAFDLWGQGTLVTVSS

SEQ ID NO: 713 - protein, 109 aa, Artificial Sequence
  Description of Artificial Sequence Synthetic polypeptide
SYVLTQPPSVSVAPGQTAKITCGGNNIGSKSVHWYRQKPGQAPVLVVYDDSDRPSGIPERFSGSNSGNTATLTISRVEAGDEADYYCHVWDSYSDPNYVFGSGTKVTVL

SEQ ID NO: 714 - protein, 117 aa, Artificial Sequence
  Description of Artificial Sequence Synthetic polypeptide
QVQLVESGGGVVQPGRSLRLSCAASGFTFSNYPMHWVRQAPGKGLAWVALISYDGRTKYYADSVKGRFTISRDNSMNTVYLQMNSLRAEDTAVYYCAREGLEYLNYWGQGTLVTVSS

SEQ ID NO: 715 - protein, 108 aa, Artificial Sequence
  Description of Artificial Sequence Synthetic polypeptide
EIVLTQSPGTLSLSPGERATLSCRASQSISSNSLAWYQQKPGQAPRLLSYGTSNRATGIPDRFSGSGSGTDFTLTISRLEPEDFAVYYCQQYGSSPLTFGPGTKVDIK

SEQ ID NO: 716 - protein, 126 aa, Artificial Sequence
  Description of Artificial Sequence Synthetic polypeptide
QVQLVQSGTEVKKPGASVKVSCKASGYTFTSHDINWLRQATGQGLEWLGWMNPNSATTGYAQKFQGRVTLTRDISISTAYMELSSLRSDDTAVYYCARNRHYYYADGPTRDAFDIWGQGTVVTVSS

SEQ ID NO: 717 - protein, 106 aa, Artificial Sequence
  Description of Artificial Sequence Synthetic polypeptide
SYELTQTPTVSVSPGQTASITCSGDELGHKYTSWYQQKPGQSPVLVVYQDTKRPSGIPERFSGSNSGNTATLTISGTQVMDEADYYCQAWDSGTVLLGGGTKLTVL

SEQ ID NO: 718 - protein, 119 aa, Artificial Sequence
  Description of Artificial Sequence Synthetic polypeptide
QVQLVESGGGVVQPGRSLRLSCEASGFTFSSSAMHWVRQAPGKGLEWVAIISYHGTNKDYSDSVRGRFTISRDNSKNTLYLQMNGLRGDDTAVYYCAKEAAVSGNFDSWGQGTMVTVSS

SEQ ID NO: 719 - protein, 111 aa, Artificial Sequence
  Description of Artificial Sequence Synthetic polypeptide
QLVLTQSPSASASLGASVKLTCTLISGPSNYAIAWHRQQPQKGPRYLMKVNSDGSHTKGDGIPDRFSGSSSGTERYLIISNLQSEDEADYYCQTWGSGIYVFGPGTKVTVL

SEQ ID NO: 720 - protein, 118 aa, Artificial Sequence
  Description of Artificial Sequence Synthetic polypeptide
EVQLVESGGDLAKPGGSLRLSCEASGFTFSNAWMSWVRQAPGKGLEWVGQIKPHTDGATTDYAAPVKGRFTISRDDSKSTLYLRMNILRIEDTAMYYCTTGPRLSLMWGQGTRVTVSS

SEQ ID NO: 721 - protein, 113 aa, Artificial Sequence
  Description of Artificial Sequence Synthetic polypeptide
DIVMTQSPDSLAVSLGERATINCKSSQSVLYTSNNRNFLAWYQQKPGQPPKLLIHWASTRESGVPDRFSGSGSGTDFTLTINSLQAEDVAVYYCQQYYSIPPMFGQGTKVEIK

SEQ ID NO: 722 - protein, 124 aa, Artificial Sequence
  Description of Artificial Sequence Synthetic polypeptide
EVQLLESGGGLVQPGGSLRLSCAGSGFTFSNYAMSWVRQAPGKGLEWVSDINDRGETTHYADSVKGRFTISRDNSKKKLFLQMNDLRAEDTAIYYCTRAYVVLSVYAPKGFDYWGQGTLVSVSS

SEQ ID NO: 723 - protein, 107 aa, Artificial Sequence
  Description of Artificial Sequence Synthetic polypeptide
DIQMTQSPSILSASEGDRVTITCRASQSVSNWLAWYQQKSGRAPKLLIYKASSLETGVPSRFSGSGSGTEFTLTINNLQPDDFATYYCQHYNGYPYTFGQGTKLEIK

SEQ ID NO: 724 - protein, 122 aa, Artificial Sequence
  Description of Artificial Sequence Synthetic polypeptide
QVQLVQSGAEVKKPGSSVKVSCRPSGGTFSNDILSWVRQAPGQRLEWMGRIIPLVNMTNIAQKFQGRLTLVADKSTAIVHLELGSLRAEDTAIYYCVRDLPSAGFPAPFDIWGQGTRVTVSA

SEQ ID NO: 725 - protein, 108 aa, Artificial Sequence
  Description of Artificial Sequence Synthetic polypeptide
EIVLTQSPGSLSLSPGARATVSCRASQSLNSASLVWYQQRVGQAPRLLMFGASNRATGIPDRFSGSGSGTDFTLTISRLEPEDFAVYYCQQYAFSPPTFGQGTKVEIK

SEQ ID NO: 726 - protein, 123 aa, Artificial Sequence
  Description of Artificial Sequence Synthetic polypeptide
QVQLQESGPGLVAPSQTLSLICDVSGDSVTSGAYSWSWIRQHPVKGLEWIAYINYSGRTNYNPSLQSRLVISVDRSKNQFSLRLSSVTAADTAVYYCARANVVGYSGFYFDYWGQGTLVAVSS

SEQ ID NO: 727 - protein, 107 aa, Artificial Sequence
  Description of Artificial Sequence Synthetic polypeptide
DIQMTQSPSSLSASVGDTVTITCRASQSVGAYLNWYQQKPGKAPKLLVFAGSSLQTGVPSRFSGRGSRTEFTLTITNLQREDFASYYCQQSLGDFLTFGGGTKVEIK

SEQ ID NO: 728 - protein, 124 aa, Artificial Sequence
  Description of Artificial Sequence Synthetic polypeptide
QVQLVQSGAEVKKPGASVIVSCRASGFTFSLYTMHWVRQAPGQGLEWMGWISADNSNTKYSQKYQDRVTITMDTSATTLFMEISSLRSEDTAVYYCTRGPAYYFDSSGFAFDIWGQGTLVTVSS

SEQ ID NO: 729 - protein, 107 aa, Artificial Sequence
  Description of Artificial Sequence Synthetic polypeptide
EIVLTQSPGTLSLSPGERATLSCRASQSVSRTYLGWYQQRPGLAPRLLIYGTSSRATGIPDRFSGSGSGTDFTLTISRLEPEDFATYYCQHFGSSLTFGGGTKVEIK

SEQ ID NO: 730 - protein, 121 aa, Artificial Sequence
  Description of Artificial Sequence Synthetic polypeptide
QVQLVESGGGVVQPGRSLRLSCAASGFTFNNYAMHWVRQAPGKGLEWVALISDDGSKKYFADALKGRFTIFRDNSKNTLYLQMNSLRAEDTAVYYCARDPTPLGEYYFDYWGQGTLVTVSS

SEQ ID NO: 731 - protein, 107 aa, Artificial Sequence
  Description of Artificial Sequence Synthetic polypeptide
DIQMTQSPSSLSASVGDRVTITCRASQTISGYLNWYQQKPGKAPNLLIYTASSLESGVPSRFSGSGSETDFTLTISSLQPEDFATYYCQQSYRMPLTFGGGTRVSLK

SEQ ID NO: 732 - protein, 126 aa, Artificial Sequence
  Description of Artificial Sequence Synthetic polypeptide
QVQLVQSGAEVRKPGASVKVSCKASGYTFTSHDINWVRQAPGQGLEWMGWMNPNSDTTGYAQKFQGRVTMARDISMSTAYMELSSLTSEDTAVYYCARGRHNYYTNGPTRDGFDIWGQGTMVTVSS

SEQ ID NO: 733 - protein, 106 aa, Artificial Sequence
  Description of Artificial Sequence Synthetic polypeptide
SYELTQPPSVSVSPGQTATITCSGDDLGNQYANWFQQRPGQSPVLVIYQDNKRPSGIPERFSGSSSGNTATLTISGTQPLDEADYYCQAWDSRTYVFGTGTKVTVL

SEQ ID NO: 734 - protein, 120 aa, Artificial Sequence
  Description of Artificial Sequence Synthetic polypeptide
QVQLQESGPGVVKPSETLSLTCTVSRGAITDYHLNWIRQPPGKGLEWIGYISSSGSTNYNPSLKSRVTISQVMSGTQFSLKLTSMSAADTALYYCASSSGRYWGGALDLWGQGTQVIVSS

SEQ ID NO: 735 - protein, 113 aa, Artificial Sequence
  Description of Artificial Sequence Synthetic polypeptide
DIVMTQSPDSLAVPLGERATINCKSSQSLLYTSNNKNSLAWYQQKPGQPPKLLLFWASTREFRVPDRFSGSGSGTDFTLTISSLQAEDVAVYYCQQYYTAPYTFGPGTKLEIK

SEQ ID NO: 736 - protein, 118 aa, Artificial Sequence
  Description of Artificial Sequence Synthetic polypeptide
QVQLVQSGAEVKKPGSSVKVSCKASGGTLTRYTISWVRQAPGQGLEWMGRIIPFLDIANYAPRFQGRVTMTADKSTSTAYMELSSLTSQDTAIYYCARDHSGNYFDLWGQGTLVTVSS

SEQ ID NO: 737 - protein, 107 aa, Artificial Sequence
  Description of Artificial Sequence Synthetic polypeptide
EIVMTQSPATLSVSPGERATLSCRASQSVTSNLAWYQRKPGQAPRLLIYGASTGATDIPGRFSGSGSGTEFTLTISGLQSEDFAVYYCQQYDHWPRTFGQGTKVEIK

SEQ ID NO: 738 - protein, 123 aa, Artificial Sequence
  Description of Artificial Sequence Synthetic polypeptide
QVQLQESGPGLVAPSQTLSLICDVSGDSVTSGAHSWSWIRQHPVKGLEWIAYINYSGRTNYNPSLQSRLVISVDRSKNQFSLRLSSVTAADTAVYYCARANVVGYSGYYFDYWGQGTLVAVSS

SEQ ID NO: 739 - protein, 107 aa, Artificial Sequence
  Description of Artificial Sequence Synthetic polypeptide
DIQMTQSPSSLSASVGDRVTITCRASQSVGAYLNWYQQKPGKAPKLLIFAGSSLQTGVPSRFSGRGSRTEFTLTISNLQREDFATYYCQQSLGDFLTFGGGTKVEIK

SEQ ID NO: 740 - protein, 124 aa, Artificial Sequence
  Description of Artificial Sequence Synthetic polypeptide
QVQLVESGGAVVQPGRSLRLSCAASGFTFTSFSMHWVRQAPGRGLEWVAVISHDGIIKYYADSVKGRFTISRDNSKNTVFLQMNSLRPEDTAVFYCARDFPGSRGFFRYYFDHWGQGTLVTVSS

SEQ ID NO: 741 - protein, 107 aa, Artificial Sequence
  Description of Artificial Sequence Synthetic polypeptide
DIQMTQSPSSLSASVGGSVTITCRVDQSISNDLHWYQQKPGKAPKLLISSASSLQSGVPSRFSGSGSGTDFTLTITSLQPEDFATYYCQQSYTTPPLFGQGTTLEIK

SEQ ID NO: 742 - protein, 120 aa, Artificial Sequence
  Description of Artificial Sequence Synthetic polypeptide
QVQLVESGGGVVQPGRSLRLSCAASGFVFRFSGMHWIRQAPGKGLQWVAHISDDGSQKYYADSVTGRFAISRDNSRNTLFLEMTGLRGDDTAVYYCARGDYGVSYFLAHWGLGELVIVSS

SEQ ID NO: 743 - protein, 105 aa, Artificial Sequence
  Description of Artificial Sequence Synthetic polypeptide
EIVLTQSPATLSLSPGERATLSCRASQNIYNYLAWYQQKPGQAPRLVIYDASNRATGIPARFSGSGSETDFTLTISSLEPEDFAVYYCQQRSDWTFGQGTKLEIK

SEQ ID NO: 744 - protein, 117 aa, Artificial Sequence
  Description of Artificial Sequence Synthetic polypeptide
EVQLLESGGGLVQPGGSLRLSCAASGYTFSSYAMSWVRQAPGKGLEWVSGISGSGANTYYADSVKGRFTISRDNSQNTLYLQMNSLRAEDTAVYYCARGLYTTGDYWGQGTLVTVSS

SEQ ID NO: 745 - protein, 107 aa, Artificial Sequence
  Description of Artificial Sequence Synthetic polypeptide
DIQMTQSPSSLSASVGDRVTITCRASQSMSNSLNWYQQKPGKAPKLLIYAASSLQSGVPSRFSGSRSGSVFTLTISNLQPEDFATYYCQQSYSTPITFGQGTRLEIK

SEQ ID NO: 746 - protein, 121 aa, Artificial Sequence
  Description of Artificial Sequence Synthetic polypeptide
QVQLVQSGAEVKKPGASVRISCKPSGRAFLTYDINWVRQAPGQGLEWMAGMNPGSGNAAYAPKFQGRLTMIRNTSISTDYLELSSLTSEDTAVYYCARGEVSMRWRWLDPWGQGSLVTVTS

SEQ ID NO: 747 - protein, 107 aa, Artificial Sequence
  Description of Artificial Sequence Synthetic polypeptide
DIQMTQSPSSLSASVGDRVTITCRASQTISNFLNWYQQKPGRAPKVLIYAALNLQSGVPSRFRGSGSGTDFTLTINNLQPEDFAIYYCQQSYITPWTFGQGTKVEIK

SEQ ID NO: 748 - protein, 120 aa, Artificial Sequence
  Description of Artificial Sequence Synthetic polypeptide
QVQLQQWGAGLLKPSETLSLTCAVSGGSFSGYHWTWIRQPPGKGLEWIGEVDHGGNTNYNPSLMSRVTISVDMSTKQFSLRLTSMTAADTAVYYCARGFGVVINNWFDPWGQGTLVTVTS

SEQ ID NO: 749 - protein, 107 aa, Artificial Sequence
  Description of Artificial Sequence Synthetic polypeptide
DIQMTQSPSSLSASVGDRVTITCRASQTISSYLNWYQQKPGKAPNLLIYTVSTLQSGVPSRFSGSGSGTDFTLTISSLQPEDFATYYCQQSYGAPYTFGQGTKLEIK

SEQ ID NO: 750 - protein, 120 aa, Artificial Sequence
  Description of Artificial Sequence Synthetic polypeptide
QLQLQESGPGLVKPSETLSLTCTVSGGSISSSTYYWGWIRQPPGKGLEWIGTIYYSGSTDYNPSLKSRLTISVDTSKNQFSLRLRSVTAADTAVYYCARLVSPYDQFDYWGQGTLVTVSS

SEQ ID NO: 751 - protein, 107 aa, Artificial Sequence
  Description of Artificial Sequence Synthetic polypeptide
DIQMTQSPSSLSASVGDRVTITCRASQTITTYLSWYLQKPGKAPKLLIYAASSWQSGVPSRFSGSGSGTDFTLTISSLQPEDFATYYCQQSYSTPYTFGQGTKLEIK

SEQ ID NO: 752 - protein, 119 aa, Artificial Sequence
  Description of Artificial Sequence Synthetic polypeptide
QVQLQESGPGLVKPSEILSLTCTVSRFSINSGYYWGWIRQPPGKGLEWIGCISHAGSIYSNPSLESRVTLSLDTSKNQFSLKLTSVIAADTATYYCAASSNWHNALEYWGQGTLVVVSS

SEQ ID NO: 753 - protein, 110 aa, Artificial Sequence
  Description of Artificial Sequence Synthetic polypeptide
QSVLTQPPSASGTPGQTVTISCSGSRSNMGINWVYWYQQLPGTAPKLVIYRNSQRPSGVPDRFSGSQSGTSASLAISGLRSEDEAVYYCAVWDDSLDGRLFGGGTKLTVL

SEQ ID NO: 754 - protein, 119 aa, Artificial Sequence
  Description of Artificial Sequence Synthetic polypeptide
QVQLQESGPGLVKPSEILSLTCTVSRYSINSGYYWGWIRQPPGKGLEWVGCVSHAGVTYYNPSLESRVAVSLDTSKNQFSLKMTSVSAADTAMYYCAASSNWHNALEYWGQGTLVAVSS

SEQ ID NO: 755 - protein, 110 aa, Artificial Sequence
  Description of Artificial Sequence Synthetic polypeptide
QSVLTQPPSASGTPGQSVTISCSGSSSNMGINWVYWYQQLPGTAPKLLIYRNSQRPSGVPDRFSGSQSGTSASLAISGLRSEDEGHYYCAVWDDVVSGRLFGGGTKLTVL

SEQ ID NO: 756 - protein, 116 aa, Artificial Sequence
  Description of Artificial Sequence Synthetic polypeptide
EVQLVESGGDLVQPGGSLRLSCEASAFTFSDYAMTWVRQAPGKGLEWVSSINTSGGRTYYADSVKGRFTISRDNSKNTLYLQMKSLRAEDTAVYFCARFFYVMDVWGQGTTVTVSS

SEQ ID NO: 757 - protein, 107 aa, Artificial Sequence
  Description of Artificial Sequence Synthetic polypeptide
AIQMTQSPSSLSASVGDRVTITCRASQGVRNDLGWYQQKPGKAPKLLINAASGLQSGVPSRFSGSGSDTDFTLTISSLQPEDFATYYCLQVYSYPFTFGPGTKVDIK

SEQ ID NO: 758 - protein, 130 aa, Artificial Sequence
  Description of Artificial Sequence Synthetic polypeptide
EVQLVESGGGLVQTGGSLRLACAASGFTFSSYAMSWVRQAPGKGLEWVSTITESGDDAYYADSVKGRLTISRDNSKNTLFLHLNGLRSEDTARYYCAKDMGLYKSSWYGNFFYYYYMDVWGEGTTVTVSS

SEQ ID NO: 759 - protein, 110 aa, Artificial Sequence
  Description of Artificial Sequence Synthetic polypeptide
QSALTQPASVSGSPGQSITISCTGSSSNIGSFDLISWYQQHPGKAPKFIIYEGDKRPSGVSYRFSGSKSGSTASLTISGLQAEDEAYYYCCSYARSRTLVFGGGTKVTVL

SEQ ID NO: 760 - protein, 121 aa, Artificial Sequence
  Description of Artificial Sequence Synthetic polypeptide
EVQLVESGGDLVLPGGSLRLSCEASGFMFSSYAMNWVRQAPGKGLERVSGISGSGGNTYYADSVKGRFTISRDNSKNIVYLQMNSLRAEDTAVYYCAKDLGNDYFYYIDVWGKGTTVMVSS

SEQ ID NO: 761 - protein, 110 aa, Artificial Sequence
  Description of Artificial Sequence Synthetic polypeptide
QSVLTQPPSVSGAPGQRVTISCTGTNFNIGAGYDVQWYQQLPGAAPKLLIHGNTYRPSGVPDRFSGSKSGTSASLAITGLQAEDEADYYCQSYDSSLSMVFGGGTKLTVL

SEQ ID NO: 762 - protein, 126 aa, Artificial Sequence
  Description of Artificial Sequence Synthetic polypeptide
QVQLVQSGAIMKKPGSSVKVSCKASGGTFTNFAFSWVRQAPGQGLEWMGGIIPFVGPANYAPQFQGRVTITADESTTTAYMELNSLRSEDTAVYYCARVRGFGETAYFYHYYMDVWGKGTTVTVSS

SEQ ID NO: 763 - protein, 112 aa, Artificial Sequence
  Description of Artificial Sequence Synthetic polypeptide
QTVVTQEPSLSVSPGGTVTLTCGLSSGSVSTSHFPSWYQQIPGQPPRTLIYSTITRSSGVPDRFSGSILGNKATLTITGAQADDECDYYCVLYLGGGIPHWVFGGGTKLTVL

SEQ ID NO: 764 - protein, 118 aa, Artificial Sequence
  Description of Artificial Sequence Synthetic polypeptide
QVQLVQSGAEVKKPGASVKVSCKASEDTFTNYYMHWVRQAPGQGLEWMGIINPSGGSTDYAQKFQGRVTMTRDTSTSTVYMELSSLRSEDTAMYYCARQGRTAACDYWGQGTLVTVSS

SEQ ID NO: 765 - protein, 108 aa, Artificial Sequence
  Description of Artificial Sequence Synthetic polypeptide
DIQMTQSPSSVSASVGDRVTITCRASQGIRSWLAWYQQKPGKAPKLLIYAASTLQSGVPSRFSGSGSGTDFTLTISSLQPEDFATYYCQQSNSFSLFTFGPGTRVDIK

SEQ ID NO: 766 - protein, 120 aa, Artificial Sequence
  Description of Artificial Sequence Synthetic polypeptide
EVQLVESGGGLVKPGGSLRLSCSVSAFSISGYNMHWVRQAPGKGLEWVSSITGGSNYINYADSVKGRFTISRDNAQNSLYLQMKSLRAEDTAVYYCARETQPSPGSFDSWGQGTLVTVSS

SEQ ID NO: 767 - protein, 111 aa, Artificial Sequence
  Description of Artificial Sequence Synthetic polypeptide
QSVLTQPPSVSGAPGQRVTISCTGSSSNIGAGYDVNWYQQLPGTAPKLLIYGNYNRPSGVPDRFSASKSGTSASLAITGLQAEDEADYWCQSYDTSLSGWIFGGGTKLTVL

SEQ ID NO: 768 - protein, 122 aa, Artificial Sequence
  Description of Artificial Sequence Synthetic polypeptide
QVQLVQSGAEVKKPGASVKVSCKASAYTFTSYGITWVRQAPGQGLEWMGWISAYRGNTKYAQKLQGRVTMTTDTSTSTASMELRSLRSDDTAVYYCARTSYYDSSYYPIDYWGQGTLVTVSS

SEQ ID NO: 769 - protein, 108 aa, Artificial Sequence
  Description of Artificial Sequence Synthetic polypeptide
SYVLTQPPSVSVAPGQTARITCGGNNIGSKSVQWYQQKPGQAPVLVVYDDSDRPSGIPERFSGSNSGNTATLTISRVEAGDEADYYCQVWDSSGDRVVFGGGTKLTVL

SEQ ID NO: 770 - protein, 127 aa, Artificial Sequence
  Description of Artificial Sequence Synthetic polypeptide
QVQLVESGGGVVQPGRSLRLSCAASGLTFSTYVMHWVRQAPGKGLEWVAVISYDGTNEHYADSVKGRFTISRDNSKNTLYLQMNSLRPEDTAVYYCARGTADYDVWSGSAWGGFDYWGQGTLVTVSS

SEQ ID NO: 771 - protein, 107 aa, Artificial Sequence
  Description of Artificial Sequence Synthetic polypeptide
DIQMTQSPSSLSASVGDRVTITCRASQSISSYLNWYQQKPGKAPKLLMYTVSSLPSGVPSRFSGSGSGTDFTLTISSLQPEDFATYYCQQSYNIPYTFGQGTKLEIK

SEQ ID NO: 772 - protein, 119 aa, Artificial Sequence
  Description of Artificial Sequence Synthetic polypeptide
QITLKESGPTLVKPTQTLTLTCTFSGFSLNTGGVSVGWIRQPPGKALEWLALIYWDGDKRYSPSLKSRLTITKDTSKNQVVFTMTNMDPVDTATYYCAHRENSAYIDFWGQGTLVTVSS

SEQ ID NO: 773 - protein, 112 aa, Artificial Sequence
  Description of Artificial Sequence Synthetic polypeptide
QSVLTQPPSVSAAPGQKVTISCSGSSSNIGNNYVSWYQQLPGAAPKLLIYDNNKRPSGIPDRFSGSKSGTSASLGITGLQTGDEADYYCGTWDSSLSVGFYVFGTGTKVTVL

SEQ ID NO: 774 - protein, 119 aa, Artificial Sequence
  Description of Artificial Sequence Synthetic polypeptide
EVQLVESGGGLVKPGGSLRLSCAASGFTLTSYSMNWVRQAPGKGLEWVSSISSSSSYIHYADSVKGRFTISRDNAKNSVYLQMNSLRAEDTAAYYCARVARELDPFDMWGQGTVVTVAS

SEQ ID NO: 775 - protein, 111 aa, Artificial Sequence
  Description of Artificial Sequence Synthetic polypeptide
QSVLTQPPSVSGAPGQRVTISCTGSSSNIGAGYDVHWYQQLPGTAPKLLVYANNNRPSGVPDRFSGSKSGTSASLAITGLQADDEADYYCQSYDRSLSGSVFGGGTKLTVL

SEQ ID NO: 776 - protein, 117 aa, Artificial Sequence
  Description of Artificial Sequence Synthetic polypeptide
QVQLVQSGAEVQKPGASVKVSCKASGYTFTRHAIHWVRQAPGQRLEWIGWVHGGNENTKYSQKLQGRVTITRDTSASTAYMELNSLRYEDTAVYYCATAMPGAFNMWGQGTVVTVSS

SEQ ID NO: 777 - protein, 108 aa, Artificial Sequence
  Description of Artificial Sequence Synthetic polypeptide
DIQMTQSPSTLSASVGDRVTITCRASQSISSWVAWYQQKPGKAPKLLIYKAFSLETGVPSRFSGSGSGTEFTLTISSLQPDDCATYYCQQYGSFSEFTFGQGTKLEIK

SEQ ID NO: 778 - protein, 120 aa, Artificial Sequence
  Description of Artificial Sequence Synthetic polypeptide
EVQLVESGGGLVKPGGSLRLSCAASGFSITSYGMHWVRQAPGKGLEWVSSISSSSSYISYRDSVKGRFTVSRDNAGNSLSLQMNSLRGEDTAVYHCARDAGLVPPWMDSWGQGTLVIVSS

SEQ ID NO: 779 - protein, 111 aa, Artificial Sequence
  Description of Artificial Sequence Synthetic polypeptide
QSVLTQPPSVSGAAGQRVTISCTGSSSNLGTGYDVHWYRQQPGTAPKLIMFGNTNRPSGVPGRFSGSRSGTSASLAITGLQADDEADYYCQSYDSSLGGLVFGGGTKLTVL

SEQ ID NO: 780 - protein, 119 aa, Artificial Sequence
  Description of Artificial Sequence Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDVQQQLALDYWGQGTLVTVSS

SEQ ID NO: 781 - protein, 109 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPKWTFGQGTKVEIK

SEQ ID NO: 782 - protein, 120 aa, Artificial Sequence
  Description of Artificial Sequence Synthetic polypeptide
EVQLVESGGGLVQPGGSLRLSCAASGFTFSDYSMNWVRQAPGQGLEWISYISSTGSTIYYADSVRGRFTISRDNAKNSLYLQMNTLRAEDTAVYYCARGKTTVTIWFDPWGQGTLVTVSS

SEQ ID NO: 783 - protein, 107 aa, Artificial Sequence
  Description of Artificial Sequence Synthetic polypeptide
SYVLTQPPSVSVAPGQTARITCGGNNIGSKSVHWYQQKPGQAPVLVVYDDSDRPSGIPERFSGSNSGNTATLTISRVEAGDEADYYCQVWHSSSDWVFGGGTRLTVL

SEQ ID NO: 784 - protein, 121 aa, Artificial Sequence
  Description of Artificial Sequence Synthetic polypeptide
QVQLQESGPQLVKPSQTLSLTCTVSGDSISSSDYCWSWIRQSPGKGLEWIGHSVYSGDTQYNPSLKSRLSISVDTSKNQFSLRLRSVTAADTAVYYCARGNDFWSGSFDYWGQGALVIVSS

SEQ ID NO: 785 - protein, 107 aa, Artificial Sequence
  Description of Artificial Sequence Synthetic polypeptide
DIQMTQSPSSLSASVGDRVTITCRASQSIGTYLNWYQQKPGKAPKVLIYAASSLQSGVPSRFSGRGSGTDFTLTVSSLQPEDFASYYCQQSYTKPYTFGQGTKLEIK

SEQ ID NO: 786 - protein, 121 aa, Artificial Sequence
  Description of Artificial Sequence Synthetic polypeptide
EVQLVESGGGLVKPGGSLRISCVASEFTLSGYAMNWVRQVPGKGLEWVSSISSSSSYINYADFVKGRFTISRDNAKNSVFLQLNSLRPEDTALYYCVRERPTAILPYLDLWGRGTLVTVSS

SEQ ID NO: 787 - protein, 111 aa, Artificial Sequence
  Description of Artificial Sequence Synthetic polypeptide
QSVLTQPPSVSGAPGQRVTISCTGSSSNIGAGYDVQWYQQSPGTAPKLLIHGNNNRPSGVPDRFSGSKSGTSASLAITGLQAEDEADYYCQSYDRSLSGWVFGGGTKLTVL

SEQ ID NO: 788 - protein, 127 aa, Artificial Sequence
  Description of Artificial Sequence Synthetic polypeptide
QVQLQESGPGLVKPSETVSLTCTVSGGSISSNNWWSWVRQSPGKGLEWIGEIYHSGSTNFNPSLKSRVTISVDKSKNHFSLKLGSLTAADTAVYYCARRPNDYGDYSYYYYYYMDVWGKGTTVTVSS

SEQ ID NO: 789 - protein, 106 aa, Artificial Sequence
  Description of Artificial Sequence Synthetic polypeptide
EIVLTQSPATLSLSPGESATLSCRASQSINRNLAWYQQKPGQAPRLLIYDASNRATGIPARFSGSGSGTDFTLTISSLEPEDFAVYYCQQRSHWVTFGQGTRLEIK

SEQ ID NO: 790 - protein, 124 aa, Artificial Sequence
  Description of Artificial Sequence Synthetic polypeptide
QVQLVQSGPEVRKPGASVKVSCRTSGYNFPAYGISWLRQAPGQGFEWMGWISAYNVNTNYAQNLQDRVAMTTDTSTSTAFLELRSLTSDDTGMYYCARVPRPPLSGTYSTADFWGQGTLVTVSS

SEQ ID NO: 791 - protein, 107 aa, Artificial Sequence
  Description of Artificial Sequence Synthetic polypeptide
DIQMTQSPSTLSASVGDRVTITCRASQTINNWLAWYQQKPGKAPKLLIYKASSLESGVPSRFSGSGSGTEFTLTISSLQPEDFATYYCQQYISYSRTFGQGTKVEVK

SEQ ID NO: 792 - protein, 124 aa, Artificial Sequence
  Description of Artificial Sequence Synthetic polypeptide
QVQLQQWGAGLLKPSETLSLTCAVYGGSFSGYYWSWIRQSPGKGLEWIGEINHSGSTNYNLSFKSRVTISIDTSKNQFSLKVTSVTAADTAVYFCARGPIRYSSYYYYYGLDVWGQGTTVTVSS

SEQ ID NO: 793 - protein, 108 aa, Artificial Sequence
  Description of Artificial Sequence Synthetic polypeptide
EIVLTQSPDTLSLSPGERATLSCRASQSVSSSYLAWYQQKPGQAPRLIIYSTFIRVTGFPDRFSGSGSGTDFTLTISRLEPEDFAVYYCQQYGSSPRTFGQGTKVEIK

SEQ ID NO: 794 - protein, 122 aa, Artificial Sequence
  Description of Artificial Sequence Synthetic polypeptide
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDSGSYYYYYYMDVWGKGTTVTVSS

SEQ ID NO: 795 - protein, 108 aa, Artificial Sequence [sequence omitted]

SEQ ID NO: 796 - protein, 118 aa, Artificial Sequence
  Description of Artificial Sequence Synthetic polypeptide
QLQLQESGPGLVKPSETLSLTCTVSGGSISSSSYFWGWIRQPPGKGLEWIGSVFYSGSTYYNPSLKSRVTISVDTPKNQFSLRLSSVTAADTAVYYCARHSWDRWVYWGQGTLVTVSS

SEQ ID NO: 797 - protein, 107 aa, Artificial Sequence
  Description of Artificial Sequence Synthetic polypeptide
DIQMTQSPSSVSASVGDRVTITCRASQDISSWLAWYQQKPGKAPKLLIYAASSLQSGVPSRFSGSGSGTDFTLTISSLQPEDFATYYCQQANSFPLTFGGGTKVEIK

SEQ ID NO: 798 - protein, 118 aa, Artificial Sequence
  Description of Artificial Sequence Synthetic polypeptide
EVQLVQSGAEVKKPGESLKISCKGSGYNFANYWIGWVRQMPGKGLEWMGIIYPGDSDTRRNPSFQGHVTMSVDQSISTAYLQWSSLKASDTAMYYCARPLAVSAFDIWGQGTMVTVSS

SEQ ID NO: 799 - protein, 107 aa, Artificial Sequence
  Description of Artificial Sequence Synthetic polypeptide
DIQMTQSPSSLSASVGDKLTITCRASQSISNYLNWYQQKSGKAPKLLIYAASSLQSGVPSRFSGSGSGTDFTLTISSLQPEDFATYYCQQSASTPHTFGQGTKVEIV

SEQ ID NO: 800 - protein, 122 aa, Artificial Sequence
  Description of Artificial Sequence Synthetic polypeptide
EVQLVESGGGLVQPGGSLRLSCVASGFGFRSYWMHWVRQAPGKGLVWVSRINNDGSSASYADSVKGRFTISRDNAKNTLYLQMNSLRAEDTALYYCAREFGVLDYHYGMDVWGQGTTVTVSS

SEQ ID NO: 801 - protein, 110 aa, Artificial Sequence
  Description of Artificial Sequence Synthetic polypeptide
QSVLTQPPSASGTPGQRVTISCSGSSSDIGSNSVIWYQQLPGTAPKLLIYSSIQRPSGVPDRFSGSKSGTSASLAISGLQSEDEADYYCATWDDSLNGPVFGGGTKLTVL

SEQ ID NO: 802 - protein, 124 aa, Artificial Sequence
  Description of Artificial Sequence Synthetic polypeptide
QVQLVQSGAEVKKPGSSVKVSCKAYGGTLSNYAISWVRQAPGQGLHWMGRIIPVMNVANYADNFQGRVTITADKSTSTAYMELSSLRSEDAAIYYCATARGLGSLEGGDAFDVWGQGTMLTVSS

SEQ ID NO: 803 - protein, 113 aa, Artificial Sequence
  Description of Artificial Sequence Synthetic polypeptide
DIVMTQSPDSVAVSLGERATISCKSSLSLLYSSNNKNYLVWYQQKPGQPPKLLFYWASTRESGVPDRFSASGSGTDFTLTISSLQAEDVAVYYCQQYYSTPITFGQGTRLEIR

SEQ ID NO: 804 - protein, 121 aa, Artificial Sequence
  Description of Artificial Sequence Synthetic polypeptide
QLQLQESGPGLVKPWETLSLTCIVSGGSISRSTHYWGWIRQSPEMGLEWIGSIYYTGNTYYNPSFKSRVTISVDTSRNQLSLRVNSVSAADTARYYCARHETYDYGVFNIWGQGAMVTVSA

SEQ ID NO: 805 - protein, 107 aa, Artificial Sequence
  Description of Artificial Sequence Synthetic polypeptide
EIVLTQSPGTLSLSPGERAILSCRASQTVSSDYLAWYQQRPGQAPRLLIYDASSRATGVPDRFRGSGSGTDFTLTISRLEPEDFGVYYCQQYGSPYTFGQGTKLEIK

SEQ ID NO: 806 - protein, 121 aa, Artificial Sequence
  Description of Artificial Sequence Synthetic polypeptide
QVQLQESGPGLVKPSETLSLTCTVSGGSLSSYCWSWIRQPPGKGLEWIGYIYYSGGTNYNPSLKSRVTISVDTSKNQFSLNLSSVTAADTAVYYCARGEASGYLNYYFDYWGQGTLVAVSS

SEQ ID NO: 807 - protein, 105 aa, Artificial Sequence
  Description of Artificial Sequence Synthetic polypeptide
SYELTQPPSVSVSPGQTASITCSGHKLGDKYAYWYQQKPGQSPVLVIYQDNKRPSGIPERFSGSNSGNTATLTISGTQAMDEADYYCQAWDSGTVFGGGTKLTVL

SEQ ID NO: 808 - protein, 122 aa, Artificial Sequence
  Description of Artificial Sequence Synthetic polypeptide
EVQLVESGGGLVQPGGSLRLSCAASGFSFGSYYMHWVRQAPGKGLVWVSRTSSDGSTTSYADSVKGRFSISRDNAKNTLYLQMDSLRAEDTAVYYCARELGILDYYYGMDVWGQGTTVTVSS

SEQ ID NO: 809 - protein, 110 aa, Artificial Sequence
  Description of Artificial Sequence Synthetic polypeptide
QSVLTQPPSASGTPGQRVTISCSGSSSNIGRNPVSWYQQLPGTAPKHLIYKNSQRPSGVPDRFSGSKSGTSASLAISGLQSEDEADYYCAAWDDSLNGPVFGGGTKLTVL

SEQ ID NO: 810 - protein, 123 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDAPFGRDDGYGMDVWGQGTTVTVSS

SEQ ID NO: 811 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGPGTKVDIK

SEQ ID NO: 812 - protein, 131 aa, Artificial Sequence
  Description of Artificial Sequence Synthetic polypeptide
EVQLVESGGDLVEPGGSLRLSCAASGFTFESAWMSWVRQAPGKGLEWVGRIKSKLDHGTTDYAAPVKGRFTLSRDDSKNTLFLQMNNLKTEDTGVYYCATDRSDVRFLEWLGNYQFGMDVWGQGTTVAVSS

SEQ ID NO: 813 - protein, 110 aa, Artificial Sequence
  Description of Artificial Sequence Synthetic polypeptide
EIVLTQSPGTLSLSPGERATLSCRASHSVSSSYLAWYQQKPGQAPRLLIFGASSRATGIPDRFSGSGSGTDFTLTISRLEPEDFAVYYCQQYLTSPPGWTFGQGTKVEIK

SEQ ID NO: 814 - protein, 123 aa, Artificial Sequence
  Description of Artificial Sequence Synthetic polypeptide
QVQLVQSGAEVKQPGASVKVSCKASGYTFSGYYIQWVRQAPGQGLEWMGWINPNSGGTNFAQKFQGRVTMTSDTSTSAAYMELSSLRSDDTAVYYCVRGQQLTRQYYFGMDVWGQGTTVTVSS

SEQ ID NO: 815 - protein, 110 aa, Artificial Sequence
  Description of Artificial Sequence Synthetic polypeptide
QSVLTQPPSVSGAPGQRVIISCSGSSSNIRSNAVNWYQQLPGKAPKLLIFYDGLLPSGVSDRFSGSKTGNSASLAISGLQSEDEADYYCAAWDDSLNGPVFGGGTKLTVL

SEQ ID NO: 816 - protein, 119 aa, Artificial Sequence
  Description of Artificial Sequence Synthetic polypeptide
EVQLVESGGGLVQPGGSLRLSCAASGFPFNFYWMHWVRQAPREGLVWVSRINNDGSSPNYADSVWGRFTISRDNAKNTLYLQMHSLRAEDTAVYYCARAARFGDYVDQWGQGTLVTVSS

SEQ ID NO: 817 - protein, 107 aa, Artificial Sequence
  Description of Artificial Sequence Synthetic polypeptide
AIQLTQSPSSLSASMGDRVTITCRASQGISSAFAWYQQKPGKAPKLLIYDASTLESGVPSRFSGSGSGTDFTLTISSLQPEDFATYYCQQFNSYPLTFGGGTKVEIK

SEQ ID NO: 818 - protein, 124 aa, Artificial Sequence
  Description of Artificial Sequence Synthetic polypeptide
QVQLQESGPGLVKPSETLSLTCSVSGVSISSHYWSWIRQPAGKGLEWIGRLYTSGTTTYNPSLNSRVTMSVDTTNNRLSLRLTSVTAADTAVYYCARDVRLTVFGALHNWFDPWGQGILVTVSS

SEQ ID NO: 819 - protein, 108 aa, Artificial Sequence
  Description of Artificial Sequence Synthetic polypeptide
DIQMTQSPSSLSASVGDRVTITCRASQSIGTHLNWYQQQRGRAPKLLVYAASGLQSGVPSRFSGSGSGTYFTLTISSLQPEDFATYYCQQSYSTPSITFGQGSRLDIK

SEQ ID NO: 820 - protein, 122 aa, Artificial Sequence
  Description of Artificial Sequence Synthetic polypeptide
QVQLVESGGGVVQSGKSLGLSCIASGFIFSNYALHWVRQAPGKGLEWLSVISYDGTNKYYANSVRGRFTVSRDNSKNTLFLHMNSLGVDDTAMYYCARDPERLADLGRMDFWGRGTLVAVLS

SEQ ID NO: 821 - protein, 113 aa, Artificial Sequence
  Description of Artificial Sequence Synthetic polypeptide
DIVMTQSPDSLAVSLGERATINCKSSQSVLFSPNNKNYLAWYQQKPGQPPKLLIYWASTREYGVPERFSGSGSGTDFTLTISSLQAEDVAVYYCQQYNSNPYTFGQGTKLEIR

SEQ ID NO: 822 - protein, 121 aa, Artificial Sequence
  Description of Artificial Sequence Synthetic polypeptide
QVQLQESGPGLVKPSETLSLTCTVSGGSVSTYYWTWIRQPPGKGLDWIGYIYHNGNINYNPSLKSRVTMSVDTSKNQFSLKLTSVTAADTALYYCATVSFIGYSLYYFDRWGQGTLVTVSS

SEQ ID NO: 823 - protein, 107 aa, Artificial Sequence
  Description of Artificial Sequence Synthetic polypeptide
DIQMTQSPSSLSASVGDRVTITCRASQNIASFLNWYQQRPGKAPDLLIYAASTLQTGVPSRFSGSGSGTDFTLAISSLQPEDFATYYCQQSYNTPFTFGQGTKLEIK

SEQ ID NO: 824 - protein, 121 aa, Artificial Sequence
  Description of Artificial Sequence Synthetic polypeptide
QVQLQESGPGLVKPSQTLSLTCTVSGASVSSEYYYWSWIRQPPGKGLEWIGYIYHNGDTYYNPSLKGRGIISMDTSKNQFSLKLISVTAADTAVYYCARLAVAGVTGFHYWGLGTLVTVSS

SEQ ID NO: 825 - protein, 107 aa, Artificial Sequence
  Description of Artificial Sequence Synthetic polypeptide
EIVLTQSPDTLSLSPGERATLSCRASRSVRNYLAWYQQKPGQAPRLLIYDASNRAPGIPARFSGSGSETDFTLTISSLEPEDFAVYYCQQRSNWGLTFGGGTKVEIK

SEQ ID NO: 826 - protein, 124 aa, Artificial Sequence
  Description of Artificial Sequence Synthetic polypeptide
EVQLVESGGGLIQPGGSLRLSCTASGFTVSSKYMSWVRQAPGKGLEWVSLIYDGGSTYYADSVKGRFLISRDNAKNTLYLQMHSLRAEDTAVYYCARDRGTYSGYDWYSGMDVWGQGTTVTVSS

SEQ ID NO: 827 - protein, 107 aa, Artificial Sequence
  Description of Artificial Sequence Synthetic polypeptide
DIQLTQSPSFLSASVGDRITITCRASQGISSYLAWYQQKPGKAPKLLIYAASTLQSGVPSRFSGSGSGTEFNVTISSLQPEDFATYYCQQLKSYPGTFGQGTKVEIE

SEQ ID NO: 828 - protein, 122 aa, Artificial Sequence
  Description of Artificial Sequence Synthetic polypeptide
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCARDASGLSWYDWYFDLWGRGTLVTVSS

SEQ ID NO: 829 - protein, 107 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKLEIK

SEQ ID NO: 830 - protein, 116 aa, Artificial Sequence
  Description of Artificial Sequence Synthetic polypeptide
QVQLVESGGGVVQPGRSLRLSCVASGFPFDSFAMHWVRQAPGKGLEWVAIVWYDGGDKFYSESVKGRFSISRDNSRNTVFLQMNSLRAEDTAVYYCARGMITFDYWGPGTLVTVSS

SEQ ID NO: 831 - protein, 109 aa, Artificial Sequence
  Description of Artificial Sequence Synthetic polypeptide
EIVLTQSPGTLSLSPGERATLSCRASQNVDSSYLAWYQQKPGQAPRLLMYGASTRVAGIPDRFSGSGSGTDFTLTISRLEPEDFVVYYCQQYGTSPTWTFGQGTKVEIK

SEQ ID NO: 832 - protein, 125 aa, Artificial Sequence
  Description of Artificial Sequence Synthetic polypeptide
EVQLVESGGGLVKPGGSLRLSCAGSGFTFSSYSMNWVRQAPGKGLEWVSSISASSSYIYYADSLKGRFTISRDNAKNSLYLQMNSLRAEDTAVYYCARANYGSGNYYNPKWFDPWGQGTLVTVSS

SEQ ID NO: 833 - protein, 111 aa, Artificial Sequence
  Description of Artificial Sequence Synthetic polypeptide
QSVLTQPPSVSGAPGQRVTISCTGSSSNIGAGYDVHWYQQFPGTAPKVLIYGNNNRPSGVPDRFSGSKSGTSASLAISGLQAEDEADYYCQSYDSSLSGSIFGGGTKLTVL

SEQ ID NO: 834 - protein, 122 aa, Artificial Sequence
  Description of Artificial Sequence Synthetic polypeptide
EVQLVESGGSVIQPGGSLRLSCAASGFSVRTNYMNWVRQAPGKGLEWVSVIYSDGRTYYSDSVKGRFTISRDNSMNNVFLQMNSLRAEDTAVYYCARDPTFYSSSWWYLDHWGQGTQVTVSS

SEQ ID NO: 835 - protein, 108 aa, Artificial Sequence
  Description of Artificial Sequence Synthetic polypeptide
SYELTQALSVSVARGQTAKITCGGNNIGVRNVHWYQQKPGQAPVLVIYRDNIRPSGIPERFSGSNSGNMAALTISSAQEGDEADYYCQVWDSNTAHVIFGGGTKLTVL

SEQ ID NO: 836 - protein, 127 aa, Artificial Sequence
  Description of Artificial Sequence Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARYYYDSSGYYYYYYYYMDVWGKGTTVTVSS

SEQ ID NO: 837 - protein, 108 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 838 - protein, 121 aa, Artificial Sequence
  Description of Artificial Sequence Synthetic polypeptide
EVQLLESGGGLVQPGGSLRLSCAASGFSFSSYVMSWVRQAPGKGLEWVSTVSAGGGSTHYADSVKGRFTISRDNSKNTLYLQMNSLRAEDTAVFYCAKDKYPHYYYGMDVWGQGTTVSVSS

SEQ ID NO: 839 - protein, 110 aa, Artificial Sequence
  Description of Artificial Sequence Synthetic polypeptide
QSVLTQPPSASETPGQRVTISCSGSSSNIGRNYVYWYQQLPGTAPKLLIYRNNQRPSGVPDRFSGSESGTSASLAISGLRSEDEADYYCAAWDDSLSGYVFGTGTKVTVL

SEQ ID NO: 840 - protein, 120 aa, Artificial Sequence
  Description of Artificial Sequence Synthetic polypeptide
EVQLVESGGGVVRPGGSLRLSCAASGFRFDDYAMNWVRQAPGKGLEWVSGINWIGGTTKYADSVKGRFTISRDNTKNSLYLQMNSLRAEDTALYYCARGVGYYYYYMDVWGRGTTVTVSS

SEQ ID NO: 841 - protein, 108 aa, Artificial Sequence
  Description of Artificial Sequence Synthetic polypeptide
EIVLTQSPGTLSLSPGERATLSCRATQSVSSNYLAWYQQKPGQAPRLLIYDASSRAIGIPDRFSGSGSGTDFTLTISRLESEDFAVYYCQQYGSSPETFGQGTKVEIK

SEQ ID NO: 842 - protein, 131 aa, Artificial Sequence
  Description of Artificial Sequence Synthetic polypeptide
QVQLVQSGAEVKKPGSSVKVSCKASGGTINSYAISWVRQAPGQGPEWLGGIIPIFGTANYAQNFPGRVTITADASTSTVYMELSSLRSDDTAIYYCARGTRYIAVMVPSMQDYYFFGMDVWGQGTTVTVSS

SEQ ID NO: 843 - protein, 109 aa, Artificial Sequence
  Description of Artificial Sequence Synthetic polypeptide
DIQLTQSPSFLSASVGDRVTITCRASQGISSYLAWYQQKPGQAPKLLIYAASTLQSGVPSRFSGSGSGTEFTLTISSLQPEDFATYYCQQVTSYPFSITFGQGTRLEIK

SEQ ID NO: 844 - protein, 122 aa, Artificial Sequence
  Description of Artificial Sequence Synthetic polypeptide
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARITGEGLPQAFDYWGQGTLVTVSS

SEQ ID NO: 845 - protein, 106 aa, Artificial Sequence
  Description of Artificial Sequence Synthetic polypeptide
SYELTQPPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 846 - protein, 125 aa, Artificial Sequence
  Description of Artificial Sequence Synthetic polypeptide
QVQLVESGGGVVQPGGSLRLSCAASGFTFSRYAMHWVRQAPGKGLEWVAVMSHDGSNRYYADSVKGRFTISRDNSKNTLYLQMNSLRAEDTAVYYCAKDPGGLGYSYGLGYFDYWGQGTLVTVSS

SEQ ID NO: 847 - protein, 108 aa, Artificial Sequence
  Description of Artificial Sequence Synthetic polypeptide
EIVMTQSPATLSVSPGERATLSCRASQSVTNNLAWYQQKPGQAPRLLIYGASTRATGIPARISGSGSGTEFTLTINSLQSEDFAVYYCQQYNGWPPWTFGQGTKVEIK

SEQ ID NO: 848 - protein, 123 aa, Artificial Sequence
  Description of Artificial Sequence Synthetic polypeptide
EVQLVESGGGLVEPGGSLRLSCAASGFMFTSYAMHWVRQAPGRGLEWVSSITGGGNFVEYAVSVQGRFTVSRDNNQNSLYLQMNSLRVEDTAMYYCAREDYNSGANLHWFDPWGQGTLVTVSS

SEQ ID NO: 849 - protein, 111 aa, Artificial Sequence
  Description of Artificial Sequence Synthetic polypeptide
QSVLTQPPSVSGAPGQRVTISCTGSSSNIGAFFDVHWYSHLPGTAPKLLIYGDRNRPSGVPERFSGSKSGASASLTITGLRAEDEADYYCQSYDRGLSAWVFGGGTRVTVL

SEQ ID NO: 850 - protein, 119 aa, Artificial Sequence
  Description of Artificial Sequence Synthetic polypeptide
EVQLVESGGGLVKPGGSLRLSCAAFGFSLSGYGMNWVRQAPGKGLQWVSSITASSSYITYRDSLRGRFTISRDNAQNSLYLDMTALRVEDTAVYYCATNGKLPPYFDHWGQGVLVVVSS

SEQ ID NO: 851 - protein, 111 aa, Artificial Sequence
  Description of Artificial Sequence Synthetic polypeptide
QSVLTQPPSVSGAPGQRVTISCTGSGSNIGAGYDVHWYQQLPGTAPKLLIYDNNHRASGVPDRVSASWSGTSASLAITGLQAEDEADYYCQSYDRNLEGVVFGGGTKLTVL

SEQ ID NO: 852 - protein, 127 aa, Artificial Sequence
  Description of Artificial Sequence Synthetic polypeptide
EVQLVESGGGLVRPGGSLRLSCAASGFSLTTDEVNWVRQAPGKGPEWVSFISASGHTIYYADSVKGRFTISRDNGRNSVYLQMNSLRAEDTAVYYCARVGRLGGVGYYYALGPFDLWGQGTMVTVSS

SEQ ID NO: 853 - protein, 108 aa, Artificial Sequence
  Description of Artificial Sequence Synthetic polypeptide
EIVLTQSPGTLSLSPGERATLSCRASQSITSNYLAWYQQKPGQAPRLLIYGASSRATGIPDRFSGSGSGTDFTLTISRLQPEDFAVYYCQQYGSSLYIFGQGTKLEIK

SEQ ID NO: 854 - protein, 127 aa, Artificial Sequence
  Description of Artificial Sequence Synthetic polypeptide
EVQLVESGGALVRPGGSLRVSCEASGFNFRTHWMHWVRQAPGKGLVWVARTNGDGSVTNYADSVKGRFTFFRDNAKNTLFLQMTSLRVDDTAVYYCARERLDYSSTRGHFPGDFDSWGQGTLVTVSS

SEQ ID NO: 855 - protein, 113 aa, Artificial Sequence
  Description of Artificial Sequence Synthetic polypeptide
DIVMTQSPDSLAVSLGERATINCKSSESVLYTSNSKNYLAWYQQKPGHPPKVLIYWASTRESGVPDRFSGSGSGTDFSLSISSLQAEDVAVYYCQQYFTIPRTFGQGTKVEIK

SEQ ID NO: 856 - protein, 127 aa, Artificial Sequence
  Description of Artificial Sequence Synthetic polypeptide
QVQLVQSGAEVKKPGASVKVSCKASGYTFNGHYLHWVRQAPGQGLEWMGWINPNSGGTDYAQRFQGRVTMTRGTSISTAYMELSRLTSDDTAVYFCARDNFGFWSASSGRHNWLDSWGQGTLVIVSS

SEQ ID NO: 857 - protein, 111 aa, Artificial Sequence
  Description of Artificial Sequence Synthetic polypeptide
NFMLTQPHSVSESPGKTVTISCTRSTGNIASNYVQWYQQRPGSSPTTVIYEDNQRPSAVPDRFSGSIDSSSNSASLTVSGLRTEDEADYYCQSYDSGHHVVFGGGTKLTVL

SEQ ID NO: 858 - protein, 131 aa, Artificial Sequence
  Description of Artificial Sequence Synthetic polypeptide
QVQLVQSGSEVKRPGASVRVSCTASGYTFSNYGVSWMRQAPGHGLEWMGWISTHNQNVRYSQNFQGRVTVTTDTSTSTAYMDLRGLKPDDTAVYFCARMIPVGTTYTPYWTGWTPGGMDIWGQGTTVIVSS

SEQ ID NO: 859 - protein, 110 aa, Artificial Sequence
  Description of Artificial Sequence Synthetic polypeptide
QSVLTQPPSASGTPGQRVIMSCSGTTSNIGRNFVYWYQQLPKEAPTLLIYGNDQRPAGVPDRFSGSTSGTSASLAISGLRSGDEADYYCATWDDSVNGPVFGGGTKLTVL

SEQ ID NO: 860 - protein, 123 aa, Artificial Sequence
  Description of Artificial Sequence Synthetic polypeptide
QLQLQESGSGPGLVKPSETLSLICNASGDSVTRNAYYWGWLRQPPGKGLEWIGIMYYSGSTFYNPSLKSRLTISVDTSKNQFSLKLRSVTAADTATYYCGEATLTTYDAFDVWGHGTMVTVSS

SEQ ID NO: 861 - protein, 110 aa, Artificial Sequence
  Description of Artificial Sequence Synthetic polypeptide
QSVLTQPPSVSAAPGQRVTISCSGGNSNIGKNYVSWYQQVPGTAPKLLIYENNKRPSGIPDRFSGSKSGTSATLGITGLQTGDEADYYCGAWDSSLNGVLFGGGTRLTVL

SEQ ID NO: 862 - protein, 123 aa, Artificial Sequence
  Description of Artificial Sequence Synthetic polypeptide
EVQLLESGGGLVQPGGSLRLSCAASGFNFNTYAMSWVRQAPGKGLKWISTISGSGGVKYYADSVKGRFTISRDNSGSILYLQMNRLRADDTAVYYCAKSQNPQLVFFDSFDMWGQGSMVTVSS

SEQ ID NO: 863 - protein, 110 aa, Artificial Sequence
  Description of Artificial Sequence Synthetic polypeptide
QSVLTQAPSASAAPGQKVTISCSGSTSNIGNSYVSWYQQVPGTAPKLLIYEDNQRPSGIPDRFSGSKSGTSATLGITGLQTGDEADYYCGTWDNSLTEVVFGGGTKLTVL

SEQ ID NO: 864 - protein, 116 aa, Artificial Sequence
  Description of Artificial Sequence Synthetic polypeptide
QLQLQESGPSETLSLTCTVSGGSISSSSYYWGWIRQPPGKGLEWIGSIYYSGSTYYNPSLKSRVTISVDTSKNQFPLKLSSVTATDTAVYYCARLGVGLYEAFDIWGQGTMVTVSS

SEQ ID NO: 865 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFAIYYCQQSYSTPNTFGQGTRLEIK

SEQ ID NO: 866 - protein, 119 aa, Artificial Sequence
  Description of Artificial Sequence Synthetic polypeptide
QVQLVQSGAEVKKPGASVKVSCKASGYIFSNYRMHWVRQAPGQRLEWMGWITAGSGNTRYSEQFQGRVAFTSDTDATTVSMELSGLRFEDTAVYFCAREYNWNDAFPYWGQGTLVIVSS

SEQ ID NO: 867 - protein, 108 aa, Artificial Sequence
  Description of Artificial Sequence Synthetic polypeptide
SYVLTQPPSVSVAPGQTARIPCGGNNIGSKSVHWYQQKSGRAPVLVVYDDSDRPPGIPDRFSGSNSGNTATLTINKIEAGDEADYYCHVSDDNSDHVFFGGGTQLTVL

SEQ ID NO: 868 - protein, 117 aa, Artificial Sequence
  Description of Artificial Sequence Synthetic polypeptide
QVQLVESGGGVVQPGKSLRLSCAASGFTFRSYTMFWVRQAPGKGLEWVAIILHDGTNRYYTDSVKGRFTISRDNTKNTVYLQMNSLRPEDTAMYYCARESLEWMLDWGQGTLVTVSS

SEQ ID NO: 869 - protein, 109 aa, Artificial Sequence
  Description of Artificial Sequence Synthetic polypeptide
EIVMTQSPATLSVSPGERATLSCRASQSLSGNLAWFQQKPGQAPRLLIYGTSTRATGIPARFSGSGSGTEFTLTISSLQSEDFAVYYCQQYNNWPPVFTFGPGTKVDIK

SEQ ID NO: 870 - protein, 118 aa, Artificial Sequence
  Description of Artificial Sequence Synthetic polypeptide
QVQLVQSGPEVRKPGASVIISCKASGYSFISYYIHWVRQAPGQGLEWMGIINPSGGSTEYAQKYQGRLTITRDTSTTTVSMTLSSLRSDDTAVYFCAREQAVGAYDIWGQGTMVTVSS

SEQ ID NO: 871 - protein, 109 aa, Artificial Sequence
  Description of Artificial Sequence Synthetic polypeptide
EIVLTQSPGTLSLSPGERATLSCRAGQLINGNYLAWYQKKPGQAPSLLIYDVSRRAAGVPDRFTGSGSGTDFTLTISRLEPDDFAVYYCQHYGRSPSPTFGGGTKVEIK

SEQ ID NO: 872 - protein, 122 aa, Artificial Sequence
  Description of Artificial Sequence Synthetic polypeptide
EVQLLESGGGFVQPGGSLRLSCVVTGFTFSNYAMCWVRQAPGKGPEWVSGIGGSVGTTYYADSVKGRFTISRDNSKNTLYLQMSGLRAEDTARYYCAKVPSFFVFEDGFDMWGQGTVVTVSS

SEQ ID NO: 873 - protein, 110 aa, Artificial Sequence
  Description of Artificial Sequence Synthetic polypeptide
QSVLTQPPSASGNPGQRVTISCYGSGSNIGNNYVHWFRQLPGTAPRLLIYNTDILVPGVPDRFSGSESGTSASLAISGLQSGDEADYFCAAWDDTLNGYVFGPGTTVTVL

SEQ ID NO: 874 - protein, 121 aa, Artificial Sequence
  Description of Artificial Sequence Synthetic polypeptide
QVQLVQSGTEVQTPGASVKVSCKTSGYIFTGYFMHWVRQAPGQGFEWMGWVNPYSGATTYAQKFQGRVTMTRDTSISTVYMELSRLTSDDTAIYYCAKDTSNSGPDLLEHWGQGTLVIVSS

SEQ ID NO: 875 - protein, 109 aa, Artificial Sequence
  Description of Artificial Sequence Synthetic polypeptide
EIVLTQSPATLSLSPGERATLSCRASQSISNFLVWYQRKPGQAPRLLIFDVSKRATGIPTRFSGSGSGTDFTLTISSLEPEDFAVYYCQQRSNWPPGYTFGQGTKLEIK

SEQ ID NO: 876 - protein, 123 aa, Artificial Sequence
  Description of Artificial Sequence Synthetic polypeptide
QLQLQESGPRLVKPSETLSLTCSVSGGSISSSSLYWGWIRQSPGKGLEWIGTIYYGGSSDYNPSLKSRLTMSVDTSKNHFSLRLSSVTAADTAVYYCARVGSPVTTTVWFDPWGQGTLVTVSS

SEQ ID NO: 877 - protein, 108 aa, Artificial Sequence
  Description of Artificial Sequence Synthetic polypeptide
DIQMTQSPSSLAASIGDRVTITCRASQTISTYLNWYQQKPGKAPKLLIYAASTLQSGVPSRFSGSGSGTDFTLTISSLQPEDFATYYCQQTYSSLALTFGGGTKVEIR

SEQ ID NO: 878 - protein, 129 aa, Artificial Sequence
  Description of Artificial Sequence Synthetic polypeptide
EVQLVESGGTLVQPGGSLRLSCAASGFRFNAFSMNWVRQAPGKGLEWVSYIDSRSNIIYYADSVKGRFTISRDNAKTSLYLRMSSLRDEDTAVYYCAKALCVVNYDFWSGLLGCAMDVWGQGTTVTVSS

SEQ ID NO: 879 - protein, 112 aa, Artificial Sequence
  Description of Artificial Sequence Synthetic polypeptide
QSVLTQPPSVSGAPGQRVTISCTGTTSNIGAGFDVHWYQQVPGTAPKLLIYANTDRPSGVPDRFSASKSGTSASLAITGLQVEDEAHYYCQTYDTTLSGSTVFGGGTKLTVL

SEQ ID NO: 880 - protein, 125 aa, Artificial Sequence
  Description of Artificial Sequence Synthetic polypeptide
RVTLRESGPALLKPTQTLTLTCTFSGFSLNTYGMSVNWIRQPPGKALEWLSRIDWDGDKYFNTSLKTRLTISKDTSKNQVVLTMTNMEPVDTATYYCAQSVIVPAQSFVSWFDPWGPGTLVTVSS

SEQ ID NO: 881 - protein, 108 aa, Artificial Sequence
  Description of Artificial Sequence Synthetic polypeptide
EIVMTQSPATLSVSPGERATLSCRASQSVRSNLAWYQQKPGQAPRLLIYGASSRATGIPARFSGSGSGTEFTLTISSLQSEDLAIYYCQQYNNWPPGAFGQGTKVEIK

SEQ ID NO: 882 - protein, 129 aa, Artificial Sequence
  Description of Artificial Sequence Synthetic polypeptide
EVQLVESGGTLVQPGGSLRLSCAASGFKFNAFSMNWVRQAPGKGLEWVSYIDSRSNIIYYADSVKGRFTISRDNAKNSLYLRMSSLRDEDTAVYYCAKALCVVNYDFWSGLLGCAMDVWGQGTTVTVSS

SEQ ID NO: 883 - protein, 112 aa, Artificial Sequence
  Description of Artificial Sequence Synthetic polypeptide
QSVLTQPPSVSGAPGQRVTISCTGNTSNIGAGFDVHWYQQVPGTAPKLLIYANTDRPSGVPDRFSASKSGTSASLAITGLQAEDEADYYCQTYDRSLSGSTVFGGGTKLTVL

SEQ ID NO: 884 - protein, 129 aa, Artificial Sequence
  Description of Artificial Sequence Synthetic polypeptide
QVQLVQSGAEMKKPGSSVKVSCKTSGGSFSSYPVSWVRQAPGQGPEWMGGIIPLSGTTHYAQMFQGRVSITADDSTRTVFMELTSLRSGDTAVYYCARSGAPTYYYDSSGFHGGAFHFWGQGTVVTVSS

SEQ ID NO: 885 - protein, 115 aa, Artificial Sequence
  Description of Artificial Sequence Synthetic polypeptide
DIVMTQSPDSLAVSLGERATINCKSSQSVLYSSNSKSYLAWYQQKPGQPPKLLIYWASSRESGVPDRFSGSGSGTDFTLTISSLQAEDVAVYYCQQFYTSPHTFGQGTKLEIKRT

SEQ ID NO: 886 - protein, 121 aa, Artificial Sequence
  Description of Artificial Sequence Synthetic polypeptide
QVQLVESGGGLVQPGRSLRLSCAASGFTFSSYAMHWVRQAPGKGLEWVAIITYDGSGKYYADSLEGRITISRDDPKSMLYLHMRSLTAEDTAVYYCVKSGSFSSAFWLEDWGQGTLVTVSS

SEQ ID NO: 887 - protein, 107 aa, Artificial Sequence
  Description of Artificial Sequence Synthetic polypeptide
EIVLTQSPATLSLSPGERATLSCRASQSISNFVAWYQQKPGQAPRLLIYDASNGATGTPARFSGSGSGTDFTLTISSLEPEDFAVYYCQQRSTWTFGQGTKVEIKRT

SEQ ID NO: 888 - protein, 119 aa, Artificial Sequence
  Description of Artificial Sequence Synthetic polypeptide
QVQLVQSGPEVRKPGSSVRVSCKASGGTFTRLTITWVRQAPGQGLEWMGRIIPAVDITNYAPKFQGRVTITADKSTGAVYMDLISLTSDDTAIYFCATVLDWSNALDIWGQGTMVTVSS

SEQ ID NO: 889 - protein, 114 aa, Artificial Sequence
  Description of Artificial Sequence Synthetic polypeptide
DIVMTQTPLSLSVTPGQPASISCKSSESLLNSDGKTYLFWYLQKPGQSPQVLIYDLSSRFSGVPERFSGSGSGTDFTLKISRVEPEDVGIYYCMQGLHLPWTFGRGTRLEIKRT

SEQ ID NO: 890 - protein, 125 aa, Artificial Sequence
  Description of Artificial Sequence Synthetic polypeptide
QVQLQESGPGLVKPSGTLSLTCTVSGGSVNSDHWWTWVRQSPETGLEWIGELNHIGTFNYNPSFKSRATISIDTSKNQFSLTLTSVAAADTAVYYCARRPPPLPEYFYYYFMDVWGNGTTVTVSS

SEQ ID NO: 891 - protein, 109 aa, Artificial Sequence
  Description of Artificial Sequence Synthetic polypeptide
DIQMTQSPSSLSASVGDRVTITCRTSQSIRGHINWYQQKPGKGPKLLIYGASSLQSGVPSRFSGSGSGTDFTLTISSLQAEDFATYYCQQSYSNPYSFGRGTKLEIKRT

SEQ ID NO: 892 - protein, 118 aa, Artificial Sequence
  Description of Artificial Sequence Synthetic polypeptide
QVQLQESGPGLVKPSETLSLTCSVSGASIGGYYWGWIRQPPGKGLEWLGYIYYSGRTVYSPSLRGRLALSLDMSRQQLSLRLRSVAAADTAVYYCASLTITPGWFDSWGPGTLVTVSS

SEQ ID NO: 893 - protein, 111 aa, Artificial Sequence
  Description of Artificial Sequence Synthetic polypeptide
QSVLTQPPSVSGAPGQRVTISCTGSSSNIGAGYDVHWYQQFPGSAPKLLIYGNTKRPSGVPDRFSGSTSGSSASLAITGLQTEDEAEYYCQSYERNLDGALFGGGTKLSVL

SEQ ID NO: 894 - protein, 117 aa, Artificial Sequence
  Description of Artificial Sequence Synthetic polypeptide
QVQLVQSGAEVKKPGASVKVSCKASGYNFDNYYMSWVRQAPGQGLEWLGIIDPSGGRISYAPKFQGRVTLTRDTSTSTVYMELRGLRFEDTALYYCARLEATHEGPWGQGTLVTVSS

SEQ ID NO: 895 - protein, 111 aa, Artificial Sequence
  Description of Artificial Sequence Synthetic polypeptide
QSVLTQPPSASGTPGQRVTISCSGSNSNIGSSTVNWYQQLPGTAPKLLIYSDNHRPSGVPDRFSGSRSGTSASLVISGLHSEDEADYYCGTWDDGLNGPYVFGTGTKVTVL

SEQ ID NO: 896 - protein, 118 aa, Artificial Sequence
  Description of Artificial Sequence Synthetic polypeptide
QVQLVQSGPEVKKPGASVKISCKASEYTFTNHYLHWVRQAPGQGLEWLGIINPTGGSTYYAGDFQGRVTLTRDASTTTVYMELSSVKSEDTAIYYCARDRGMRYFDYWGQGTLVTVSS

SEQ ID NO: 897 - protein, 112 aa, Artificial Sequence
  Description of Artificial Sequence Synthetic polypeptide
QSVLTQPPSVSGAPGQRVSISCTGISSNIGATYDVHWYQQLPGTAPKLLIYGNNNRPSGVPDRFSGSKSGTSASLAITGLQAEDEAVYYCQSYDSVLSGSVIFGGGTILTVL

SEQ ID NO: 898 - protein, 127 aa, Artificial Sequence
  Description of Artificial Sequence Synthetic polypeptide
EVQLVESGGGLVQLGGSLRLSCAGSGFSFSTFWMSWVRQAPGKGLEWLANIKPDGSEKFYVDSVKGRITLSRDNANNSLYLQMRNLRDDDTAVYYCARDRGYCRGGSCYRRNAFDLWGQGTMVTVSS

SEQ ID NO: 899 - protein, 111 aa, Artificial Sequence
  Description of Artificial Sequence Synthetic polypeptide
QSALTQPPSASGSPGQSVTISCTGTSGDVGGYTFVSWYQQYPGKAPKLIIYEVTKRPSGVPDRFSGSKSGNTASLTVSGLQTEDEADYFCSSYSSTNNLRVFGGGTKLTVL

SEQ ID NO: 900 - protein, 122 aa, Artificial Sequence
  Description of Artificial Sequence Synthetic polypeptide
QVQLVQSGAEVKKPGSSVKVSCNASGGIFDTNVINWVRQAPGQGLEWMGGIIPPFGTALYAHQFQGRLTLTVDESTNTAYMDLSSLRSDDTAVYFCARDRTVIKYADGMDVWGQGTTVTVSS

SEQ ID NO: 901 - protein, 109 aa, Artificial Sequence
  Description of Artificial Sequence Synthetic polypeptide
DIQMTQSPSSLSASVGDKVAITCRASQTISPYLNWYQQKPGKAPNLLIYGTNKLQSGVPLRFSGSGSGTDFTLTINGLQPEDFATYFCQQSHTTPLTFGGGTKVEIKRT

SEQ ID NO: 902 - protein, 122 aa, Artificial Sequence
  Description of Artificial Sequence Synthetic polypeptide
QVQLVQSGAEVKKPGSSVKVSCKASGGTLSNYAISWVRQAPGQGLEWMGGIIPIFGTAMYAHNFQDRVTFTADESTNTVYMAVSRLTSDDTAVYYCAQDRTVTKYAHGLDVWGQGTTVTVSS

SEQ ID NO: 903 - protein, 109 aa, Artificial Sequence
  Description of Artificial Sequence Synthetic polypeptide
DIQMTQSPSSLSASVGDRITITCPASQSLLTYLNWYQQKPGKAPKLLIYSASELPSGVPSRFSGSGSGTEFTLTVTGLQPDDFATYYCQQSHTTPLTFGGGTKVEIKRT

SEQ ID NO: 904 - protein, 120 aa, Artificial Sequence
  Description of Artificial Sequence Synthetic polypeptide
QVQLVESGGGVVQPGRSLRLSCAASGFTFTTYTMHWVRQAPGKGLEWVALISSDGNNKYYADSVKGRFTISRDNSKNTLYLQMSSLRAEDTAVYYCARGYYSGGDAFDVWGQGTMVTVSS

SEQ ID NO: 905 - protein, 109 aa, Artificial Sequence
  Description of Artificial Sequence Synthetic polypeptide
DIQMTQSPSTLSASVGDRVTITCRASQTITIWLAWYQQKPGKAPKLLIYDAVSLESGVPSRFRGSGSGTDFTLTISSLQPDDFATYYCQQYNNYPYTFGQGTKLEIKRT

SEQ ID NO: 906 - protein, 231 aa, Artificial Sequence
  Description of Artificial Sequence Synthetic polypeptide
QVQLVESGGGVVQPGRSLRLSCAASGFTFTTYTMHWVRQAPGKGLEWVALISSDGNNKYYADSVKGRFTISRDNSKNTLYLQMSSLRAEDTAVYYCARGYYSGGDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKHHHHHH

SEQ ID NO: 907 - protein, 224 aa, Artificial Sequence
  Description of Artificial Sequence Synthetic polypeptide
DIQMTQSPSTLSASVGDRVTITCRASQTITIWLAWYQQKPGKAPKLLIYDAVSLESGVPSRFRGSGSGTDFTLTISSLQPDDFATYYCQQYNNYPYTFGQGTKLEIKRTVAAPSVFITPPSDEQLKSGTASVVCLLNNFYPREAKVQWKVDNALQSGNSQESVTEQDSKDSTYSLSSTLTLSKADYEKHKVYACEVTHQGLSSPVTKSFNRGECNSAEKGNTGL

SEQ ID NO: 908 - protein, 4 aa, Artificial Sequence
  Description of Artificial Sequence Synthetic peptide
RKRR

SEQ ID NO: 909 - protein, 8 aa, Artificial Sequence
  Description of Artificial Sequence Synthetic peptide
FVLGAIAL

SEQ ID NO: 910 - protein, 543 aa, Artificial Sequence
  Description of Artificial Sequence Synthetic polypeptide
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAARMKQIEDKIEEILSKIYHIENEIARIKKLIGEAGSGLVPRGSHHHHHHSAWSHPQFEK

SEQ ID NO: 911 - protein, 512 aa, Artificial Sequence
  Description of Artificial Sequence Synthetic polypeptide
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAEKGNTGLVPRGSHHHHHHSAWSHPQFEK

SEQ ID NO: 912 - protein, 540 aa, Artificial Sequence
  Description of Artificial Sequence Synthetic polypeptide
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRKRRGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAEKGNTGSAIGGYIPEAPRDGQAYVRKDGEWVLLSTFLGGLVPRGSHHHHHHSAWSHPQFEK

SEQ ID NO: 913 - protein, 543 aa, Artificial Sequence
  Description of Artificial Sequence Synthetic polypeptide
MSWKVMIIISLLITPQHGLKESYLEESCSTITEGYLSVLRTGWYTNVFTLEVGDVENLTCTDGPSLIKTELDLTKSALRELKTVSADQLAREEQIENPRRRRFVLGAIALGVATAAAVTAGIAIAKTIRLESEVNAIKGALKTTNEAVSTLGNGVRVLATAVRELKEFVSKNLTSAINKNKCDIPDLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAARMKQIEDKIEEILSKIYHIENEIARIKKLIGEAGSGLVPRGSHHHHHHSAWSHPQFEK

SEQ ID NO: 914 - protein, 6 aa, Artificial Sequence
  Description of Artificial Sequence Synthetic 6xHis tag
HHHHHH

SEQ ID NO: 915 - DNA, 24 bp, Artificial Sequence
  Description of Artificial Sequence Synthetic primer
acaggtgcccactcccaggtgcag

SEQ ID NO: 916 - DNA, 27 bp, Artificial Sequence
  Description of Artificial Sequence Synthetic primer
ctgaccatcccttcatgggtcttgtcc

SEQ ID NO: 917 - DNA, 26 bp, Artificial Sequence
  Description of Artificial Sequence Synthetic primer
aaggtgtccagtgtgargtgcagctg

SEQ ID NO: 918 - DNA, 24 bp, Artificial Sequence
  Description of Artificial Sequence Synthetic primer
agatgggtcctgtcccaggtgcag

SEQ ID NO: 919 - DNA, 25 bp, Artificial Sequence
  Description of Artificial Sequence Synthetic primer
caaggagtctgttccgaggtgcagc

SEQ ID NO: 920 - DNA, 19 bp, Artificial Sequence
  Description of Artificial Sequence Synthetic primer
gctcacgtccaccaccacg

SEQ ID NO: 921 - DNA, 24 bp, Artificial Sequence
  Description of Artificial Sequence Synthetic primer
atgagggtcccygctcagctcctg

SEQ ID NO: 922 - DNA, 24 bp, Artificial Sequence
  Description of Artificial Sequence Synthetic primer
ctcttcctcctgctactctggctc

SEQ ID NO: 923 - DNA, 27 bp, Artificial Sequence
  Description of Artificial Sequence Synthetic primer
caggtcttcatttctctgttgctctgg

SEQ ID NO: 924 - DNA, 24 bp, Artificial Sequence
  Description of Artificial Sequence Synthetic primer
gttcacctcctcagcttcctcctc

SEQ ID NO: 925 - DNA, 21 bp, Artificial Sequence
  Description of Artificial Sequence Synthetic primer
ctctgggttccagcctccagg

SEQ ID NO: 926 - DNA, 28 bp, Artificial Sequence
  Description of Artificial Sequence Synthetic primer
acactctcccctgttgaagctctttgtg

SEQ ID NO: 927 - DNA, 23 bp, Artificial Sequence
  Description of Artificial Sequence Synthetic primer
ggtcctgggcccagtctgtgctg

SEQ ID NO: 928 - DNA, 23 bp, Artificial Sequence
  Description of Artificial Sequence Synthetic primer
ggtcctgggcycagtctgccctg

SEQ ID NO: 929 - DNA, 23 bp, Artificial Sequence
  Description of Artificial Sequence Synthetic primer
gctctgwggcctcctatgagctg

SEQ ID NO: 930 - DNA, 23 bp, Artificial Sequence
  Description of Artificial Sequence Synthetic primer
ggtctctctcscagcytgtgctg

SEQ ID NO: 931 - DNA, 27 bp, Artificial Sequence
  Description of Artificial Sequence Synthetic primer
gttcttgggccaattttatgctgactc

SEQ ID NO: 932 - DNA, 23 bp, Artificial Sequence
  Description of Artificial Sequence Synthetic primer
ggtccaattcycaggctgtggtg

SEQ ID NO: 933 - DNA, 23 bp, Artificial Sequence
  Description of Artificial Sequence Synthetic primer
gagtggattctcagactgtggtg

SEQ ID NO: 934 - DNA, 24 bp, Artificial Sequence
  Description of Artificial Sequence Synthetic primer
gtgtcagtggtccaggcagggctg

SEQ ID NO: 935 - DNA, 24 bp, Artificial Sequence
  Description of Artificial Sequence Synthetic primer
cattcygyaggggcmactgtcttc

SEQ ID NO: 936 - DNA, 56 bp, Artificial Sequence
  Description of Artificial Sequence Synthetic primer
acactctttccctacacgacgctcttccgatctcaggtgcagctggtgcagtctgg

SEQ ID NO: 937 - DNA, 56 bp, Artificial Sequence
  Description of Artificial Sequence Synthetic primer
acactctttccctacacgacgctcttccgatctcagatcaccttgaaggagtctgg

SEQ ID NO: 938 - DNA, 56 bp, Artificial Sequence
  Description of Artificial Sequence Synthetic primer
acactctttccctacacgacgctcttccgatctgaggtgcagctggtgsagtctgg

SEQ ID NO: 939 - DNA, 56 bp, Artificial Sequence
  Description of Artificial Sequence Synthetic primer
acactctttccctacacgacgctcttccgatctcaggtgcagctgcagsagtcggg

SEQ ID NO: 940 - DNA, 56 bp, Artificial Sequence
  Description of Artificial Sequence Synthetic primer
acactctttccctacacgacgctcttccgatctcaggtacagctgcagcagtcagg

SEQ ID NO: 941 - DNA, 51 bp, Artificial Sequence
  Description of Artificial Sequence Synthetic primer
gtgactggagttcagacgtgtgctcttccgatcttgaccaggcagcccagg

SEQ ID NO: 942 - DNA, 51 bp, Artificial Sequence
  Description of Artificial Sequence Synthetic primer
acactctttccctacacgacgctcttccgatctgacatccagwtgacccag

SEQ ID NO: 943 - DNA, 51 bp, Artificial Sequence
  Description of Artificial Sequence Synthetic primer
acactctttccctacacgacgctcttccgatctgccatccggttgacccag

SEQ ID NO: 944 - DNA, 51 bp, Artificial Sequence
  Description of Artificial Sequence Synthetic primer
acactctttccctacacgacgctcttccgatctgatattgtgatgacycag

SEQ ID NO: 945 - DNA, 51 bp, Artificial Sequence
  Description of Artificial Sequence Synthetic primer
acactctttccctacacgacgctcttccgatctgaaattgtgttgacrcag

SEQ ID NO: 946 - DNA, 51 bp, Artificial Sequence
  Description of Artificial Sequence Synthetic primer
acactctttccctacacgacgctcttccgatctgaaatagtgatgacgcag

SEQ ID NO: 947 - DNA, 51 bp, Artificial Sequence
  Description of Artificial Sequence Synthetic primer
acactctttccctacacgacgctcttccgatctgacatcgtgatgacccag

SEQ ID NO: 948 - DNA, 51 bp, Artificial Sequence
  Description of Artificial Sequence Synthetic primer
acactctttccctacacgacgctcttccgatctgaaacgacactcacgcag

SEQ ID NO: 949 - DNA, 51 bp, Artificial Sequence
  Description of Artificial Sequence Synthetic primer
acactctttccctacacgacgctcttccgatctgaaattgtgctgactcag

SEQ ID NO: 950 - DNA, 51 bp, Artificial Sequence
  Description of Artificial Sequence Synthetic primer
acactctttccctacacgacgctcttccgatctgatgttgtgatgacacag

SEQ ID NO: 951 - DNA, 51 bp, Artificial Sequence
  Description of Artificial Sequence Synthetic primer
gtgactggagttcagacgtgtgctcttccgatctaagacagatggtgcagc

SEQ ID NO: 952 - DNA, 51 bp, Artificial Sequence
  Description of Artificial Sequence Synthetic primer
acactctttccctacacgacgctcttccgatctcagtctgtgctgackcag

SEQ ID NO: 953 - DNA, 51 bp, Artificial Sequence
  Description of Artificial Sequence Synthetic primer
acactctttccctacacgacgctcttccgatctcagtctgccctgactcag

SEQ ID NO: 954 - DNA, 51 bp, Artificial Sequence
  Description of Artificial Sequence Synthetic primer
acactctttccctacacgacgctcttccgatcttcctatgagctgacwcag

SEQ ID NO: 955 - DNA, 51 bp, Artificial Sequence
  Description of Artificial Sequence Synthetic primer
acactctttccctacacgacgctcttccgatctcagcytgtgctgactcag

SEQ ID NO: 956 - DNA, 51 bp, Artificial Sequence
  Description of Artificial Sequence Synthetic primer
acactctttccctacacgacgctcttccgatctaattttatgctgactcag

SEQ ID NO: 957 - DNA, 51 bp, Artificial Sequence
  Description of Artificial Sequence Synthetic primer
acactctttccctacacgacgctcttccgatctcagrctgtggtgactcag

SEQ ID NO: 958 - DNA, 51 bp, Artificial Sequence
  Description of Artificial Sequence Synthetic primer
acactctttccctacacgacgctcttccgatctcaggcagggctgactcag

SEQ ID NO: 959 - DNA, 52 bp, Artificial Sequence
  Description of Artificial Sequence Synthetic primer
gtgactggagttcagacgtgtgctcttccgatctgggygggaacagagtgac

SEQ ID NO: 960 - DNA, 59 bp, Artificial Sequence
  Description of Artificial Sequence Synthetic primer
aatgatacggcgaccaccgagatctacacctctctatacactctttccctacacgacgc

SEQ ID NO: 961 - DNA, 59 bp, Artificial Sequence
  Description of Artificial Sequence Synthetic primer
aatgatacggcgaccaccgagatctacactatcctctacactctttccctacacgacgc

SEQ ID NO: 962 - DNA, 59 bp, Artificial Sequence
  Description of Artificial Sequence Synthetic primer
aatgatacggcgaccaccgagatctacacgtaaggagacactctttccctacacgacgc

SEQ ID NO: 963 - DNA, 59 bp, Artificial Sequence
  Description of Artificial Sequence Synthetic primer
aatgatacggcgaccaccgagatctacacactgcataacactctttccctacacgacgc

SEQ ID NO: 964 - DNA, 59 bp, Artificial Sequence
  Description of Artificial Sequence Synthetic primer
aatgatacggcgaccaccgagatctacacaaggagtaacactctttccctacacgacgc

SEQ ID NO: 965 - DNA, 59 bp, Artificial Sequence
  Description of Artificial Sequence Synthetic primer
aatgatacggcgaccaccgagatctacacctaagcctacactctttccctacacgacgc

SEQ ID NO: 966 - DNA, 59 bp, Artificial Sequence
  Description of Artificial Sequence Synthetic primer
aatgatacggcgaccaccgagatctacaccgtctaatacactctttccctacacgacgc

SEQ ID NO: 967 - DNA, 59 bp, Artificial Sequence
  Description of Artificial Sequence Synthetic primer
aatgatacggcgaccaccgagatctacactctctccgacactctttccctacacgacgc

SEQ ID NO: 968 - DNA, 55 bp, Artificial Sequence
  Description of Artificial Sequence Synthetic primer
caagcagaagacggcatacgagattcgccttagtgactggagttcagacgtgtgc

SEQ ID NO: 969 - DNA, 55 bp, Artificial Sequence
  Description of Artificial Sequence Synthetic primer
caagcagaagacggcatacgagatctagtacggtgactggagttcagacgtgtgc

SEQ ID NO: 970 - DNA, 55 bp, Artificial Sequence
  Description of Artificial Sequence Synthetic primer
caagcagaagacggcatacgagatttctgcctgtgactggagttcagacgtgtgc

SEQ ID NO: 971 - DNA, 55 bp, Artificial Sequence
  Description of Artificial Sequence Synthetic primer
caagcagaagacggcatacgagatgctcaggagtgactggagttcagacgtgtgc

SEQ ID NO: 972 - DNA, 55 bp, Artificial Sequence
  Description of Artificial Sequence Synthetic primer
caagcagaagacggcatacgagataggagtccgtgactggagttcagacgtgtgc

SEQ ID NO: 973 - DNA, 55 bp, Artificial Sequence
  Description of Artificial Sequence Synthetic primer
caagcagaagacggcatacgagatcatgcctagtgactggagttcagacgtgtgc

SEQ ID NO: 974 - DNA, 55 bp, Artificial Sequence
  Description of Artificial Sequence Synthetic primer
caagcagaagacggcatacgagatgtagagaggtgactggagttcagacgtgtgc

SEQ ID NO: 975 - DNA, 55 bp, Artificial Sequence
  Description of Artificial Sequence Synthetic primer
caagcagaagacggcatacgagatcagcctcggtgactggagttcagacgtgtgc

SEQ ID NO: 976 - DNA, 55 bp, Artificial Sequence
  Description of Artificial Sequence Synthetic primer
caagcagaagacggcatacgagattgcctcttgtgactggagttcagacgtgtgc

SEQ ID NO: 977 - DNA, 55 bp, Artificial Sequence
  Description of Artificial Sequence Synthetic primer
caagcagaagacggcatacgagattcctctacgtgactggagttcagacgtgtgc

SEQ ID NO: 978 - DNA, 55 bp, Artificial Sequence
  Description of Artificial Sequence Synthetic primer
caagcagaagacggcatacgagattcatgagcgtgactggagttcagacgtgtgc

SEQ ID NO: 979 - DNA, 55 bp, Artificial Sequence
  Description of Artificial Sequence Synthetic primer
caagcagaagacggcatacgagatcctgagatgtgactggagttcagacgtgtgc

SEQ ID NO: 980 - DNA, 59 bp, Artificial Sequence
  Description of Artificial Sequence Synthetic primer
aatgatacggcgaccaccgagatctacactcgactagacactctttccctacacgacgc

SEQ ID NO: 981 - DNA, 59 bp, Artificial Sequence
  Description of Artificial Sequence Synthetic primer
aatgatacggcgaccaccgagatctacacttctagctacactctttccctacacgacgc

SEQ ID NO: 982 - DNA, 59 bp, Artificial Sequence
  Description of Artificial Sequence Synthetic primer
aatgatacggcgaccaccgagatctacaccctagagtacactctttccctacacgacgc

SEQ ID NO: 983 - DNA, 59 bp, Artificial Sequence
  Description of Artificial Sequence Synthetic primer
aatgatacggcgaccaccgagatctacacgcgtaagaacactctttccctacacgacgc

SEQ ID NO: 984 - DNA, 59 bp, Artificial Sequence
  Description of Artificial Sequence Synthetic primer
aatgatacggcgaccaccgagatctacacctattaagacactctttccctacacgacgc

SEQ ID NO: 985 - DNA, 59 bp, Artificial Sequence
  Description of Artificial Sequence Synthetic primer
aatgatacggcgaccaccgagatctacacaaggctatacactctttccctacacgacgc

SEQ ID NO: 986 - DNA, 59 bp, Artificial Sequence
  Description of Artificial Sequence Synthetic primer
aatgatacggcgaccaccgagatctacacgagccttaacactctttccctacacgacgc

SEQ ID NO: 987 - DNA, 59 bp, Artificial Sequence
  Description of Artificial Sequence Synthetic primer
aatgatacggcgaccaccgagatctacacttatgcgaacactctttccctacacgacgc

SEQ ID NO: 988 - DNA, 55 bp, Artificial Sequence
  Description of Artificial Sequence Synthetic primer
caagcagaagacggcatacgagattagcgagtgtgactggagttcagacgtgtgc

SEQ ID NO: 989 - DNA, 55 bp, Artificial Sequence
  Description of Artificial Sequence Synthetic primer
caagcagaagacggcatacgagatgtagctccgtgactggagttcagacgtgtgc

SEQ ID NO: 990 - DNA, 55 bp, Artificial Sequence
  Description of Artificial Sequence Synthetic primer
caagcagaagacggcatacgagattactacgcgtgactggagttcagacgtgtgc

SEQ ID NO: 991 - DNA, 55 bp, Artificial Sequence
  Description of Artificial Sequence Synthetic primer
caagcagaagacggcatacgagataggctccggtgactggagttcagacgtgtgc

SEQ ID NO: 992 - DNA, 55 bp, Artificial Sequence
  Description of Artificial Sequence Synthetic primer
caagcagaagacggcatacgagatgcagcgtagtgactggagttcagacgtgtgc

SEQ ID NO: 993 - DNA, 55 bp, Artificial Sequence
  Description of Artificial Sequence Synthetic primer
caagcagaagacggcatacgagatctgcgcatgtgactggagttcagacgtgtgc

SEQ ID NO: 994 - DNA, 55 bp, Artificial Sequence
  Description of Artificial Sequence Synthetic primer
caagcagaagacggcatacgagatgagcgctagtgactggagttcagacgtgtgc

SEQ ID NO: 995 - DNA, 55 bp, Artificial Sequence
  Description of Artificial Sequence Synthetic primer
caagcagaagacggcatacgagatcgctcagtgtgactggagttcagacgtgtgc

SEQ ID NO: 996 - DNA, 55 bp, Artificial Sequence
  Description of Artificial Sequence Synthetic primer
caagcagaagacggcatacgagatgtcttagggtgactggagttcagacgtgtgc

SEQ ID NO: 997 - DNA, 55 bp, Artificial Sequence
  Description of Artificial Sequence Synthetic primer
caagcagaagacggcatacgagatactgatcggtgactggagttcagacgtgtgc

SEQ ID NO: 998 - DNA, 55 bp, Artificial Sequence
  Description of Artificial Sequence Synthetic primer
caagcagaagacggcatacgagattagctgcagtgactggagttcagacgtgtgc

SEQ ID NO: 999 - DNA, 55 bp, Artificial Sequence
  Description of Artificial Sequence Synthetic primer
caagcagaagacggcatacgagatgacgtcgagtgactggagttcagacgtgtgc

SEQ ID NO: 1000 - protein, 10 aa, Human metapneumovirus
ENPRQSRFVL

SEQ ID NO: 1001 - protein, 10 aa, Artificial Sequence
  Description of Artificial Sequence Synthetic peptide
ENPRRRRFVL

SEQ ID NO: 1002 - protein, 523 aa, Artificial Sequence
  Description of Artificial Sequence Synthetic polypeptide
LKESYLEESCSTITEGYLSVLRTGWYTNVFTLEVGDVENLTCTDGPSLIKTELDLTKSALRELKTVSADQLAREEQIENPRRRRFVLGAIALGVATAAAVTAGIAIAKTIRLESEVNAIKGALKTTNEAVSTLGNGVRVLATAVRELKEFVSKNLTSAINKNKCDIPDLKMAVSFSQFNRRFLNVVRQFSDNAGITPAISLDLMTDAELARAVSYMPTSAGQIKLMLENRAMVRRKGFGILIGVYGSSVYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAARMKQIEDKIEEILSKIYHIENEIARIKKLIGEAGSGLVPRGSHHHHHHSAWSHPQFE

SEQ ID NO: 1003 - DNA, 1617 bp, Human metapneumovirus
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagacaatcaaggtttgtcctaggtgcaatagctcttggtgttgccacagcagcagcagtcacggcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggcttcattattgtaataattttgattgctgttcttgggttaaccatgatttcagtgagcatcatcatcataatcaaaaaaacaaggaaacccacaggggcacctccagagctgaatggtgttaccaacggcggttttataccgcatagt

SEQ ID NO: 1004 - DNA, 1629 bp, Artificial Sequence
  Description of Artificial Sequence Synthetic non-optimized CDS for SEQ ID NO 910
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagacaatcaaggtttgtcctaggtgcaatagctcttggtgttgccacagcagcagcagtcacggcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagcccggatgaagcagatcgaggataagattgaggaaatcctgagcaaaatctaccacatcgagaatgagatcgcccggatcaagaagctgatcggagaggccggcagcggcctggtgcctagaggcagccaccaccatcatcaccacagcgcctggtcccaccctcagttcgagaag

SEQ ID NO: 1005 - DNA, 1629 bp, Artificial Sequence
  Description of Artificial Sequence Synthetic non-optimized CDS for SEQ ID NO 910
atgagctggaaagtcatgatcatcatcagcctgctgatcacccctcagcacggcctgaaagagagctacctggaagagtcctgctccaccatcaccgagggctacctgagcgtgctgagaaccggctggtacaccaacgtgttcaccctggaagtgggcgacgtggaaaacctgacctgcaccgacggccccagcctgatcaagaccgagctggatctgaccaagagcgccctgagagaactgaaaaccgtgtccgccgaccagctggccagagaggaacagatcgagaaccccagacagagcagattcgtgctgggcgccattgccctgggagtggctacagctgctgccgtgacagccggcattgccattgccaagaccatccggctggaaagcgaagtgaacgccatcaaaggcgccctgaaaaccaccaacgaggccgtgtctaccctgggcaacggcgtcagagtgctggccacagccgtgcgcgagctgaaagaatttgtgtccaagaacctgaccagcgccatcaacaagaacaagtgcgacattgccgacctgaagatggccgtgtccttcagccagttcaaccggcggttcctgaacgtcgtgcggcagttcagcgacaacgccggcatcacccctgccatctccctggacctgatgaccgacgccgaactggccagggctgtgtcctacatgcctacctctgccggccagatcaagctgatgctggaaaacagagccatggtccgacggaagggcttcggcatcctgatcggcgtgtacggcagctccgtgatctacatggtgcagctgcctatcttcggcgtgatcgacaccccttgctggatcatcaaggccgctcccagctgctccgagaaggacggcaattacgcctgcctgctgagagaggaccagggctggtactgcaagaacgccggctccaccgtgtactaccccaacgacaaggactgcgagacacggggcgaccacgtgttctgtgacaccgccgctggcatcaacgtggccgagcagagcagagagtgcaacatcaacatcagcaccaccaactacccctgcaaggtgtccaccggcagacaccccatctctatggtggccctgtctcctctgggagccctggtggcctgctacaagggcgtgtcctgtagcatcggcagcaacagagtgggcatcatcaagcagctgcccaagggctgctcctacatcaccaaccaggacgccgacaccgtgaccatcgacaacaccgtgtatcagctgagcaaggtggaaggcgagcagcacgtgatcaagggcagacccgtgtccagcagcttcgaccccatcaagttccccgaggaccagttcaatgtggccctggaccaggtgttcgagagcatcgagaacagccaggctctggtggaccagtccaacaagatcctgaactccgccgcccggatgaagcagatcgaggataagattgaggaaatcctgagcaaaatctaccacatcgagaatgagatcgcccggatcaagaagctgatcggagaggccggcagcggcctggtgcctagaggcagccaccaccatcatcaccacagcgcctggtcccaccctcagttcgagaag

SEQ ID NO: 1006 - DNA, 1536 bp, Artificial Sequence
  Description of Artificial Sequence Synthetic non-optimized CDS for SEQ ID NO 911
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagacaatcaaggtttgtcctaggtgcaatagctcttggtgttgccacagcagcagcagtcacggcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggcctggtgcctagaggcagccaccaccatcatcaccacagcgcctggtcccaccctcagttcgagaag

SEQ ID NO: 1007 - DNA, 1536 bp, Artificial Sequence
  Description of Artificial Sequence Synthetic optimized CDS for SEQ ID NO 911
atgagctggaaagtcatgatcatcatcagcctgctgatcacccctcagcacggcctgaaagagagctacctggaagagtcctgctccaccatcaccgagggctacctgagcgtgctgagaaccggctggtacaccaacgtgttcaccctggaagtgggcgacgtggaaaacctgacctgcaccgacggccccagcctgatcaagaccgagctggatctgaccaagagcgccctgagagaactgaaaaccgtgtccgccgaccagctggccagagaggaacagatcgagaaccccagacagagcagattcgtgctgggcgccattgccctgggagtggctacagctgctgccgtgacagccggcattgccattgccaagaccatccggctggaaagcgaagtgaacgccatcaaaggcgccctgaaaaccaccaacgaggccgtgtctaccctgggcaacggcgtcagagtgctggccacagccgtgcgcgagctgaaagaatttgtgtccaagaacctgaccagcgccatcaacaagaacaagtgcgacattgccgacctgaagatggccgtgtccttcagccagttcaaccggcggttcctgaacgtcgtgcggcagttcagcgacaacgccggcatcacccctgccatctccctggacctgatgaccgacgccgaactggccagggctgtgtcctacatgcctacctctgccggccagatcaagctgatgctggaaaacagagccatggtccgacggaagggcttcggcatcctgatcggcgtgtacggcagctccgtgatctacatggtgcagctgcctatcttcggcgtgatcgacaccccttgctggatcatcaaggccgctcccagctgctccgagaaggacggcaattacgcctgcctgctgagagaggaccagggctggtactgcaagaacgccggctccaccgtgtactaccccaacgacaaggactgcgagacacggggcgaccacgtgttctgtgacaccgccgctggcatcaacgtggccgagcagagcagagagtgcaacatcaacatcagcaccaccaactacccctgcaaggtgtccaccggcagacaccccatctctatggtggccctgtctcctctgggagccctggtggcctgctacaagggcgtgtcctgtagcatcggcagcaacagagtgggcatcatcaagcagctgcccaagggctgctcctacatcaccaaccaggacgccgacaccgtgaccatcgacaacaccgtgtatcagctgagcaaggtggaaggcgagcagcacgtgatcaagggcagacccgtgtccagcagcttcgaccccatcaagttccccgaggaccagttcaatgtggccctggaccaggtgttcgagagcatcgagaacagccaggctctggtggaccagtccaacaagatcctgaactccgccgagaagggcaacaccggcctggtgcctagaggcagccaccaccatcatcaccacagcgcctggtcccaccctcagttcgagaag

SEQ ID NO: 1008 - DNA, 1620 bp, Artificial Sequence
  Description of Artificial Sequence Synthetic non-optimized CDS for SEQ ID NO 912
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagacaatcaaggaagcggaggggtgttgccacagcagcagcagtcacggcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggctctgccatcggcggctatatccccgaggcccctagagatggacaggcctacgtgcggaaggatggcgagtgggtgctgctgagcacattcctgggcggcctggtgcctagaggcagccaccaccatcatcaccacagcgcctggtcccaccctcagttcgagaag

SEQ ID NO: 1009 - DNA, 1620 bp, Artificial Sequence
  Description of Artificial Sequence Synthetic codon-optimized CDS for SEQ ID NO 912
atgagctggaaagtcatgatcatcatcagcctgctgatcacccctcagcacggcctgaaagagagctacctggaagagtcctgctccaccatcaccgagggctacctgagcgtgctgagaaccggctggtacaccaacgtgttcaccctggaagtgggcgacgtggaaaacctgacctgcaccgacggccccagcctgatcaagaccgagctggatctgaccaagagcgccctgagagaactgaaaaccgtgtccgccgaccagctggccagagaggaacagatcgagaaccccagacagagcagaaagcggaggggagtggctacagctgctgccgtgacagccggcattgccattgccaagaccatccggctggaaagcgaagtgaacgccatcaaaggcgccctgaaaaccaccaacgaggccgtgtctaccctgggcaacggcgtcagagtgctggccacagccgtgcgcgagctgaaagaatttgtgtccaagaacctgaccagcgccatcaacaagaacaagtgcgacattgccgacctgaagatggccgtgtccttcagccagttcaaccggcggttcctgaacgtcgtgcggcagttcagcgacaacgccggcatcacccctgccatctccctggacctgatgaccgacgccgaactggccagggctgtgtcctacatgcctacctctgccggccagatcaagctgatgctggaaaacagagccatggtccgacggaagggcttcggcatcctgatcggcgtgtacggcagctccgtgatctacatggtgcagctgcctatcttcggcgtgatcgacaccccttgctggatcatcaaggccgctcccagctgctccgagaaggacggcaattacgcctgcctgctgagagaggaccagggctggtactgcaagaacgccggctccaccgtgtactaccccaacgacaaggactgcgagacacggggcgaccacgtgttctgtgacaccgccgctggcatcaacgtggccgagcagagcagagagtgcaacatcaacatcagcaccaccaactacccctgcaaggtgtccaccggcagacaccccatctctatggtggccctgtctcctctgggagccctggtggcctgctacaagggcgtgtcctgtagcatcggcagcaacagagtgggcatcatcaagcagctgcccaagggctgctcctacatcaccaaccaggacgccgacaccgtgaccatcgacaacaccgtgtatcagctgagcaaggtggaaggcgagcagcacgtgatcaagggcagacccgtgtccagcagcttcgaccccatcaagttccccgaggaccagttcaatgtggccctggaccaggtgttcgagagcatcgagaacagccaggctctggtggaccagtccaacaagatcctgaactccgccgagaagggcaacaccggctctgccatcggcggctatatccccgaggcccctagagatggacaggcctacgtgcggaaggatggcgagtgggtgctgctgagcacattcctgggcggcctggtgcctagaggcagccaccaccatcatcaccacagcgcctggtcccaccctcagttcgagaag

SEQ ID NO: 1010 - DNA, 1629 bp, Artificial Sequence
  Description of Artificial Sequence Synthetic non-optimized CDS for SEQ ID NO 913
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagaaggagaaggtttgtcctaggtgcaatagctcttggtgttgccacagcagcagcagtcacggcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattcccgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagctcgtatgaagcaaatcgaagacaaaatcgaagaaatcctcagcaaaatctatcatatcgagaatgagatcgctaggatcaagaagctcatcggcgaggctggttctggactggtgcctagaggcagccatcaccatcaccaccactccgcttggagccacccccaattcgagaag

SEQ ID NO: 1011 - DNA, 1629 bp, Artificial Sequence
  Description of Artificial Sequence Synthetic codon-optimized CDS for SEQ ID NO 913
atgagctggaaggtcatgatcattatttctttactgatcaccccccagcacggtttaaaggagtcctatctggaggagagctgctccaccatcacagagggatatttaagcgttttaaggaccggctggtacaccaatgtgttcactttagaggtgggcgatgtcgagaatctgacttgtacagacggaccctctttaatcaagaccgagctggacctcacaaagtccgctttaagagagctgaagacagtgagcgccgatcagctggctcgtgaggagcagatcgagaatcctcgtaggagaaggtttgtgctcggcgccatcgctctgggagtggctacagctgctgccgtgacagccggaatcgccatcgccaagaccattcgtctcgagagcgaagtcaacgccatcaagggcgccctcaagaccaccaacgaggctgtctccacactgggcaacggcgtgagagtgctggctaccgccgtgagggagctcaaggagttcgtgtccaagaatttaaccagcgccatcaacaaaaacaagtgcgacatccccgatctgaaaatggctgtcagcttcagccagttcaatcgtaggttcctcaacgtggttcgtcagtttagcgacaatgctggcattacccccgccatctctttagatttaatgaccgatgccgaactggctcgtgctgtgagctatatgcccacatccgccggacagattaagctcatgctcgaaaatcgtgccatggtgaggagaaagggcttcggcattttaatcggcgtgtatggttcttccgtcatttacatggtgcagctccccatctttggcgtgattgacaccccttgttggatcatcaaggccgctccttcttgttctgaaaaagacggcaactacgcttgtttactgagagaggatcaaggttggtattgtaagaacgccggcagcaccgtgtactaccccaatgacaaggactgcgagactcgtggcgaccacgtgttctgcgataccgctgccggcattaacgtcgccgagcagagcagagagtgcaatatcaacatcagcaccaccaactatccttgtaaggtcagcaccggtcgtcatcctatctccatggtcgctttaagccctttaggcgctctcgtggcttgttacaagggcgtgtcttgttccattggcagcaatagggtgggcatcatcaagcagctgcctaagggttgttcttatatcaccaaccaagatgccgacaccgtgacaattgataacaccgtgtaccagctgagcaaggtggagggcgagcaacacgtgatcaaaggaaggcccgtcagctcctccttcgatcccatcaaattccccgaggaccagtttaacgtggctttagaccaagttttcgagtccatcgagaattcccaagctttagtggatcagagcaacaagattttaaacagcgccgctcgtatgaagcaaatcgaagacaaaatcgaagaaatcctcagcaaaatctatcatatcgagaatgagatcgctaggatcaagaagctcatcggcgaggctggttctggactggtgcctagaggcagccatcaccatcaccaccactccgcttggagccacccccaattcgagaag

SEQ ID NO: 1012 - protein, 27 aa, Artificial Sequence
  Description of Artificial Sequence Synthetic foldon domain
GYIPEAPRDGQAYVRKDGEWVLLSTFL

SEQ ID NO: 1013 - protein, 6 aa, Artificial Sequence
  Description of Artificial Sequence Synthetic thrombin cleavage sequence
LVPRGS

SEQ ID NO: 1014 - protein, 6 aa, Artificial Sequence
  Description of Artificial Sequence Synthetic 6xHis tag
HHHHHH

SEQ ID NO: 1015 - protein, 10 aa, Artificial Sequence
  Description of Artificial Sequence Synthetic Strep II tag
SAWSHPQFEK

SEQ ID NO: 1016 - protein, 4 aa, Artificial Sequence
  Description of Artificial Sequence Synthetic Factor Xa cleavage sequence
IEGR

SEQ ID NO: 1017 - protein, 4 aa, Artificial Sequence
  Description of Artificial Sequence Synthetic Factor Xa cleavage sequence
IDGR

SEQ ID NO: 1018 - protein, 8 aa, Artificial Sequence
  Description of Artificial Sequence Synthetic cleavage sequence
LEVLFQGP

SEQ ID NO: 1019 - protein, 5 aa, Artificial Sequence
  Description of Artificial Sequence Synthetic enterokinase cleavage sequence
DDDDK

SEQ ID NO: 1020 - protein, 25 aa, Artificial Sequence
  Description of Artificial Sequence Synthetic genenase I cleavage sequence
TQLAYFTDSKNPGAAHYDTFADSLR

SEQ ID NO: 1021 - protein, 6 aa, Artificial Sequence
  Description of Artificial Sequence Synthetic TEV protease cleavage sequence
ENLYFQ

SEQ ID NO: 1022 - protein, 37 aa, Artificial Sequence
  Description of Artificial Sequence Synthetic GCN4 domain
ARMKQIEDKIEEILSKIYHIENEIARIKKLIGEAGSG

SEQ ID NO: 1023 - protein, 59 aa, Artificial Sequence
  Description of Artificial Sequence Synthetic human collagen type XV domain
GSRNLVTAFSNMDDMLQKAHLVIEGTFIYLRDSTEFFIRVRDGWKKLQLGELIPIPADS

SEQ ID NO: 1024 - protein, 58 aa, Artificial Sequence
  Description of Artificial Sequence Synthetic human collagen type XVIII domain
GVRLWATRQAMLGQVHEVPEGWLIFVAEQEELYVRVQNGFRKVQLEARTPLPRGTDNE

SEQ ID NO: 1025 - protein, 4 aa, Artificial Sequence
  Description of Artificial Sequence Synthetic peptide
  MOD_RES (2)..(2): Any amino acid
RXRR

SEQ ID NO: 1026 - protein, 4 aa, Artificial Sequence
  Description of Artificial Sequence Synthetic peptide
  MOD_RES (2)..(2): Any amino acid
RXKR

SEQ ID NO: 1027 - protein, 4 aa, Artificial Sequence
  Description of Artificial Sequence Synthetic peptide
RGRR

SEQ ID NO: 1028 - protein, 4 aa, Artificial Sequence
  Description of Artificial Sequence Synthetic peptide
RARR

SEQ ID NO: 1029 - protein, 4 aa, Artificial Sequence
  Description of Artificial Sequence Synthetic peptide
RLRR

SEQ ID NO: 1030 - protein, 4 aa, Artificial Sequence
  Description of Artificial Sequence Synthetic peptide
RMRR

SEQ ID NO: 1031 - protein, 4 aa, Artificial Sequence
  Description of Artificial Sequence Synthetic peptide
RFRR

SEQ ID NO: 1032 - protein, 4 aa, Artificial Sequence
  Description of Artificial Sequence Synthetic peptide
RWRR

SEQ ID NO: 1033 - protein, 4 aa, Artificial Sequence
  Description of Artificial Sequence Synthetic peptide
RKRR

SEQ ID NO: 1034 - protein, 4 aa, Artificial Sequence
  Description of Artificial Sequence Synthetic peptide
RQRR

SEQ ID NO: 1035 - protein, 4 aa, Artificial Sequence
  Description of Artificial Sequence Synthetic peptide
RERR

SEQ ID NO: 1036 - protein, 4 aa, Artificial Sequence
  Description of Artificial Sequence Synthetic peptide
RSRR

SEQ ID NO: 1037 - protein, 4 aa, Artificial Sequence
  Description of Artificial Sequence Synthetic peptide
RPRR

SEQ ID NO: 1038 - protein, 4 aa, Artificial Sequence
  Description of Artificial Sequence Synthetic peptide
RVRR

SEQ ID NO: 1039 - protein, 4 aa, Artificial Sequence
  Description of Artificial Sequence Synthetic peptide
RIRR

SEQ ID NO: 1040 - protein, 4 aa, Artificial Sequence
  Description of Artificial Sequence Synthetic peptide
RCRR

SEQ ID NO: 1041 - protein, 4 aa, Artificial Sequence
  Description of Artificial Sequence Synthetic peptide
RYRR

SEQ ID NO: 1042 - protein, 4 aa, Artificial Sequence
  Description of Artificial Sequence Synthetic peptide
RHRR

SEQ ID NO: 1043 - protein, 4 aa, Artificial Sequence
  Description of Artificial Sequence Synthetic peptide
RRRR

SEQ ID NO: 1044 - protein, 4 aa, Artificial Sequence
  Description of Artificial Sequence Synthetic peptide
RNRR

SEQ ID NO: 1045 - protein, 4 aa, Artificial Sequence
  Description of Artificial Sequence Synthetic peptide
RDRR

SEQ ID NO: 1046 - protein, 4 aa, Artificial Sequence
  Description of Artificial Sequence Synthetic peptide
RTRR

SEQ ID NO: 1047 - protein, 4 aa, Artificial Sequence
  Description of Artificial Sequence Synthetic peptide
RGKR

SEQ ID NO: 1048 - protein, 4 aa, Artificial Sequence
  Description of Artificial Sequence Synthetic peptide
RAKR

SEQ ID NO: 1049 - protein, 4 aa, Artificial Sequence
  Description of Artificial Sequence Synthetic peptide
RLKR

SEQ ID NO: 1050 - protein, 4 aa, Artificial Sequence
  Description of Artificial Sequence Synthetic peptide
RMKR

SEQ ID NO: 1051 - protein, 4 aa, Artificial Sequence
  Description of Artificial Sequence Synthetic peptide
RFKR

SEQ ID NO: 1052 - protein, 4 aa, Artificial Sequence
  Description of Artificial Sequence Synthetic peptide
RWKR

SEQ ID NO: 1053 - protein, 4 aa, Artificial Sequence
  Description of Artificial Sequence Synthetic peptide
RKKR

SEQ ID NO: 1054 - protein, 4 aa, Artificial Sequence
  Description of Artificial Sequence Synthetic peptide
RQKR

SEQ ID NO: 1055 - protein, 4 aa, Artificial Sequence
  Description of Artificial Sequence Synthetic peptide
REKR

SEQ ID NO: 1056 - protein, 4 aa, Artificial Sequence
  Description of Artificial Sequence Synthetic peptide
RSKR

SEQ ID NO: 1057 - protein, 4 aa, Artificial Sequence
  Description of Artificial Sequence Synthetic peptide
RPKR

SEQ ID NO: 1058 - protein, 4 aa, Artificial Sequence
  Description of Artificial Sequence Synthetic peptide
RVKR

SEQ ID NO: 1059 - protein, 4 aa, Artificial Sequence
  Description of Artificial Sequence Synthetic peptide
RIKR

SEQ ID NO: 1060 - protein, 4 aa, Artificial Sequence
  Description of Artificial Sequence Synthetic peptide
RCKR

SEQ ID NO: 1061 - protein, 4 aa, Artificial Sequence
  Description of Artificial Sequence Synthetic peptide
RYKR

SEQ ID NO: 1062 - protein, 4 aa, Artificial Sequence
  Description of Artificial Sequence Synthetic peptide
RHKR

SEQ ID NO: 1063 - protein, 4 aa, Artificial Sequence
  Description of Artificial Sequence Synthetic peptide
RRKR

SEQ ID NO: 1064 - protein, 4 aa, Artificial Sequence
  Description of Artificial Sequence Synthetic peptide
RNKR

SEQ ID NO: 1065 - protein, 4 aa, Artificial Sequence
  Description of Artificial Sequence Synthetic peptide
RDKR

SEQ ID NO: 1066 - protein, 4 aa, Artificial Sequence
  Description of Artificial Sequence Synthetic peptide
RTKR

SEQ ID NO: 1067 - protein, 4 aa, Artificial Sequence
  Description of Artificial Sequence Synthetic peptide
RQSR

(19) United States
(12) Patent Application Publication
(10) Pub. No.: US 2024/0327502 A1
(43) Pub. Date: Oct. 3, 2024

(54) ANTIBODIES THAT BIND METAPNEUMOVIRUS, ANTIGENIC METAPNEUMOVIRUS PROTEINS, AND USES THEREOF

(71) Applicant: MERCK SHARP & DOHME LLC, Rahway, NJ (US)

(72) Inventors: Zhifeng CHEN, Warrington, PA (US); Kara S. COX, Harleysville, PA (US); Arthur FRIDMAN, Lake Ariel, PA (US); Jennifer Dawn GALLI, Telford, PA (US); Hua-Poo SU, Phoenixville, PA (US); Aimin TANG, Lansdale, PA (US); Kalpit VORA, Yardley, PA (US); Zhiyun WEN, Devon, PA (US); Xiao XIAO, Wellesley, MA (US); Lan ZHANG, Chalfont, PA (US)

(73) Assignee: MERCK SHARP & DOHME LLC, Rahway, NJ (US)

(21) Appl. No.: 18/264,824

(22) PCT Filed: Feb. 8, 2022
(86) PCT No.: PCT/US2022/015565

§ 371 Date: Aug. 9, 2023

Related U.S. Application Data
(60) Provisional application No. 63/148,920, filed on Feb. 12, 2021.
(60) Provisional application No. 63/276,172, filed on Nov. 5, 2021.

(51) Int. Cl. C07K 16/10; A61K 39/00; A61K 39/12; A61P 31/14; C07K 14/005; C12N 7/00

(52) U.S. Cl. CPC C07K 16/10; A61K 39/12; A61P 31/14; C07K 14/005; C12N 7/00; A61K 2039/572; C07K 2317/565; C07K 2317/76; C07K 2317/92; C12N 2760/18322; C12N 2760/18334

(57) ABSTRACT

The present invention relates to anti-human metapneumovirus (hMPV) antibodies, as well as use of such antibodies in the treatment of viral infections. The invention also includes antigenic hMPV proteins and immunogenic compositions comprising such antigenic hMPV proteins, and the use of such antigenic hMPV proteins and related compositions for preventing or treating viral infection in a subject.

### FIELD OF THE INVENTION

The present invention relates to antibodies or antigen binding fragments thereof that specifically bind and/or neutralize human metapneumovirus. Another aspect of the invention relates to compositions and kits comprising the antibodies or antigen binding fragments thereof. Another aspect of the invention relates to methods for treating diseases, for example respiratory infections, by administering the antibodies or antigen binding fragments thereof. Another aspect of the invention relates to antigenic human metapneumovirus F proteins. Another aspect of the invention relates to methods for preventing or treating diseases, for example respiratory infections, by administering antigenic human metapneumovirus F proteins.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Jan. 27, 2022, is named 25170-WO-PCT_SL.txt and is 495,474 bytes in size.

### BACKGROUND OF THE INVENTION

Human metapneumovirus (hMPV) belongs to the Pneumoviridae family and is closely related to respiratory syncytial virus (RSV). It is a major pathogen causing acute lower respiratory infection in young children, the elderly and immunocompromised adults. HMPV encodes two major surface glycoproteins, the fusion (F) and glycoprotein (G) proteins. The surface F glycoprotein, which is matured after a proteolytic cleavage process, mediates viral fusion by structure rearrangements and is the primary target of neutralizing antibodies against hMPV. Current understanding of the specificity and functionality of hMPV-F antibody responses induced by natural infection remains elusive. Therapeutic treatments to treat diseases associated with hMPV are needed.

### SUMMARY OF INVENTION

The present disclosure provides antibodies and antigen binding fragments thereof capable of bindings to and/or neutralizing metapneumovirus (MPV), and methods, pharmaceutical compositions, uses and kits of binding to and/or neutralizing an MPV using an antibody or antigen binding fragment thereof. In various embodiments, the MPV is mammalian. For example, the mammalian MPV comprises a human MPV (hMPV). In various embodiments, the hMPV comprises a surface glycoprotein. For example, the glycoprotein comprises a F glycoprotein and/or a G glycoprotein.

The present disclosure provides an antibody or antigen binding fragment thereof which specifically binds to hMPV comprising at least one CDR, three CDRs or six CDRs described in Tables 1-3. The present disclosure provides an antibody or antigen binding fragment thereof which specifically binds to hMPV comprising at least one variable region/domain described in Tables 1-3. For example, the antibody or antigen binding fragment thereof which specifically binds to hMPV comprises a heavy chain variable region, light chain variable region, or heavy chain variable region and light chain variable region.

An aspect of the invention provides an antibody or antigen binding fragment thereof which specifically binds hMPV.

In various embodiments, the antibody or antigen binding fragment thereof comprises a variable region comprising three heavy chain complementarity determining regions (CDRs), i.e., CDR1, CDR2, and CDR3. In various embodiments, the antibody or antigen binding fragment thereof comprises a variable region comprising any one CDR comprising the amino acid sequences of SEQ ID NOs: 2-340. In various embodiments, the antibody or antigen binding fragment thereof comprises a variable region comprising three light chain CDRs CDR1, CDR2, and CDR3 comprising amino acid sequence selected from the group consisting of: SEQ ID NOs: 341-649.

In various embodiments, the antibody or antigen binding fragment thereof comprises a CDR1 comprising an amino acid sequence selected from the group consisting of SEQ ID NOs: 2, 5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, 260, 263, 266, 269, 272, 275, 278, 281, 284, 287, 290, 293, 296, 299, 302, 305, 308, 311, 314, 317, 320, 323, 326, 329, 332, 335, and 338. In various embodiments, the CDR1 is a heavy chain variable region CDR1.

In various embodiments, the antibody or antigen binding fragment thereof comprises a CDR2 comprising an amino acid sequence selected from the group consisting of SEQ ID NOs: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, and 339. In various embodiments, the CDR2 is a heavy chain variable region CDR2.

In various embodiments, the antibody or antigen binding fragment thereof comprises a CDR3 comprising an amino acid sequence selected from the group consisting of SEQ ID NOs: 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, 259, 262, 265, 268, 271, 274, 277, 280, 283, 286, 289, 292, 295, 298, 301, 304, 307, 310, 313, 316, 319, 322, 325, 328, 331, 334, 337, and 340. In various embodiments, the CDR3 is a heavy chain variable region CDR3.

In various embodiments, the antibody or antigen binding fragment thereof comprises at least one heavy chain variable region and/or at least one light chain variable region.

In various embodiments, the antibody or antigen binding fragment thereof comprises a heavy chain variable region comprising three CDRs selected from the group consisting of:
(a) a variable region comprising CDR1, CDR2, and CDR3 regions comprising the amino acid sequences of SEQ ID NOs: 2, 3, and 4, respectively;
(b) a variable region comprising CDR1, CDR2, and CDR3 regions comprising the amino acid sequences of SEQ ID NOs: 5, 6, and 7, respectively;
(c) a variable region comprising CDR1, CDR2, and CDR3 regions comprising the amino acid sequences of SEQ ID NOs: 8, 9, and 10, respectively;
(d) a variable region comprising CDR1, CDR2, and CDR3 regions comprising the amino acid sequences of SEQ ID NOs: 11, 12, and 13, respectively;
(e) a variable region comprising CDR1, CDR2, and CDR3 regions comprising the amino acid sequences of SEQ ID NOs: 14, 15, and 16, respectively;
(f) a variable region comprising CDR1, CDR2, and CDR3 regions comprising the amino acid sequences of SEQ ID NOs: 17, 18, and 19, respectively;
(g) a variable region comprising CDR1, CDR2, and CDR3 regions comprising the amino acid sequences of SEQ ID NOs: 20, 21, and 22, respectively;
(h) a variable region comprising CDR1, CDR2, and CDR3 regions comprising the amino acid sequences of SEQ ID NOs: 23, 24, and 25, respectively;
(i) a variable region comprising CDR1, CDR2, and CDR3 regions comprising the amino acid sequences of SEQ ID NOs: 26, 27, and 28, respectively;
(j) a variable region comprising CDR1, CDR2, and CDR3 regions comprising the amino acid sequences of SEQ ID NOs: 29, 30, and 31, respectively;
(k) a variable region comprising CDR1, CDR2, and CDR3 regions comprising the amino acid sequences of SEQ ID NOs: 32, 33, and 34, respectively;
(l) a variable region comprising CDR1, CDR2, and CDR3 regions comprising the amino acid sequences of SEQ ID NOs: 35, 36, and 37, respectively;
(m) a variable region comprising CDR1, CDR2, and CDR3 regions comprising the amino acid sequences of SEQ ID NOs: 38, 39, and 40, respectively;
(n) a variable region comprising CDR1, CDR2, and CDR3 regions comprising the amino acid sequences of SEQ ID NOs: 41, 42, and 43, respectively;
(o) a variable region comprising CDR1, CDR2, and CDR3 regions comprising the amino acid sequences of SEQ ID NOs: 44, 45, and 46, respectively;
(p) a variable region comprising CDR1, CDR2, and CDR3 regions comprising the amino acid sequences of SEQ ID NOs: 47, 48, and 49, respectively;
(q) a variable region comprising CDR1, CDR2, and CDR3 regions comprising the amino acid sequences of SEQ ID NOs: 50, 51, and 52, respectively;
(r) a variable region comprising CDR1, CDR2, and CDR3 regions comprising the amino acid sequences of SEQ ID NOs: 53, 54, and 55, respectively;
(s) a variable region comprising CDR1, CDR2, and CDR3 regions comprising the amino acid sequences of SEQ ID NOs: 56, 57, and 58, respectively;
(t) a variable region comprising CDR1, CDR2, and CDR3 regions comprising the amino acid sequences of SEQ ID NOs: 59, 60, and 61, respectively;
(u) a variable region comprising CDR1, CDR2, and CDR3 regions comprising the amino acid sequences of SEQ ID NOs: 62, 63, and 64, respectively;
(v) a variable region comprising CDR1, CDR2, and CDR3 regions comprising the amino acid sequences of SEQ ID NOs: 65, 66, and 67, respectively;
(w) a variable region comprising CDR1, CDR2, and CDR3 regions comprising the amino acid sequences of SEQ ID NOs: 68, 69, and 70, respectively;
(x) a variable region comprising CDR1, CDR2, and CDR3 regions comprising the amino acid sequences of SEQ ID NOs: 71, 72, and 73, respectively;
(y) a variable region comprising CDR1, CDR2, and CDR3 regions comprising the amino acid sequences of SEQ ID NOs: 74, 75, and 76, respectively;
(z) a variable region comprising CDR1, CDR2, and CDR3 regions comprising the amino acid sequences of SEQ ID NOs: 77, 78, and 79, respectively;
(aa) a variable region comprising CDR1, CDR2, and CDR3 regions comprising the amino acid sequences of SEQ ID NOs: 80, 81, and 82, respectively;
(bb) a variable region comprising CDR1, CDR2, and CDR3 regions comprising the amino acid sequences of SEQ ID NOs: 83, 84, and 85, respectively;
(cc) a variable region comprising CDR1, CDR2, and CDR3 regions comprising the amino acid sequences of SEQ ID NOs: 86, 87, and 88, respectively;
(dd) a variable region comprising CDR1, CDR2, and CDR3 regions comprising the amino acid sequences of SEQ ID NOs: 89, 90, and 91, respectively;
(ee) a variable region comprising CDR1, CDR2, and CDR3 regions comprising the amino acid sequences of SEQ ID NOs: 92, 93, and 94, respectively;
(ff) a variable region comprising CDR1, CDR2, and CDR3 regions comprising the amino acid sequences of SEQ ID NOs: 95, 96, and 97, respectively;
(gg) a variable region comprising CDR1, CDR2, and CDR3 regions comprising the amino acid sequences of SEQ ID NOs: 98, 99, and 100, respectively;
(hh) a variable region comprising CDR1, CDR2, and CDR3 regions comprising the amino acid sequences of SEQ ID NOs: 101, 102, and 103, respectively;
(ii) a variable region comprising CDR1, CDR2, and CDR3 regions comprising the amino acid sequences of SEQ ID NOs: 104, 105, and 106; respectively;
(jj) a variable region comprising CDR1, CDR2, and CDR3 regions comprising the amino acid sequences of SEQ ID NOs: 107, 108, and 109, respectively;
(kk) a variable region comprising CDR1, CDR2, and CDR3 regions comprising the amino acid sequences of SEQ ID NOs: 110, 111, and 112, respectively;
(ll) a variable region comprising CDR1, CDR2, and CDR3 regions comprising the amino acid sequences of SEQ ID NOs: 113, 114, and 115, respectively;
(mm) a variable region comprising CDR1, CDR2, and CDR3 regions comprising the amino acid sequences of SEQ ID NOs: 116, 117, and 118, respectively;
(nn) a variable region comprising CDR1, CDR2, and CDR3 regions comprising the amino acid sequences of SEQ ID NOs: 119, 120, and 121, respectively;
(oo) a variable region comprising CDR1, CDR2, and CDR3 regions comprising the amino acid sequences of SEQ ID NOs: 122, 123, and 124, respectively;
(pp) a variable region comprising CDR1, CDR2, and CDR3 regions comprising the amino acid sequences of SEQ ID NOs: 125, 126, and 127, respectively;
(qq) a variable region comprising CDR1, CDR2, and CDR3 regions comprising the amino acid sequences of SEQ ID NOs: 128, 129, and 130, respectively;
(rr) a variable region comprising CDR1, CDR2, and CDR3 regions comprising the amino acid sequences of SEQ ID NOs: 131, 132, and 133, respectively;
(ss) a variable region comprising CDR1, CDR2, and CDR3 regions comprising the amino acid sequences of SEQ ID NOs: 134, 135, and 136, respectively;
(tt) a variable region comprising CDR1, CDR2, and CDR3 regions comprising the amino acid sequences of SEQ ID NOs: 137, 138, and 139, respectively;
(uu) a variable region comprising CDR1, CDR2, and CDR3 regions comprising the amino acid sequences of SEQ ID NOs: 140, 141, and 142, respectively;
(vv) a variable region comprising CDR1, CDR2, and CDR3 regions comprising the amino acid sequences of SEQ ID NOs: 143, 144, and 145, respectively;
(ww) a variable region comprising CDR1, CDR2, and CDR3 regions comprising the amino acid sequences of SEQ ID NOs: 146, 147, and 148, respectively;
(xx) a variable region comprising CDR1, CDR2, and CDR3 regions comprising the amino acid sequences of SEQ ID NOs: 149, 150, and 151, respectively;
(yy) a variable region comprising CDR1, CDR2, and CDR3 regions comprising the amino acid sequences of SEQ ID NOs: 152, 153, and 154, respectively;
(zz) a variable region comprising CDR1, CDR2, and CDR3 regions comprising the amino acid sequences of SEQ ID NOs: 155, 156, and 157, respectively;
(aaa) a variable region comprising CDR1, CDR2, and CDR3 regions comprising the amino acid sequences of SEQ ID NOs: 158, 159, and 160, respectively;
(bbb) a variable region comprising CDR1, CDR2, and CDR3 regions comprising the amino acid sequences of SEQ ID NOs: 161, 162, and 163, respectively;
(ccc) a variable region comprising CDR1, CDR2, and CDR3 regions comprising the amino acid sequences of SEQ ID NOs: 164, 165, and 166, respectively;
(ddd) a variable region comprising CDR1, CDR2, and CDR3 regions comprising the amino acid sequences of SEQ ID NOs: 167, 168, and 169, respectively;
(eee) a variable region comprising CDR1, CDR2, and CDR3 regions comprising the amino acid sequences of SEQ ID NOs: 170, 171, and 172, respectively;
(fff) a variable region comprising CDR1, CDR2, and CDR3 regions comprising the amino acid sequences of SEQ ID NOs: 173, 174, and 175, respectively;
(ggg) a variable region comprising CDR1, CDR2, and CDR3 regions comprising the amino acid sequences of SEQ ID NOs: 176, 177, and 178, respectively;
(hhh) a variable region comprising CDR1, CDR2, and CDR3 regions comprising the amino acid sequences of SEQ ID NOs: 179, 180, and 181, respectively;
(iii) a variable region comprising CDR1, CDR2, and CDR3 regions comprising the amino acid sequences of SEQ ID NOs: 182, 183, and 184, respectively;
(jjj) a variable region comprising CDR1, CDR2, and CDR3 regions comprising the amino acid sequences of SEQ ID NOs: 185, 186, and 187, respectively;
(kkk) a variable region comprising CDR1, CDR2, and CDR3 regions comprising the amino acid sequences of SEQ ID NOs: 188, 189, and 190, respectively;
(lll) a variable region comprising CDR1, CDR2, and CDR3 regions comprising the amino acid sequences of SEQ ID NOs: 191, 192, and 193, respectively;
(mmm) a variable region comprising CDR1, CDR2, and CDR3 regions comprising the amino acid sequences of SEQ ID NOs: 194, 195, and 196, respectively;
(nnn) a variable region comprising CDR1, CDR2, and CDR3 regions comprising the amino acid sequences of SEQ ID NOs: 197, 198, and 199, respectively;
(ooo) a variable region comprising CDR1, CDR2, and CDR3 regions comprising the amino acid sequences of SEQ ID NOs: 200, 201, and 202, respectively;
(ppp) a variable region comprising CDR1, CDR2, and CDR3 regions comprising the amino acid sequences of SEQ ID NOs: 203, 204, and 205, respectively;
(qqq) a variable region comprising CDR1, CDR2, and CDR3 regions comprising the amino acid sequences of SEQ ID NOs: 206, 207, and 208, respectively;
(rrr) a variable region comprising CDR1, CDR2, and CDR3 regions comprising the amino acid sequences of SEQ ID NOs: 209, 210, and 211, respectively;
(sss) a variable region comprising CDR1, CDR2, and CDR3 regions comprising the amino acid sequences of SEQ ID NOs: 212, 213, and 214, respectively;
(ttt) a variable region comprising CDR1, CDR2, and CDR3 regions comprising the amino acid sequences of SEQ ID NOs: 215, 216, and 217, respectively;
(uuu) a variable region comprising CDR1, CDR2, and CDR3 regions comprising the amino acid sequences of SEQ ID NOs: 218, 219, and 220, respectively;
(vvv) a variable region comprising CDR1, CDR2, and CDR3 regions comprising the amino acid sequences of SEQ ID NOs: 221, 222, and 223, respectively;
(www) a variable region comprising CDR1, CDR2, and CDR3 regions comprising the amino acid sequences of SEQ ID NOs: 224, 225, and 226, respectively;
(xxx) a variable region comprising CDR1, CDR2, and CDR3 regions comprising the amino acid sequences of SEQ ID NOs: 227, 228, and 229, respectively;
(yyy) a variable region comprising CDR1, CDR2, and CDR3 regions comprising the amino acid sequences of SEQ ID NOs: 230, 231, and 232, respectively;
(zzz) a variable region comprising CDR1, CDR2, and CDR3 regions comprising the amino acid sequences of SEQ ID NOs: 233, 234, and 235, respectively;
(aaaa) a variable region comprising CDR1, CDR2, and CDR3 regions comprising the amino acid sequences of SEQ ID NOs: 236, 237, and 238, respectively;
(bbbb) a variable region comprising CDR1, CDR2, and CDR3 regions comprising the amino acid sequences of SEQ ID NOs: 239, 240, and 241, respectively;
(cccc) a variable region comprising CDR1, CDR2, and CDR3 regions comprising the amino acid sequences of SEQ ID NOs: 242, 243, and 244, respectively;
(dddd) a variable region comprising CDR1, CDR2, and CDR3 regions comprising the amino acid sequences of SEQ ID NOs: 245, 246, and 247, respectively;
(eeee) a variable region comprising CDR1, CDR2, and CDR3 regions comprising the amino acid sequences of SEQ ID NOs: 248, 249, and 250, respectively;
(ffff) a variable region comprising CDR1, CDR2, and CDR3 regions comprising the amino acid sequences of SEQ ID NOs: 251, 252, and 253, respectively;
(gggg) a variable region comprising CDR1, CDR2, and CDR3 regions comprising the amino acid sequences of SEQ ID NOs: 254, 255, and 256, respectively;
(hhhh) a variable region comprising CDR1, CDR2, and CDR3 regions comprising the amino acid sequences of SEQ ID NOs: 257, 258, and 259, respectively;
(iiii) a variable region comprising CDR1, CDR2, and CDR3 regions comprising the amino acid sequences of SEQ ID NOs: 260, 261, and 262, respectively;
(jjjj) a variable region comprising CDR1, CDR2, and CDR3 regions comprising the amino acid sequences of SEQ ID NOs: 263, 264, and 265, respectively;
(kkkk) a variable region comprising CDR1, CDR2, and CDR3 regions comprising the amino acid sequences of SEQ ID NOs: 266, 267, and 268, respectively;
(llll) a variable region comprising CDR1, CDR2, and CDR3 regions comprising the amino acid sequences of SEQ ID NOs: 269, 270, and 271, respectively;
(mmmm) a variable region comprising CDR1, CDR2, and CDR3 regions comprising the amino acid sequences of SEQ ID NOs: 272, 273, and 274, respectively;
(nnnn) a variable region comprising CDR1, CDR2, and CDR3 regions comprising the amino acid sequences of SEQ ID NOs: 275, 276, and 277, respectively;
(oooo) a variable region comprising CDR1, CDR2, and CDR3 regions comprising the amino acid sequences of SEQ ID NOs: 278, 279, and 280, respectively;
(pppp) a variable region comprising CDR1, CDR2, and CDR3 regions comprising the amino acid sequences of SEQ ID NOs: 281, 282, and 283, respectively;
(qqqq) a variable region comprising CDR1, CDR2, and CDR3 regions comprising the amino acid sequences of SEQ ID NOs: 284, 285, and 286, respectively;
(rrrr) a variable region comprising CDR1, CDR2, and CDR3 regions comprising the amino acid sequences of SEQ ID NOs: 287, 288, and 289, respectively;
(ssss) a variable region comprising CDR1, CDR2, and CDR3 regions comprising the amino acid sequences of SEQ ID NOs: 290, 291, and 292, respectively;
(tttt) a variable region comprising CDR1, CDR2, and CDR3 regions comprising the amino acid sequences of SEQ ID NOs: 293, 294, and 295, respectively;
(uuuu) a variable region comprising CDR1, CDR2, and CDR3 regions comprising the amino acid sequences of SEQ ID NOs: 296, 297, and 298, respectively;
(vvvv) a variable region comprising CDR1, CDR2, and CDR3 regions comprising the amino acid sequences of SEQ ID NOs: 299, 300, and 301, respectively;
(wwww) a variable region comprising CDR1, CDR2, and CDR3 regions comprising the amino acid sequences of SEQ ID NOs: 302, 303, and 304, respectively;
(xxxx) a variable region comprising CDR1, CDR2, and CDR3 regions comprising the amino acid sequences of SEQ ID NOs: 305, 306, and 307, respectively;
(yyyy) a variable region comprising CDR1, CDR2, and CDR3 regions comprising the amino acid sequences of SEQ ID NOs: 308, 309, and 310, respectively;
(zzzz) a variable region comprising CDR1, CDR2, and CDR3 regions comprising the amino acid sequences of SEQ ID NOs: 311, 312, and 313, respectively;
(aaaaa) a variable region comprising CDR1, CDR2, and CDR3 regions comprising the amino acid sequences of SEQ ID NOs: 314, 315, and 316, respectively;
(bbbbb) a variable region comprising CDR1, CDR2, and CDR3 regions comprising the amino acid sequences of SEQ ID NOs: 317, 318, and 319, respectively;
(ccccc) a variable region comprising CDR1, CDR2, and CDR3 regions comprising the amino acid sequences of SEQ ID NOs: 320, 321, and 322, respectively;
(ddddd) a variable region comprising CDR1, CDR2, and CDR3 regions comprising the amino acid sequences of SEQ ID NOs: 323, 324, and 325, respectively;
(eeeee) a variable region comprising CDR1, CDR2, and CDR3 regions comprising the amino acid sequences of SEQ ID NOs: 326, 327, and 328, respectively;
(fffff) a variable region comprising CDR1, CDR2, and CDR3 regions comprising the amino acid sequences of SEQ ID NOs: 329, 330, and 331, respectively;
(ggggg) a variable region comprising CDR1, CDR2, and CDR3 regions comprising the amino acid sequences of SEQ ID NOs: 332, 333, and 334, respectively;
(hhhhh) a variable region comprising CDR1, CDR2, and CDR3 regions comprising the amino acid sequences of SEQ ID NOs: 335, 336, and 337, respectively; and
(iiiii) a variable region comprising CDR1, CDR2, and CDR3 regions comprising the amino acid sequences of SEQ ID NOs: 338, 339, and 340, respectively.

In various embodiments, the antibody or antigen binding fragment thereof comprises a CDR1 comprising an amino acid sequence selected from the group consisting of SEQ ID NOs: 341, 344, 347, 350, 353, 356, 359, 362, 365, 368, 371, 374, 377, 380, 383, 386, 389, 392, 395, 398, 401, 404, 407, 410, 413, 416, 419, 422, 425, 428, 431, 434, 437, 440, 443, 446, 449, 452, 455, 458, 461, 464, 467, 470, 473, 476, 479, 482, 485, 488, 491, 494, 497, 500, 503, 506, 509, 512, 515, 518, 521, 524, 527, 530, 533, 536, 539, 542, 545, 548, 551, 554, 557, 560, 563, 566, 569, 572, 575, 578, 581, 584, 587, 590, 593, 596, 599, 602, 605, 608, 611, 614, 617, 620, 623, 626, 629, 632, 635, 638, 641, 644, 647, 650, 653, 656, 659, 662, 665, 668, 671, 674, and 677. In various embodiments, the CDR1 is a light chain variable region CDR1.

In various embodiments, the antibody or antigen binding fragment thereof comprises a CDR2 comprising an amino acid sequence selected from the group consisting of SEQ ID NOs: 342, 345, 348, 351, 354, 357, 360, 363, 366, 369, 372, 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, 486, 489, 492, 495, 498, 501, 504, 507, 510, 513, 516, 519, 522, 525, 528, 531, 534, 537, 540, 543, 546, 549, 552, 555, 558, 561, 564, 567, 570, 573, 576, 579, 582, 585, 588, 591, 594, 597, 600, 603, 606, 609, 612, 615, 618, 621, 624, 627, 630, 633, 636, 639, 642, 645, 648, 651, 654, 657, 660, 663, 666, 669, 672, 675, and 678. In various embodiments, the CDR2 is a light chain variable region CDR2.

In various embodiments, the antibody or antigen binding fragment thereof comprises a CDR3 comprising an amino acid sequence selected from the group consisting of SEQ ID NOs: 343, 346, 349, 352, 355, 358, 361, 364, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 520, 523, 526, 529, 532, 535, 538, 541, 544, 547, 550, 553, 556, 559, 562, 565, 568, 571, 574, 577, 580, 583, 586, 589, 592, 595, 598, 601, 604, 607, 610, 613, 616, 619, 622, 625, 628, 631, 634, 637, 640, 643, 646, 649, 652, 655, 658, 661, 664, 667, 670, 673, 676, and 679. In various embodiments, the CDR3 is a light chain variable region CDR3.

In various embodiments, the antibody or antigen binding fragment thereof comprises a light chain variable region comprising three CDRs selected from the group consisting of:
(a) a variable region comprising CDR1, CDR2, and CDR3 regions comprising the amino acid sequences of SEQ ID NOs: 341, 342, and 343, respectively;
(b) a variable region comprising CDR1, CDR2, and CDR3 regions comprising the amino acid sequences of SEQ ID NOs: 344, 345, and 346, respectively;
(c) a variable region comprising CDR1, CDR2, and CDR3 regions comprising the amino acid sequences of SEQ ID NOs: 347, 348, and 349, respectively;
(d) a variable region comprising CDR1, CDR2, and CDR3 regions comprising the amino acid sequences of SEQ ID NOs: 350, 351, and 352, respectively;
(e) a variable region comprising CDR1, CDR2, and CDR3 regions comprising the amino acid sequences of SEQ ID NOs: 353, 354, and 355, respectively;
(f) a variable region comprising CDR1, CDR2, and CDR3 regions comprising the amino acid sequences of SEQ ID NOs: 356, 357, and 358, respectively;
(g) a variable region comprising CDR1, CDR2, and CDR3 regions comprising the amino acid sequences of SEQ ID NOs: 359, 360, and 361, respectively;
(h) a variable region comprising CDR1, CDR2, and CDR3 regions comprising the amino acid sequences of SEQ ID NOs: 362, 363, and 364, respectively;
(i) a variable region comprising CDR1, CDR2, and CDR3 regions comprising the amino acid sequences of SEQ ID NOs: 365, 366, and 367, respectively;
(j) a variable region comprising CDR1, CDR2, and CDR3 regions comprising the amino acid sequences of SEQ ID NOs: 368, 369, and 370, respectively;
(k) a variable region comprising CDR1, CDR2, and CDR3 regions comprising the amino acid sequences of SEQ ID NOs: 371, 372, and 373, respectively;
(l) a variable region comprising CDR1, CDR2, and CDR3 regions comprising the amino acid sequences of SEQ ID NOs: 374, 375, and 376, respectively;
(m) a variable region comprising CDR1, CDR2, and CDR3 regions comprising the amino acid sequences of SEQ ID NOs: 377, 378, and 379, respectively;
(n) a variable region comprising CDR1, CDR2, and CDR3 regions comprising the amino acid sequences of SEQ ID NOs: 380, 381, and 382, respectively;
(o) a variable region comprising CDR1, CDR2, and CDR3 regions comprising the amino acid sequences of SEQ ID NOs: 383, 384, and 385, respectively;
(p) a variable region comprising CDR1, CDR2, and CDR3 regions comprising the amino acid sequences of SEQ ID NOs: 386, 387, and 388, respectively;
(q) a variable region comprising CDR1, CDR2, and CDR3 regions comprising the amino acid sequences of SEQ ID NOs: 389, 390, and 391, respectively;
(r) a variable region comprising CDR1, CDR2, and CDR3 regions comprising the amino acid sequences of SEQ ID NOs: 392, 393, and 394, respectively;
(s) a variable region comprising CDR1, CDR2, and CDR3 regions comprising the amino acid sequences of SEQ ID NOs: 395, 396, and 397, respectively;
(t) a variable region comprising CDR1, CDR2, and CDR3 regions comprising the amino acid sequences of SEQ ID NOs: 398, 399, and 400, respectively;
(u) a variable region comprising CDR1, CDR2, and CDR3 regions comprising the amino acid sequences of SEQ ID NOs: 401, 402, and 403, respectively;
(v) a variable region comprising CDR1, CDR2, and CDR3 regions comprising the amino acid sequences of SEQ ID NOs: 404, 405, and 406, respectively;
(w) a variable region comprising CDR1, CDR2, and CDR3 regions comprising the amino acid sequences of SEQ ID NOs: 407, 408, and 409, respectively;
(x) a variable region comprising CDR1, CDR2, and CDR3 regions comprising the amino acid sequences of SEQ ID NOs: 410, 411, and 412, respectively;
(y) a variable region comprising CDR1, CDR2, and CDR3 regions comprising the amino acid sequences of SEQ ID NOs: 413, 414, and 415, respectively;
(z) a variable region comprising CDR1, CDR2, and CDR3 regions comprising the amino acid sequences of SEQ ID NOs: 416, 417, and 418, respectively;
(aa) a variable region comprising CDR1, CDR2, and CDR3 regions comprising the amino acid sequences of SEQ ID NOs: 419, 420, and 421, respectively;
(bb) a variable region comprising CDR1, CDR2, and CDR3 regions comprising the amino acid sequences of SEQ ID NOs: 422, 423, and 424, respectively;
(cc) a variable region comprising CDR1, CDR2, and CDR3 regions comprising the amino acid sequences of SEQ ID NOs: 425, 426, and 427, respectively;
(dd) a variable region comprising CDR1, CDR2, and CDR3 regions comprising the amino acid sequences of SEQ ID NOs: 428, 429, and 430, respectively;
(ee) a variable region comprising CDR1, CDR2, and CDR3 regions comprising the amino acid sequences of SEQ ID NOs: 431, 432, and 433, respectively;
(ff) a variable region comprising CDR1, CDR2, and CDR3 regions comprising the amino acid sequences of SEQ ID NOs: 434, 435, and 436, respectively;
(gg) a variable region comprising CDR1, CDR2, and CDR3 regions comprising the amino acid sequences of SEQ ID NOs: 437, 438, and 439, respectively;
(hh) a variable region comprising CDR1, CDR2, and CDR3 regions comprising the amino acid sequences of SEQ ID NOs: 440, 441, and 442, respectively;
(ii) a variable region comprising CDR1, CDR2, and CDR3 regions comprising the amino acid sequences of SEQ ID NOs: 443, 444, and 445, respectively;
(jj) a variable region comprising CDR1, CDR2, and CDR3 regions comprising the amino acid sequences of SEQ ID NOs: 446, 447, and 448, respectively;
(kk) a variable region comprising CDR1, CDR2, and CDR3 regions comprising the amino acid sequences of SEQ ID NOs: 449, 450, and 451, respectively;
(ll) a variable region comprising CDR1, CDR2, and CDR3 regions comprising the amino acid sequences of SEQ ID NOs: 452, 453, and 454, respectively;
(mm) a variable region comprising CDR1, CDR2, and CDR3 regions comprising the amino acid sequences of SEQ ID NOs: 455, 456, and 457, respectively;
(nn) a variable region comprising CDR1, CDR2, and CDR3 regions comprising the amino acid sequences of SEQ ID NOs: 458, 459, and 460, respectively;
(oo) a variable region comprising CDR1, CDR2, and CDR3 regions comprising the amino acid sequences of SEQ ID NOs: 461, 462, and 463, respectively;
(pp) a variable region comprising CDR1, CDR2, and CDR3 regions comprising the amino acid sequences of SEQ ID NOs: 464, 465, and 466, respectively;
(qq) a variable region comprising CDR1, CDR2, and CDR3 regions comprising the amino acid sequences of SEQ ID NOs: 467, 468, and 469, respectively;
(rr) a variable region comprising CDR1, CDR2, and CDR3 regions comprising the amino acid sequences of SEQ ID NOs: 470, 471, and 472, respectively;
(ss) a variable region comprising CDR1, CDR2, and CDR3 regions comprising the amino acid sequences of SEQ ID NOs: 473, 474, and 475, respectively;
(tt) a variable region comprising CDR1, CDR2, and CDR3 regions comprising the amino acid sequences of SEQ ID NOs: 476, 477, and 478, respectively;
(uu) a variable region comprising CDR1, CDR2, and CDR3 regions comprising the amino acid sequences of SEQ ID NOs: 479, 480, and 481, respectively;
(vv) a variable region comprising CDR1, CDR2, and CDR3 regions comprising the amino acid sequences of SEQ ID NOs: 482, 483, and 484, respectively;
(ww) a variable region comprising CDR1, CDR2, and CDR3 regions comprising the amino acid sequences of SEQ ID NOs: 485, 486, and 487, respectively;
(xx) a variable region comprising CDR1, CDR2, and CDR3 regions comprising the amino acid sequences of SEQ ID NOs: 488, 489, and 490, respectively;
(yy) a variable region comprising CDR1, CDR2, and CDR3 regions comprising the amino acid sequences of SEQ ID NOs: 491, 492, and 493, respectively;
(zz) a variable region comprising CDR1, CDR2, and CDR3 regions comprising the amino acid sequences of SEQ ID NOs: 494, 495, and 496, respectively;
(aaa) a variable region comprising CDR1, CDR2, and CDR3 regions comprising the amino acid sequences of SEQ ID NOs: 497, 498, and 499, respectively;
(bbb) a variable region comprising CDR1, CDR2, and CDR3 regions comprising the amino acid sequences of SEQ ID NOs: 500, 501, and 502, respectively;
(ccc) a variable region comprising CDR1, CDR2, and CDR3 regions comprising the amino acid sequences of SEQ ID NOs: 503, 504, and 505, respectively;
(ddd) a variable region comprising CDR1, CDR2, and CDR3 regions comprising the amino acid sequences of SEQ ID NOs: 506, 507, and 508, respectively;
(eee) a variable region comprising CDR1, CDR2, and CDR3 regions comprising the amino acid sequences of SEQ ID NOs: 509, 510, and 511, respectively;
(fff) a variable region comprising CDR1, CDR2, and CDR3 regions comprising the amino acid sequences of SEQ ID NOs: 512, 513, and 514, respectively;
(ggg) a variable region comprising CDR1, CDR2, and CDR3 regions comprising the amino acid sequences of SEQ ID NOs: 515, 516, and 517, respectively;
(hhh) a variable region comprising CDR1, CDR2, and CDR3 regions comprising the amino acid sequences of SEQ ID NOs: 518, 519, and 520, respectively;
(iii) a variable region comprising CDR1, CDR2, and CDR3 regions comprising the amino acid sequences of SEQ ID NOs: 521, 522, and 523, respectively;
(jjj) a variable region comprising CDR1, CDR2, and CDR3 regions comprising the amino acid sequences of SEQ ID NOs: 524, 525, and 526, respectively;
(kkk) a variable region comprising CDR1, CDR2, and CDR3 regions comprising the amino acid sequences of SEQ ID NOs: 527, 528, and 529, respectively;
(lll) a variable region comprising CDR1, CDR2, and CDR3 regions comprising the amino acid sequences of SEQ ID NOs: 530, 531, and 532, respectively;
(mmm) a variable region comprising CDR1, CDR2, and CDR3 regions comprising the amino acid sequences of SEQ ID NOs: 533, 534, and 535, respectively;
(nnn) a variable region comprising CDR1, CDR2, and CDR3 regions comprising the amino acid sequences of SEQ ID NOs: 536, 537, and 538, respectively;
(ooo) a variable region comprising CDR1, CDR2, and CDR3 regions comprising the amino acid sequences of SEQ ID NOs: 539, 540, and 541, respectively;
(ppp) a variable region comprising CDR1, CDR2, and CDR3 regions comprising the amino acid sequences of SEQ ID NOs: 542, 543, and 544, respectively;
(qqq) a variable region comprising CDR1, CDR2, and CDR3 regions comprising the amino acid sequences of SEQ ID NOs: 545, 546, and 547, respectively;
(rrr) a variable region comprising CDR1, CDR2, and CDR3 regions comprising the amino acid sequences of SEQ ID NOs: 548, 549, and 550, respectively;
(sss) a variable region comprising CDR1, CDR2, and CDR3 regions comprising the amino acid sequences of SEQ ID NOs: 551, 552, and 553, respectively;
(ttt) a variable region comprising CDR1, CDR2, and CDR3 regions comprising the amino acid sequences of SEQ ID NOs: 554, 555, and 556, respectively;
(uuu) a variable region comprising CDR1, CDR2, and CDR3 regions comprising the amino acid sequences of SEQ ID NOs: 557, 558, and 559, respectively;
(vvv) a variable region comprising CDR1, CDR2, and CDR3 regions comprising the amino acid sequences of SEQ ID NOs: 560, 561, and 562, respectively;
(www) a variable region comprising CDR1, CDR2, and CDR3 regions comprising the amino acid sequences of SEQ ID NOs: 563, 564, and 565, respectively;
(xxx) a variable region comprising CDR1, CDR2, and CDR3 regions comprising the amino acid sequences of SEQ ID NOs: 566, 567, and 568, respectively;
(yyy) a variable region comprising CDR1, CDR2, and CDR3 regions comprising the amino acid sequences of SEQ ID NOs: 569, 570, and 571, respectively;
(zzz) a variable region comprising CDR1, CDR2, and CDR3 regions comprising the amino acid sequences of SEQ ID NOs: 572, 573, and 574, respectively;
(aaaa) a variable region comprising CDR1, CDR2, and CDR3 regions comprising the amino acid sequences of SEQ ID NOs: 575, 576, and 577, respectively;
(bbbb) a variable region comprising CDR1, CDR2, and CDR3 regions comprising the amino acid sequences of SEQ ID NOs: 578, 579, and 580, respectively;
(cccc) a variable region comprising CDR1, CDR2, and CDR3 regions comprising the amino acid sequences of SEQ ID NOs: 581, 582, and 583, respectively;
(dddd) a variable region comprising CDR1, CDR2, and CDR3 regions comprising the amino acid sequences of SEQ ID NOs: 584, 585, and 586, respectively;
(eeee) a variable region comprising CDR1, CDR2, and CDR3 regions comprising the amino acid sequences of SEQ ID NOs: 587, 588, and 589, respectively;
(ffff) a variable region comprising CDR1, CDR2, and CDR3 regions comprising the amino acid sequences of SEQ ID NOs: 590, 591, and 592, respectively;
(gggg) a variable region comprising CDR1, CDR2, and CDR3 regions comprising the amino acid sequences of SEQ ID NOs: 593, 594, and 595, respectively;
(hhhh) a variable region comprising CDR1, CDR2, and CDR3 regions comprising the amino acid sequences of SEQ ID NOs: 596, 597, and 598, respectively;
(iiii) a variable region comprising CDR1, CDR2, and CDR3 regions comprising the amino acid sequences of SEQ ID NOs: 599, 600, and 601; respectively;
(jjjj) a variable region comprising CDR1, CDR2, and CDR3 regions comprising the amino acid sequences of SEQ ID NOs: 602, 603, and 604, respectively;
(kkkk) a variable region comprising CDR1, CDR2, and CDR3 regions comprising the amino acid sequences of SEQ ID NOs: 605, 606, and 607, respectively;
(llll) a variable region comprising CDR1, CDR2, and CDR3 regions comprising the amino acid sequences of SEQ ID NOs: 608, 609, and 610, respectively;
(mmmm) a variable region comprising CDR1, CDR2, and CDR3 regions comprising the amino acid sequences of SEQ ID NOs: 611, 612, and 613, respectively;
(nnnn) a variable region comprising CDR1, CDR2, and CDR3 regions comprising the amino acid sequences of SEQ ID NOs: 614, 615, and 616, respectively;
(oooo) a variable region comprising CDR1, CDR2, and CDR3 regions comprising the amino acid sequences of SEQ ID NOs: 617, 618, and 619, respectively;
(pppp) a variable region comprising CDR1, CDR2, and CDR3 regions comprising the amino acid sequences of SEQ ID NOs: 620, 621, and 622, respectively;
(qqqq) a variable region comprising CDR1, CDR2, and CDR3 regions comprising the amino acid sequences of SEQ ID NOs: 623, 624, and 625, respectively;
(rrrr) a variable region comprising CDR1, CDR2, and CDR3 regions comprising the amino acid sequences of SEQ ID NOs: 626, 627, and 628, respectively;
(ssss) a variable region comprising CDR1, CDR2, and CDR3 regions comprising the amino acid sequences of SEQ ID NOs: 629, 630, and 631, respectively;
(tttt) a variable region comprising CDR1, CDR2, and CDR3 regions comprising the amino acid sequences of SEQ ID NOs: 632, 633, and 634, respectively;
(uuuu) a variable region comprising CDR1, CDR2, and CDR3 regions comprising the amino acid sequences of SEQ ID NOs: 635, 636, and 637, respectively;
(vvvv) a variable region comprising CDR1, CDR2, and CDR3 regions comprising the amino acid sequences of SEQ ID NOs: 638, 639, and 640, respectively;
(wwww) a variable region comprising CDR1, CDR2, and CDR3 regions comprising the amino acid sequences of SEQ ID NOs: 641, 642, and 643, respectively;
(xxxx) a variable region comprising CDR1, CDR2, and CDR3 regions comprising the amino acid sequences of SEQ ID NOs: 644, 645, and 646, respectively;
(yyyy) a variable region comprising CDR1, CDR2, and CDR3 regions comprising the amino acid sequences of SEQ ID NOs: 647, 648, and 649, respectively;
(zzzz) a variable region comprising CDR1, CDR2, and CDR3 regions comprising the amino acid sequences of SEQ ID NOs: 650, 651, and 652, respectively;
(aaaaa) a variable region comprising CDR1, CDR2, and CDR3 regions comprising the amino acid sequences of SEQ ID NOs: 653, 654, and 655, respectively;
(bbbbb) a variable region comprising CDR1, CDR2, and CDR3 regions comprising the amino acid sequences of SEQ ID NOs: 656, 657, and 658, respectively;
(ccccc) a variable region comprising CDR1, CDR2, and CDR3 regions comprising the amino acid sequences of SEQ ID NOs: 659, 660, and 661, respectively;
(ddddd) a variable region comprising CDR1, CDR2, and CDR3 regions comprising the amino acid sequences of SEQ ID NOs: 662, 663, and 664, respectively;
(eeeee) a variable region comprising CDR1, CDR2, and CDR3 regions comprising the amino acid sequences of SEQ ID NOs: 665, 666, and 667, respectively;
(fffff) a variable region comprising CDR1, CDR2, and CDR3 regions comprising the amino acid sequences of SEQ ID NOs: 668, 669, and 670, respectively;
(ggggg) a variable region comprising CDR1, CDR2, and CDR3 regions comprising the amino acid sequences of SEQ ID NOs: 671, 672, and 673, respectively;
(hhhhh) a variable region comprising CDR1, CDR2, and CDR3 regions comprising the amino acid sequences of SEQ ID NOs: 674, 675, and 676, respectively; and
(iiiii) a variable region comprising CDR1, CDR2, and CDR3 regions comprising the amino acid sequences of SEQ ID NOs: 677, 678, and 679, respectively;

In various embodiments, the antibody or antigen binding fragment thereof comprises a heavy chain variable region sequence selected from the group consisting of SEQ ID NOs: 680, 682, 684, 686, 688, 690, 692, 694, 696, 698, 700, 702, 704, 706, 708, 710, 712, 714, 716, 718, 720, 722, 724, 726, 728, 730 732, 734, 736, 738, 740, 742, 744, 746, 748, 750, 752 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, and 904.

In various embodiments, the antibody or antigen binding fragment thereof comprises a heavy chain variable region sequence which is at least 80%, 85%, 90%, 95%, 98%, or 99% identical to an amino acid sequence selected from the group consisting of SEQ ID NOs: 680, 682, 684, 686, 688, 690, 692, 694, 696, 698, 700, 702, 704, 706, 708, 710, 712, 714, 716, 718, 720, 722, 724, 726, 728, 730 732, 734, 736, 738, 740, 742, 744, 746, 748, 750, 752 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, and 904.

In various embodiments, the antibody or antigen binding fragment thereof comprises a light chain variable region sequence selected from the group consisting of SEQ ID NOs: 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745,747, 749, 751, 753, 755, 757, 759, 761, 763, 765, 767, 769, 771, 773, 775,777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, and 905.

In various embodiments, the antibody or antigen binding fragment thereof comprises a light variable region sequence which is at least 80%, 85%, 90%, 95%, 98%, or 99% identical to an amino acid sequence selected from the group consisting of SEQ ID NOs: 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745,747, 749, 751, 753, 755, 757, 759, 761, 763, 765, 767, 769, 771, 773, 775,777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, and 905.

In various embodiments, the antibody or antigen binding fragment thereof comprises a heavy chain variable region sequence selected from the group consisting of SEQ ID NOs: 680, 682, 684, 686, 688, 690, 692 694, 696, 698, 700, 702, 704, 706, 708, 710, 712, 714, 716, 718, 720, 722, 724, 726, 728, 730 732, 734, 736, 738, 740, 742, 744, 746, 748, 750, 752 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, and 904; and comprises a light chain variable region sequence selected from the group consisting of SEQ ID NOs: 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745,747, 749, 751, 753, 755, 757, 759, 761, 763, 765, 767, 769, 771, 773, 775,777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, and 905.

In various embodiments, the antibody or antigen binding fragment thereof comprises a heavy chain variable region comprising the amino acid sequence of SEQ ID NO: 680 and comprises a light chain variable region comprising the amino acid sequence of SEQ ID NO: 681.

In various embodiments, the antibody or antigen binding fragment thereof comprises a comprises a heavy chain variable region comprising the amino acid sequence of SEQ ID NO: 682 and comprises a light chain variable region comprising the amino acid sequence of SEQ ID NO: 683.

In various embodiments, the antibody or antigen binding fragment thereof comprises a heavy chain variable region comprising the amino acid sequence of SEQ ID NO: 684 and comprises a light chain variable region comprising the amino acid sequence of SEQ ID NO: 685.

In various embodiments, the antibody or antigen binding fragment thereof comprises a heavy chain variable region comprising the amino acid sequence of SEQ ID NO: 686 and comprises a light chain variable region comprising the amino acid sequence of SEQ ID NO: 687.

In various embodiments, the antibody or antigen binding fragment thereof comprises a heavy chain variable region comprising the amino acid sequence of SEQ ID NO: 688 and comprises a light chain variable region comprising the amino acid sequence of SEQ ID NO: 689.

In various embodiments, the antibody or antigen binding fragment thereof comprises a heavy chain variable region comprising the amino acid sequence of SEQ ID NO: 690 and comprises a light chain variable region comprising the amino acid sequence of SEQ ID NO: 691.

In various embodiments, the antibody or antigen binding fragment thereof comprises a heavy chain variable region comprising the amino acid sequence of SEQ ID NO: 692 and comprises a light chain variable region comprising the amino acid sequence of SEQ ID NO: 693.

In various embodiments, the antibody or antigen binding fragment thereof comprises a heavy chain variable region comprising the amino acid sequence of SEQ ID NO: 694 and comprises a light chain variable region comprising the amino acid sequence of SEQ ID NO: 695.

In various embodiments, the antibody or antigen binding fragment thereof comprises a heavy chain variable region comprising the amino acid sequence of SEQ ID NO: 696 and comprises a light chain variable region comprising the amino acid sequence of SEQ ID NO: 697.

In various embodiments, the antibody or antigen binding fragment thereof comprises a heavy chain variable region comprising the amino acid sequence of SEQ ID NO: 698 and comprises a light chain variable region comprising the amino acid sequence of SEQ ID NO: 699.

In various embodiments, the antibody or antigen binding fragment thereof comprises a heavy chain variable region comprising the amino acid sequence of SEQ ID NO: 700 and comprises a light chain variable region comprising the amino acid sequence of SEQ ID NO: 701.

In various embodiments, the antibody or antigen binding fragment thereof comprises a heavy chain variable region comprising the amino acid sequence of SEQ ID NO: 702 and comprises a light chain variable region comprising the amino acid sequence of SEQ ID NO: 703.

In various embodiments, the antibody or antigen binding fragment thereof comprises a heavy chain variable region comprising the amino acid sequence of SEQ ID NO: 704 and comprises a light chain variable region comprising the amino acid sequence of SEQ ID NO: 705.

In various embodiments, the antibody or antigen binding fragment thereof comprises a heavy chain variable region comprising the amino acid sequence of SEQ ID NO: 706 and comprises a light chain variable region comprising the amino acid sequence of SEQ ID NO: 707.

In various embodiments, the antibody or antigen binding fragment thereof comprises a heavy chain variable region comprising the amino acid sequence of SEQ ID NO: 708 and comprises a light chain variable region comprising the amino acid sequence of SEQ ID NO: 709.

In various embodiments, the antibody or antigen binding fragment thereof comprises a heavy chain variable region comprising the amino acid sequence of SEQ ID NO: 710 and comprises a light chain variable region comprising the amino acid sequence of SEQ ID NO: 711.

In various embodiments, the antibody or antigen binding fragment thereof comprises a heavy chain variable region comprising the amino acid sequence of SEQ ID NO: 712 and comprises a light chain variable region comprising the amino acid sequence of SEQ ID NO: 713.

In various embodiments, the antibody or antigen binding fragment thereof comprises a heavy chain variable region comprising the amino acid sequence of SEQ ID NO: 714 and comprises a light chain variable region comprising the amino acid sequence of SEQ ID NO: 715.

In various embodiments, the antibody or antigen binding fragment thereof comprises a heavy chain variable region comprising the amino acid sequence of SEQ ID NO: 716 and comprises a light chain variable region comprising the amino acid sequence of SEQ ID NO: 717.

In various embodiments, the antibody or antigen binding fragment thereof comprises a heavy chain variable region comprising the amino acid sequence of SEQ ID NO: 718 and comprises a light chain variable region comprising the amino acid sequence of SEQ ID NO: 719.

In various embodiments, the antibody or antigen binding fragment thereof comprises a heavy chain variable region comprising the amino acid sequence of SEQ ID NO: 720 and comprises a light chain variable region comprising the amino acid sequence of SEQ ID NO: 721.

In various embodiments, the antibody or antigen binding fragment thereof comprises a heavy chain variable region comprising the amino acid sequence of SEQ ID NO: 722 and comprises a light chain variable region comprising the amino acid sequence of SEQ ID NO: 723.

In various embodiments, the antibody or antigen binding fragment thereof comprises a heavy chain variable region comprising the amino acid sequence of SEQ ID NO: 724 and comprises a light chain variable region comprising the amino acid sequence of SEQ ID NO: 725.

In various embodiments, the antibody or antigen binding fragment thereof comprises a heavy chain variable region comprising the amino acid sequence of SEQ ID NO: 726 and comprises a light chain variable region comprising the amino acid sequence of SEQ ID NO: 727.

In various embodiments, the antibody or antigen binding fragment thereof comprises a heavy chain variable region comprising the amino acid sequence of SEQ ID NO: 728 and comprises a light chain variable region comprising the amino acid sequence of SEQ ID NO: 729.

In various embodiments, the antibody or antigen binding fragment thereof comprises a heavy chain variable region comprising the amino acid sequence of SEQ ID NO: 730 and comprises a light chain variable region comprising the amino acid sequence of SEQ ID NO: 731.

In various embodiments, the antibody or antigen binding fragment thereof comprises a heavy chain variable region comprising the amino acid sequence of SEQ ID NO: 732 and comprises a light chain variable region comprising the amino acid sequence of SEQ ID NO: 733.

In various embodiments, the antibody or antigen binding fragment thereof comprises a heavy chain variable region comprising the amino acid sequence of SEQ ID NO: 734 and comprises a light chain variable region comprising the amino acid sequence of SEQ ID NO: 735.

In various embodiments, the antibody or antigen binding fragment thereof comprises a heavy chain variable region comprising the amino acid sequence of SEQ ID NO: 736 and comprises a light chain variable region comprising the amino acid sequence of SEQ ID NO: 737.

In various embodiments, the antibody or antigen binding fragment thereof comprises a heavy chain variable region comprising the amino acid sequence of SEQ ID NO: 738 and comprises a light chain variable region comprising the amino acid sequence of SEQ ID NO: 739.

In various embodiments, the antibody or antigen binding fragment thereof comprises a heavy chain variable region comprising the amino acid sequence of SEQ ID NO: 740 and comprises a light chain variable region comprising the amino acid sequence of SEQ ID NO: 741.

In various embodiments, the antibody or antigen binding fragment thereof comprises a heavy chain variable region comprising the amino acid sequence of SEQ ID NO: 742 and comprises a light chain variable region comprising the amino acid sequence of SEQ ID NO: 743.

In various embodiments, the antibody or antigen binding fragment thereof comprises a heavy chain variable region comprising the amino acid sequence of SEQ ID NO: 744 and comprises a light chain variable region comprising the amino acid sequence of SEQ ID NO: 745.

In various embodiments, the antibody or antigen binding fragment thereof comprises a heavy chain variable region comprising the amino acid sequence of SEQ ID NO: 746 and comprises a light chain variable region comprising the amino acid sequence of SEQ ID NO: 747.

In various embodiments, the antibody or antigen binding fragment thereof comprises a heavy chain variable region comprising the amino acid sequence of SEQ ID NO: 748 and comprises a light chain variable region comprising the amino acid sequence of SEQ ID NO: 749.

In various embodiments, the antibody or antigen binding fragment thereof comprises a heavy chain variable region comprising the amino acid sequence of SEQ ID NO: 750 and comprises a light chain variable region comprising the amino acid sequence of SEQ ID NO: 751.

In various embodiments, the antibody or antigen binding fragment thereof comprises a heavy chain variable region comprising the amino acid sequence of SEQ ID NO: 752 and comprises a light chain variable region comprising the amino acid sequence of SEQ ID NO: 753.

In various embodiments, the antibody or antigen binding fragment thereof comprises a heavy chain variable region comprising the amino acid sequence of SEQ ID NO: 754 and comprises a light chain variable region comprising the amino acid sequence of SEQ ID NO: 755.

In various embodiments, the antibody or antigen binding fragment thereof comprises a heavy chain variable region comprising the amino acid sequence of SEQ ID NO: 756 and comprises a light chain variable region comprising the amino acid sequence of SEQ ID NO: 757.

In various embodiments, the antibody or antigen binding fragment thereof comprises a heavy chain variable region comprising the amino acid sequence of SEQ ID NO: 758 and comprises a light chain variable region comprising the amino acid sequence of SEQ ID NO: 759.

In various embodiments, the antibody or antigen binding fragment thereof comprises a heavy chain variable region comprising the amino acid sequence of SEQ ID NO: 760 and comprises a light chain variable region comprising the amino acid sequence of SEQ ID NO: 761.

In various embodiments, the antibody or antigen binding fragment thereof comprises a heavy chain variable region comprising the amino acid sequence of SEQ ID NO: 762 and comprises a light chain variable region comprising the amino acid sequence of SEQ ID NO: 763.

In various embodiments, the antibody or antigen binding fragment thereof comprises a heavy chain variable region comprising the amino acid sequence of SEQ ID NO: 764 and comprises a light chain variable region comprising the amino acid sequence of SEQ ID NO: 765.

In various embodiments, the antibody or antigen binding fragment thereof comprises a heavy chain variable region comprising the amino acid sequence of SEQ ID NO: 766 and comprises a light chain variable region comprising the amino acid sequence of SEQ ID NO: 767.

In various embodiments, the antibody or antigen binding fragment thereof comprises a heavy chain variable region comprising the amino acid sequence of SEQ ID NO: 768 and comprises a light chain variable region comprising the amino acid sequence of SEQ ID NO: 769.

In various embodiments, the antibody or antigen binding fragment thereof comprises a heavy chain variable region comprising the amino acid sequence of SEQ ID NO: 770 and comprises a light chain variable region comprising the amino acid sequence of SEQ ID NO: 771.

In various embodiments, the antibody or antigen binding fragment thereof comprises a heavy chain variable region comprising the amino acid sequence of SEQ ID NO: 772 and comprises a light chain variable region comprising the amino acid sequence of SEQ ID NO: 773.

In various embodiments, the antibody or antigen binding fragment thereof comprises a heavy chain variable region comprising the amino acid sequence of SEQ ID NO: 774 and comprises a light chain variable region comprising the amino acid sequence of SEQ ID NO: 775.

In various embodiments, the antibody or antigen binding fragment thereof comprises a heavy chain variable region comprising the amino acid sequence of SEQ ID NO: 776 and comprises a light chain variable region comprising the amino acid sequence of SEQ ID NO: 777.

In various embodiments, the antibody or antigen binding fragment thereof comprises a heavy chain variable region comprising the amino acid sequence of SEQ ID NO: 778 and comprises a light chain variable region comprising the amino acid sequence of SEQ ID NO: 779.

In various embodiments, the antibody or antigen binding fragment thereof comprises a heavy chain variable region comprising the amino acid sequence of SEQ ID NO: 780 and comprises a light chain variable region comprising the amino acid sequence of SEQ ID NO: 781.

In various embodiments, the antibody or antigen binding fragment thereof comprises a heavy chain variable region comprising the amino acid sequence of SEQ ID NO: 782 and comprises a light chain variable region comprising the amino acid sequence of SEQ ID NO: 783.

In various embodiments, the antibody or antigen binding fragment thereof comprises a heavy chain variable region comprising the amino acid sequence of SEQ ID NO: 784 and comprises a light chain variable region comprising the amino acid sequence of SEQ ID NO: 785.

In various embodiments, the antibody or antigen binding fragment thereof comprises a heavy chain variable region comprising the amino acid sequence of SEQ ID NO: 786 and comprises a light chain variable region comprising the amino acid sequence of SEQ ID NO: 787.

In various embodiments, the antibody or antigen binding fragment thereof comprises a heavy chain variable region comprising the amino acid sequence of SEQ ID NO: 788 and comprises a light chain variable region comprising the amino acid sequence of SEQ ID NO: 789.

In various embodiments, the antibody or antigen binding fragment thereof comprises a heavy chain variable region comprising the amino acid sequence of SEQ ID NO: 790 and comprises a light chain variable region comprising the amino acid sequence of SEQ ID NO: 791.

In various embodiments, the antibody or antigen binding fragment thereof comprises a heavy chain variable region comprising the amino acid sequence of SEQ ID NO: 792 and comprises a light chain variable region comprising the amino acid sequence of SEQ ID NO: 793.

In various embodiments, the antibody or antigen binding fragment thereof comprises a heavy chain variable region comprising the amino acid sequence of SEQ ID NO: 794 and comprises a light chain variable region comprising the amino acid sequence of SEQ ID NO: 795.

In various embodiments, the antibody or antigen binding fragment thereof comprises a heavy chain variable region comprising the amino acid sequence of SEQ ID NO: 796 and comprises a light chain variable region comprising the amino acid sequence of SEQ ID NO: 797.

In various embodiments, the antibody or antigen binding fragment thereof comprises a heavy chain variable region comprising the amino acid sequence of SEQ ID NO: 798 and comprises a light chain variable region comprising the amino acid sequence of SEQ ID NO: 799.

In various embodiments, the antibody or antigen binding fragment thereof comprises a heavy chain variable region comprising the amino acid sequence of SEQ ID NO: 800 and comprises a light chain variable region comprising the amino acid sequence of SEQ ID NO: 801.

In various embodiments, the antibody or antigen binding fragment thereof comprises a heavy chain variable region comprising the amino acid sequence of SEQ ID NO: 802 and comprises a light chain variable region comprising the amino acid sequence of SEQ ID NO: 803.

In various embodiments, the antibody or antigen binding fragment thereof comprises a heavy chain variable region comprising the amino acid sequence of SEQ ID NO: 804 and comprises a light chain variable region comprising the amino acid sequence of SEQ ID NO: 805.

In various embodiments, the antibody or antigen binding fragment thereof comprises a heavy chain variable region comprising the amino acid sequence of SEQ ID NO: 806 and comprises a light chain variable region comprising the amino acid sequence of SEQ ID NO: 807.

In various embodiments, the antibody or antigen binding fragment thereof comprises a heavy chain variable region comprising the amino acid sequence of SEQ ID NO: 808 and comprises a light chain variable region comprising the amino acid sequence of SEQ ID NO: 809.

In various embodiments, the antibody or antigen binding fragment thereof comprises a heavy chain variable region comprising the amino acid sequence of SEQ ID NO: 810 and comprises a light chain variable region comprising the amino acid sequence of SEQ ID NO: 811.

In various embodiments, the antibody or antigen binding fragment thereof comprises a heavy chain variable region comprising the amino acid sequence of SEQ ID NO: 812 and comprises a light chain variable region comprising the amino acid sequence of SEQ ID NO: 813.

In various embodiments, the antibody or antigen binding fragment thereof comprises a heavy chain variable region comprising the amino acid sequence of SEQ ID NO: 814 and comprises a light chain variable region comprising the amino acid sequence of SEQ ID NO: 815.

In various embodiments, the antibody or antigen binding fragment thereof comprises a heavy chain variable region comprising the amino acid sequence of SEQ ID NO: 816 and comprises a light chain variable region comprising the amino acid sequence of SEQ ID NO: 817.

In various embodiments, the antibody or antigen binding fragment thereof comprises a heavy chain variable region comprising the amino acid sequence of SEQ ID NO: 818 and comprises a light chain variable region comprising the amino acid sequence of SEQ ID NO: 819.

In various embodiments, the antibody or antigen binding fragment thereof comprises a heavy chain variable region comprising the amino acid sequence of SEQ ID NO: 820 and comprises a light chain variable region comprising the amino acid sequence of SEQ ID NO: 821.

In various embodiments, the antibody or antigen binding fragment thereof comprises a heavy chain variable region comprising the amino acid sequence of SEQ ID NO: 822 and comprises a light chain variable region comprising the amino acid sequence of SEQ ID NO: 823.

In various embodiments, the antibody or antigen binding fragment thereof comprises a heavy chain variable region comprising the amino acid sequence of SEQ ID NO: 824 and comprises a light chain variable region comprising the amino acid sequence of SEQ ID NO: 825.

In various embodiments, the antibody or antigen binding fragment thereof comprises a heavy chain variable region comprising the amino acid sequence of SEQ ID NO: 826 and comprises a light chain variable region comprising the amino acid sequence of SEQ ID NO: 827.

In various embodiments, the antibody or antigen binding fragment thereof comprises a heavy chain variable region comprising the amino acid sequence of SEQ ID NO: 828 and comprises a light chain variable region comprising the amino acid sequence of SEQ ID NO: 829.

In various embodiments, the antibody or antigen binding fragment thereof comprises a heavy chain variable region comprising the amino acid sequence of SEQ ID NO: 830 and comprises a light chain variable region comprising the amino acid sequence of SEQ ID NO: 831.

In various embodiments, the antibody or antigen binding fragment thereof comprises a heavy chain variable region comprising the amino acid sequence of SEQ ID NO: 832 and comprises a light chain variable region comprising the amino acid sequence of SEQ ID NO: 833.

In various embodiments, the antibody or antigen binding fragment thereof comprises a heavy chain variable region comprising the amino acid sequence of SEQ ID NO: 834 and comprises a light chain variable region comprising the amino acid sequence of SEQ ID NO: 835.

In various embodiments, the antibody or antigen binding fragment thereof comprises a heavy chain variable region comprising the amino acid sequence of SEQ ID NO: 836 and comprises a light chain variable region comprising the amino acid sequence of SEQ ID NO: 837.

In various embodiments, the antibody or antigen binding fragment thereof comprises a heavy chain variable region comprising the amino acid sequence of SEQ ID NO: 838 and comprises a light chain variable region comprising the amino acid sequence of SEQ ID NO: 839.

In various embodiments, the antibody or antigen binding fragment thereof comprises a heavy chain variable region comprising the amino acid sequence of SEQ ID NO: 840 and comprises a light chain variable region comprising the amino acid sequence of SEQ ID NO: 841.

In various embodiments, the antibody or antigen binding fragment thereof comprises a heavy chain variable region comprising the amino acid sequence of SEQ ID NO: 842 and comprises a light chain variable region comprising the amino acid sequence of SEQ ID NO: 843.

In various embodiments, the antibody or antigen binding fragment thereof comprises a heavy chain variable region comprising the amino acid sequence of SEQ ID NO: 844 and comprises a light chain variable region comprising the amino acid sequence of SEQ ID NO: 845.

In various embodiments, the antibody or antigen binding fragment thereof comprises a heavy chain variable region comprising the amino acid sequence of SEQ ID NO: 846 and comprises a light chain variable region comprising the amino acid sequence of SEQ ID NO: 847.

In various embodiments, the antibody or antigen binding fragment thereof comprises a heavy chain variable region comprising the amino acid sequence of SEQ ID NO: 848 and comprises a light chain variable region comprising the amino acid sequence of SEQ ID NO: 849.

In various embodiments, the antibody or antigen binding fragment thereof comprises a heavy chain variable region comprising the amino acid sequence of SEQ ID NO: 850 and comprises a light chain variable region comprising the amino acid sequence of SEQ ID NO: 851.

In various embodiments, the antibody or antigen binding fragment thereof comprises a heavy chain variable region comprising the amino acid sequence of SEQ ID NO: 852 and comprises a light chain variable region comprising the amino acid sequence of SEQ ID NO: 853.

In various embodiments, the antibody or antigen binding fragment thereof comprises a heavy chain variable region comprising the amino acid sequence of SEQ ID NO: 854 and comprises a light chain variable region comprising the amino acid sequence of SEQ ID NO: 855.

In various embodiments, the antibody or antigen binding fragment thereof comprises a heavy chain variable region comprising the amino acid sequence of SEQ ID NO: 856 and comprises a light chain variable region comprising the amino acid sequence of SEQ ID NO: 857.

In various embodiments, the antibody or antigen binding fragment thereof comprises a heavy chain variable region comprising the amino acid sequence of SEQ ID NO: 858 and comprises a light chain variable region comprising the amino acid sequence of SEQ ID NO: 859.

In various embodiments, the antibody or antigen binding fragment thereof comprises a heavy chain variable region comprising the amino acid sequence of SEQ ID NO: 860 and comprises a light chain variable region comprising the amino acid sequence of SEQ ID NO: 861.

In various embodiments, the antibody or antigen binding fragment thereof comprises a heavy chain variable region comprising the amino acid sequence of SEQ ID NO: 862 and comprises a light chain variable region comprising the amino acid sequence of SEQ ID NO: 863.

In various embodiments, the antibody or antigen binding fragment thereof comprises a heavy chain variable region comprising the amino acid sequence of SEQ ID NO: 864 and comprises a light chain variable region comprising the amino acid sequence of SEQ ID NO: 865.

In various embodiments, the antibody or antigen binding fragment thereof comprises a heavy chain variable region comprising the amino acid sequence of SEQ ID NO: 866 and comprises a light chain variable region comprising the amino acid sequence of SEQ ID NO: 867.

In various embodiments, the antibody or antigen binding fragment thereof comprises a heavy chain variable region comprising the amino acid sequence of SEQ ID NO: 868 and comprises a light chain variable region comprising the amino acid sequence of SEQ ID NO: 869.

In various embodiments, the antibody or antigen binding fragment thereof comprises a heavy chain variable region comprising the amino acid sequence of SEQ ID NO: 870 and comprises a light chain variable region comprising the amino acid sequence of SEQ ID NO: 871.

In various embodiments, the antibody or antigen binding fragment thereof comprises a heavy chain variable region comprising the amino acid sequence of SEQ ID NO: 872 and comprises a light chain variable region comprising the amino acid sequence of SEQ ID NO: 873.

In various embodiments, the antibody or antigen binding fragment thereof comprises a heavy chain variable region comprising the amino acid sequence of SEQ ID NO: 874 and comprises a light chain variable region comprising the amino acid sequence of SEQ ID NO: 875.

In various embodiments, the antibody or antigen binding fragment thereof comprises a heavy chain variable region comprising the amino acid sequence of SEQ ID NO: 876 and comprises a light chain variable region comprising the amino acid sequence of SEQ ID NO: 877.

In various embodiments, the antibody or antigen binding fragment thereof comprises a heavy chain variable region comprising the amino acid sequence of SEQ ID NO: 878 and comprises a light chain variable region comprising the amino acid sequence of SEQ ID NO: 879.

In various embodiments, the antibody or antigen binding fragment thereof comprises a heavy chain variable region comprising the amino acid sequence of SEQ ID NO: 880 and comprises a light chain variable region comprising the amino acid sequence of SEQ ID NO: 881.

In various embodiments, the antibody or antigen binding fragment thereof comprises a heavy chain variable region comprising the amino acid sequence of SEQ ID NO: 882 and comprises a light chain variable region comprising the amino acid sequence of SEQ ID NO: 883.

In various embodiments, the antibody or antigen binding fragment thereof comprises a heavy chain variable region comprising the amino acid sequence of SEQ ID NO: 884 and comprises a light chain variable region comprising the amino acid sequence of SEQ ID NO: 885.

In various embodiments, the antibody or antigen binding fragment thereof comprises a heavy chain variable region comprising the amino acid sequence of SEQ ID NO: 886 and comprises a light chain variable region comprising the amino acid sequence of SEQ ID NO: 887.

In various embodiments, the antibody or antigen binding fragment thereof comprises a heavy chain variable region comprising the amino acid sequence of SEQ ID NO: 888 and comprises a light chain variable region comprising the amino acid sequence of SEQ ID NO: 889.

In various embodiments, the antibody or antigen binding fragment thereof comprises a heavy chain variable region comprising the amino acid sequence of SEQ ID NO: 890 and comprises a light chain variable region comprising the amino acid sequence of SEQ ID NO: 891.

In various embodiments, the antibody or antigen binding fragment thereof comprises a heavy chain variable region comprising the amino acid sequence of SEQ ID NO: 892 and comprises a light chain variable region comprising the amino acid sequence of SEQ ID NO: 893.

In various embodiments, the antibody or antigen binding fragment thereof comprises a heavy chain variable region comprising the amino acid sequence of SEQ ID NO: 894 and comprises a light chain variable region comprising the amino acid sequence of SEQ ID NO: 895.

In various embodiments, the antibody or antigen binding fragment thereof comprises a heavy chain variable region comprising the amino acid sequence of SEQ ID NO: 896 and comprises a light chain variable region comprising the amino acid sequence of SEQ ID NO: 897.

In various embodiments, the antibody or antigen binding fragment thereof comprises a heavy chain variable region comprising the amino acid sequence of SEQ ID NO: 898 and comprises a light chain variable region comprising the amino acid sequence of SEQ ID NO: 899.

In various embodiments, the antibody or antigen binding fragment thereof comprises a heavy chain variable region comprising the amino acid sequence of SEQ ID NO: 900 and comprises a light chain variable region comprising the amino acid sequence of SEQ ID NO: 901.

In various embodiments, the antibody or antigen binding fragment thereof comprises a heavy chain variable region comprising the amino acid sequence of SEQ ID NO: 902 and comprises a light chain variable region comprising the amino acid sequence of SEQ ID NO: 903.

In various embodiments, the antibody or antigen binding fragment thereof comprises a heavy chain variable region comprising the amino acid sequence of SEQ ID NO: 904 and comprises a light chain variable region comprising the amino acid sequence of SEQ ID NO: 905.

In various embodiments, the antibody or antigen binding fragment thereof comprises the amino acid sequence of SEQ ID NO: 906 and/or the amino acid sequence of SEQ ID NO: 907.

In various embodiments, the antibody or antigen binding fragment thereof of any of the preceding claims that neutralizes hMPV A and/or hMPV B. In specific embodiments the antibody or antigen binding fragment thereof neutralizes hMPV A. In specific embodiments the antibody or antigen binding fragment thereof neutralizes hMPV B.

In various embodiments, the antibody or antigen binding fragment thereof inhibits hMPV infection.

In various embodiments, the antibody or antigen binding fragment thereof neutralizes hMPV with a neutralizing potency (IC₅₀) of 10 microgram per milliliter (μg/mL) or less, 9 μg/mL or less, 8 μg/mL or less, 7 μg/mL or less, 6 μg/mL or less, 5 μg/mL or less, 4 μg/mL or less, 3 μg/mL or less, 2 μg/mL or less, or 1 μg/mL or less.

In various embodiments, the antibody or antigen binding fragment thereof neutralizes hMPV with a neutralizing potency (IC50) described herein, e.g., Tables 3A-C.

In various embodiments, the antibody or antigen binding fragment thereof binds and neutralizes hMPV in vitro.

In various embodiments, the antibody or antigen binding fragment thereof neutralizes hMPV in vivo.

In various embodiments, the antibody or antigen binding fragment thereof binds to a hMPV F protein or antigen or a hMPV prefusion F protein or antigen with a K_{D} value of about 1×10⁻⁹ M to about 1×10⁻¹² M as determined by surface plasmon resonance (e.g., BIACORE) or a similar technique (e.g., KinExa and OCTET). In various embodiments, the antibody or antigen binding fragment thereof binds to a processed F antigen. In various embodiments, the antibody or antigen binding fragment thereof binds to an unprocessed F antigen. In various embodiments, the antibody or antigen binding fragment thereof binds to a processed PreF antigen. In various embodiments, the antibody or antigen binding fragment thereof binds to an unprocessed PreF antigen.

In various embodiments, the antibody or antigen binding fragment thereof binds, cross-reacts and/or neutralizes RSV and/or an RSV antigen.

In various embodiments, the antibody or antigen binding fragment thereof is an antibody.

For example, the antibody is a chimeric, human or humanized antibody.

An aspect of the invention provides an antibody or antigen binding fragment thereof that binds to the same epitope on hMPV as any antibody or antigen binding fragment thereof described herein, e.g., an antibody or antigen fragment thereof as described in Tables 1-4.

In various embodiments, the antibody or antigen binding fragment thereof binds to an epitope of hMPV of SEQ ID NO: 1, SEQ ID NO: 910, SEQ ID NO: 911, SEQ ID NO: 912, and/or SEQ ID NO: 913.

An aspect of the invention provides an antibody or antigen binding fragment thereof which binds to one or more sites: Ø, I, II, III, III′, IV, IV′, V, α, and/or β of hMPV (SEQ ID NO: 1). In various embodiments, the antibody or antigen binding fragment thereof binds to site Ø of hMPV (SEQ ID NO: 1). In various embodiments, the antibody or antigen binding fragment thereof binds to site I of hMPV (SEQ ID NO: 1). In various embodiments, the antibody or antigen binding fragment thereof binds to site II of hMPV (SEQ ID NO: 1). In various embodiments, the antibody or antigen binding fragment thereof binds to site III of hMPV (SEQ ID NO: 1). In various embodiments, the antibody or antigen binding fragment thereof binds to site III′ of hMPV (SEQ ID NO: 1). In various embodiments, the antibody or antigen binding fragment thereof binds to site IV of hMPV (SEQ ID NO: 1). In various embodiments, the antibody or antigen binding fragment thereof binds to site IV′ of hMPV (SEQ ID NO: 1). In various embodiments, the antibody or antigen binding fragment thereof binds to site V of hMPV (SEQ ID NO: 1). In various embodiments, the antibody or antigen binding fragment thereof binds to site α of hMPV (SEQ ID NO: 1). In various embodiments, the antibody or antigen binding fragment thereof binds to site β of hMPV (SEQ ID NO: 1).

An aspect of the invention provides an antibody or antigen binding fragment thereof which neutralizes hMPV and/or binds to one or more sites: Ø, I, II, III, III′, IV, IV′, V, α, and/or β site of hMPV (SEQ ID NO: 1). In various embodiments, the antibody or antigen binding fragment thereof binds to site Ø of hMPV (SEQ ID NO: 1). In various embodiments, the antibody or antigen binding fragment thereof binds to site I of hMPV (SEQ ID NO: 1). In various embodiments, the antibody or antigen binding fragment thereof binds to site II of hMPV (SEQ ID NO: 1). In various embodiments, the antibody or antigen binding fragment thereof binds to site III of hMPV (SEQ ID NO: 1). In various embodiments, the antibody or antigen binding fragment thereof binds to site III′ of hMPV (SEQ ID NO: 1). In various embodiments, the antibody or antigen binding fragment thereof binds to site IV of hMPV (SEQ ID NO: 1). In various embodiments, the antibody or antigen binding fragment thereof binds to site IV′ of hMPV (SEQ ID NO: 1). In various embodiments, the antibody or antigen binding fragment thereof binds to site V of hMPV (SEQ ID NO: 1). In various embodiments, the antibody or antigen binding fragment thereof binds to site α of hMPV (SEQ ID NO: 1). In various embodiments, the antibody or antigen binding fragment thereof binds to site β of hMPV (SEQ ID NO: 1).

In various embodiments, the antibody or antigen binding fragment thereof binds to the particular antigen site described above and have greater than 80% identity (i.e., greater than 80, 85, 90, 95, 97, 98, 99, 100% identity) to the VH and/or VL of the disclosed antibodies or antigen binding fragments thereof described herein, for example, in Table 3B, Table 3C, or Table 3D.

An aspect of the invention provides a multi-specific molecule comprising a first binding region comprising the antibody or antigen binding fragment thereof described herein and a second binding region. In various embodiments, the multi-specific comprises a bispecific molecule. For example, the second binding region binds to a viral-associated antigen. In various embodiments, the second binding region binds to an antigen or ligand associated with a viral condition or viral disease.

An aspect of the invention provides a nucleic acid comprising a nucleotide sequence that encodes any antibody or antigen binding fragment thereof described herein or any bispecific molecule described herein.

An aspect of the invention provides an expression vector comprising any nucleic acid described herein. In one example, the expression vector comprises a nucleic acid encoding at least one of the polypeptides in Table 3D (SEQ ID NOs: 680-905).

An aspect of the invention provides a cell transformed with any expression vector described herein.

An aspect of the invention provides a pharmaceutical composition for preventing, treating or detecting a virus comprising: any antibody or antigen binding fragment described herein, or any bispecific molecule described; and a pharmaceutically acceptable carrier. In various embodiments, the pharmaceutical composition comprises one or more additional prophylactic agent or therapeutic agent. In various embodiments of the pharmaceutical composition, the one or more additional therapeutic agent is selected from the group consisting of: an immunomodulator, a hormone, a cytotoxic agent, an enzyme, a radionuclide, a second antibody conjugated to at least one immunomodulator, an enzyme, a radioactive label, a hormone, an antisense oligonucleotide, or a cytotoxic agent, and a combination thereof.

An aspect of the invention provides a kit for preventing, treating or detecting a virus comprising: any antibody or antigen binding fragment thereof described, any multi-specific molecule described (e.g., bispecific molecule), and/or any pharmaceutical composition described herein; and instructions for use.

An aspect of the invention provides a method of producing an antibody or antigen binding fragment thereof comprising: culturing a host cell comprising a polynucleotide encoding the amino acid sequences of any antibody or antigen binding fragments thereof described herein under conditions favorable to expression of the polynucleotide; and optionally, recovering the antibody or antigen binding fragment thereof from the host cell and/or culture medium.

An aspect of the invention provides a method of selectively detecting hMPV comprising administering or contacting to a sample any antibody or antigen binding fragment thereof described herein and detecting the presence of the antibody or antigen binding fragment thereof. In various embodiments of the method, the sample comprises a bodily fluid. For example, the sample comprises a serum from a subject.

An aspect of the invention provides a method for treating a subject having a viral disease, comprising administering to a subject in need thereof a therapeutically effective amount of any antibody or antigen binding fragment thereof described herein, any multi-specific (e.g., bispecific) molecule described herein, and/or any pharmaceutical composition described herein.

In various embodiments of the method, the viral disease is selected from the group consisting of: an MPV infection (e.g., an hMPV infection), a respiratory virus), clinical symptoms associated with MPV infection, Lower Respiratory tract infections (LTRI) leading to acute respiratory tract infection leading to Bronchiolitis, Pneumonia). In various embodiments of the method, the MPV infection comprises an infection of the lungs and/or respiratory tract. In various embodiments of the method, the respiratory virus is an upper respiratory virus. In various embodiments of the method, the virus disease is associated with hMPV type A. In various embodiments, the virus disease is associated with hMPV type B. In various embodiments, the respiratory virus is a human respiratory syncytial virus (hRSV).

In various embodiments, the method further comprises administering one or more additional therapies.

An aspect of the invention provides any antibody or antigen binding fragment thereof described herein, any multi-specific (e.g., bispecific) molecule described herein, and/or any pharmaceutical composition described herein, for use in the preparation of a medicament to: bind hMPV; neutralize hMPV; and/or treat a disorder or condition associated with hMPV in a subject in need thereof.

An aspect of the invention also provides isolated hMPV F polypeptides, nucleic acids encoding such isolated polypeptides, and immunogenic compositions comprising such isolated hMPV F polypeptides. Such isolated hMPV F polypeptides and immunogenic compositions may be used to immunize a subject and prevent or treat an hMPV infection.

In various embodiments of an isolated hMPV F polypeptide, the invention provides an isolated hMPV F polypeptide comprising residues 1-490 of SEQ ID NO: 911, or a variant thereof, wherein the isolated hMPV F polypeptide does not include residues 491-539 of SEQ ID NO: 1. In various embodiments, the variant comprises an amino acid sequence having 95-99% identity to residues 1-490 of SEQ ID NO: 911. In various embodiments of the isolated hMPV F polypeptide comprising SEQ ID NO: 911, or a variant thereof, the variant amino acid sequence of the isolated hMPV F polypeptide does not vary at residues 491-512 of SEQ ID NO: 911. In various embodiments of the isolated hMPV F polypeptide, the variant amino acid sequence varies from SEQ ID NO: 911 by 25, 24, 23, 22, 21, 20, 19, 18, 17, 16, 15, 14, 13, 12, 11, 10, 9, 8, 7, 6, 5, 4, 3, 2, or 1 amino acids. In various embodiments of the isolated hMPV F polypeptide, the variant amino acid sequence varies from SEQ ID NO: 911 by 1-15 amino acids. In various embodiments of the isolated hMPV F polypeptide, the variant amino acid sequence varies from SEQ ID NO: 911 by 1-10 amino acids. In various embodiments of the isolated hMPV F polypeptide, the variant amino acid sequence varies from SEQ ID NO: 911 by 1-5 amino acids. In various embodiments of the isolated hMPV F polypeptide, the variant amino acid sequence varies from SEQ ID NO: 911 by 1-3 amino acids.

In various embodiments of the isolated hMPV F polypeptide, the isolated hMPV F polypeptide consists of SEQ ID NO: 911.

In various embodiments of an isolated hMPV F polypeptide, the isolated hMPV F polypeptide comprises residues 1-490 of SEQ ID NO: 911, or a variant thereof, and a trimerization domain, wherein the isolated hMPV F polypeptide does not include residues 491-539 of SEQ ID NO: 1. In various embodiments, the trimerization domain is a GCN4 domain, a T4 foldon domain, a human type XV collagen domain, or a human type XVIII collagen domain. In various embodiments, the GCN4 domain has the amino acid sequence of SEQ ID NO: 1022, the T4 foldon domain has the amino acid sequence of SEQ ID NO: 1012, the human type XV collagen domain has the amino acid sequence of SEQ ID NO: 1023, and the human type XVIII collagen domain has the amino acid sequence of SEQ ID NO: 1024.

In various embodiments of an isolated hMPV F polypeptide, a protease cleavage sequence at residues 99-102 is replaced with a furin-cleavage sequence. In various embodiments, the furin-cleavage sequence is SEQ ID NO: 1025 or 1026. In various embodiments, the furin-cleavage sequence is SEQ ID NO: 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, or 1066. In various embodiments, the furin-cleavage sequence is SEQ ID NO: 1033.

In various embodiments of an isolated hMPV F polypeptide, the invention provides an isolated hMPV F polypeptide comprising residues 1-490 of SEQ ID NO: 911, or a variant thereof, wherein the isolated hMPV F polypeptide does not include residues 491-539 of SEQ ID NO: 1, wherein the isolated hMPV F polypeptide has a mutation at residue 185. In various embodiments, the mutation is A185P.

In various embodiments of an isolated hMPV F polypeptide, the isolated hMPV F polypeptide comprising residues 1-521 of SEQ ID NO: 910, residues 1-518 of SEQ ID NO: 912, or residues 1-521 of SEQ ID NO: 913, or variants thereof, wherein the variant of the isolated hMPV F polypeptide comprising residues 1-521 of SEQ ID NO: 910 does not vary at residues 486-517 of SEQ ID NO: 910, wherein the variant of the isolated hMPV F polypeptide comprising residues 1-518 of SEQ ID NO: 912 does not vary at residues 102-105 and 490-516 of SEQ ID NO: 912, wherein the variant of the isolated hMPV F polypeptide comprising residues 1-521 of SEQ ID NO: 913 does not vary at residues 99-102, 185, and 485-521 of SEQ ID NO: 913. In various embodiments, the variant comprises an amino acid sequence having 95-99% identity to residues 1-521 of SEQ ID NO: 910, 95-99% identity to residues 1-58 of SEQ ID NO: 912, or 95-99% identity to residues 1-521 of SEQ ID NO: 91, wherein the variant of the isolated hMPV F polypeptide comprising residues 1-521 of SEQ ID NO: 910 does not vary at residues 486-517, wherein the variant of the isolated hMPV F polypeptide comprising residues 1-518 of SEQ ID NO: 912 does not vary at residues 102-105 and 490-516, and wherein the variant of the isolated hMPV F polypeptide comprising residues 1-521 of SEQ ID NO: 913 does not vary at residues 99-102, 185, and 485-521.

In various embodiments of the isolated hMPV F polypeptide, the variant of the isolated hMPV F polypeptide comprising residues 1-521 of SEQ ID NO: 910 varies by 25, 24, 23, 22, 21, 20, 19, 18, 17, 16, 15, 14, 13, 12, 11, 10, 9, 8, 7, 6, 5, 4, 3, 2, or 1 amino acids, wherein the variant of the isolated hMPV F polypeptide comprising residues 1-521 of SEQ ID NO: 910 does not vary at residues 486-517. In various embodiments of the isolated hMPV F polypeptide, the variant amino acid sequence varies from SEQ ID NO: 910 by 1-15 amino acids, wherein the variant of the isolated hMPV F polypeptide comprising residues 1-521 of SEQ ID NO: 910 does not vary at residues 486-517. In various embodiments of the isolated hMPV F polypeptide, the variant amino acid sequence varies from SEQ ID NO: 910 by 1-10 amino acids, wherein the variant of the isolated hMPV F polypeptide comprising residues 1-521 of SEQ ID NO: 910 does not vary at residues 486-517. In various embodiments of the isolated hMPV F polypeptide, the variant amino acid sequence varies from SEQ ID NO: 910 by 1-5 amino acids, wherein the variant of the isolated hMPV F polypeptide comprising residues 1-521 of SEQ ID NO: 910 does not vary at residues 486-517. In various embodiments of the isolated hMPV F polypeptide, the variant amino acid sequence varies from SEQ ID NO: 910 by 1-3 amino acids, wherein the variant of the isolated hMPV F polypeptide comprising residues 1-521 of SEQ ID NO: 910 does not vary at residues 486-517.

In various embodiments of the isolated hMPV F polypeptide, the isolated hMPV F polypeptide consists of residues 1-521 of SEQ ID NO: 910.

In various embodiments of the isolated hMPV F polypeptide, the variant of the isolated hMPV F polypeptide comprising residues 1-518 of SEQ ID NO: 912 varies by 25, 24, 23, 22, 21, 20, 19, 18, 17, 16, 15, 14, 13, 12, 11, 10, 9, 8, 7, 6, 5, 4, 3, 2, or 1 amino acids, wherein the variant of the isolated hMPV F polypeptide comprising residues 1-518 of SEQ ID NO: 912 does not vary at residues 102-105 and 490-516. In various embodiments of the isolated hMPV F polypeptide, the variant amino acid sequence varies from SEQ ID NO: 912 by 1-15 amino acids, wherein the variant of the isolated hMPV F polypeptide comprising residues 1-518 of SEQ ID NO: 912 does not vary at residues 102-105 and 490-516. In various embodiments of the isolated hMPV F polypeptide, the variant amino acid sequence varies from SEQ ID NO: 912 by 1-10 amino acids, wherein the variant of the isolated hMPV F polypeptide comprising residues 1-518 of SEQ ID NO: 912 does not vary at residues 102-105 and 490-516. In various embodiments of the isolated hMPV F polypeptide, the variant amino acid sequence varies from SEQ ID NO: 912 by 1-5 amino acids, wherein the variant of the isolated hMPV F polypeptide comprising residues 1-518 of SEQ ID NO: 912 does not vary at residues 102-105 and 490-516. In various embodiments of the isolated hMPV F polypeptide, the variant amino acid sequence varies from SEQ ID NO: 912 by 1-3 amino acids, wherein the variant of the isolated hMPV F polypeptide comprising residues 1-518 of SEQ ID NO: 912 does not vary at residues 102-105 and 490-516.

In various embodiments of the isolated hMPV F polypeptide, the isolated hMPV F polypeptide consists of residues 1-518 of SEQ ID NO: 912.

In various embodiments of the isolated hMPV F polypeptide, the variant of the isolated hMPV F polypeptide comprising residues 1-521 of SEQ ID NO: 913 varies by 25, 24, 23, 22, 21, 20, 19, 18, 17, 16, 15, 14, 13, 12, 11, 10, 9, 8, 7, 6, 5, 4, 3, 2, or 1 amino acids, wherein the variant of the isolated hMPV F polypeptide comprising residues 1-521 of SEQ ID NO: 913 does not vary at residues 99-102, 185, and 485-521. In various embodiments of the isolated hMPV F polypeptide, the variant amino acid sequence varies from SEQ ID NO: 913 by 1-15 amino acids, wherein the variant of the isolated hMPV F polypeptide comprising residues 1-521 of SEQ ID NO: 913 does not vary at residues 99-102, 185, and 485-521. In various embodiments of the isolated hMPV F polypeptide, the variant amino acid sequence varies from SEQ ID NO: 913 by 1-10 amino acids, wherein the variant of the isolated hMPV F polypeptide comprising residues 1-521 of SEQ ID NO: 913 does not vary at residues 99-102, 185, and 485-521. In various embodiments of the isolated hMPV F polypeptide, the variant amino acid sequence varies from SEQ ID NO: 913 by 1-5 amino acids, wherein the variant of the isolated hMPV F polypeptide comprising residues 1-521 of SEQ ID NO: 913 does not vary at residues 99-102, 185, and 485-521. In various embodiments of the isolated hMPV F polypeptide, the variant amino acid sequence varies from SEQ ID NO: 913 by 1-3 amino acids, wherein the variant of the isolated hMPV F polypeptide comprising residues 1-521 of SEQ ID NO: 913 does not vary at residues 99-102, 185, and 485-521.

In various embodiments of the isolated hMPV F polypeptide, the isolated hMPV F polypeptide consists of residues 1-521 of SEQ ID NO: 913.

In various embodiments of the isolated hMPV F polypeptide, the isolated hMPV F polypeptide consists of residues 1-521 of SEQ ID NO: 910, residues 1-518 of SEQ ID NO: 912, or residues 1-521 of SEQ ID NO: 913.

In various embodiments of an isolated hMPV F polypeptide, the isolated hMPV F polypeptide comprises a cleavage sequence and at least one affinity tag sequence. In various embodiments, the cleavage sequence is a thrombin cleavage sequence. In various embodiments, the affinity tag sequence is a 6×His sequence (SEQ ID NO: 1014) or a strep tag II sequence.

In various embodiments of an isolated hMPV F polypeptide, the isolated hMPV F polypeptide comprises the amino acid sequence of SEQ ID NO: 910, SEQ ID NO: 912, or SEQ ID NO: 913. In various embodiments, the isolated hMPV F polypeptide consists of the amino acid sequence of SEQ ID NO: 910, SEQ ID NO: 912, or SEQ ID NO: 913.

An aspect of the invention also provides nucleic acids encoding isolated hMPV F polypeptides, isolated hMPV F polypeptides, and immunogenic compositions comprising such isolated hMPV F polypeptides that encode variants of hMPV F protein. Such isolated hMPV F polypeptides and immunogenic compositions may be used to immunize a subject and prevent or treat an hMPV infection

An aspect of the invention also provides an isolated nucleic acid encoding any one of the isolated hMPV F polypeptides encoding an hMPV F protein described above. In some embodiments, the isolated nucleic acid is codon-optimized.

An aspect of the invention provides an isolated nucleic acid comprising a nucleotide sequence of SEQ ID NO: 1006, or a variant thereof, wherein the variant nucleotide sequence of the isolated nucleic acid encodes the amino acid sequence of SEQ ID NO: 911, and wherein the isolated nucleic acid does not include residues 1453-1600 of SEQ ID NO: 1003. In various embodiments, the variant nucleotide sequence of the isolated nucleic acid is SEQ ID NO: 1007.

An aspect of the invention also provides an isolated nucleic acid comprising a nucleotide sequence of SEQ ID NO: 1004 or a variant thereof, SEQ ID NO: 1008 or a variant thereof, or SEQ ID NO: 1010 or a variant thereof, wherein the variant of the isolated nucleic acid comprising SEQ ID NO: 1004 encodes the amino acid sequence of SEQ ID NO: 910, wherein the variant of the isolated nucleic acid comprising SEQ ID NO: 1008 encodes the amino acid sequence of SEQ ID NO: 912, and wherein the variant of the isolated nucleic acid comprising SEQ ID NO: 1010 encodes the amino acid sequence of SEQ ID NO: 913. In various embodiments, the variant of the isolated nucleic acid comprising SEQ ID NO: 1004 is SEQ ID NO: 1005, the variant of the isolated nucleic acid comprising SEQ ID NO: 1008 is SEQ ID NO: 1009, and the variant of the isolated nucleic acid comprising SEQ ID NO: 1010 is SEQ ID NO: 1011. In various embodiments, the variant comprises a nucleic acid sequence having 95-99% identity to the nucleic sequence of SEQ ID NO: 1004, SEQ ID NO: 1008, or SEQ ID NO: 1010.

An aspect of the invention also provides an immunogenic composition comprising an isolated antigenic polypeptide, the isolated antigenic peptide consisting of residues 1-490 of SEQ ID NO: 911, or a variant thereof, and a pharmaceutically acceptable carrier. In various embodiments, the variant comprises an amino acid sequence having 95-99% identity to residues 1-490 of SEQ ID NO: 911.

An aspect of the invention also provides an immunogenic composition comprising an isolated antigenic polypeptide, the isolated antigenic peptide comprising residues 1-521 of SEQ ID NO: 910, residues 1-518 of SEQ ID NO: 912, or residues 1-521 of SEQ ID NO: 913, or a variant thereof, wherein the variant of the isolated hMPV F polypeptide comprising residues 1-521 of SEQ ID NO: 910 does not vary at residues 486-517, wherein the variant of the isolated hMPV F polypeptide comprising residues 1-518 of SEQ ID NO: 912 does not vary at residues 102-105 and 490-516, and wherein the variant of the isolated hMPV F polypeptide comprising residues 1-521 of SEQ ID NO: 913 does not vary at residues 99-102, 185, and 485-521. In various embodiments, the isolated antigenic polypeptide consists of residues 1-521 of SEQ ID NO: 910, residues 1-518 of SEQ ID NO: 912, or residues 1-521 of SEQ ID NO: 913, or a variant thereof, wherein the variant of the isolated hMPV F polypeptide comprising residues 1-521 of SEQ ID NO: 910 does not vary at residues 486-517, wherein the variant of the isolated hMPV F polypeptide comprising residues 1-518 of SEQ ID NO: 912 does not vary at residues 102-105 and 490-516, and wherein the variant of the isolated hMPV F polypeptide comprising residues 1-521 of SEQ ID NO: 913 does not vary at residues 99-102, 185, and 485-521. In various embodiments, the variant comprises an amino acid sequence having 95-99% identity to residues 1-521 of SEQ ID NO: 910, residues 1-518 of SEQ ID NO: 912, or residues 1-521 of SEQ ID NO: 913, wherein the variant of the isolated hMPV F polypeptide comprising residues 1-521 of SEQ ID NO: 910 does not vary at residues 486-517, wherein the variant of the isolated hMPV F polypeptide comprising residues 1-518 of SEQ ID NO: 912 does not vary at residues 102-105 and 490-516, and wherein the variant of the isolated hMPV F polypeptide comprising residues 1-521 of SEQ ID NO: 913 does not vary at residues 99-102, 185, and 485-521.

In various embodiments of the immunogenic compositions, the immunogenic composition is multivalent. In various embodiments, the immunogenic composition further comprises an adjuvant. In various embodiments, the immunogenic composition does not comprise an adjuvant.

The invention also provides a method of inducing an immune response in a subject, the method comprising administering to the subject any one of the immunogenic compositions above in an amount effective to produce an antigen-specific immune response in the subject. In various embodiments, the antigen-specific immune response comprises a T cell response or a B cell response. In various embodiments, the subject is administered a single dose of the immunogenic composition. In various embodiments, the subject is administered a booster dose of the immunogenic composition. In various embodiments, the immunogenic composition is administered to the subject by intradermal injection or intramuscular injection. In various embodiments, the subject has been exposed to hMPV, wherein the subject is infected with hMPV, or wherein the subject is at risk of infection from hMPV.

The invention also provides any one of the immunogenic compositions above for use in inducing an antigen-specific immune response in a subject. The invention also provides any one of the immunogenic compositions above for use in preventing or treating hMPV infection in a subject.

The invention provides any one of the isolated hMPV F polypeptides above for use in inducing an antigen-specific immune response in a subject.

The invention provides any one of the isolated hMPV F polypeptides above for use in preventing or treating human metapneumovirus (hMPV) infection in a subject.

The invention provides use of any one of the isolated hMPV F polypeptides or the isolated nucleic acid encoding such polypeptides in the manufacture of a medicament for inducing an antigen-specific immune response in a subject.

The invention provides use of any one of the isolated hMPV F polypeptides above or the isolated nucleic acid encoding such polypeptides or immunogenic compositions comprising such polypeptides in the manufacture of a medicament for preventing or treating human metapneumovirus (hMPV) infection in a subject.

### BRIEF DESCRIPTION OF THE DRAWINGS

FIG. 1A is a drawing showing the workflow of antibody discovery by single memory B cell sorting, culturing, and cloning.

FIG. 1B is a representative set of images showing the B-cell sorting gating strategy. Enriched B-cells were gated using light Back Scatter (BSC) and Forward Scatter (FSC) followed by forward scatter height (FSC-H) by area to exclude debris and doublet cells. Next, cells were gated on CD3 followed by CD19+IgG+ cells. These cells were then used to gate on antigen specific cells (hMPV F+).

FIG. 2A is a schematic diagram of hMPV unprocessed wildtype PreF, unprocessed stabilized PreF, and processed stabilized PreF. FIG. 2A discloses the amino acid sequence of SEQ ID NO: 1000 (ENPRQSRFVL) and the amino acid sequence of SEQ ID NO: 1001 (ENPRRRRFVL), respectively, in order of appearance. SEQ ID NO: 1000 (ENPRQSRFVL) is in SEQ ID NO: 910. SEQ ID NO: 1001 (ENPRRRRFVL) is in SEQ ID NO: 913. FIG. 2B is a picture of a denatured SDS-PAGE gel of the constructs described in FIG. 2A.

FIG. 3A, FIG. 3B, and FIG. 3C show CDR H3 lengths and somatic hypermutations (SHM) data for the hMPV F-specific mAbs. FIG. 3A is a drawing showing the germline frequency of VH and VK/VL described in Table 3A, Table 3B, Table 3C and Table 3D. FIG. 3B is a graph showing CDR H3 lengths in the VH. FIG. 3C is a graph showing nucleotide substitutions of in the VH (excluding CDRH3), and VK/VL. Horizontal bars (identified by arrows) indicate the median. The analysis was based on Kabat delineation system [1].

FIG. 4A is a graph showing antibody M4B06 IgG neutralization assay data for hMPV A and B strains. Error bars indicate the standard deviation of three replicates. FIG. 4B is a graph showing biolayer interferometry (BLI) binding of M4B06 IgG to unprocessed PreF trimer, processed PreF trimer, and PostF trimer. FIG. 4C is a set of graphs and an IC50 table showing binding of M4B06 IgG to unprocessed hMPV PreF WT and PreF carrying monoclonal antibody resistant mutant (MARM) mutants, determined by ELISA with Expi293 cell culture supernatants containing expressed antigens. Error bars indicate the standard deviation of two replicates. FIG. 4D is a graph and EC50 table showing neutralization assay data for M4B06 IgG and Fab, as well as an irrelevant control antibody (DD1L), to hMPV PreF and three types of MARM viruses. Error bars indicate the standard deviation of three replicates. FIG. 4E is a drawing showing the M4B06 epitopes identified by MARM and hydrogen/deuterium-exchange mass spectrometry (HDX-MS) were mapped on an hMPV PreF structure, with epitope residues identified by shading. FIG. 4F is a drawing showing a CryoEM map of three M4B06 Fabs in complex with a processed PreF trimer at 3.75 angstrom (A). The hMPV PreF model with the same shading as FIG. 4E was fit in the density.

FIG. 5 is an epitope map of hMPV PreF trimer protein and M4B06 by HDX-MS. FIG. 5 discloses SEQ ID NO: 1002, which corresponds to amino acids 19-542 of SEQ ID NO: 913.

FIG. 6A is a drawing and FIG. 6B are a set of graphs showing the diverse epitopes of antigenic sites II and V. FIG. 6A is an epitope map showing the contact residues for different site II and V mAbs mapped on an hMPV PreF trimer structure [2]. FIG. 6B are graphs showing binding of selected site II and V mAbs to unprocessed hMPV PreF and epitope-knock out mutants, determined by ELISA with Expi293 cell culture supernatants containing expressed antigens. Error bars indicate the standard deviation of two replicates.

FIG. 7A is a graph showing binding of antibody M2D2 (positive control) IgG to unprocessed hMPV PreF and PreF carrying MARM mutants. Error bars indicates the standard deviation of two replicates. FIG. 7B is a graph showing binding of M1A04 IgG to unprocessed hMPV PreF and epitope-knock out mutants. FIG. 7C is a graph showing binding of M2A05 IgG to unprocessed hMPV PreF and epitope-knock out mutants. The binding was determined by ELISA with Expi293 cell culture supernatants containing expressed antigens.

FIG. 8A, FIG. 8B and FIG. 8C are a set of drawings and a graph showing epitope mapping of hMPV F-specific mAbs. FIG. 8A is a heatmap drawing showing epitope binning of selected mAbs with the unprocessed hMPV PreF antigen. Seventy-three isolated mAbs which showed apparent BLI binding response (>0.2 nm) with the tested antigen were included. The heatmap shows epitope binning from a sandwich-based BLI binding assay, with markers/shading indicating more competition between the 1^{st} and the 2^{nd} antibodies. Top side-bars showed the neutralization potency (IC₅₀) of mAbs to hMPV A, hMPV B, RSV A, and RSV B virus strains. FIG. 8B is a drawing showing major antigenic sites II, III, III′, IV, IV′, V (internal antigenic site α not visible, antigenic site β not yet clearly characterized) that were mapped on the hMPV PreF trimer protein structure. The DS7-site was also labeled as a reference. FIG. 8C is a graph showing percentage of antibodies targeting each antigenic site, grouped by neutralization potency. For each mAb, the stronger neutralization potency of hMPV A and B were chosen for grouping.

FIG. 9 is a set of pictures showing the negative staining EM and 2D average of engineered hMPV postF trimer protein. Most class averages show particles with a distinct head portion (top area) and an elongated tail portion (middle section). In some averages (bottom/tail section) the tail portion appears longer with a small distinct portion at the end of the tail. Visible in some averages was an indentation in the ‘head’ portion of the particle (identified with an arrow). All figures to scale.

FIG. 10A, FIG. 10B, FIG. 10C, FIG. 10D, and FIG. 10E are a set of graphs showing correlations among neutralization potency, antigenic sites, and binding-specificity to different hMPV F conformations. FIG. 10A and FIG. 10B are graphs showing BLI binding response of hMPV-F specific mAbs to unprocessed hMPV PreF trimer and PostF trimer. Every dot represents an isolated hMPV F-specific antibody, distinguished by neutralization potency (FIG. 10A) or mapped antigenic site: V, II, III, III′, IV, IV′, site α, site β, or uncharacterized (FIG. 10B). FIG. 10C and FIG. 10D are graphs showing BLI binding response of hMPV-F specific mAbs to unprocessed hMPV PreF and processed stabilized hMPV PreF (115BV) antigens. Every dot represents an isolated hMPV F-specific antibody, shaded by neutralization potency (FIG. 10C) or mapped antigenic site: V, II, III, III′, IV, IV′, site α, site β, or uncharacterized (FIG. 10D). FIG. 10E is a graph showing correlations between neutralization potency (after Log₁₀ transformation) and ratio of processed PreF/unprocessed PreF BLI binding. Dashed line and R-square number indicate the linear regression. Every dot represents an isolated hMPV F-specific antibody, which is shaded and identified the mapped antigenic site (V, II, III, III′, IV, IV′, site α, site β, or uncharacterized) to which it binds.

FIG. 11A is a drawing showing the format of magnetic bead-based serum absorption assay that was used to analyze the unprocessed hMPV PreF, processed hMPV PreF, hMPV PostF, and RSV PreF trimers.

FIG. 11B is a graph showing the averaged ELISA titers from four donors after antigen depletion, normalized by titers of un-depleted serum. Error bar shows SEM.

FIG. 12A, FIG. 12B and FIG. 12C are a set of graphs showing differential scanning fluorimetry (DSF) spectra of: unprocessed hMPV PreF protein (1.5 mg/ml; FIG. 12A); processed stabilized hMPV PreF protein expressed without furin (2.5 mg/ml; FIG. 12B); and unprocessed stabilized hMPV PreF protein expressed without furin cleavage (2.7 mg/ml; FIG. 12C). Spectra were recorded at a scan rate of 1° C./min in duplicate. Graphs depict the ratio of the fluorescence between 350 nm and 330 nm (top) as well as the first derivative of this ratio (bottom).

FIG. 13 is a graph showing a distribution of isolated hMPV-F specific mAbs and their mapped antigenic sites (II, V, III, III′, IV, IV′, site α, or uncharacterized), grouped by donors. The left Y-axis indicates the number of mAbs discovered from each donor. The right Y-axis indicates the serum neutralization titer (fold-dilution) of each donor. Markers/dots indicated the neutralization titers of donors' sera in fold dilution. One donor was not included as only one mAb was isolated from this donor.

FIG. 14A and FIG. 14B are a set of drawings showing unprocessed hMPV PreF monomer (FIG. 14A) and processed stabilized hMPV PreF trimer from a side view and top a view (FIG. 14B). The N-terminus segment of F1 near a cleavage site is identified.

FIGS. 15 A-E are a set of drawings and a table showing the interaction of antibody M8C10 and MPV. FIG. 15A is a stereo view/drawing of the MPV-F:M8C10 complex (top) with the MPV-F trimer (PDB: code SwbO) shown from the same perspective for reference (bottom). FIG. 15B is a set of individual views/drawings of the MPV-F:M8C10 complex (top panels) from the perspective of the Fab (left), from the side (center), and from the top (right). The same perspectives of the MPV-F trimer are shown below for each panel. FIG. 15C is a list of the sequence of the M8C10 heavy chain (HC) and light chain (LC) are shown with the CDR regions highlighted and shaded as follows, HC CDR1, HC CDR2, HC CDR3, LC CDR1, LC CDR2, LC CDR3. FIG. 15C also includes a drawing of the surface representation of the complex is shown on the right with the CDRs shaded as the sequence, with the F protein, the heavy chain, and the light chain identified. FIG. 15D is a drawing showing the interactions mediated by the four interacting CDR regions. FIG. 15E is a listing of the residues involved in the interaction between MPV-F and M8C10, with residues in the CDRs shaded.

FIG. 16A is a series of graphs and FIG. 16B is a series of drawings showing epitope residue mutations on M8C10 binding. FIG. 16A is a set of graphs showing ELISA binding analysis of MPV-F mutations. Binding to M8C10 is shown in blue over a range of concentrations. As a control, M2D2, a site IV binder, is shown in red. FIG. 16B is a set of drawings showing interactions mediated by R198, N202, and A216, based on the structure.

FIG. 17 is a set of drawings showing a comparison of M8C10 binding epitopes and MPV458 binding epitopes for hMPV F monomer. Crystal structures M8C10 (lower) and MPV458 (upper) in complex with hMPV F monomer were superimposed by hMPV F, showing their distinct epitopes. Indicated area indicate the direct contacting residues of hMPV F with M8C10. An inlet to the left shows a hMPV F trimer structure with one protomer (left) and the other two protomers shaded (right).

### DETAILED DESCRIPTION

Pneumoviruses including respiratory syncytial virus (RSV) and hMPV are common causes of acute lower respiratory infection (ALRI) [1-6]. Like RSV, the infection of hMPV leads to a variety of symptoms including coughing, wheezing, pneumonia, and bronchiolitis. The high-risk populations, including infants, young children, elderly people, and immunocompromised patients, are more likely to develop severe symptoms that may potentially require hospitalization [7-14]. Co-infection with other respiratory pathogens is common. Recently, cases of co-infection of SARS-CoV-2 and hMPV have also been reported [15-17], and such co-infection with the hMPV appeared to affect monocytes and dampen interferon response in a severe COVID-19 patient [15]. Despite of these medical burdens, currently there is no approved hMPV vaccine or neutralizing antibody available for therapeutic or prophylactic purpose.

The surface glycoprotein F of hMPV mediates the subsequent fusion of viral and cellular membranes [18, 19]. F protein is highly conserved in sequence between different hMPV subtypes, and it appears to be more critical for virus infection than other surface proteins G and SH [20, 21]. It has also been reported that F is the primary viral antigen of hMPV that elicits neutralizing and protective antibodies against hMPV infection, whereas antibodies elicited by G and SH proteins are not protective [22-24]. Therefore, the F protein is an attractive target for neutralizing antibody and vaccine development against hMPV infection.

The hMPV F protein is a class I viral fusion protein that presents as a homo-trimer on viral surface as well as the membrane of host cells. The precursor of F (F₀) is synthesized as an intact polypeptide and subsequently subjected to proteolytic processing to become functional [19]. Unlike RSV which has two furin-cleavage sites that can be cleaved twice intracellularly during transport to the cell membrane [25], hMPV only has one protease cleavage site being cleaved at the cell surface or in the virus like particle by transmembrane proteases such as TMPRSS2 [26]. Cleavage of both RSV and hMPV F generates two disulfide-linked chains (F₂ and F₁). The fusion peptide, which is located at the N-terminus of F₁ right after the protease cleavage site, is buried inside a hydrophobic cavity and interact with adjacent monomers, presumably stabilizing the trimer conformation [27, 28]. This globular “prefusion” (PreF) form of proteolytically processed F trimer is metastable and can be triggered into a more stable rod-shaped “postfusion” (PostF) form, through a series of conformational changes [19]. Protein engineering efforts have been made to stabilize hMPV and RSV F in PreF and PostF conformations by introducing additional mutations or modifying protease cleavage sites [27-32]. Despite only about 35% sequence identity, the structures of both stabilized PreF and PostF proteins between RSV F and hMPV F are remarkably conserved [27-31, 33, 34].

Over the past years, a large number of monoclonal antibodies (mAbs) targeting RSV F antigens have been isolated from human B cell repertoire and characterized by various approaches [33, 35-47]. According to these results, six major antigenic sites on RSV F have been defined (Ø, I, II, III, IV, V). PreF-specific antibodies targeting sites Ø and V have been shown to be immunodominant and accountable for a large proportion of neutralizing activity in human sera and antigen-specific memory B cell repertoire of adults [48, 49], suggesting that PreF is a better vaccine candidate of RSV to elicit higher neutralizing antibody (nAb) responses than PostF. In contrast, the immunogenicity potential of stabilized hMPV PreF and PostF appears to be comparable, likely due to the large glycan shield that is only observed at the site Ø of hMPV F protein [28]. Although a limited number of hMPV-F specific mAbs have been discovered from mice immunization [50], phage display [51], and human B cells [52], a comprehensive understanding of human antibody recognition to hMPV F during natural infection remains elusive.

In this study, we reported a large panel of hMPV-F specific mAbs isolated from memory B cells of multiple healthy human donors. Some isolated mAbs exhibited ultra-high neutralization potency in vitro. Investigators have characterized one of the most potent mAbs that recognizes a PreF-specific epitope on site V. Further in-depth characterization of isolated mAbs revealed diverse antigenic sites on hMPV F antigens, including overlapping but distinct epitopes on pre-defined site II and V, and four novel antigenic sites that have not been reported previously. See also Huang et al., 2019 Front. Immunol, pgs. 1-8; Rossey et al., 2018 Trends in Microbiology, vol. 26, issue 3, pgs. 209-219.
Antigenic Sites of hMPV Site Ø—amino acids 54-84; 165-197 of SEQ ID NO: 1
Site I—amino acids 19-36; 283-289; 347-358 of SEQ ID NO: 1
Site II—amino acids 156, and 224-247 of SEQ ID NO: 1
Site III—amino acids 37-45; 271-282; 312-320; and 335-346 of SEQ ID NO: 1;
Site III′—Competitive binding to hMPV F antigen protein with site III and DS7-site antibodies (M2B6, DS7), do not compete with site II antibodies (M1D2 and M1C7s).
Site IV—amino acids 386-439 of SEQ ID NO: 1
Site IV′—amino acids 294-298 of SEQ ID NO: 1
Site V—amino acids 46-53; 116-164; 233, and 257-270 of SEQ ID NO: 1
Site α—amino acids 82, 198, 202, 207, 211, 214-216, 218, 219, 220, 224-229, 233, 257, 258, 261, 262, 338, and 405 of SEQ ID NO: 1
Site β—Not well-characterized. Most of mAbs in this group only compete with themselves DS7-Site (ref. DOI: 10.1038/nsmb.2250): amino acids 20-26, 31, 33-34, 282, 284, 312, 345, 348-349, 351, 353, 356, 358, 411, 413, and 414 of SEQ ID NO: 1.

Unlike RSV, the antibody responses to hMPV F appear to be less dominant against the apex of the antigen. Furthermore, a panel of mAbs that bind specifically to the uncleaved PreF have been identified, suggesting the potential immunogenic differences between unprocessed and processed F antigens. This is the first disclosure revealing the comprehensive antigenic epitopes of hMPV F-specific mAbs elicited from natural infection.

### Definitions

As used herein, “Human metapneumovirus” or “hMPV” refers to the single-stranded, negative-sense ribonucleic acid (RNA) virus of the family Pneumoviridae, with a genome of about 13 kb. As used herein, “hMPV F protein,” refers to a fusion glycoprotein F0 that has the amino acid sequence set forth in UniProtKB—G3KCK8 (G3KCK8_9MONO; SEQ ID NO: 1).

```
(SEQ ID NO: 1)
    10 20 30 40 50
MSWKVMIIIS LLITPQHGLK ESYLEESCST ITEGYLSVLR TGWYTNVFTL
    60 70 80 90 100
EVGDVENLTC TDGPSLIKTE LDLTKSALRE LKTVSADQLA REEQIENPRQ
    110 120 130 140 150
SRFVLGAIAL GVATAAAVTA GIAIAKTIRL ESEVNAIKGA LKTTNEAVST
    160 170 180 190 200
LGNGVRVLAT AVRELKEFVS KNLTSAINKN KCDIADLKMA VSFSQFNRRF
    210 220 230 240 250
LNVVRQFSDN AGITPAISLD LMTDAELARA VSYMPTSAGQ IKLMLENRAM
    260 270 280 290 300
VRRKGFGILI GVYGSSVIYM VOLPIFGVID TPCWIIKAAP SCSEKDGNYA
    310 320 330 340 350
CLLREDQGWY CKNAGSTVYY PNDKDCETRG DHVFCDTAAG INVAEQSREC
    360 370 380 390 400
NINISTTNYP CKVSTGRHPI SMVALSPLGA LVACYKGVSC SIGSNRVGII
    410 420 430 440 450
KQLPKGCSYI TNQDADTVTI DNTVYQLSKV EGEQHVIKGR PVSSSFDPIK
    460 470 480 490 500
FPEDQFNVAL DQVFESIENS QALVDQSNKI LNSAEKGNTG FIIVIILIAV
    510 520 530
LGLTMISVSI IIIIKKTRKP TGAPPELNGV TNGGFIPHS
```

As used herein, the term “antibody” refers to any form of immunoglobulin molecule that exhibits the desired biological or binding activity. An antibody that “specifically binds to” hMPV is an antibody that exhibits preferential binding to hMPV as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins. Thus, the term “antibody” is used in the broadest sense and specifically covers, but is not limited to, monoclonal antibodies (including full length monoclonal antibodies), polyclonal antibodies, multi-specific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain typically includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are typically joined by a “J” region of about 12 or more amino acids, with the heavy chain also typically including a “D” region of about 10 more amino acids. See generally, Fundamental Immunology Ch. 7 (Paul, W., ed., 2nd ed. Raven Press, N.Y. (1989).

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Antibodies typically bind specifically to their cognate antigen with high affinity, reflected by a dissociation constant (K_{D}) of 10⁻⁵ to 10⁻¹² M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens.

As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

As used herein, unless otherwise indicated, “antibody fragment” or “antigen binding fragment” or “antigen binding fragment thereof” refers to a fragment of an antibody that retains the ability to bind specifically to the antigen, e.g., fragments that retain one or more CDR regions and the ability to specifically bind to the antigen.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., hMPV).

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

Antigen binding fragments thereof within the scope of the present invention also include F(ab′)2 fragments which may be produced by enzymatic cleavage of an IgG by, for example, pepsin. Fab fragments may be produced by, for example, reduction of F(ab′)2 with dithiothreitol or mercaptoethylamine. A Fab fragment is a VL-CL chain appended to a VH-CH1 chain by a disulfide bridge. A F(ab′)2 fragment is two Fab fragments which, in turn, are appended by two disulfide bridges. The Fab portion of an F(ab′)2 molecule includes a portion of the Fc region between which disulfide bridges are located.

The term “acceptor human framework” refers to a framework comprising the amino acid sequence of a light chain variable domain (VL) framework or a heavy chain variable domain (VH) framework derived from a human immunoglobulin framework or a human consensus framework. An acceptor human framework “derived from” a human immunoglobulin framework or a human consensus framework may have the same amino acid sequence as the naturally-occurring human immunoglobulin framework or human consensus framework, or it may have amino acid sequence changes compared to wild-type naturally-occurring human immunoglobulin framework or human consensus framework. In some embodiments, the number of amino acid changes are 10, 9, 8, 7, 6, 5, 4, 3, or 2, or 1. In some embodiments, the VL acceptor human framework is identical in sequence to the VL human immunoglobulin framework sequence or human consensus framework sequence. In some embodiments, the VH acceptor human framework is identical in sequence to the VH human immunoglobulin framework sequence or human consensus framework sequence.

The term “binding protein” as used herein also refers to a non-naturally occurring (or recombinant) protein that specifically binds to at least one target antigen. In various embodiments, the binding protein comprises an anti-hMPV antibody or antigen binding fragment thereof described herein. For example, the binding protein is a multi-specific molecule (e.g., multi-specific antibody) comprising the anti-hMPV antibody or antigen binding fragment thereof, and a second molecule (e.g., a second antibody or antigen binding fragment thereof).

A “multi-specific antibody” is an antibody (e.g., bispecific antibodies, trispecific antibodies) that recognizes two or more different antigens or epitopes. In various embodiments, multi-specific antibodies normally will bind at least two antigens or epitopes (i.e., bispecific antibodies, BsAbs) or more than two antigens. In various embodiments, multi-specific antibodies with additional specificities such as trispecific antibodies are encompassed herein. Examples of BsAbs include those with one arm directed against a viral antigen #1 and the other arm directed against a viral antigen #2. For example, the BsAbs may bind to hMPV strain A and hMPV strain B.

Techniques for making multi-specific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making bispecific antibody fragments (see, e.g., Holliger, P. et al., Proc. Natl. Acad. Sci. USA 90 (1993) 6444-6448); and using single-chain Fv (scFv) dimers (see, e.g. Gruber, M et al., J. Immunol. 152 (1994) 5368-5374); and preparing trispecific antibodies as described, e.g., in Tutt, A. et al., J. Immunol. 147 (1991) 60-69). Engineered antibodies with three or more functional antigen binding sites, including “Octopus antibodies”, are also included herein (see, e.g. US 2006/0025576; and Proc Natl Acad Sci USA. 2011 Jul. 5; 108(27): 11187-92).

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Bifunctional antibodies include, for example, heterodimeric antibody conjugates (e.g., two antibodies or antibody fragments joined together with each having different specificities), antibody/cell surface-binding molecule conjugates (e.g., an antibody conjugated to a non-antibody molecule such as a receptor), and hybrid antibodies (e.g., an antibody having binding sites for two different antigens). Bispecific antibodies include those generated by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (Holliger et al. (1993) Proc. Natl. Acad. Sci. USA 90(14): 6444-6448), among others.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the VH and VL regions of the recombinant antibodies are sequences that, while derived from and related to human germline VH and VL sequences, may not naturally exist within the human antibody germline repertoire in vivo.

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

Antigen binding fragments (including scFvs) of such immunoglobulins are also encompassed by the term “monoclonal antibody” as used herein. Monoclonal antibodies are highly specific, being directed against a single antigenic site. Furthermore, in contrast to conventional (polyclonal) antibody preparations, which typically include different antibodies directed against different epitopes on the antigen, each monoclonal antibody is directed against a single epitope. Monoclonal antibodies can be prepared using any art recognized technique and those described herein such as, for example, a hybridoma method, a transgenic animal, recombinant DNA methods (see, e.g., U.S. Pat. No. 4,816,567), or using phage antibody libraries using the techniques described in, for example, U.S. Pat. No. 7,388,088 and PCT Pub. No. WO 00/31246). Monoclonal antibodies include chimeric antibodies, human antibodies, and humanized antibodies and may occur naturally or be produced recombinantly.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the VH and VL domains of antibody, wherein these domains are present in a single polypeptide chain. Generally, the Fv polypeptide further comprises a polypeptide linker. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one embodiment, the present invention provides single domain antibodies comprising two VH domains with modifications such that single domain antibodies are formed.

As used herein, the term “diabodies” refers to small antibody fragments with two antigen binding sites, which fragments comprise a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes, such as substitutions, deletions and insertions, glycosylation or deglycosylation, and adding multiple Fc. Changes to the Fc may be utilized to alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only.

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may also be referred to as “heavy chain variable region”, “heavy chain variable domain”, “VH” or “V_{H}” in the instant disclosure. The variable region of the light chain may be referred to as “light chain variable region”, “heavy chain variable domain”, “VL” or “V_{L}” in the instant disclosure. Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system. See Kabat et al., (1991) Sequences of Proteins of Immunological Interest, 5th Ed. Public Health Service, National Institutes of Health, Bethesda, Md.) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, (1987) J. Mol. Biol. 196: 901-917).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂, IgG₃, and IgG₄ constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al., ILAR Journal 37: 132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof Andrew C.R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1
CDRs in antibody and antigen binding fragments thereof

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing. Amino acid residues in antibodies can also be defined using other numbering systems, such as Chothia, enhanced Chothia, IMGT, Kabat/Chothia composite, Honegger (AHo), Contact, or any other conventional antibody numbering scheme.

As used herein, the term “isolated” used in the context of polypeptides or polynucleotides refers to polypeptides or polynucleotides that are at least partially free of other biological molecules from the cells or cell cultures in which they are produced. Such biological molecules include other nucleic acids, proteins, lipids, carbohydrates, or other material such as cellular debris and growth medium. It may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the polypeptides or polynucleotides.

An “isolated antibody,” as used herein, is intended to refer to an antibody which is substantially free of other antibodies having different antigenic specificities.

As used herein, “isotype” refers to the antibody class (e.g., IgG (including IgG1, IgG2, IgG3, and IgG4), IgM, IgA (including IgA1 and IgA2), IgD, and IgE antibody) that is encoded by the heavy chain constant region genes of the antibody.

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

The term “epitope” or “antigenic determinant” refers to a site on an antigen (e.g., hMPV) to which an immunoglobulin or antibody specifically binds. Epitopes can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, or 22 amino acids in a unique spatial conformation.

The term “epitope mapping” refers to the process of identifying the molecular determinants on the antigen involved in antibody-antigen recognition. Methods for determining what epitopes are bound by a given antibody are well known in the art and include, for example, immunoblotting and immunoprecipitation assays, wherein overlapping or contiguous peptides from, e.g., hMPV are tested for reactivity with a given antibody (e.g., anti-hMPV antibody); x-ray crystallography; antigen mutational analysis, two-dimensional nuclear magnetic resonance; yeast display; and hydrogen/deuterium exchange-mass spectrometry (HDX-MS) (see, e.g., Epitope Mapping Protocols in Methods in Molecular Biology, Vol. 66, G. E. Morris, Ed. (1996)). See also Champe et al. (1995) J. Biol. Chem. 270:1388-1394.

The term “binds to the same epitope” with reference to two or more antibodies means that the antibodies bind to the same segment or same segments of amino acid residues, as determined by a given method. Techniques for determining whether antibodies bind to the “same epitope on hMPV” with the antibodies described herein include, for example, epitope mapping methods, such as x-ray analyses of crystals of antigen:antibody complexes, which provides atomic resolution of the epitope, and HDX-MS. Other methods monitor the binding of the antibody to antigen fragments thereof (e.g. proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081), yeast display of mutant target sequence variants, or analysis of chimeras. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known binding competition experiments, e.g., BIACORE® surface plasmon resonance (SPR) analysis. In certain embodiments, an antibody competes with, and inhibits binding of another antibody to a target by at least 50%, 60%, 70%, 80%, 90% or 100%. The level of inhibition or competition may be different depending on which antibody is the “blocking antibody” (i.e., the antibody that when combined with an antigen blocks another immunologic reaction with the antigen). Competition assays can be conducted as described, for example, in Ed Harlow and David Lane, Cold Spring Harb. Protoc. 2006; doi:10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope, or to adjacent epitopes (e.g., as evidenced by steric hindrance). Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include competition for binding to cells expressing hMPV, e.g., by flow cytometry. Other methods include: SPR (e.g., BIACORE®), solid phase direct or indirect radioimmunoassay (RIA), bio-layer interferometry (BLI) analysis (e.g., OCTET analysis using BLI), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., SPR using a predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Any K_{D} greater than about 10⁻⁴ M is generally considered to indicate nonspecific binding.

The term “k_{assoc}” or “kₐ”, as used herein, refers to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e., k_{d}/kₐ) and is expressed as a molar concentration (M). K_{D} values for antibodies or antigen binding fragments thereof can be determined using methods well established in the art. A preferred method for determining the K_{D} of an antibody or antigen binding fragment thereof is by using SPR, preferably using a biosensor system such as a Biacore® system or flow cytometry and Scatchard analysis, or bio-layer interferometry.

The term “EC50” in the context of an in vitro or in vivo assay using an antibody or antigen binding fragment thereof refers to the concentration of an antibody or antigen binding fragment thereof that induces a response that is 50% of the maximal response, i.e., halfway between the maximal response and the baseline.

The term “cross-reacts,” as used herein, refers to the ability of an antibody or antigen binding fragment thereof described herein to bind to metapneumovirus from a different species. For example, an antibody or antigen binding fragment thereof described herein that binds hMPV may also bind other family members in Pneumoviridae family (e.g., human RSV, bovine RSV, or murine pneumonia virus), or another species of MPV (e.g., avian MPV). Cross-reactivity may be measured by detecting a specific reactivity with purified antigen in binding assays (e.g., SPR, ELISA, bio-layer interferometry) or binding to, or otherwise functionally interacting with, cells physiologically expressing the antigen (e.g., HT1080 cells overexpressing hMPV). Methods for determining cross-reactivity include standard binding assays as described herein, for example, by bio-layer interferometry or flow cytometric techniques.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies or antibody binding fragments thereof (e.g., VH, VL, CDR3), is intended to refer to a nucleic acid molecule in which the nucleotide sequences are essentially free of other genomic nucleotide sequences, e.g., those encoding antibodies or antibody binding fragments thereof that bind antigens other than hMPV, which other sequences may naturally flank the nucleic acid in human genomic DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

Also provided are “conservative sequence modifications” of the sequences set forth herein, i.e., amino acid sequence modifications which do not abrogate the binding of the antibody or antigen binding fragment thereof encoded by the nucleotide sequence or containing the amino acid sequence, to the antigen. Such conservative sequence modifications include conservative nucleotide and amino acid substitutions, as well as nucleotide and amino acid additions and deletions. For example, modifications can be introduced into a sequence in a table herein (e.g., Table 3B, Table 3C, Table 3D or in Examples 1-6) by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-hMPV antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another embodiment, mutations can be introduced randomly along all or part of an anti-hMPV antibody coding sequence or anti-hMPV antigen binding fragment thereof coding sequence, such as by saturation mutagenesis, and the resulting modified anti-hMPV antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and more preferably at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, more preferably at least about 98% to 99.5% of the amino acids, or more preferably at least about 98% to 99.9% of the amino acids.

The percent identity between two sequences is a function of the number of identical positions shared by the sequences (i.e., % homology=# of identical positions/total # of positions×100), accounting for the number of gaps and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at www.gcg.com), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) NucleicAcids Res. 25(17):3389-3402. When utilizing BLAST and Gapped BLAST programs, the default parameters of the respective programs (e.g., XBLAST and NBLAST) can be used. See www.ncbi.nlm.nih.gov.

As used herein, the term “variant” in the context of recombinant hMPV F proteins refers to a molecule that differs in its amino acid sequence or nucleic acid sequence relative to a native sequence or a reference sequence. Sequence variants may possess substitutions, deletions, insertions, or a combination of any two or three of the foregoing, at certain positions within the sequence, as compared to a native sequence or a reference sequence. Ordinarily, variants possess at least 50% identity to a native sequence or a reference sequence. In some embodiments, variants share at least 80% identity or at least 90%, 91%, 92%, 93%, 94%, 95%, 96%, 97%, 98% or 99% identity with a native sequence or a reference sequence. In some embodiments, variants share at least between 90%-99%, 91%-99%, 92%-99%, 93%-99%, 94%-99%, 95%-99%, 96%-99%, 97%-99%, or 98%-99% identity with a native sequence or a reference sequence. In some embodiments, a variant differs from the native sequence by 10, 9, 8, 7, 6, 5, 4, 3, 2, or 1 amino acids or nucleic acids.

As used herein, the term “analog” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences; in particular, the polypeptide sequences disclosed herein are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble or linked to a solid support.

“Substitutional variants” when referring to polypeptides, are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild type molecules). The term “identity,” as known in the art, refers to the degree of sequence relatedness between two sequences of polynucleotide or polypeptide molecules as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods.

The term “percent identity” or “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, accounting for the number of gaps and the length of each gap, which needs to be introduced for optimal alignment of the two sequences. The comparison of sequences and determination of percent identity between two sequences can be accomplished using a mathematical algorithm in an alignment tool (e.g. the Needleman-Wunsch algorithm in an online tool).

As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see www.ebi.ac.uk/Tools/psa/emboss_needle/) or the global alignment tool “BLAST®»Global Alignment” from the National Center for Biotechnology Information (NCBI) (see blast.ncbi.nlm.nih.gov/Blast.cgi?PROGRAM=blastn&PAGE_TYPE=BlastSearch&PROG_DEF AULTS=on&BLAST_INIT=GlobalAln&BLAST_SPEC=GlobalAln&BLAST_PROGRAMS=bl astn). Both of these global alignment tools incorporate the Needleman-Wunsch algorithm (Needleman, S. B. & Wunsch, C. D. (1970) “A general method applicable to the search for similarities in the amino acid sequences of two proteins.” J. Mol. Biol. 48:443-453). In a preferred embodiment, a global alignment of nucleotide sequences using BLAST Global Alignment uses the following default parameters: match score=2; mismatch score=−3; Gap Cost Existence score=5; Gap Cost Extension Score=2. In a preferred embodiment, a global alignment of protein sequences using BLAST Global Alignment uses the following default parameters: Gap Cost Existence=11; Gap Cost Extension=1.

The term “recombinant host cell” (or simply “host cell”), as used herein, is intended to refer to a cell that comprises a nucleic acid that is not naturally present in the cell, and may be a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “inhibition” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity (e.g., inhibition of HMPV).

For example, as used herein, the term “inhibits hMPV activity” includes any measurable decrease in hMPV activity, e.g., an inhibition of hMPV activity by at least about 10%, for example, at least about 20%, at least about 30%, at least about 40%, at least about 50%, at least about 60%, at least about 70%, at least about 80%, at least about 90%, at least about 99%, or about 100%, relative to a control (e.g., a control antibody). The inhibition may be specific to a single mechanism of hMPV activity or may be generalizable to all mechanisms of hMPV activity.

The term “multivalent”, used in the context of an immunogenic composition herein (e.g. “multivalent immunogenic composition”) refers to a pharmaceutical preparation comprising more than one active agent that provides active immunity to a disease or pathological condition caused by more than one strain of pathogen. For example, a multivalent immunogenic composition against hMPV may protect against more than one strain of hMPV.

An “adjuvant,” as used herein, is a substance that serves to enhance the immunogenicity of a composition of the disclosure. An adjuvant may enhance an immune response to an antigen that is weakly immunogenic when administered alone, e.g., inducing no or weak antibody titers or cell-mediated immune response, increase antibody titers to the antigen, and/or lower the dose of the antigen effective to achieve an immune response in the individual. Thus, adjuvants are often given to boost the immune response and are well known to the skilled artisan.

As used herein, the term “prevent” or “preventing” means to administer a prophylactic agent, such as a composition containing any of the polypeptides of the present invention, to a subject or patient at risk of becoming infected by human metapneumovirus (hMPV). Preventing includes reducing the likelihood or severity of a subsequent hMPV infection, ameliorating symptoms associated with a subsequent hMPV infection, and inducing immunity to protect against hMPV infection. Typically, the agent is administered in an amount effective to neutralize hMPV in the body in order to block infection. The amount of a prophylactic agent that is effective to ameliorate any particular disease symptom may vary according to factors such as the age, and weight of the patient, and the ability of the agent to elicit a desired response in the subject. Whether a disease symptom has been ameliorated can be assessed by any clinical measurement typically used by physicians or other skilled healthcare providers to assess the severity or progression status of that symptom or in certain instances will ameliorate the need for hospitalization.

The terms “treat,” “treating,” and “treatment,” as used herein, refer to therapeutic or preventative measures described herein. The methods of “treatment” employ administration to a subject with a tumor or cancer or a subject who is predisposed to having such a disease or disorder, an anti-hMPV antibody (e.g., anti-human hMPV antibody) or antigen binding fragment thereof described herein, in order to prevent, cure, delay, reduce the severity of, or ameliorate one or more symptoms of the disease or disorder or recurring disease or disorder, or in order to prolong the survival of a subject beyond that expected in the absence of such treatment.

“Immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating hMPV or an condition associated with hMPV.

As used herein, “immune cell” refers to the subset of blood cells known as white blood cells, which include mononuclear cells such as lymphocytes, monocytes, macrophages, and granulocytes.

As used herein, “administering” refers to the physical introduction of a molecule (e.g., an antibody or antigen binding fragment thereof that binds hMPV) or of a composition comprising a therapeutic agent (e.g., an anti-hMPV antibody or antigen binding fragment thereof) to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent (e.g., an anti-hMPV antibody or antigen binding fragment thereof) to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, “adjunctive” or “combined” administration (co-administration) includes simultaneous administration of the agents and/or compounds in the same or different dosage form, or separate administration of the compounds (e.g., sequential administration). For example, at least one agent comprises an anti-hMPV antibody or antigen binding fragment thereof. Thus, a first antibody or antigen binding fragment thereof, e.g., an anti-hMPV antibody or antigen binding fragment thereof, and a second, third, or more antibodies or antigen binding fragments thereof can be simultaneously administered in a single formulation. Alternatively, the first and second (or more) antibodies or antigen binding fragments thereof can be formulated for separate administration and are administered concurrently or sequentially.

“Combination” therapy, as used herein, means administration of two or more therapeutic agents in a coordinated fashion, and includes, but is not limited to, concurrent dosing. Specifically, combination therapy encompasses both co-administration (e.g. administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. (See, e.g., Kohrt et al. (2011) Blood 117:2423). For example, the anti-hMPV antibody can be administered first followed by (e.g., immediately followed by) the administration of a second antibody (e.g., an anti-viral antibody) or antigen binding fragment thereof, or vice versa. In one embodiment, the anti-hMPV antibody or antigen binding fragment thereof is administered prior to administration of the second antibody or antigen binding fragment thereof. In another embodiment, the anti-hMPV antibody or antigen binding fragment thereof is administered, for example, a few minutes (e.g., within about 30 minutes) or at least one hour of the second antibody or antigen binding fragment thereof. Such concurrent or sequential administration preferably results in both antibodies or antigen binding fragments thereof being simultaneously present in treated patients.

The term “effective dose” or “effective dosage” is defined as an amount sufficient to achieve or at least partially achieve a desired effect, e.g., viral neutralization and viral binding. A “therapeutically effective amount” or “therapeutically effective dosage” of a drug (e.g., anti-hMPV antibody or antigen binding fragment thereof) is any amount of the drug or therapeutic agent that, when used alone or in combination with another therapeutic agent, promotes disease regression evidenced by a decrease in severity of disease symptoms, an increase or therapeutic agent in frequency and duration of disease symptom-free periods, or a prevention of impairment or disability due to the disease affliction. A therapeutically effective amount or dosage of a drug or therapeutic agent includes a “prophylactically effective amount” or a “prophylactically effective dosage”, which is any amount of the drug or therapeutic agent that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, for the treatment of viral infections such as hMPV, a therapeutically effective amount or dosage of the drug or therapeutic agent (e.g., anti-hMPV antibody or antigen binding fragment thereof) inhibits viral activity, viral load, or virus diseases symptoms by at least about 20%, by at least about 30% by at least about 40%, by at least about 50%, by at least about 60%, by at least above 70%, by at least about 80%, or by at least about 90% relative to untreated subjects. In some embodiments, a therapeutically effective amount or dosage of the drug or therapeutic agent completely inhibits viral activity. The ability of a compound or therapeutic agent, including an antibody, to inhibit viral activity can be evaluated using the assays described herein. Alternatively, this property of a composition comprising the compound or therapeutic agent can be evaluated by examining the ability of the composition to inhibit viral activity; such inhibition can be measured in vitro by assays known to the skilled practitioner.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, cats, dogs, cows, chickens, amphibians, reptiles, etc.

The term “sample” refers to tissue, bodily fluid, or a cell (or a fraction of any of the foregoing) taken from a patient or a subject. Normally, the tissue or cell will be removed from the patient, but in vivo diagnosis is also contemplated. Other samples, including urine, tears, serum, plasma, cerebrospinal fluid, feces, sputum, cell extracts etc. can also be useful for particular cancers.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to ±10% from the specified value. Unless otherwise indicated, all numbers expressing quantities of ingredients, properties such as molecular weight, reaction conditions, etc., used herein are to be understood as being modified by the term “about”.

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone.

As used herein, the terms “ug” and “microgram” are used interchangeably with “μg” in the following sections. As used herein, the terms “uM” and “micromolar” are used interchangeably with “μM” in the following sections.

Various aspects described herein are described in further detail in the following subsections.

### I. Anti-hMPV Antibodies

In one aspect, provided herein is an isolated anti-hMPV antibody (i.e., an antibody that binds hMPV) or antigen binding fragment thereof.

In one aspect, provided herein is an isolated anti-hMPV antibody (e.g., recombinant humanized, chimeric, or human antibody) or antigen binding fragment thereof which comprises:

Functional features of the anti-hMPV antibodies or antigen binding fragment thereof provided herein are described below in more detail.

In some embodiments, the anti-hMPV antibody or antigen binding fragment thereof described herein binds to a portion of hMPV (e.g., site α, site β, site II, site III, site IV and site V) with a K_{D} described in the following Examples.

An antibody or antigen binding fragment thereof that exhibits one or more of the functional properties described above (e.g., biochemical, immunochemical, cellular, physiological or other biological activities), as determined using methods known to the art and described herein, will be understood to relate to a statistically significant difference in the particular activity relative to that seen in the absence of the antibody (e.g., or when a control antibody of irrelevant specificity is present). Preferably, the anti-hMPV antibody-induced increases in a measured parameter effects a statistically significant increase by at least 10% of the measured parameter, more preferably by at least 20%, 30%, 40%, 50%, 60%, 70%, 80%, 90%, 95%, or 100% (i.e., 2 fold), 3 fold, 5 fold or 10 fold. Conversely, anti-hMPV antibody-induced decreases in a measured parameter (e.g., hMPV activity) effects a statistically significant decrease by at least 10%, 20%, 30%, 40%, 50%, 60%, 70%, 80%, 90%, 95%, 97%, 98%, 99%, or 100%.

Also provided herein are anti-hMPV antibodies that bind to the same epitope on hMPV as any of the antibodies described herein. These antibodies have the ability to cross-compete for binding to hMPV with any of the antibodies described herein.

Antibodies disclosed herein include all known forms of antibodies and other protein scaffolds with antibody-like properties. For example, the antibody can be a human antibody, a humanized antibody, a bispecific antibody, an immunoconjugate, a chimeric antibody, or a protein scaffold with antibody-like properties, such as fibronectin or ankyrin repeats.

In some embodiments, the antibody is a bispecific antibody comprising a first and second binding region, wherein the first binding region comprises the binding specificity (e.g., antigen binding region) of an anti-hMPV antibody described herein, and a second binding region that does not bind to hMPV. In some embodiments, the second binding region binds to a protein that is not expressed on platelets.

The antibody also can be a Fab, F(ab′)₂, scFv, AFFIBODY, avimer, nanobody, single chain antibody, or a domain antibody. The antibody also can have any isotype, including any of the following isotypes: IgG1, IgG2, IgG3, IgG4, IgM, IgA1, IgA2, IgAsec, IgD, and IgE. Full-length antibodies can be prepared from V_{H} and V_{L} sequences using standard recombinant DNA techniques and nucleic acid encoding the desired constant region sequences to be operatively linked to the variable region sequences.

In certain embodiments, the antibodies described herein may have effector function or may have reduced or no effector function. In certain embodiments, the antibodies comprise an effector-less or mostly effector-less Fc, e.g., IgG2 or IgG4. Generally, variable regions described herein may be linked to an Fc comprising one or more modification, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. Each of these embodiments is described in further detail below. The numbering of residues in the Fc region is that of the EU index of Kabat.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, modifications can be made in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g. of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated (e.g., asparagine) may confer cytolytic response. Such residues may be deleted or substituted with non-glycosylated residues (e.g., alanine). In other embodiments, sites involved in interaction with complement, such as the C1q binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in PCT Publication numbers WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. More specifically, one or more amino acid mutations are introduced into the CH2-CH3 domain interface region of the Fc-hinge fragment thereof such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al. In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al. In another example, one or more amino acid residues within amino acid positions 231 and 239 are altered to thereby alter the ability of the antibody to fix complement. This approach is described further in PCT Publication number WO 94/29351 by Bodmer et al.

In yet another example, the Fc region may be modified to increase antibody dependent cellular cytotoxicity (ADCC) and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F/324T. Other modifications for enhancing Fc□R and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (PCT Patent Publication number WO00/42072).

Other Fc modifications that can be made to Fes are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 3305, 331 S, 2205, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCT Patent Publication numbers WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor FcγRIIb may also be used. Such variants may provide an Fc fusion protein with immunomodulatory activities related to FcγRIIb⁺ cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

In certain embodiments, the antibody is modified to increase its biological half-life. Various approaches are possible. For example, this may be done by increasing the binding affinity of the Fc region for FcRn. For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al., Journal of Biological Chemistry, 2001, 276(9):6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/31 IS (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In certain embodiments, hybrid IgG isotypes with particular biological characteristics may be used. For example, an IgG1/IgG3 hybrid variant may be constructed by substituting IgG1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed that comprises one or more substitutions, e.g., one or more of the following amino acid substitutions: 233E, 234L, 235L, -236G (referring to an insertion of a glycine at position 236), and 327A.

Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/1332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/1332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S.

When using an IgG4 constant domain, it is usually preferable to include the substitution S228P, which mimics the hinge sequence in IgG1 and thereby stabilizes IgG4 molecules.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Glycosylation of the constant region on N297 may be prevented by mutating the N297 residue to another residue, e.g., N297A, and/or by mutating an adjacent amino acid, e.g., 298 to thereby reduce glycosylation on N297.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication number WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication number WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment thereof. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, European patent number EP 0 154 316 by Nishimura et al. and European patent number EP 0 401 384 by Ishikawa et al.

The affinities and binding properties of an Fc region for its ligand may be determined by a variety of in vitro assay methods (biochemical or immunological based assays) known in the art including, but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

II. Antibodies which Bind to Same Epitope as or Cross-Compete with Anti-hMPV Antibodies

Anti-hMPV antibodies which bind to the same or similar epitopes to the antibodies disclosed herein (and thus also cross-compete with the antibodies disclosed herein) may be raised using immunization protocols. The resulting antibodies can be screened for high affinity binding to hMPV. Selected antibodies can then be studied, e.g., in yeast display assay in which sequence variants of hMPV are presented on the surface of yeast cells, or by hydrogen-deuterium exchange experiments, to determine the precise epitope bound by the antibody.

The epitope to which an antibody binds can be determined using art-recognized methods. An hMPV antibody is considered to bind to the same epitope as a reference anti-hMPV antibody if it, e.g., contacts one or more of the same residues on hMPV as the reference antibody; contacts one or more of the same residues within at least one region of hMPV as the reference antibody; contacts a majority of residues within at least one region of hMPV as the reference antibody; contacts a majority of the same residues within each region of hMPV as the reference antibody; contacts a majority of the same residues along the entire length of hMPV as the reference antibody; contacts all of the same distinct regions of hMPV as the reference antibody; contacts all of the same residues at any one region on hMPV as the reference antibody; or contacts all of the same residues at all of the same regions of hMPV as the reference antibody.

Techniques for determining antibodies that bind to the “same epitope on hMPV” with the hMPV antibodies described herein include x-ray analyses of crystals of antigen:antibody complexes, which provides atomic resolution of the epitope. Other methods monitor the binding of the antibody to antigen binding fragments thereof or mutated variations of the antigen where loss of binding due to an amino acid modification within the antigen sequence indicates the epitope component. Methods may also rely on the ability of an antibody of interest to affinity isolate specific short peptides (either in native three-dimensional form or in denatured form) from combinatorial phage display peptide libraries or from a protease digest of the target protein. The peptides are then regarded as leads for the definition of the epitope corresponding to the antibody used to screen the peptide library. For epitope mapping, computational algorithms have also been developed that have been shown to map conformational discontinuous epitopes.

The epitope or region comprising the epitope can also be identified by screening for binding to a series of overlapping peptides spanning hMPV. Alternatively, the method of Jespers et al. (1994) Biotechnology 12:899 may be used to guide the selection of antibodies having the same epitope and therefore similar properties to the hMPV antibodies described herein. Using phage display, first, the heavy chain of the anti-hMPV antibody is paired with a repertoire of (e.g., human) light chains to select a hMPV-binding antibody, and then the new light chain is paired with a repertoire of (e.g., human) heavy chains to select a (e.g., human) hMPV-binding antibody having the same epitope or epitope region as an anti-hMPV antibody described herein. Alternatively, variants of an antibody described herein can be obtained by mutagenesis of cDNA sequences encoding the heavy and light chains of the antibody.

Alanine scanning mutagenesis, as described by Cunningham & Wells (1989) Science 244: 1081, or some other form of point mutagenesis of amino acid residues in hMPV may also be used to determine the functional epitope for an anti-hMPV antibody.

The epitope or epitope region (an “epitope region” is a region comprising the epitope or overlapping with the epitope) bound by a specific antibody may also be determined by assessing binding of the antibody to peptides comprising hMPV fragments. A series of overlapping peptides encompassing the hMPV sequence may be synthesized and screened for binding, e.g. in a direct ELISA, a competitive ELISA (where the peptide is assessed for its ability to prevent binding of an antibody to hMPV bound to a well of a microtiter plate), or on a chip. Such peptide screening methods may not be capable of detecting some discontinuous functional epitopes.

An epitope may also be identified by MS-based protein footprinting, such as HDX-MS and Fast Photochemical Oxidation of Proteins (FPOP), structural methods such as X-ray crystal structure determination, molecular modeling, and nuclear magnetic resonance spectroscopy.

Single particle cryo-electron microscopy (SP-Cryo-EM) can also be used to identify the epitope to which an antibody or antigen binding fragment thereof binds. SP-Cryo-EM is a technique for macromolecular structure analysis which uses a high intensity electron beam to image biological specimens in their native environment at cryogenic temperature. In recent years, SP-cryo-EM has emerged as a complementary technique to crystallography and NMR for determining near-atomic level structures suitable for application in drug discovery (Renaud et al. Nat Rev Drug Discov 2018; 17:471-92; Scapin et al. Cell Chem Biol 2018; 25:1318-25; Ceska et al. Biochemical Society Transactions 2019: p. BST20180267). In addition to high resolution information, SP-Cryo-EM has the further advantage of allowing access to larger and more complex biological systems, with the possibility of characterizing multiple conformational or compositional solution states from the same sample, providing insights into more biologically relevant states of the macromolecule. For imaging, a small volume of sample (e.g., 3 microliter, 3 μl, aliquot) is applied onto a grid and flash-frozen in a liquid ethane bath. The frozen grid is then loaded into the microscope and hundreds to thousands of images of different areas of the grids are collected. These images contain two-dimensional projections of the biological macromolecule (particles): using mathematical tools and GPU powered algorithms, the particles are identified, extracted, and classified; in the subsequent step, the different classes are used to compute one or more 3D reconstructions, corresponding to different conformations, oligomerization or binding states if they coexist in the same sample. The individual reconstructions can then be refined to high resolution.

### III. Nucleic Acid Molecules

Also provided herein are nucleic acid molecules that encode the anti-hMPV antibodies or antigen binding fragments thereof, as well as the hMPV F proteins and variants thereof described herein. The nucleic acids may be present in whole cells, in a cell lysate, or in a partially purified or substantially pure form. A nucleic acid described herein can be, for example, DNA or RNA and may or may not contain intronic sequences. In certain embodiments, the nucleic acid is a cDNA molecule. The nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some embodiments, provided herein are nucleic acid molecules that encode the VH and/or VL sequences, or heavy and/or light chain sequences, of any of the anti-hMPV antibodies or antigen binding fragments thereof described herein. Host cells comprising the nucleotide sequences (e.g., nucleic acid molecules) described herein are encompassed herein. Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

Also provided herein are nucleic acid molecules with conservative substitutions that do not alter the resulting amino acid sequence upon translation of the nucleic acid molecule.

### IV. Methods of Production

Monoclonal antibodies that bind hMPV can be prepared using a wide variety of techniques known in the art including the use of hybridoma, recombinant, and phage display technologies, or a combination thereof.

Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties).

Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol, 222:581-597 (1991) describe the isolation of murine and human antibodies, respectively, using phage libraries. Subsequent publications describe the production of high affinity (e.g., nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods described above using antibody libraries derived from human immunoglobulin sequences. See also, U.S. Pat. Nos. 4,444,887 and 4,716,111; and PCT publication numbers WO 98/46645, WO 98/50433, WO 98/24893, WO 98/16654, WO 96/34096, WO 96/33735, and WO 91/10741, the contents of which are herein incorporated by reference in their entireties. Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. Phage display technology (McCafferty et al., Nature 348:552-553 (1990)) also can be used to produce human antibodies and antibody binding fragments thereof in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

Chimeric antibodies can be prepared based on the sequence of a murine monoclonal antibody. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.).

Humanized forms of anti-hMPV antibodies (e.g., humanized forms of mouse anti-hMPV antibodies) are chimeric antibodies which contain minimal sequence derived from non-human immunoglobulin. Humanized antibodies are typically human immunoglobulins (recipient antibody) in which residues from a CDR or hypervariable region of the recipient are replaced by residues from a CDR or hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin consensus sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The framework and CDR regions of a humanized antibody need not correspond precisely to the parental sequences, e.g., the donor antibody CDR or the consensus framework can be mutagenized by substitution, insertion and/or deletion of at least one amino acid residue so that the CDR or framework residue at that site does not correspond exactly to either the donor antibody or the consensus framework. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see e.g., Winnaker, From Genes to Clones (Veriagsgesellschaft, Weinheim, Germany 1987). In a family of immunoglobulins, each position in the consensus sequence is occupied by the amino acid occurring most frequently at that position in the family. Where two amino acids occur equally frequently, either can be included in the consensus sequence. As used herein, “Vernier zone” refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and can impact on the structure of CDRs and the affinity of the antibody. Human immunoglobulin (Ig) sequences that can be used as a recipient are well known in the art.

Framework residues in the human framework regions can be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, including, but not limited to, those described in Jones et al., Nature 321:522 (1986); Verhoeyen et al., Science 239: 1534 (1988), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al., PNAS 91:969-973 (1994); PCT publication number WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530, 101, 5,585,089, 5,225,539; 4,816,567, each entirely incorporated herein by reference.

The anti-hMPV antibodies generated using the methods described above can be tested for desired functions, such as particular binding specificities, binding affinities, targeted cell populations, using methods known in the art and described in the Examples, for example, art-recognized protein-protein binding assays, biochemical screening assays, immunoassays, and cell-based assays. An aspect of the invention provides molecules that may be used to screen for an antibody or antigen binding fragment thereof that binds hMPV. Exemplary assays include, but are not limited to, immunoprecipitation or by an in vitro binding assay, such as radioimmunoassay (RIA), FACS, enzyme-linked immunoabsorbent assay (ELISA), bio-layer interferometry (e.g., ForteBio assay), and Scatchard analysis.

### Antibody Engineering

Further included are embodiments in which the anti-hMPV antibodies or antigen-binding fragments thereof described herein (e.g., antibodies or antigen binding fragments thereof in Examples 1-6) are engineered antibodies to include modifications to framework residues within the variable domains of the parental monoclonal antibody, e.g., to improve the properties of the antibody or antigen binding fragment thereof. Typically, such framework modifications are made to decrease the immunogenicity of the antibody or antigen binding fragment thereof. This is usually accomplished by replacing non-CDR residues in the variable domains (i.e., framework residues) in a parental (e.g., rodent) antibody or antigen binding fragment thereof with analogous residues from the immune repertoire of the species in which the antibody is to be used, e.g., human residues in the case of human therapeutics. Such an antibody or antigen binding fragment thereof is referred to as a “humanized” antibody or antigen binding fragment thereof. In some embodiments, it is desirable to increase the affinity, or alter the specificity of an engineered (e.g., humanized) antibody. One approach is to “backmutate” one or more framework residues to the corresponding germline sequence. More specifically, an antibody or antigen binding fragment thereof that has undergone somatic mutation can contain framework residues that differ from the germline sequence from which the antibody is derived. Such residues can be identified by comparing the antibody or antigen binding fragment thereof framework sequences to the germline sequences from which the antibody or antigen binding fragment thereof is derived. Another approach is to revert to the original parental (e.g., rodent) residue at one or more positions of the engineered (e.g. humanized) antibody, e.g. to restore binding affinity that may have been lost in the process of replacing the framework residues. (See, e.g., U.S. Pat. Nos. 5,693,762, 5,585,089 and 5,530,101.)

In certain embodiments, the anti-hMPV antibodies and antigen binding fragments thereof are engineered (e.g., modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al., (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR”s or “hyper variable” loops. Commercial scientific software such as MOE (Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chain(s) to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain embodiments, the antibodies of the present disclosure do not contain deamidation or asparagine isomerism sites. For example, an asparagine (Asn) residue may be changed to Gln or Ala to reduce the potential for formation of isoaspartate at any Asn-Gly sequences, particularly within a CDR.

A similar problem may occur at an Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734. In various embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for hMPV, or other desired biological activity to unacceptable levels.

### Antibody Engineering of the Fc Region

The antibodies (i.e., anti-hMPV antibodies) and antigen binding fragments thereof disclosed herein can also be engineered to include modifications within the Fc region, typically to alter one or more properties of the antibody, such as serum half-life, complement fixation, Fc receptor binding, and/or effector function (e.g., antigen-dependent cellular cytotoxicity). Furthermore, the antibodies and antigen binding fragments thereof disclosed herein can be chemically modified (e.g., one or more chemical moieties can be attached to the antibody) or be modified to alter its glycosylation, again to alter one or more properties of the antibody or antigen binding fragment thereof. Each of these embodiments is described in further detail below. The numbering of residues in the Fc region is that of the EU index of Kabat.

The antibodies and antigen binding fragments thereof disclosed herein also include antibodies and antigen binding fragments thereof with modified (or blocked) Fc regions to provide altered effector functions. See, e.g., U.S. Pat. No. 5,624,821; and PCT Publication numbers WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc regions. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

In one embodiment, the antibody or antigen binding fragment thereof of the invention is an IgG4 isotype antibody or antigen binding fragment thereof comprising a Serine to Proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system).

In one embodiment of the invention, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody.

In another embodiment, the Fc hinge region of an antibody or antigen binding fragment thereof of the invention is mutated to decrease the biological half-life of the antibody or antigen binding fragment thereof. More specifically, one or more amino acid mutations are introduced into the CH2-CH3 domain interface region of the Fc-hinge fragment such that the antibody or antigen binding fragment thereof has impaired Staphylococcal protein A (SpA) binding relative to native Fc-hinge domain SpA binding. This approach is described in further detail in U.S. Pat. No. 6,165,745.

In another embodiment, the antibody or antigen binding fragment thereof of the invention is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody or antigen binding fragment thereof. For example, one or more amino acids selected from amino acid residues 234, 235, 236, 237, 297, 318, 320 and 322 can be replaced with a different amino acid residue such that the antibody has an altered affinity for an effector ligand and retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication number WO1994/29351.

In yet another example, the Fc region is modified to decrease the ability of the antibody or antigen binding fragment thereof of the invention to mediate antibody dependent cellular cytotoxicity (ADCC) and/or to decrease the affinity of the antibody or antigen binding fragment thereof for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication number WO 00/42072. Moreover, the binding sites on human IgG1 for FcγR1, FcγRII, FcγRIII and FcRn have been mapped and variants with improved binding have been described (see Shields et al. (2001) J. Biol. Chem. 276:6591-6604).

In one embodiment of the invention, the Fc region is modified to decrease the ability of the antibody of the invention to mediate effector function and/or to increase anti-inflammatory properties by modifying residues 243 and 264. In one embodiment, the Fc region of the antibody or antigen binding fragment thereof is modified by changing the residues at positions 243 and 264 to alanine. In one embodiment, the Fc region is modified to decrease the ability of the antibody or antigen binding fragment thereof to mediate effector function and/or to increase anti-inflammatory properties by modifying residues 243, 264, 267 and 328.

### Altered Effector Function

In some embodiments, the Fc region of an anti-hMPV antibody is modified to increase or reduce the ability of the antibody or antigen binding fragment thereof to mediate effector function and/or to increase/decrease their binding to the Fc gamma receptors (FcγRs).

The interaction between the constant region of an antigen binding protein and various Fc receptors (FcR) including FcγRI (CD64), FcγRII (CD32) and FcγRIII (CD16) is believed to mediate the effector functions, such as ADCC and CDC, of the antigen binding protein. The Fc receptor is also important for antibody cross-linking, which can be important for anti-tumor immunity.

Effector function can be measured in a number of ways including for example via binding of the FcγRIII to Natural Killer cells or via FcγRI to monocytes/macrophages to measure for ADCC effector function. For example, an antigen binding protein of the present invention can be assessed for ADCC effector function in a Natural Killer cell assay. Examples of such assays can be found in Shields et al., 2001 J. Biol. Chem., Vol. 276, p 6591-6604; Chappel et al., 1993 J. Biol. Chem., Vol 268, p 25124-25131; Lazar et al., 2006 PNAS, 103; 4005-4010.

Human IgG1 constant regions containing specific mutations or altered glycosylation on residue Asn297 have been shown to reduce binding to Fc receptors. In other cases, mutations have also been shown to enhance ADCC and CDC (Lazar et al. PNAS 2006, 103; 4005-4010; Shields et al. J Biol Chem 2001, 276; 6591-6604; Nechansky et al. Mol Immunol, 2007, 44; 1815-1817).

In one embodiment of the present invention, such mutations are in one or more of positions selected from 239, 332 and 330 (IgG1), or the equivalent positions in other IgG isotypes. Examples of suitable mutations are S239D and 1332E and A330L. In one embodiment, the antigen binding protein of the invention herein described is mutated at positions 239 and 332, for example S239D and 1332E or in a further embodiment it is mutated at three or more positions selected from 239 and 332 and 330, for example S239D and 1332E and A330L. (EU index numbering).

In an alternative embodiment of the present invention, there is provided an antibody comprising a heavy chain constant region with an altered glycosylation profile such that the antigen binding protein has enhanced effector function. For example, wherein the antibody has enhanced ADCC or enhanced CDC or wherein it has both enhanced ADCC and CDC effector function. Examples of suitable methodologies to produce antigen binding proteins with an altered glycosylation profile are described in PCT Publication numbers WO2003011878 and WO2006014679 and European patent number EP1229125.

In a further aspect, the present invention provides “non-fucosylated” or “afucosylated” antibodies. Non-fucosylated antibodies harbor a tri-mannosyl core structure of complex-type N-glycans of Fc without fucose residue. These glycoengineered antibodies that lack core fucose residue from the Fe N-glycans may exhibit stronger ADCC than fucosylated equivalents due to enhancement of FcγRIIIa binding capacity.

The present invention also provides a method for the production of an antibody according to the invention comprising the steps of: a) culturing a recombinant host cell comprising an expression vector comprising the isolated nucleic acid as described herein, wherein the recombinant host cell does not comprise an alpha-1,6-fucosyltransferase; and b) recovering the antigen binding protein. The recombinant host cell may not normally contain a gene encoding an alpha-1,6-fucosyltransferase (for example yeast host cells such as Pichia sp.) or may have been genetically modified to inactivate an alpha-1,6-fucosyltransferase. Recombinant host cells which have been genetically modified to inactivate the FUT8 gene encoding an alpha-1,6-fucosyltransferase are available. See, e.g., the POTELLIGENT™ technology system available from BioWa, Inc. (Princeton, N.J.) in which CHOK1SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects ofthe POTELLIGENT™ technology system are described in U.S. Pat. Nos. U.S. Pat. Nos. 7,214,775 and 6,946,292, and PCT Publication numbers WO0061739 and WO0231240. Those of ordinary skill in the art will also recognize other appropriate systems.

It will be apparent to those skilled in the art that such modifications may not only be used alone but may be used in combination with each other in order to further enhance or decrease effector function.

Production of Antibodies with Modified Glycosylation

In still another embodiment, the antibodies or antigen binding fragments thereof of the invention comprise a particular glycosylation pattern. For example, an afucosylated or an aglycosylated antibody or antigen binding fragment thereof can be made (i.e., the antibody lacks fucose or glycosylation, respectively). The glycosylation pattern of an antibody or antigen binding fragment thereof may be altered to, for example, increase the affinity or avidity of the antibody or fragment for hMPV. Such modifications can be accomplished by, for example, altering one or more of the glycosylation sites within the antibody or antigen binding fragment thereof sequence. For example, one or more amino acid substitutions can be made that result in removal of one or more of the variable region framework glycosylation sites to thereby eliminate glycosylation at that site. Such aglycosylation may increase the affinity or avidity of the antibody or antigen binding fragment thereof for antigen. See, e.g., U.S. Pat. Nos. 5,714,350 and 6,350,861.

Antibodies and antigen binding antigen binding fragments thereof disclosed herein may further include those produced in lower eukaryote host cells, in particular fungal host cells such as yeast and filamentous fungi have been genetically engineered to produce glycoproteins that have mammalian- or human-like glycosylation patterns (See for example, Choi et al., (2003) Proc. Natl. Acad. Sci. 100: 5022-5027; Hamilton et al., (2003) Science 301: 1244-1246; Hamilton et al., (2006) Science 313: 1441-1443; Nett et al., Yeast 28(3):237-52 (2011); Hamilton et al., Curr Opin Biotechnol. October; 18(5):387-92 (2007)). A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures (See for example, Li et al., (2006) Nat. Biotechnol. 24: 210-215).

In particular embodiments, the antibodies and antigen binding fragments thereof disclosed herein further include those produced in lower eukaryotic host cells and which comprise fucosylated and non-fucosylated hybrid and complex N-glycans, including bisected and multi-antennary species, including but not limited to N-glycans such as GlcNAc₍₁₋₄₎Man₃GlcNAc₂; Gal₍₁₋₄₎GlcNAc₍₁₋₄₎Man₃GlcNAc₂; NANA₍₁₋₄₎Gal₍₁₋₄₎GlcNAc₍₁₋₄₎Man₃GlcNAc₂.

In particular embodiments, the antibodies and antigen binding fragments thereof provided herein may comprise antibodies or antigen binding fragments thereof having at least one hybrid N-glycan selected from the group consisting of GlcNAcMan₅GlcNAc₂; GalGlcNAcMan₅GlcNAc₂; and NANAGalGlcNAcMan₅GlcNAc₂. In particular aspects, the hybrid N-glycan is the predominant N-glycan species in the composition.

In particular embodiments, the antibodies and antigen binding fragments thereof provided herein comprise antibodies and antigen binding fragments thereof having at least one complex N-glycan selected from the group consisting of GlcNAcMan₃GlcNAc₂; GalGlcNAcMan₃GlcNAc₂; NANAGalGlcNAcMan₃GlcNAc₂; GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂. In particular aspects, the complex N-glycan are the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In one embodiment, the antibody and antigen binding fragments thereof provided herein comprise complex N-glycans, wherein at least 50%, 60%, 70%, 80%, 90%, 95%, 97%, 98%, 99%, or 100% of the complex N-glycans comprise the structure NANA₂Gal₂GlcNAc₂Man₃GlcNAc₂, wherein such structure is afucosylated. Such structures can be produced, e.g., in engineered Pichia pastoris host cells.

In particular embodiments, the N-glycan is fucosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan. Therefore, in particular aspects of the above the glycoprotein compositions, the glycoform is in an α1,3-linkage or α1,6-linkage fucose to produce a glycoform selected from the group consisting of Man₅GlcNAc₂(Fuc), GlcNAcMan₅GlcNAc₂(Fuc), Man₃GlcNAc₂(Fuc), GlcNAcMan₃GlcNAc₂(Fuc), GlcNAc₂Man₃GlcNAc₂(Fuc), GalGlcNAc₂Man₃GlcNAc₂(Fuc), Gal₂GlcNAc₂Man₃GlcNAc₂(Fuc), NANAGal₂GlcNAc₂Man₃GlcNAc₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)Man₅GlcNAc₂, GlcNAc(Fuc)Man₃GlcNAc₂, GlcNAc₂(Fuc₁₋₂)Man₃GlcNAc₂, GalGlcNAc₂(Fuc₁₋₂)Man₃GlcNAc₂, Gal₂GlcNAc₂(Fuc₁₋₂)Man₃GlcNAc₂, NANAGal₂GlcNAc₂(Fuc₁₋₂)Man₃GlcNAc₂, and NANA₂Gal₂GlcNAc₂(Fuc₁₋₂)Man₃GlcNAc₂; or in an α1,2-linkage fucose to produce a glycoform selected from the group consisting of Gal(Fuc)GlcNAc₂Man₃GlcNAc₂, Gal₂(Fuc₁₋₂)GlcNAc₂Man₃GlcNAc₂, NANAGal₂(Fuc₁₋₂)GlcNAc₂Man₃GlcNAc₂, and NANA₂Gal₂(Fuc₁₋₂)GlcNAc₂Man₃GlcNAc₂.

In further aspects, the antibodies (e.g., humanized antibodies) or antigen binding fragments thereof comprise high mannose N-glycans, including but not limited to, Man₈GlcNAc₂, Man₇GlcNAc₂, Man₆GlcNAc₂, Man₅GlcNAc₂, Man₄GlcNAc₂, or N-glycans that consist of the Man₃GlcNAc₂ N-glycan structure.

In further aspects of the above, the complex N-glycans further include fucosylated and non-fucosylated bisected and multi-antennary species.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms”.

With respect to complex N-glycans, the terms “G-2”, “G-1”, “G0”, “G1”, “G2”, “Al”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G₁” refers to an N-glycan structure that can be characterized as GalGlcNAc₂Man₃GlcNAc₂; the term “G₂” refers to an N-glycan structure that can be characterized as Gal₂GlcNAc₂Man₃GlcNAc₂; the term “Al” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G-2”, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multi-antennary N-glycans, the term “multi-antennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multi-antennary N-glycans can be characterized by the formulas GlcNAc₍₂₋₄₎Man₃GlcNAc₂, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues. With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multi-antennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

### Antibody Physical Properties

The antibodies and antigen binding fragments thereof disclosed herein may further contain one or more glycosylation sites in either the light or heavy chain immunoglobulin variable region. Such glycosylation sites may result in increased immunogenicity of the antibody or antigen binding fragment thereof or an alteration of the PK of the antibody due to altered antigen binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence.

Each antibody or antigen binding fragment thereof will have a characteristic melting temperature, with a higher melting temperature indicating greater overall stability in vivo (Krishnamurthy R and Manning MC (2002) Curr Pharm Biotechnol 3:361-71). In general, the T_{M1} (the temperature of initial unfolding) may be greater than 60° C., greater than 65° C., or greater than 70° C. The melting point of an antibody or antigen binding fragment thereof can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9). In a further embodiment, antibodies and antigen binding fragments thereof are selected that do not degrade rapidly. Degradation of an antibody or antigen binding fragment thereof can be measured using capillary electrophoresis (CE) and MALDI-MS (Alexander A J and Hughes D E (1995) Anal Chem 67:3626-32).

In a further embodiment, antibodies and antigen binding fragments thereof are selected that have minimal aggregation effects, which can lead to the triggering of an unwanted immune response and/or altered or unfavorable pharmacokinetic properties. Generally, antibodies and antigen binding fragments thereof are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

### V. Multi-Specific Antibodies

Multi-specific antibodies (e.g., bispecific antibodies) provided herein include at least one binding region for a particular epitope on hMPV as described herein, and at least one other binding region (e.g., a viral antigen). Multi-specific antibodies can be prepared as full-length antibodies or antigen binding fragments thereof (e.g. F(ab′)₂ antibodies).

Methods for making multi-specific antibodies are well known in the art (see, e.g., PCT Publication numbers WO 05117973 and WO 06091209). For example, production of full length multi-specific antibodies can be based on the co-expression of two paired immunoglobulin heavy chain-light chains, where the two chains have different specificities. Various techniques for making and isolating multi-specific antibody fragments directly from recombinant cell culture have also been described. For example, multi-specific antibodies can be produced using leucine zippers. Another strategy for making multi-specific antibody fragments by the use of single-chain Fv (scFv) dimers has also been reported.

Examples of suitable multi-specific molecule platforms include, but are not limited to, Dual Targeting (DT)-Ig (GSK/Domantis), Two-in-one Antibody (Genentech), Cross-linked Mabs (Karmanos Cancer Center), Fcab and mAb2 (F-Star), CovX-body (CovX/Pfizer), Dual Variable Domain (DVD)-Ig (Abbott), IgG-like Bispecific (ImClone/Eli Lilly), Ts2Ab (Medlmmune/AZ) and BsAb (Zymogenetics), HERCULES (Biogen Idec), TvAb (Roche), ScFv/Fc Fusions, SCORPION (Emergent BioSolutions/Trubion, Zymogenetics/BMS), Dual Affinity Retargeting Technology (Fc-DART) (MacroGenics), Dual(ScFv)2-Fab (National Research Center for Antibody Medicine—China), F(ab)2 (Medarex/AMGEN), Dual-Action or Bis-Fab (Genentech), Dock-and-Lock (DNL) (ImmunoMedics), Bivalent Bispecific (Biotecnol), SEED (EMD Serono), mAb2 (F-star), Fab-Fv (UCB-Celltech), Bispecific T Cell Engager (BiTE) (Micromet, Tandem Diabody (Tandab) (Affimed), Dual Affinity Retargeting Technology (DART) (MacroGenics), Single-chain Diabody (Academic), TCR-like Antibodies (AIT, ReceptorLogics), COMBODY (Epigen Biotech), dual targeting nanobodies (Ablynx), and Fc-engineered IgG1 (Xencor). Different bispecific formats are described in Spiess et al., 2015 Molecular Immunology, vol. 67, issue 2, Part A, pages 95-106.

In a particular embodiment, the multi-specific antibody comprises a first antibody (or binding portion thereof) which binds to hMPV derivatized or linked to another functional molecule, e.g., another peptide or protein (e.g., another antibody or ligand for a receptor) to generate a multi-specific molecule that binds to hMPV and a non-hMPV target molecule. An antibody may be derivatized or linked to more than one other functional molecule to generate multi-specific molecules that bind to more than two different binding sites and/or target molecules. To create a multi-specific molecule, an antibody disclosed herein can be functionally linked (e.g., by chemical coupling, genetic fusion, noncovalent association or otherwise) to one or more other binding molecules, such as another antibody or antigen binding fragment thereof, antibody fragment, peptide, receptor, or binding mimetic, such that a multi-specific molecule results.

Accordingly, multi-specific molecules, for example, bispecific antibodies and bifunctional antibodies, comprising at least one first binding specificity for a particular epitope on hMPV and a second binding specificity for a second target are contemplated. In some embodiments, the second target is the second binding region specifically binds to a viral-associated antigen. Viral-associated antigens are well known in the art.

In some embodiments, the antibody is a trispecific antibody comprising a first, second, and third binding region, wherein the first binding region comprises the binding specificity (e.g., antigen binding region) of an anti-hMPV antibody described herein, and the second and third binding regions bind to two different targets (or different epitopes on the same target), for example, the targets described above.

In one embodiment, the multi-specific molecules comprise as a binding specificity at least one antibody, or an antibody fragment thereof, including, e.g., a Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778.

The multi-specific molecules can be prepared by conjugating the constituent binding specificities, e.g., the anti-FcR and anti-hMPV binding specificities, using methods known in the art. For example, each binding specificity of the multi-specific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC). Preferred conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂ or ligand x Fab fusion protein. A multi-specific molecule can be a single chain molecule comprising one single chain antibody and a binding determinant, or a single chain bispecific molecule comprising two binding determinants. Multi-specific molecules may comprise at least two single chain molecules. Methods for preparing multi-specific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the multi-specific molecules to their specific targets can be confirmed by, for example, enzyme-linked immunosorbent assay (ELISA), radioimmunoassay (RIA), fluorescence-activated cell sorting (FACS) analysis, bioassay (e.g., growth inhibition), or western blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA). The radioactive isotope can be detected by such means as the use of a a y-O counter or a scintillation counter or by autoradiography.

### VI. Antigens/Antigenic Polypeptides

In one aspect, provided herein are isolated hMPV F polypeptides and variants thereof. The hMPV F protein polypeptides and variants thereof described herein can be used to induce an active immunogenic response in a subject or to generate antibodies from B cells in vitro.

Cleavable affinity tags may be added to the sequence of the hMPV F polypeptides and variants thereof. As used herein, the term “cleavable affinity tag” refers to a protein sequence on the N or C terminus of the recombinant hMPV F polypeptides described herein for an enzymatic cleavage sequence and a polypeptide sequence which can be used to purify the recombinant hMPV F protein polypeptide by affinity chromatography. Such tags and cleavage sequences are described by Kimple et al., Current Protocols in Protein Science 9.9.1-9.9.23, August 2013. Exemplary affinity tag sequences include, but are not limited to: protein A; lacZ; polyhistidine (HHHHHH, SEQ ID NO: 1014; also called 6×His, His-tag, or His6); Glutathione-S-Transferase (GST); maltose-binding protein (MBP); calmodulin-binding protein (CBP); biotin-based tags (e.g., BCCP); streptavidin-based tags (e.g. strep tag II; SAWSHPQFEK; SEQ ID NO: 1015); mutant thioredoxin (e.g. His-Patch ThioFusion™); FLAG-tag; hemagglutinin (HA); c-myc; T7, Glu-Glu; ALFA-tag; V5-tag; Spot-Tag™; novel epitope tag (NE-tag); β-galactosidase (β-gal); alkaline phosphatase (AP); chloramphenicol acetyl transferase (CAT); horseradish peroxidase (HRP); modified haloalkane dehalogenase (HaloTag™); and modified serine protease subtilisin (Profinity eXact™).

Exemplary cleavage sequences for removing the affinity tag may include, but are not limited to: thrombin protease cleavage sequence (LVPRGS, SEQ ID NO: 1013); factor Xa cleavage sequence (SEQ ID NO: 1016, IEGR; or SEQ ID NO: 1017, IDGR); PreScission™ protease cleavage site (SEQ ID NO: 1018, LEVLFQGP); enterokinase cleavage sequence (SEQ ID NO: 1019, DDDDK); genenase I cleavage sequence (SEQ ID NO: 1020, TQLAYFTDSKNPGAAHYDTFADSLR); and TEV protease cleavage sequence (SEQ ID NO: 1021, ENLYFQ).

In some embodiments, the tag may be recognized directly by a protease and result in cleavage of the affinity tag from the rest of the polypeptide. For example, the tag small ubiquitin-like modifier (SUMO) is recognized by SUMO protease via the SUMO tag's tertiary structure. In another example, a subtilisin prodomain tag (Profinity eXact™; BioRad Laboratories, Hercules CA) is recognized and cleaved by a mutant subtilisin protein. In another example, intein-chitin binding domain tag (intein-CBD; IMPACT™ System, New England Biolabs, Beverley MA) can self-cleave upon activation by a denaturing agent such as dithiothreitol.

Trimerization domain sequences can also be added to the hMPV F polypeptides and variants described herein. Such sequences can allow for trimerization of an antigenic polypeptide. Exemplary trimerization domain sequence may include, but are not limited to: a coiled-coil trimerization domain, such as a GCN4 domain (ARMKQIEDKIEEILSKIYHIENEIARIKKLIGEAGSG; SEQ ID NO: 1022); a foldon domain from T4 fibritin (GYIPEAPRDGQAYVRKDGEWVLLSTFL; SEQ ID NO: 1012); a human type XV collagen domain (SEQ ID NO: 1023); and a human type XVIII collagen domain (SEQ ID NO: 1024).

human type XV collagen domain:
GSRNLVTAFSNMDDMLQKAHLVIEGTFIYL
RDSTEFFIRVRDGWKKLQLGELIPIPADS
(SEQ ID NO: 1023)
human type XVIII collagen domain
(SEQ ID NO: 1024):
GVRLWATRQAMLGQVHEVPEGWLIFVAEQE
ELYVRVQNGFRKVQLEARTPLPRGTDNE

The protease cleavage site at residues 99-102 of SEQ ID NO: 910 and 911 can be replaced with a furin-cleavage sequence. Furin-cleavage sequences are traditionally described by the consensus sequence RXRR (SEQ ID NO: 1025) or RXKR (SEQ ID NO: 1026), wherein X is any amino acid. In various embodiments, residues 99-102 of SEQ ID NO: 910 are substituted with SEQ ID NO: 1025 or 1026. In various embodiments residues 99-102 of SEQ ID NO: 910 are substituted with SEQ ID NO: 1027 (RGRR), SEQ ID NO: 1028 (RARR), SEQ ID NO: 1029 (RLRR), SEQ ID NO: 1030 (RMRR), SEQ ID NO: 1031 (RFRR), SEQ ID NO: 1032 (RWRR), SEQ ID NO: 1033 (RKRR), SEQ ID NO: 1034 (RQRR), SEQ ID NO: 1035 (RERR), SEQ ID NO: 1036 (RSRR), SEQ ID NO: 1037 (RPRR), SEQ ID NO: 1038 (RVRR), SEQ ID NO: 1039 (RIRR), SEQ ID NO: 1040 (RCRR), SEQ ID NO: 1041 (RYRR), SEQ ID NO: 1042 (RHRR), SEQ ID NO: 1043 (RRRR), SEQ ID NO: 1044 (RNRR), SEQ ID NO: 1045 (RDRR), SEQ ID NO: 1046 (RTRR), SEQ ID NO: 1047 (RGKR), SEQ ID NO: 1048 (RAKR), SEQ ID NO: 1049 (RLKR), SEQ ID NO: 1050 (RMKR), SEQ ID NO: 1051 (RFKR), SEQ ID NO: 1052 (RWKR), SEQ ID NO: 1053 (RKKR), SEQ ID NO: 1054 (RQKR), SEQ ID NO: 1055 (REKR), SEQ ID NO: 1056 (RSKR), SEQ ID NO: 1057 (RPKR), SEQ ID NO: 1058 (RVKR), SEQ ID NO: 1059 (RIKR), SEQ ID NO: 1060 (RCKR), SEQ ID NO: 1061 (RYKR), SEQ ID NO: 1062 (RHKR), SEQ ID NO: 1063 (RRKR), SEQ ID NO: 1064 (RNKR), SEQ ID NO: 1065 (RDKR), or SEQ ID NO: 1066 (RTKR).

### VII. Assays

The anti-hMPV antibodies or antigen binding fragments thereof disclosed herein can be tested for desired properties, e.g., those described herein, using a variety of assays known in the art.

In one embodiment, the antibodies or antigen binding fragments thereof are tested for specific binding to hMPV. Methods for analyzing binding affinity, cross-reactivity, and binding kinetics of various anti-hMPV antibodies or antigen binding fragments thereof include standard assays known in the art, for example, Biacore™ surface plasmon resonance (SPR) analysis using a Biacore™ 2000 SPR instrument (Biacore AB, Uppsala, Sweden) or bio-layer interferometry (e.g., ForteBio assay), as described in the Examples.

In one embodiment, the antibodies or antigen binding fragments thereof are tested for the ability to bind to cells that have been transfected with hMPV.

In one embodiment, the antibodies or antigen binding fragments thereof are screened for the ability to bind to the surface of beads that have been coated with hMPV.

In one embodiment, the antibodies or antigen binding fragments thereof are tested for the ability to bind or affect hMPV. In another embodiment, the antibodies or antigen binding fragments thereof are tested for their effects on hMPV (e.g., inhibition, or no effect).

### VIII. Compositions

Also provided herein are compositions (e.g., pharmaceutical compositions) comprising the anti-hMPV antibodies or antigen binding fragments thereof described herein, immunoconjugates comprising the same, or bispecific antibodies comprising the same, and a carrier (e.g., pharmaceutically acceptable carrier). Such compositions are useful for various therapeutic applications.

In some embodiments, pharmaceutical compositions disclosed herein can include other compounds, drugs, and/or agents used for the treatment of various diseases (e.g., respiratory diseases). Such compounds, drugs, and/or agents can include, for example, an anti-viral agent, and/or an anti-inflammatory agent. Exemplary compounds, drugs, and agents that can be formulated together or separately with the anti-hMPV antibodies or antigen binding fragments thereof described herein.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein may also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition may comprise a preservative or may be devoid of a preservative. Supplementary active compounds can be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody or antigen binding fragment thereof, the dosage ranges from about 0.0001 to 100 mg/kg, and more usually 0.01 to 5 or 10 mg/kg, of the host body weight. For administration of the antibody or antigen binding fragment thereof, the dosage ranges from about 0.0001 to 100 mg. An exemplary treatment regime entails administration once per week, once every two weeks, once every three weeks, once every four weeks, once a month, once every 3 months or once every three to 6 months.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The therapeutically effective dosage of an anti-hMPV antibody or antigen binding fragment thereof in various embodiments results in a decrease in severity of disease symptoms, an increase in frequency and duration of disease symptom-free periods, or a prevention of impairment or disability due to the disease affliction. In the context of cancer, a therapeutically effective dose preferably results in increased survival, and/or prevention of further deterioration of physical symptoms associated with cancer. A therapeutically effective dose may prevent or delay onset of cancer, such as may be desired when early or preliminary signs of the disease are present.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody or antigen binding fragment thereof described herein can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-hMPV antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-hMPV antibodies or antigen binding fragments thereof described herein can be formulated to ensure proper distribution in vivo. For example, the blood-brain barrier (BBB) excludes many highly hydrophilic compounds. To ensure that the therapeutic compounds described herein cross the BBB (if desired, e.g., for brain cancers), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Adjuvants

hMPV immunogenic compositions may be formulated or administered alone or in conjunction with one or more other components. For instance, hMPV immunogenic compositions may comprise other components including, but not limited to, adjuvants.

In some embodiments, hMPV immunogenic compositions of the instant disclosure do not include an adjuvant (they are adjuvant-free).

Suitable adjuvants to enhance effectiveness of the immunogenic compositions disclosed herein include, but are not limited to:
(1) aluminum salts (alum), such as aluminum hydroxide, aluminum phosphate, aluminum sulfate, etc.;
(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, MA), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) RIBI adjuvant system (RAS), (Corixa, Hamilton, MT) containing 2% Squalene, 0.2% Tween 80, and one or more bacterial cell wall components from the group consisting of 3-O-deacylated monophosphorylipid A (MPL) described in U.S. Pat. No. 4,912,094, trehalose dimycolate (TDM), and cell wall skeleton (CWS), preferably MPL+CWS (DETOX); and (d) a Montanide ISA;
(3) saponin adjuvants, such as Quil A or STIMULON QS-21 (Antigenics, Framingham, MA) (see, e.g., U.S. Pat. No. 5,057,540) may be used or particles generated therefrom such as ISCOM (immunostimulating complexes formed by the combination of cholesterol, saponin, phospholipid, and amphipathic proteins) and ISCOMATRIX (having essentially the same structure as an ISCOM but without the protein);
(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3—tetradecanoyloxytetradecanoylamino]-b-D-glucopyranoside, which is also known as 529 (formerly known as RC529), which is formulated as an aqueous form or as a stable emulsion
(5) synthetic polynucleotides such as oligonucleotides containing CpG motif(s) (U.S. Pat. No. 6,207,646); and
(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and
(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, the adjuvant is an aluminum salt. The aluminum salt adjuvant may be an alum-precipitated immunogenic composition or an alum-adsorbed immunogenic composition. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate, Aluminum Phosphate Adjuvant (APA), amorphous alumina, trihydrated alumina, or trihydroxy aluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

In certain embodiments, a commercially available Al(OH)₃ (e.g. Alhydrogel or Superfos of Denmark/Accurate Chemical and Scientific Co., Westbury, NY) is used to adsorb proteins in a ratio of 50-200 μg protein/mg aluminum hydroxide. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of antigen that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110.

### Formulations

hMPV immunogenic compositions may be formulated or administered in combination with one or more pharmaceutically-acceptable excipients. In some embodiments, immunogenic compositions comprise at least one additional active substance, such as, for example, a therapeutically-active substance, a prophylactically-active substance, or a combination of both. Immunogenic compositions may be sterile, pyrogen-free or both sterile and pyrogen-free. General considerations in the formulation and/or manufacture of pharmaceutical agents, such as immunogenic compositions, may be found, for example, in Remington: The Science and Practice of Pharmacy 21st ed., Lippincott Williams & Wilkins, 2005 (incorporated herein by reference in its entirety). In some embodiments, hMPV immunogenic compositions are administered to humans, human patients, or subjects.

Formulations of the hMPV immunogenic compositions described herein may be prepared by any method known or hereafter developed in the art of pharmacology. In general, such preparatory methods include the step of bringing the active ingredient (e.g., polypeptide or polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Compositions of this disclosure can be formulated as single dose vials, multi-dose vials or as pre-filled glass or plastic syringes.

In another embodiment, compositions of the present disclosure are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or non-aqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

The pharmaceutical composition may be isotonic, hypotonic or hypertonic. However, it is often preferred that a pharmaceutical composition for infusion or injection is essentially isotonic, when it is administered. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to NaCl, CaCl₂), KCl and MgCl₂. Examples of non-ionic isotonic agents include but are not limited to mannitol, sorbitol and glycerol.

It is also preferred that at least one pharmaceutically-acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

The buffer may, for example, be selected from the group consisting of TRIS, acetate, glutamate, lactate, maleate, tartrate, phosphate, citrate, carbonate, glycinate, histidine, glycine, succinate and triethanolamine buffer.

The buffer may be selected from USP compatible buffers for parenteral use, in particular, when the pharmaceutical formulation is for parenteral use. For example, the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The formulations of the disclosure may also contain a surfactant. Preferred surfactants include, but are not limited to: the polyoxyethylene sorbitan esters surfactants (commonly referred to as the Tweens), especially PS-20 and PS-80; copolymers of ethylene oxide (EO), propylene oxide (PO), and/or butylene oxide (BO), sold under the DOWFAX tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the TERGITOL NP series; polyoxyethylene fatty ethers derived from lauryl, cetyl, stearyl and oleyl alcohols (known as BRIJ surfactants), such as triethyleneglycol monolauryl ether (BRIJ 30); and sorbitan esters (commonly known as the SPANs), such as sorbitan trioleate (SPAN 85) and sorbitan monolaurate.

Mixtures of surfactants can be used, e.g. PS-80/Span 85 mixtures. A combination of a polyoxyethylene sorbitan ester such as polyoxyethylene sorbitan monooleate (PS-80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as PS-80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

### Modes of Administration

hMPV immunogenic compositions may be administered by any route which results in a therapeutically-effective outcome. These include, but are not limited, to intradermal, intramuscular, intranasal, and/or subcutaneous administration. The present disclosure provides methods comprising administering immunogenic compositions to a subject in need thereof. The exact amount required will vary from subject to subject, depending on the species, age, and general condition of the subject, the severity of the disease, the particular composition, its mode of administration, its mode of activity, and the like. hMPV immunogenic compositions are typically formulated in dosage unit form for ease of administration and uniformity of dosage. It will be understood, however, that the total daily usage of immunogenic compositions may be decided by the attending physician within the scope of sound medical judgment. The specific therapeutically-effective or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, hMPV immunogenic compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013/078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, hMPV immunogenic compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, hMPV immunogenic compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

Any of the hMPV immunogenic compositions described herein can be formulated into a dosage form described herein, such as an intranasal, intratracheal, or injectable (e.g., intravenous, intraocular, intravitreal, intramuscular, intradermal, intracardiac, intraperitoneal, intranasal and subcutaneous).

hMPV Immunogenic Composition Formulations and Methods of Use

Some aspects of the present disclosure provide formulations of the hMPV immunogenic composition, wherein the immunogenic composition is formulated in an effective amount to produce an antigen specific immune response in a subject (e.g., production of antibodies specific to an hMPV antigenic polypeptide). “An effective amount” is a dose of a immunogenic composition effective to produce an antigen-specific immune response. Also provided herein are methods of inducing an antigen-specific immune response in a subject.

In some embodiments, the antigen-specific immune response is characterized by measuring an anti-hMPV antigenic polypeptide antibody titer produced in a subject administered an hMPV immunogenic composition as provided herein. An antibody titer is a measurement of a concentration of antibodies within a subject, for example, antibodies that are specific to a particular antigen (e.g., an hMPV antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous immunogenic composition was effective, and to identify any recent or prior infections. In accordance with the present disclosure, an antibody titer may be used to determine the strength of an immune response induced in a subject by the hMPV immunogenic composition.

### IX. Uses and Methods

The antibodies, antibody compositions, and methods described herein have numerous in vitro and in vivo utilities.

For example, provided herein is a method of treating or preventing a viral disease associated with hMPV comprising administering to a subject in need thereof an anti-hMPV antibody or antigen binding fragment thereof described herein, such that the subject is treated, e.g., such that the viral load and/or viral activity is inhibited or reduced and/or that treatment of the viral disease is achieved.

In one embodiment, provided herein is a method of treating a viral disease associated with hMPV comprising administering to a subject in need thereof an effective amount (e.g., a therapeutically effective amount) of an anti-hMPV antibody described herein (or a bispecific antibody hMPV). In some embodiments, the subject is administered a further therapeutic agent. In some embodiments, the further therapeutic agent is an anti-viral agent, e.g., another anti-viral antibody or anti-viral small molecule

Also encompassed are methods for detecting the presence of hMPV in a sample (e.g., a blood sample), or measuring the amount of hMPV in sample, comprising contacting the sample (e.g., tissue) and a control sample (e.g., corresponding healthy tissue) with an antibody (e.g., monoclonal antibody) or antigen binding fragment thereof which specifically binds to hMPV under conditions that allow for formation of a complex or binding between the antibody or portion thereof and hMPV. The formation of a complex is then detected, wherein a difference in complex formation between the sample compared to the control sample is indicative of the presence of hMPV in the sample. The anti-hMPV antibodies or antigen binding fragments thereof described herein can also be used to purify hMPV via immunoaffinity purification.

Diagnostic applications of the anti-hMPV antibodies described herein are also contemplated.

In one embodiment, provided herein is a method of diagnosing a viral infection or viral disease associated with hMPV comprising contacting a biological sample from a patient afflicted with the viral infection or viral disease with an anti-hMPV antibody or antigen binding fragment thereof described herein, wherein positive staining with the antibody indicates the viral infection or viral disease is associated with hMPV.

### Combination Therapy

The anti-hMPV antibodies or antigen binding fragments thereof described herein can be used in combination with various treatments, prophylactic agent, or therapeutic agent (or in the context of a multi-specific antibody or bifunctional partner) known in the art for the prevention or treatment of an infection (e.g., viral infection) and/or a disease (e.g., viral disease), as described herein. In one embodiment, the prophylactic agent or therapeutic agent comprises a second antibody or fragment thereof, an immunomodulator, a hormone, a cytotoxic agent, an enzyme, a radionuclide, a second antibody conjugated to at least one immunomodulator, enzyme, radioactive label, hormone, antisense oligonucleotide, or cytotoxic agent, or a combination thereof. In various embodiments, the therapeutic agent comprises a hormonal therapy, immunotherapy, an antiviral agent, and/or an anti-inflammatory agent.

Suitable anti-viral agents for use in combination therapy with the anti-hMPV antibodies or antigen binding fragments thereof described herein include, but are not limited to, an antiviral agent (e.g., small molecules and antibodies). In various embodiments, the antiviral agent comprises an anti-RSV antibody. In various embodiments, the antiviral agent comprises a nucleotide analogues, which interfere or stop DNA or RNA synthesis. In various embodiments, the antiviral agent comprises an inhibitor of an enzyme involved in DNA or RNA synthesis (e.g., helicase, replicase). In various embodiments, the antiviral agent comprises a compound which inhibits the virus maturation steps during its replication cycle. In various embodiments, the antiviral agent comprises a compound which interferes with cell membrane binding, or virus entry in host cells (e.g., fusion or entry inhibitors). In various embodiments, the antiviral agent comprises an agent which prevents the virus from being expressed within the host cell after its entry. In various embodiments, the antiviral agent comprises a compound that block the virus' disassembly within the cell. In various embodiments, the antiviral agent comprises an agent that restricts virus propagation to other cells.

The compositions and methods as recited herein can include multiple antibodies against the same antigen (i.e., MPV), or against multiple viruses or viral agents/antigens (e.g., RSV and MPV) and/or a bacterial agent/antigen. In various embodiments of the present invention, compositions and methods disclosed herein may include an antibody against MPV (e.g., hMPV), plus an antibody against a bacterial agent/antigen, for example one that infects the respiratory system.

The compositions and methods as recited herein can include an anti-viral antibody that binds to MPV as described herein, and/or a non-immunological anti-viral agent, such as ribavirin, amantadine, rimantadine, or a neuraminidase-inhibitor.

The compositions and methods as recited herein can include anti-infectious agent used in composition with an anti-MPV antibody, including any of the antibodies or antigen binding fragments thereof described herein, may be an anti-bacterial agent, including but not limited to a macrolide, a penicillin, a cephalosporin, or a tetracycline, or may be an antifungal agent, including but not limited to amphotericin b, fluconazole, or ketoconazole, or an anti-parasitic agent, including but not limited to trimethoprim, pentamidine, or a sulfonamide. The anti-infectious agent may be an anti-viral agent such as ribavirin, amantadine, rimantadine, or a neuraminidase-inhibitor. Such additional agents can also include agents useful against other viruses as well as other agents useful against MPV.

Combinations thereof are also specifically contemplated for the methods described herein.

In some embodiments, the combination of therapeutic antibodies discussed herein can be administered concurrently as a single composition in a pharmaceutically acceptable carrier, or concurrently as separate compositions with each antibody in a pharmaceutically acceptable carrier. In another embodiment, the combination of therapeutic antibodies can be administered sequentially.

### X. Kits

Also provided are kits comprising the anti-hMPV antibodies or antigen binding fragments thereof, multi-specific molecules, or immunoconjugates disclosed herein, optionally contained in a single vial or container, and include, e.g., instructions for use in treating or diagnosing a disease (e.g., viral respiratory disease). The kits may include a label indicating the intended use of the contents of the kit. The term label includes any writing, marketing materials or recorded material supplied on or with the kit, or which otherwise accompanies the kit. Such kits may comprise the antibody, multi-specific molecule, or immunoconjugate in unit dosage form, such as in a single dose vial or a single dose pre-loaded syringe.

The present disclosure is further illustrated by the following examples, which should not be construed as further limiting. The contents of all figures and all references, Genbank sequences, patents, and published patent applications cited throughout this application are expressly incorporated herein by reference.

Commercially available reagents referred to in the Examples below were used according to manufacturer's instructions unless otherwise indicated. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### EXAMPLES

### Example 1. Materials and Methods

### Production of Recombinant Proteins

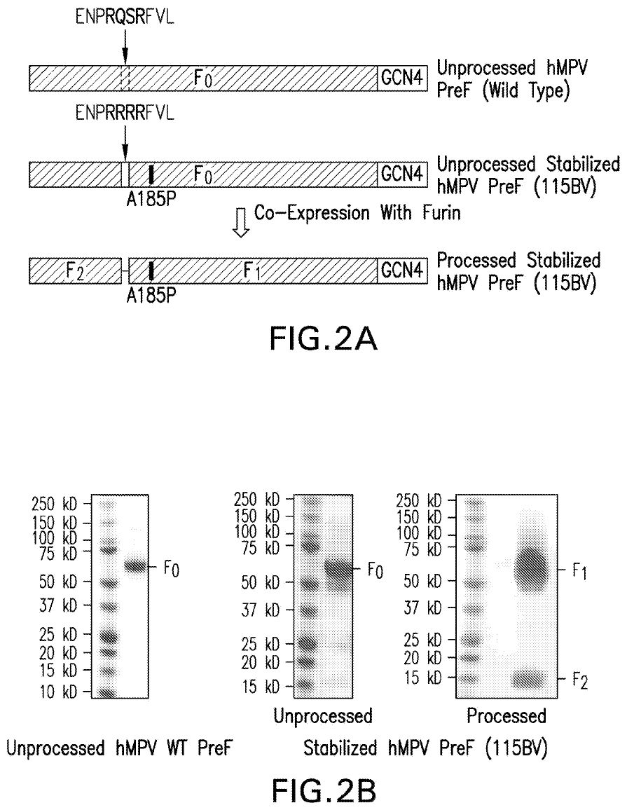

The plasmid construction and production of RSV PreF (DS-Cav1) was performed as previously described [31, 55]. The unprocessed hMPV PreF trimeric antigen was derived from a previously published F sequence of strain B2 (nucleotide sequence is SEQ ID NO: 1003, protein sequence is SEQ ID NO: 1) with a C-terminal GCN4 trimerization domain, which adopted a PreF-like structure (see SEQ ID NO: 910 below for entire sequence) [34]. The monomeric hMPV F antigen was derived from the same B2 strain but without the GCN4 domain (see SEQ ID NO: 911 below for entire sequence). The stabilized hMPV PreF construct (1 15BV) adopted the prefusion stabilizing mutations from a previous report [28], as well as a proline mutation A185P and insertion of a furin-cleavage site, on the backbone of wild-type strain B2 hMPV F trimeric antigen with C-terminal GCN4 domain (see FIG. 2A and SEQ ID NO: 913 below for entire sequence). The hMPV postF sequence was derived from strain B2, where a furin-cleavage site (RKRR; SEQ ID NO: 908) was introduced to the original F1/F2 cleavage site (RQSR; SEQ ID NO: 1067), the fusion peptide sequence FVLGAIAL (SEQ ID NO: 909) at the N-terminal of F1 was deleted, and a C-terminal foldon trimerization domain (GYIPEAPRDGQAYVRKDGEWVLLSTFL; SEQ ID NO: 1012) was added (see SEQ ID NO: 912 for entire protein sequence). The hMPV F mutants were made on the backbone of the unprocessed hMPV F trimeric antigen. All constructs had a C-terminal thrombin cleavage site (LVPRGS; SEQ ID NO: 1013), followed by a six histidine amino acid (6×His) tag (SEQ ID NO: 1014) and a strep tag II (SAWSHPQFEK; SEQ ID NO: 1015). Sequences were codon-optimized for mammalian expression (Life Technologies and Genewiz), cloned into an expression vector, and transiently transfected into Expi293 suspension cells (Life Technologies).

The Unprocessed hMPV PreF Trimeric Antigen Sequence:

(SEQ ID NO: 910)
MSWKVMIIISLLITPQHGLKESYLEESCSTITEGYLSVLRTGWYT
NVFTLEVGDVENLTCTDGPSLIKTELDLTKSALRELKTVSADQLA
REEQIENPRQSRFVLGAIALGVATAAAVTAGIAIAKTIRLESEVN
AIKGALKTTNEAVSTLGNGVRVLATAVRELKEFVSKNLTSAINKN
KCDIADLKMAVSFSQFNRRFLNVVRQFSDNAGITPAISLDLMTDA
ELARAVSYMPTSAGQIKLMLENRAMVRRKGFGILIGVYGSSVIYM
VQLPIFGVIDTPCWIIKAAPSCSEKDGNYACLLREDQGWYCKNAG
STVYYPNDKDCETRGDHVFCDTAAGINVAEQSRECNINISTTNYP
CKVSTGRHPISMVALSPLGALVACYKGVSCSIGSNRVGIIKQLPK
GCSYITNQDADTVTIDNTVYQLSKVEGEQHVIKGRPVSSSFDPIK
FPEDQFNVALDQVFESIENSQALVDQSNKILNSAARMKQIEDKIE
EILSKIYHIENEIARIKKLIGEAGSGLVPRGSHHHHHHSAWSHPQ
FEK

The Unprocessed hMPV PreF Monomeric Antigen Sequence (Modified from the Trimeric Construct Above):

(SEQ ID NO: 911)
MSWKVMIIISLLITPQHGLKESYLEESCSTITEGYLSVLRTGWYT
NVFTLEVGDVENLTCTDGPSLIKTELDLTKSALRELKTVSADQLA
REEQIENPRQSRFVLGAIALGVATAAAVTAGIAIAKTIRLESEVN
AIKGALKTTNEAVSTLGNGVRVLATAVRELKEFVSKNLTSAINKN
KCDIADLKMAVSFSQFNRRFLNVVRQFSDNAGITPAISLDLMTDA
ELARAVSYMPTSAGQIKLMLENRAMVRRKGFGILIGVYGSSVIYM
VQLPIFGVIDTPCWIIKAAPSCSEKDGNYACLLREDQGWYCKNAG
STVYYPNDKDCETRGDHVFCDTAAGINVAEQSRECNINISTTNYP
CKVSTGRHPISMVALSPLGALVACYKGVSCSIGSNRVGIIKQLPK
GCSYITNQDADTVTIDNTVYQLSKVEGEQHVIKGRPVSSSFDPIK
FPEDQFNVALDQVFESIENSQALVDQSNKILNSAEKGNTGLVPRG
SHHHHHHSAWSHPQFEK

hMPV postF Sequence:

(SEQ ID NO: 912)
MSWKVMIIISLLITPQHGLKESYLEESCSTITEGYLSVLRTGWYT
NVFTLEVGDVENLTCTDGPSLIKTELDLTKSALRELKTVSADQLA
REEQIENPRQSRKRRGVATAAAVTAGIAIAKTIRLESEVNAIKGA
LKTTNEAVSTLGNGVRVLATAVRELKEFVSKNLTSAINKNKCDIA
DLKMAVSFSQFNRRFLNVVRQFSDNAGITPAISLDLMTDAELARA
VSYMPTSAGQIKLMLENRAMVRRKGFGILIGVYGSSVIYMVQLPI
FGVIDTPCWIIKAAPSCSEKDGNYACLLREDQGWYCKNAGSTVYY
PNDKDCETRGDHVFCDTAAGINVAEQSRECNINISTTNYPCKVST
GRHPISMVALSPLGALVACYKGVSCSIGSNRVGIIKQLPKGCSYI
TNQDADTVTIDNTVYQLSKVEGEQHVIKGRPVSSSFDPIKFPEDQ
FNVALDQVFESIENSQALVDQSNKILNSAEKGNTGSAIGGYIPEA
PRDGQAYVRKDGEWVLLSTFLGGLVPRGSHHHHHHSAWSHPQFEK

The Stabilized hMPV PreF Sequence):

(SEQ ID NO: 913)
MSWKVMIIISLLITPQHGLKESYLEESCSTITEGYLSVLRTGWYT
NVFTLEVGDVENLTCTDGPSLIKTELDLTKSALRELKTVSADQLA
REEQIENPRRRRFVLGAIALGVATAAAVTAGIAIAKTIRLESEVN
AIKGALKTTNEAVSTLGNGVRVLATAVRELKEFVSKNLTSAINKN
KCDIPDLKMAVSFSQFNRRFLNVVRQFSDNAGITPAISLDLMTDA
ELARAVSYMPTSAGQIKLMLENRAMVRRKGFGILIGVYGSSVIYM
VQLPIFGVIDTPCWIIKAAPSCSEKDGNYACLLREDQGWYCKNAG
STVYYPNDKDCETRGDHVFCDTAAGINVAEQSRECNINISTTNYP
CKVSTGRHPISMVALSPLGALVACYKGVSCSIGSNRVGIIKQLPK
GCSYITNQDADTVTIDNTVYQLSKVEGEQHVIKGRPVSSSFDPIK
FPEDQFNVALDQVFESIENSQALVDQSNKILNSAARMKQIEDKIE
EILSKIYHIENEIARIKKLIGEAGSGLVPRGSHHHHHHSAWSHPQ
FEK

The processed stabilized hMPV PreF construct (115BV) was co-transfected with furin at 1:1 ratio, and other hMPV F constructs were transfected without furin. At day 3 to day 7 post transfection, supernatants were harvested for western blot to confirm expression, for direct ELISA binding assay, and for large-scale purification. The purification of all antigens were performed as previously described [55]. Briefly, harvested supernatants with his-tagged proteins were captured by Ni-Sepharose chromatography (GE Healthcare) and eluted by high imidazole concentration. After an overnight dialysis in the presence of thrombin, the His-tag was cleaved, and concentration of imidazole was reduced. Uncleaved His-tag products as well as initial Ni-Sepharose non-specific binding impurities were removed by negative Ni-Sepharose chromatography (product in flow-through). The protein antigens were further purified by size-exclusion chromatography (Superdex 200, GE Healthcare) and stored in a buffer of 50 mM 4-(2-hydroxyethyl)-1-piperazineethanesulfonic acid (HEPES) pH 7.5 with 300 mM sodium chloride (NaCl).

### Human Subjects and PBMC Preparation

Blood samples from healthy adults were purchased from a Biological Specialty Company (Colmar PA) after written informed consent. Plasma or serum samples from these donors were screened for activity in an hMPV microneutralization assay. Peripheral blood mononuclear cells (PBMCs) were purified from blood collected in EDTA tubes by density gradient centrifugation in Histopaque™ over Accuspin™ tubes (Sigma Aldrich) according to the manufacturer's instructions. PBMCs were then frozen in 90% heat-inactivated fetal bovine serum (FBS) supplemented with 10% dimethyl sulfoxide and stored in liquid nitrogen until thawed for use in assays.

### Antigen-Specific Single Memory B Cell Sorting and Culture

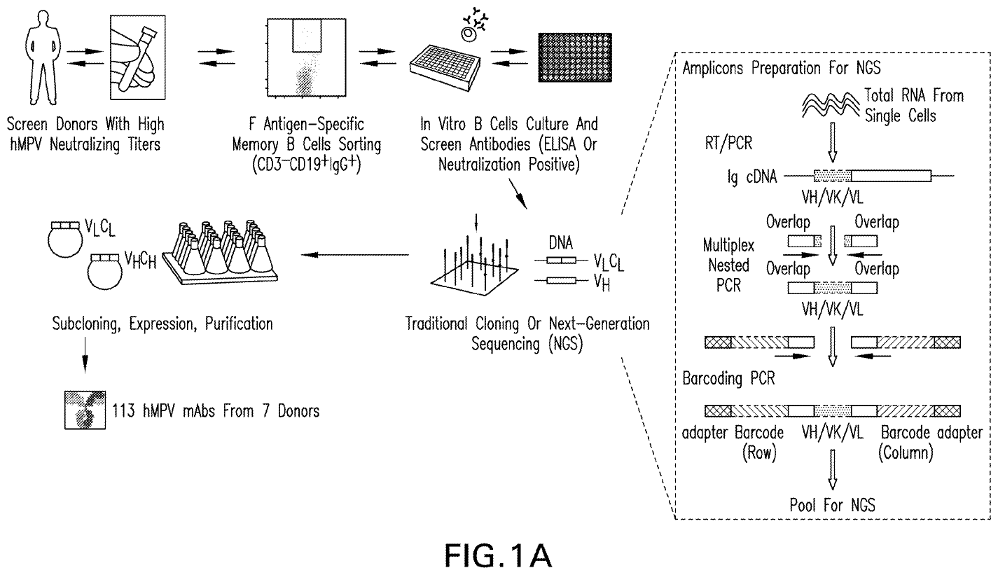

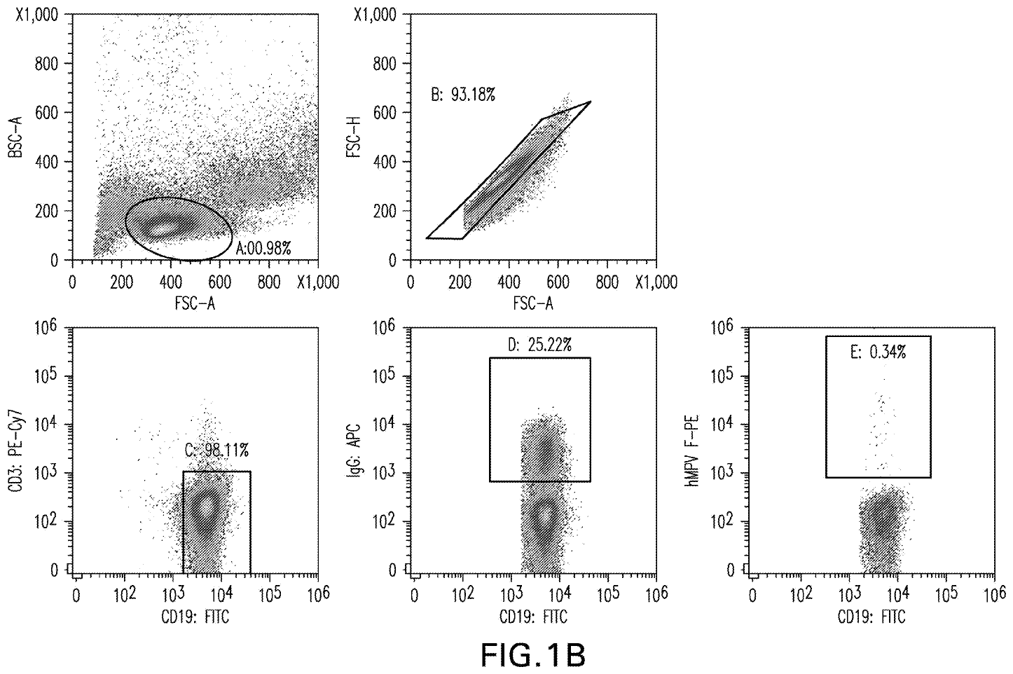

Human memory B cells from selected donors were single-sorted with specific antigens as previously described with minor modifications [53, 56]. The purified recombinant unprocessed hMPV PreF trimer and monomer proteins were biotinylated (Thermo) and used as sorting antigens. For a portion of the experiments, B cells were sorted using a mixture of biotinylated unprocessed hMPV PreF and RSV PreF (DS-Cav1) tagged with Alexa-647. Cryopreserved PBMCs were thawed on the day of sorting and the B cell population was enriched using the EasySep™ Human B-cell Enrichment Kit (Stemcell Technologies). Next, B cells were stained with biotinylated or Alexa 647 tagged F antigens and then followed by staining with a panel of monoclonal antibodies including anti-CD3 mAb-PE-Cy™ 7 (BD Biosciences), anti-CD19-FITC (BD Biosciences), anti-human IgG-APC or BV421 (BD Biosciences), and PE-streptavidin. CD3⁻/CD19⁺/IgG⁺/F⁺ cells were sorted with a BD FACS Jazz or SONY 800S cell sorter in single cell mode into a 96-well plate (FIG. 1B). The sorted memory B cells were then cultured for 14 days at 37° C., 5% carbon dioxide (CO₂) for conversion into IgG secreting cells as previously described [56]. At the end of cultures, plates were centrifuged at 2,000 rpm for 10 minutes. The culture supernatants were then transferred to new 96-well plates and screened for ELISA positive binding to hMPV PreF trimer. The cell pellets were lysed in 50 μl of RLT buffer (Qiagen) supplemented with 1% 2-mercaptoethanol (Sigma) and stored at −80° C. for immunoglobulin gene sequencing.

### Recovery of Antibody Sequences and Antibody Production

The antibody sequences from the wells with positive ELISA binding to hMPV F were further recovered by regular cloning methods as previously described [56] or by barcode-based next-generation sequencing (NGS). Specifically, for NGS cloning, after RNA extraction and the first step of reverse transcription polymerase chain reaction (RT-PCR) to amplify variable regions, 5′ and 3′ overlap sequences were attached by a second-step of multiplex nested PCR, followed by a third-step of PCR to add 5′ and 3′ barcode and adaptor sequences. The final amplicons, which contain unique 5′ and 3′ barcode combination for each well, were pooled together and sequenced by Illumina MiSeq™. Primer sequences were included and are shown in Table 2.

NGS sequences were checked for quality by FastQC v0.11.2 (bioinformatics.babraham.ac.uk/projects/fastqc) and paired-end reads were assembled using PANDAseq v2.10 [57] with a minimum overlap region of 10 base pair (bp). The assembled B cell receptor (BCR) reads were aligned to V(D)J germline sequences (IMGT v3.1.19) [58] using IgBLAST v1.9.0 [59] and to immunoglobulin heavy-chain constant (IGHC) sequences using blastn (v2.2.29) as a part of the BLAST+suite [60]. The output of IgBLAST was processed using commands from Change-O v0.3.12 [61]. Only functional sequences with E-value no more than 0.001 for the alignment of the V and J genes were retained for downstream analysis. The most abundant sequence obtained from each well were representative of the antibody sequence recovered from the well.

The naturally paired heavy and light chain variable region sequences obtained from single sorted human memory B cell cultures were sub-cloned into pTT5 vector for expression in CHO-3E7, which is a stable Chinese hamster ovary (CHO) clone expressing a truncated but functional form of Epstein-Barr virus nuclear antigen-1 (EBNA1). The CHO-3E7 cells (performed at Genscript). Briefly, CHO-3E7 cells were grown in serum-free FreeStyle™ CHO Expression Medium (Life Technologies). The recombinant plasmids encoding heavy and light chains of each antibody were transiently co-transfected into suspension CHO-3E7 cell cultures. The culture supernatants collected were used for purification with a Protein A Cleaning-in-place (CIP) column (GenScript). The purified antibodies were quality controlled (QC) tested by SDS-PAGE and western blot.

hMPV Plaque Reduction Neutralization Assay

A plaque reduction neutralization assay was developed for hMPV similarly as described previously for RSV [62]. Briefly, antibodies in serial dilutions with OptiMEM™ medium (Gibco) were first added into Poly-D coated 96 well flat bottom plates (Corning Costar) at 50 μl per well. For initial screening, the hMPV A1 and B2 viruses (ZeptoMetrix Corp) at 2000 pfu/ml were mixed with antibody at 50 μl per well and incubated for 1 h at 37° C. with 5% CO₂. Rhesus Monkey Kidney Epithelial Cells (LLC-MK2 line) LLC-MK2 is an epithelial line that was established in the 1950s from a pooled suspension prepared from renal tissue excised from six rhesus monkeys (Macaca mulatta). The LLC-MK2 cells (0.8×10⁶ to 1.2×10⁶ cells/ml in OptiMEM™ medium) were then added to the antibody/virus mixtures at 25 μl per well. After 1 hour incubation at 37° C. with 5% CO₂, the plates were centrifuged at 1,200 rpm for 10 minutes. A volume (125 μl) of OptiMEM™ medium supplemented with 1% methylcellulose was overlaid in each well. Plates were incubated at 37° C. with 5% CO₂ for 4 days. Cells were then fixed with 10% formalin (Fisher Scientific) at 100 μl per well for 30 min at room temperature. The plates were dried for 20 minutes before washing with phosphate-buffered saline with Tween @detergent (PBST). Fixed cells were treated with blocking buffer (Odyssey) for 30 minutes, followed by a 2 hour incubation with a mouse anti-hMPV mAb (EMD Millipore, 1:1000) diluted in blocking buffer at 50 μl per well. Plates were washed again by PBST and then incubated with an anti-mouse IgG Alexa 488 conjugated secondary antibody (Invitrogen, 1:500) in assay diluent at 50 μl per well for 1 hour. After washing off the excessive secondary antibodies using PBST, the plates were analyzed using an EnSight™ plate reader (PerkinElmer). The IC₅₀ values for the antibodies were calculated from a 4 parameter nonlinear fitting algorithm using a Graph Pad Prism software. For the antibodies that showed an IC₅₀<10 μg/ml in initial screening, neutralization assays with the same procedure were repeated in wider range of antibody concentration with hMPV A2 (RL_bx from Baylor) and B2 (Peru6-2003) viruses.

### Plaque Reduction Neutralization Assay

A plaque reduction neutralization assay was utilized by investigators [62]. Briefly, antibodies in serial dilutions with Eagle's Minimum Essential Medium (EMEM) medium supplemented with 2% FBS and 2 mM glutamine were first added into Poly-D coated 96 well flat bottom plates (Corning Costar) at 50 μl per well. The RSV A Long and B Washington strains at 2000 pfu/ml were mixed with antibody at 50 μl per well and incubated for 1 hour at 37° C. with 5% CO₂. The HEp-2 cells (ATCC) at 0.8×10⁶ to 1.2×10⁶ cells/ml in EMEM medium supplemented with 2% FBS and 2 mM glutamine were then added to the antibody/virus mixtures at 25 μl per well. After an 1 hour incubation at 37° C. with 5% CO₂, the plates were centrifuged at 1,200 rpm for 10 minutes. 125 μl of EMEM medium supplemented with 2% FBS, 2 mM glutamine and 1% methylcellulose were overlaid in each well. Plates were incubated at 37° C. with 5% CO₂ for 3 days. Cells were then fixed with ice-cold 80% acetone (Sigma) in phosphate-buffered saline (PBS) at 100 μl per well for 10-20 min at room temperature. The plates were dried for 20 minutes and then washed with PBST. Fixed cells were stained with a mixture of a mouse anti-F monoclonal antibody (1.25 μg/ml) and a mouse anti-N(nucleoprotein) monoclonal antibody (1.25 μg/ml). Plates were washed again by PBST and then incubated with an anti-mouse IgG Alexa 488 conjugated secondary antibody (Invitrogen, 1:500) in assay diluent at 50 μl per well for 1 hour. After washing off the excessive secondary antibodies using PBST, the plates were analyzed using an EnSight™ plate reader (PerkinElmer). The IC₅₀ values were calculated from a four-parameter nonlinear fitting algorithm using a GraphPad Prism software.

Cryo-EM Study of M4B06 and hMPV-F Complex

For sample preparation, the M4B06-hMPV PreF complex was prepared by incubating recombinantly purified M4B06 Fab with a processed hMPV PreF (115BV) trimer (PreF trimer: Fab=1:3) for 1 hour at 4° C., followed by purification using Superdex 200 Increase 10/300 GL column in buffer (25 mM HEPES pH 7.5, 150 mM NaCl). The fraction sample was flashed frozen and pre-checked with negative staining for subsequent cryo-EM study. Three microliters of the purified sample at 0.2 mg/ml were applied onto a glow-discharged holey carbon film grid with Graphene Oxide (Quantifoil, 300 mesh, R2.0/1.0). Samples were blotted for 6 seconds (s) and vitrified by plunging into liquid ethane cooled by liquid nitrogen using a Vitrobot™ Mark IV (Thermo Fischer Scientific) at 4° C. and 100% humidity.

For Cryo-EM data acquisition, a total of −1,800 movie micrographs (36 frames for each movie) were acquired using a FEI Titan Krios™ 300 KV electron microscope. The data acquisition also involved using a Gatan K3™ Summit direct electron detector, operated in super resolution mode applying a total dose of 75 e⁻/Å² during a 3 s exposure. During the 3 second period, the total dose is 75 electrons per A2 (angstrom square) of detector (A2 is unit of area size of detector). Each micrograph was acquired (with SerialEM software) at 89,000× nominal magnification (0.5225 Å/pixel at the specimen level) with a nominal defocus range of −1.5 μm to −3.0 μm.

For image processing and 3D reconstruction, all movie frames were aligned and binned by a factor of 2 using MotionCor2 [63] and their contrast transfer function (CTF) parameters were estimated with a Gctf computer program [64]. Approximately 1,000 particles were manually boxed and extracted from ˜30 far-defocus micrographs, and their two dimensional (2D) average classes were used as templates to automatically box particles from all selected micrographs with autopick in a regularized likelihood optimization (RELION) 3.0 computer program [65]. A total of −1,000,000 picked-particle images were extracted (binned by a factor of 8 to give a pixel size of 4.18 Å) and were subjected to reference-free 2D alignment with RELION 3.0. After multiple iterations of 2D classifications, particles (˜218,000) belonging to good classes were three dimensional (3D) classified against the map created from manually-picked particles as the initial model. After 2 iterations of 3D classifications, particles (˜131,000) in good classes were re-centered, re-extracted (binned by a factor of 5 to give a pixel size of 2.6 Å) and refined to yield a map at Nyquist frequency of 5.2 Å resolution. Using this map as a model, particles were further 3D classified into 6 classes (S6 Fig A) without any further origin and orientation determination. Particles (87,876) in those three classes showing better identifiable structural features were re-extracted (binned by a factor of 2 to give a pixel size of 1.045 Å) and further refined to a final map with resolution of 3.75 Å (FSC=0.143) without imposing any symmetry.

For model building, the model of a hMPV PreF trimer of B strain was built using the crystal structure of A strain as the template [28]. The model was docked into CryoEM map using a Chimera visualization program [66]. Structural figures were prepared using the Chimera program.

### Transmission Electron Microscopy and Image Analysis

Transmission electron microscopy and 2D class averaging were performed by NanoImaging Services, Inc. (San Diego, CA). Samples were prepared on continuous carbon films supported by nitrocellulose-coated 400 mesh copper grids (Ted Pella). A 3 μL drop of purified hMPV PostF protein at a concentration of 2-8 μg/mL was applied to a freshly plasma-cleaned grid for 1 minute. The drop was then blotted to a thin film using filter paper, followed by immediate staining with 1% (w/v) uranyl formate. Transmission electron microscopy was performed using a FEI Tecnai T12 electron microscope operating at 120 kilovolt (kV) equipped with an FEI Eagle 4 k×4 k charge-coupled device (CCD) camera. Negative stain grids were transferred into the electron microscope using a room temperature stage. Images of each grid were acquired at multiple scales to assess the overall distribution of the specimen. After identifying potentially, suitable target areas for imaging at lower magnifications, pairs of high magnification images were acquired at nominal magnifications of 110,000× (0.10 nm/pixel) and 67,000× (0.16 nm/pixel) using the automated image acquisition software package Leginon [67]. Images were acquired at a nominal underfocus of −2 μm to −1 μm and electron doses of approximately 30-35e/A2.

Image processing was performed using the Appion software package [68]. Contrast transfer functions of the images were estimated using a CTFFIND4 program [69]. CTFFIND is a widely-used program for the estimation of objective lens defocus parameters from transmission electron micrographs. Individual particles in the 67,000× or 110,000× high magnification images were selected using automated picking protocols [70], followed by several rounds of reference-free alignment and classification using the X-windows-based microscopy image processing package (Xmipp). See [71]. XMipp contains an algorithm that aligns the selected particles and sorts them into self-similar groups of classes.

### Generation of Monoclonal Antibody Resistant Mutants (MARMs) for M4B06

Vero kidney epithelial cells were seeded in 6-well plates in OptiPRO™ SFM medium (Gibco), which is a serum-free, animal origin-free culture medium. The medium was supplemented with 1% Pen Strep (Gibco), 2% L-Glutamine (Gibco) and 2% FBS (HyClone) at 37° C. with 5% CO₂. In the next day, the cells were infected by a mixture of hMPV A2 virus (RL_bx from Baylor) at 10 plaque forming units per cell (pfu/cell) and M4B06 Fab at 2 μg/mL, 8 μg/mL, and 40 μg/mL in three rounds respectively. In every round, cells were cultured at 37° C. with 5% CO₂ and harvested when syncytia appeared (day 6), lysed by three times of freeze/thaw in liquid nitrogen, and centrifuged at 500 g for 10 minutes. One milliliter of supernatant containing virus was used to infect fresh monolayer Vero cells. The remaining supernatant was mixed with 10× sucrose-phosphate-glutamate-albumin (SPGA) buffer (Biological Industries) and frozen for long-term storage. After three rounds of culture and infection, the virus supernatant from the last round was aliquoted into 10-fold serial dilutions and 0.5 mL of supernatant was used to incubate with fresh monolayer the Vero cells at 37° C. with 5% CO₂. After a 1 hour incubation, a 3.5 mL volume overlay (OptiMEM™ medium supplemented with 1% methylcellulose) was added, and cells were cultures at 37° C. with 5% CO₂ for 1 week.

Twenty-two single plaques were then collected separately and inoculated in fresh monolayer Vero cells prepared in a 24-well plate for 5 days at 37° C. with 5% CO₂ before harvesting. The final harvested MARM viruses were lysed in RLT buffer (Qiagen) with 1% 2-mercaptoethonal and viral RNA were extracted using a RNeasy commercial kit (Qiagen). The F genes from individual plaques were amplified by RT-PCR using viral RNAs, followed by PCR analysis with F-specific primers, gel extraction, and sequencing.

### Biolayer Interferometry (BLI)-Based Binding Assay and Epitope Binning

BLI-based assays were performed using an Octet Red 96e instrument (ForteBio, Inc.). All experiments were performed at 30° C. with shaking at 1,000 rpm. For the binding assays, the investigators utilized recombinant unprocessed hMPV PreF trimer, processed stabilized hMPV PreF trimer, and hMPV PostF trimer as distinct antigens. The antibodies and antigen proteins were diluted in the kinetics buffer (ForteBio) at concentrations of 0.5 μg/mL and 2 μg/mL, respectively. The antibodies and a reference well with empty buffer were first individually immobilized on anti-human Fc capture (AHC) biosensors (ForteBio) for 180 seconds, followed by the association step with antigen for 600 seconds. Most of antibodies' association curves were within linear range without showing saturation. For data processing, the absolute binding response (nm) of each antibody sample was calculated by substrating the binding response (nm) of the reference well (Table 4). The plots were generated using GraphPad Prism software.

For epitope binning analysis, the 73 isolated mAbs which showed apparent BLI binding response (>0.2 nm) were included in the experiments. Recombinant unprocessed hMPV PreF trimer proteins were used as antigens. All the protein samples were diluted in the kinetics buffer (ForteBio). The antibodies to be characterized or the reference mAbs to be used as internal controls (5 μg/mL) were first individually immobilized on anti-human Fc capture (AHC) biosensors (ForteBio) for 600 seconds. The sensors having the samples and antibodies were then blocked with an irrelevant mAb (5 μg/mL) for 600 seconds. The biosensors were then immersed into wells containing the F antigen (2 μg/mL). Finally, the antigen loaded biosensors were immersed into wells containing the second reference mAbs (5 μg/mL). The normalized secondary mAbs binding was calculated by dividing the binding responses of second reference mAbs binding by the binding responses of antigen loading. The normalized numbers were then plotted as a clustered heatmap by heatmap.2 package in R Studio 1.0.153. MAbs with similar competition profiles as the reference mAbs were clustered into the same categories. The neutralization IC50 values of hMPV A, B and RSV A, B were plotted as top-bars in the heatmap.

### ELISA Assay

For ELISA assays with unpurified antigens in Expi293 supernatants, 96-well Ni-NTA coated plates (Thermo Scientific) were coated with cell culture supernatants for 2 hours at room temperature. Plates were then blocked by adding 2% (v/v) bovine serum albumin (BSA) in PBS. After the blocking step, plates were incubated with serial dilutions of antibodies at room temperature for 90 minutes. Plates were washed by PBST and then incubated with horseradish peroxidase (HRP)-conjugated goat anti-human IgG (1: 2,000, Southern Biotech) for 45 minutes. Plates were washed again and developed with tetramethylbenzidine (TMB) solution (Virolabs). Any absorbance at 450 nm was detected using a plate reader (Victor III; Perkin-Elmer). The EC50 values were calculated by performing a Hill slope curve fitting using GraphPad Prism software.

### Epitope Mapping of M4B06 by HDX-MS

To optimize the sequence coverage, several preliminary experiments using various quench conditions, digestion, desalting, liquid chromatography, and mass spectrometry parameters were evaluated. Also, the optimal antigen-antibody ratio was determined in a separate titration experiment. The ratio was optimized by keeping a constant antigen concentration while changing the antibody concentration. Several peptides were identified whose deuteration levels decreased upon addition of the antibody. The optimal Ag:Ab ratio was identified when no additional decrease in deuteration levels were observed which represented equimolar amounts of antigen (Ag) and antibody (Ab) for binding.

The following represents the conditions that were determined to be optimal for the hydrogen-deuterium exchange experiments. Stock solutions of 2.6 μM hMPV prestabilized processed PreF trimer (115BV) with and without an equimolar amount of M4B06 monovalent Fab were prepared in 1×PBS. The Ag:Ab complex was prepared by incubating hMPV F with M4B06 at ambient temperature for 1 hour before cooling to 1° C. The hydrogen-deuterium exchange reactions were initiated by adding 40 μL PBS prepared in D20 to 10 μL of the hMPV F and hMPV F/M4B06 solutions. The labeling reactions were performed at 1° C. for five time points: 15 seconds, 50 seconds, 150 seconds, 500 seconds, and 5000 seconds. After these time points the samples were quenched by mixing 40 μL of the labeled solution with 20 μL of quench solution (2M urea, 1M tris(2-carboxyethyl)phosphine (TCEP), adjusted to pH 3.0 with NaOH) at 1° C. 50 μL of the quenched sample was immediately injected onto an immobilized pepsin column (Waters Enzymate BEH Pepsin column, 2.1×30 mm) held at 10° C. for on-line proteolysis using 0.05% (v/v) trifluoroacetic acid (TFA) in water solvent at a flow rate of 200 μL/minute. The flow, with the digest, continued at the same rate onto a trap column (Waters ultra-performance liquid chromatography (UPLC) ethylene bridged hybrid (BEH) 300 C18, 1.7 μm, 2.1×5 cm) held at 0° C. for desalting. The combined digestion and desalting steps lasted for 90 sec after which the trap column flow was reversed and eluted onto an analytical column (Waters Acquity™ UPLC charged surface hybrid (CSH) carbon 18(C18_, 1.7 μm, 1.0×100 mm) held at 0° C. using a gradient of solvent A: 0.05% (v/v) TFA in water and solvent B: 0.0025% (v/v) TFA in 95% acetonitrile/5% water (v/v). A linear gradient of 13% to 40% B over 9.5 min at a flow rate of 40 μL/min was employed to elute the peptides into a ThermoScientific LTQ-XL Orbitrap mass spectrometer (Thermo Fisher Scientific) operated in positive mode at a resolution of 30,000. The data was acquired over the m/z range of 350-2000 in profile mode using the following MS parameters: heated electrospray ionization (HESI) source voltage 4 kV, capillary voltage 44 V, tube lens 110 V, capillary temperature 250° C., and sheath gas flow 10. The labeling, quench, injection, digestion, desalting, and elution steps were performed using an HTC PAL robot (LEAP Technologies) controlled by HDxDirector software. The isocratic and gradient solvent flows were obtained using Waters nano-Acquity™ UPLC pumps (Waters). Prior to the exchange experiment, the identity of each peptide was confirmed by analyzing a non-deuterated RSV F sample in data-dependent MSMS mode and processed with Proteome Discoverer 1.4 software (Thermo Fisher Scientific). The HDExaminer software (Sierra Analytics) was used to determine the centroid mass of the isotopic envelope of each peptide in the labeling experiment and quantifying deuterium incorporation.

Differential Scanning Fluorimetry for Unprocessed and Processed hMPV F Proteins

Analyses were performed with unprocessed hMPV PreF protein and processed stabilized hMPV PreF protein expressed with and without furin that had been stored at −70° C. Two-fold serial dilutions of hMPV F protein in 50 mM HEPES, 300 mM NaCl at pH 7.5 were prepared from starting concentrations of 1.5 mg/ml, 2.5 mg/ml and 2.7 mg/ml, respectively to obtain samples in a concentration range between (3.125 μM-50 μM). Differential scanning fluorimetry (DSF) measurements were performed using the Prometheus NT.48 instrument (NanoTemper Technologies GmbH, Munich, Germany). In brief, experiments were performed in duplicate using high sensitivity capillaries (PR-C006). Each capillary was filled with 10 μl of protein solution and the fluorescence signal at 350 nm and 330 nm was recorded at temperatures between 20° C. to 95° C. at a temperature increase rate of 1° C./minute. Instrument software was used to calculate the ratio between the 350 nm and 330 nm signal as well as the first derivative. Maxima of the first derivative were used to obtain transition midpoints of protein unfolding events.

### Serum Absorption Assay

Serum samples of four donors were tested in the absorption assay. Each sample (50 μL) is diluted in PBS to a total volume of 970 μL and mixed with 20 μL of 0.5 mg/mL antigen or PBS buffer control for two-hour incubation at room temperature. The samples were then mixed with 10 μl of reconstituted Strep-tag II mAb (GenScript) at 0.5 mg/ml and place on a 3600 rotator at 4° C. for 2 hours. Pre-washed 80 μL of sheep anti-mouse immunoglobulin G Dynabeads™ (2 mg/ml, Life Technologies) were then added to the mixture for 1 hour incubation at 4° C. on a rotator. Dynabeads carrying antigen and antigen-bound antibodies were separated by DynaMag-2 magnet (Life Technologies), leaving absorbed supernatant for further ELISA binding assay with F antigens. ELISA titers were calculated by dilution folds, and averaged ELISA titers from four donors were plotted.

### Computational Sequence and Structural Analysis

The V gene germline usage, clonal analysis, and somatic hypermutation (SHM) were analyzed by IgBlast [59] based on Kabat delineation system [72]. The clonotype was defined to includes any V(D)J rearrangements that have the same germline V(D)J gene segments, the same productive/non-productive status and the same CDR3 nucleotide as well as amino sequence. Structural visualization was generated by PyMOL 1.7.05 (Schrödinger). The plots were generated by GraphPad Prism software.

### Example 2. Isolation of a Large Panel of hMPV F-Specific mAbs from Human Memory B Cell Repertoire

To comprehensively profile the human antibody recognition sites to hMPV F antigen, investigators isolated more than 100 mAbs from human memory B cell repertoires of multiple donors by antigen-specific single memory B cell sorting method (FIG. 1A, FIG. 1B and Table 3). A wild-type (WT) hMPV F ectodomain trimer, which showed no obvious cleavage between F1 and F2 (FIG. 2A and FIG. 2B) and likely represents a PreF conformation [34], was used as the primary bait/target for memory B cell sorting. In some cases, a WT monomeric form of hMPV F antigen or a mixture of hMPV WT trimer described above and a prefusion stabilized RSV F antigen DS-Cav1 were used (Table 3A). As a result, a total of 113 mAbs from seven donors, including 88 mAbs from three major donors, have been successfully cloned, expressed, and confirmed their binding to hMPV F antigens (see VH information data in Table 3B and VL information and data in Table 3C), three recently reported RSV/hMPV cross-neutralizing mAbs (M1C7, M2B6, M2D2) were also included [53].

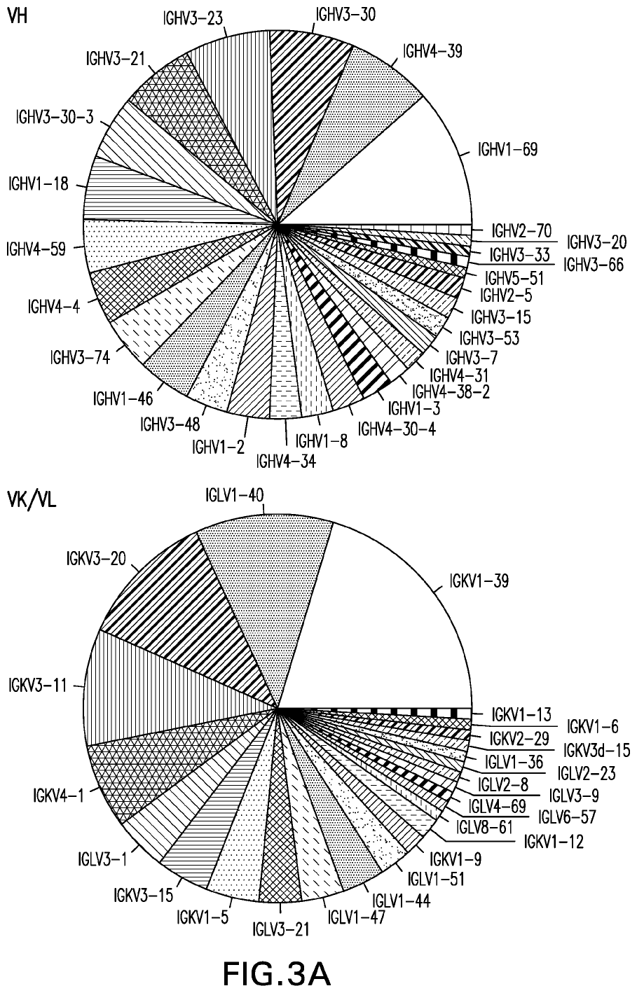

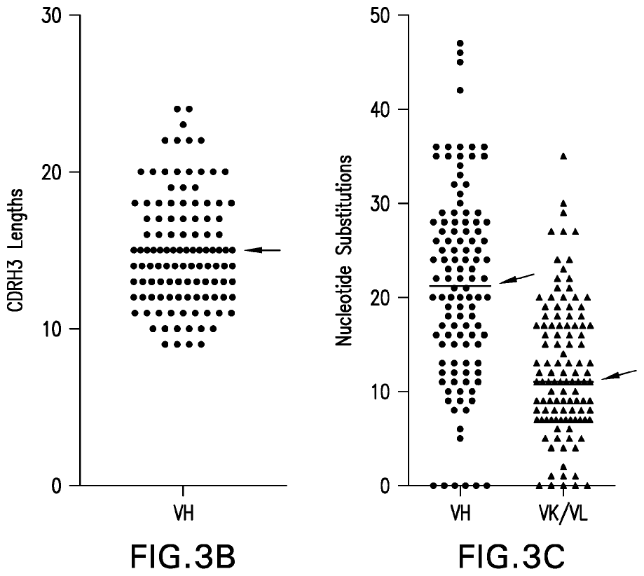

Clonal lineage analysis was performed, and the data showed that the hMPV F-specific B cell repertoires were highly diverse, with 111 out of 113 antibodies being unique. See Table 3D. The frequency of germlines was also diverse (FIG. 3A), indicating that clonal expansion was very rare in the hMPV F induced antibody response. The lengths of HCDR3 range from 9-24 (median 15) (FIG. 3B), which is similar to the recently reported RSV-F specific repertoires in adults [36]. The average levels of somatic hypermutation (SHM) varied between the donors' repertoires (FIG. 3B), ranging from 0-47 (median 21) nucleotide substitutions per VH gene and 0-35 (median 11) nucleotide substitutions per VK/VL gene (excluding CDRH3), respectively (FIG. 3C). Eighty-five out of 113 mAbs neutralized either hMPV A or B subtypes in plaque reduction assays under tested antibody concentrations. Twelve of the mAbs showed high neutralizing potency on both hMPV subtypes A and B with IC50s under 50 ng/ml (Table 3A). Six mAbs were observed to have neutralizing activity for hMPV B subtype (Table 3A), possibly because F antigen of hMPV subtype B was used for sorting. Thirteen hMPV neutralizing mAbs were observed to have cross-binding to RSV F antigens and cross-neutralization to RSV A and RSV B strains (Table 3A).

TABLE 2
Primer sequences for NGS human B cell IgG cloning

| | | |
|---|---|---|
| | SEQ | |
| Primer | ID | |
| name | NO: | Primer sequence |

RT-PCR primers

| | | | | |
|---|---|---|---|---|
| Heavy | 5′ primer | 5′ LH1/7 For | 915 | 5′ ACAGGTGCCCACTCCCAGGTGCAG |
| chain | | | | |
| | | 5′ LH2 For | 916 | 5′ CTGACCATCCCTTCATGGGTCTTGTCC |
| | | 5′ LH3 For | 917 | 5′ AAGGTGTCCAGTGTGARGTGCAGCTG |
| | | 5′ LH4/6 For | 918 | 5′ AGATGGGTCCTGTCCCAGGTGCAG |
| | | 5′ LH5 For | 919 | 5′ CAAGGAGTCTGTTCCGAGGTGCAGC |
| | 3′ primer | 3′ IgG CH2 | 920 | 5′ GCTCACGTCCACCACCACG |
| | | Rev | | |
| Kappa | 5′ primer | 5′LK1/2 For | 921 | 5′ ATGAGGGTCCCYGCTCAGCTCCTG |
| chain | | | | |
| | | 5′ LK3 For | 922 | 5′ CTCTTCCTCCTGCTACTCTGGCTC |
| | | 5′ LK4 For | 923 | 5′ CAGGTCTTCATTTCTCTGTTGCTCTGG |
| | | 5′ LK5 For | 924 | 5′ GTTCACCTCCTCAGCTTCCTCCTC |
| | | 5′ LK6 For | 925 | 5′ CTCTGGGTTCCAGCCTCCAGG |
| | 3′ primer | 3′ CKappa | 926 | 5′ ACACTCTCCCCTGTTGAAGCTCTTTGTG |
| | | Rev | | |
| Lambda | 5′ primer | 5′ Lλ1 For | 927 | 5′ GGTCCTGGGCCCAGTCTGTGCTG |
| chain | | | | |
| | | 5′ Lλ2 For | 928 | 5′ GGTCCTGGGCYCAGTCTGCCCTG |
| | | 5′ Lλ3 For | 929 | 5′ GCTCTGWGGCCTCCTATGAGCTG |
| | | 5′ Lλ4, 5, | 930 | 5′ GGTCTCTCTCSCAGCYTGTGCTG |
| | | 9 For | | |
| | | 5′ Lλ6 For | 931 | 5′ GTTCTTGGGCCAATTTTATGCTGACTC |
| | | 5′ Lλ7 For | 932 | 5′ GGTCCAATTCYCAGGCTGTGGTG |
| | | 5′ Lλ8 For | 933 | 5′ GAGTGGATTCTCAGACTGTGGTG |
| | | 5′ Lλ10 For | 934 | 5′ GTGTCAGTGGTCCAGGCAGGGCTG |
| | 3′ primer | 3′CLambda | 935 | 5′ CATTCYGYAGGGGCMACTGTCTTC |
| | | Rev | | |

Nested-PCR primers

| | | | | |
|---|---|---|---|---|
| Heavy | 5′ primer | 5′VH1, 7 | 936 | 5′ ACACTCTTTCCCTACACGACGCTCTTCCG |
| chain | | ovlp_For | | ATCTCAGGTGCAGCTGGTGCAGTCTGG |
| | | 5′VH2 | 937 | 5′ ACACTCTTTCCCTACACGACGCTCTTCCG |
| | | ovlp_For | | ATCTCAGATCACCTTGAAGGAGTCTGG |
| | | 5′VH3, 5 | 938 | 5′ ACACTCTTTCCCTACACGACGCTCTTCCG |
| | | ovlp_For | | ATCTGAGGTGCAGCTGGTGSAGTCTGG |
| | | 5′VH4 | 939 | 5′ ACACTCTTTCCCTACACGACGCTCTTCCG |
| | | ovlp_For | | ATCTCAGGTGCAGCTGCAGSAGTCGGG |
| | | 5′VH6 | 940 | 5′ ACACTCTTTCCCTACACGACGCTCTTCCG |
| | | ovlp_For | | ATCTCAGGTACAGCTGCAGCAGTCAGG |
| | 3′ primer | 3′ READ2- | 941 | 5′ GTGACTGGAGTTCAGACGTGTGCTCTTC |
| | | HR88 | | CGATCTTGACCAGGCAGCCCAGG |
| Kappa | 5′ primer | 5′ VK1a, b | 942 | 5′ ACACTCTTTCCCTACACGACGCTCTTCCG |
| chain | | ovlp_For | | ATCTGACATCCAGWTGACCCAG |
| | | 5′ VK1c | 943 | 5′ ACACTCTTTCCCTACACGACGCTCTTCCG |
| | | ovlp_For | | ATCTGCCATCCGGTTGACCCAG |
| | | 5′ VK2 | 944 | 5′ ACACTCTTTCCCTACACGACGCTCTTCCG |
| | | ovlp_For | | ATCTGATATTGTGATGACYCAG |
| | | 5′ VK3a, b | 945 | 5′ ACACTCTTTCCCTACACGACGCTCTTCCG |
| | | ovlp_For | | ATCTGAAATTGTGTTGACRCAG |
| | | 5′ VK3c | 946 | 5′ ACACTCTTTCCCTACACGACGCTCTTCCG |
| | | ovlp_For | | ATCTGAAATAGTGATGACGCAG |
| | | 5′ VK4 | 947 | 5′ ACACTCTTTCCCTACACGACGCTCTTCCG |
| | | ovlp_For | | ATCTGACATCGTGATGACCCAG |
| | | 5′ VK5 | 948 | 5′ ACACTCTTTCCCTACACGACGCTCTTCCG |
| | | ovlp_For | | ATCTGAAACGACACTCACGCAG |
| | | 5′ VK6a | 949 | 5′ ACACTCTTTCCCTACACGACGCTCTTCCG |
| | | ovlp_For | | ATCTGAAATTGTGCTGACTCAG |
| | | 5′ VK6b | 950 | 5′ ACACTCTTTCCCTACACGACGCTCTTCCG |
| | | ovlp_For | | ATCTGATGTTGTGATGACACAG |
| | 3′ primer | 3′ READ2- | 951 | 5′ GTGACTGGAGTTCAGACGTGTGCTCTTC |
| | | KR26 | | CGATCTAAGACAGATGGTGCAGC |
| Lambda | 5′ primer | 5′ VL1 | 952 | 5′ ACACTCTTTCCCTACACGACGCTCTTCCG |
| chain | | ovlp_For | | ATCTCAGTCTGTGCTGACKCAG |
| | | 5′ VL2 | 953 | 5′ ACACTCTTTCCCTACACGACGCTCTTCCG |
| | | ovlp_For | | ATCTCAGTCTGCCCTGACTCAG |
| | | 5′ VL3 | 954 | 5′ ACACTCTTTCCCTACACGACGCTCTTCCG |
| | | ovlp_For | | ATCTTCCTATGAGCTGACWCAG |
| | | 5′ VL4, 5, 9 | 955 | 5′ ACACTCTTTCCCTACACGACGCTCTTCCG |
| | | ovlp_For | | ATCTCAGCYTGTGCTGACTCAG |
| | | 5′ VL6 | 956 | 5′ ACACTCTTTCCCTACACGACGCTCTTCCG |
| | | ovlp_For | | ATCTAATTTTATGCTGACTCAG |
| | | 5′ VL7, 8 | 957 | 5′ ACACTCTTTCCCTACACGACGCTCTTCCG |
| | | ovlp_For | | ATCTCAGRCTGTGGTGACTCAG |
| | | 5′ VL10 | 958 | 5′ ACACTCTTTCCCTACACGACGCTCTTCCG |
| | | ovlp_For | | ATCTCAGGCAGGGCTGACTCAG |
| | 3′ primer | 3′ READ2- | 959 | 5′ GTGACTGGAGTTCAGACGTGTGCTCTTC |
| | | LR42 | | CGATCTGGGYGGGAACAGAGTGAC |

Barcoding-PCR primers

| | | | | |
|---|---|---|---|---|
| Heavy | 5′ primer | P5-S502- | 960 | AATGATACGGCGACCACCGAGATCTACACC |
| chain | (column) | READ1os | | TCTCTATACACTCTTTCCCTACACGACGC |
| | | P5-S503- | 961 | AATGATACGGCGACCACCGAGATCTACACT |
| | | READ1os | | ATCCTCTACACTCTTTCCCTACACGACGC |
| | | P5-S505- | 962 | AATGATACGGCGACCACCGAGATCTACACG |
| | | READ1os | | TAAGGAGACACTCTTTCCCTACACGACGC |
| | | P5-S506- | 963 | AATGATACGGCGACCACCGAGATCTACACA |
| | | READ1os | | CTGCATAACACTCTTTCCCTACACGACGC |
| | | P5-S507- | 964 | AATGATACGGCGACCACCGAGATCTACACA |
| | | READ1os | | AGGAGTAACACTCTTTCCCTACACGACGC |
| | | P5-S508- | 965 | AATGATACGGCGACCACCGAGATCTACACC |
| | | READ1os | | TAAGCCTACACTCTTTCCCTACACGACGC |
| | | P5-S510- | 966 | AATGATACGGCGACCACCGAGATCTACACC |
| | | READ1os | | GTCTAATACACTCTTTCCCTACACGACGC |
| | | P5-S511- | 967 | AATGATACGGCGACCACCGAGATCTACACT |
| | | READ1os | | CTCTCCGACACTCTTTCCCTACACGACGC |
| | 3′ primer | P7-N701- | 968 | CAAGCAGAAGACGGCATACGAGATTCGCC |
| | (Row) | READ2os | | TTAGTGACTGGAGTTCAGACGTGTGC |
| | | P7-N702- | 969 | CAAGCAGAAGACGGCATACGAGATCTAGT |
| | | READ2os | | ACGGTGACTGGAGTTCAGACGTGTGC |
| | | P7-N703- | 970 | CAAGCAGAAGACGGCATACGAGATTTCTGC |
| | | READ2os | | CTGTGACTGGAGTTCAGACGTGTGC |
| | | P7-N704- | 971 | CAAGCAGAAGACGGCATACGAGATGCTCA |
| | | READ2os | | GGAGTGACTGGAGTTCAGACGTGTGC |
| | | P7-N705- | 972 | CAAGCAGAAGACGGCATACGAGATAGGAG |
| | | READ2os | | TCCGTGACTGGAGTTCAGACGTGTGC |
| | | P7-N706- | 973 | CAAGCAGAAGACGGCATACGAGATCATGC |
| | | READ2os | | CTAGTGACTGGAGTTCAGACGTGTGC |
| | | P7-N707- | 974 | CAAGCAGAAGACGGCATACGAGATGTAGA |
| | | READ2os | | GAGGTGACTGGAGTTCAGACGTGTGC |
| | | P7-N710- | 975 | CAAGCAGAAGACGGCATACGAGATCAGCC |
| | | READ2os | | TCGGTGACTGGAGTTCAGACGTGTGC |
| | | P7-N711- | 976 | CAAGCAGAAGACGGCATACGAGATTGCCT |
| | | READ2os | | CTTGTGACTGGAGTTCAGACGTGTGC |
| | | P7-N712- | 977 | CAAGCAGAAGACGGCATACGAGATTCCTCT |
| | | READ2os | | ACGTGACTGGAGTTCAGACGTGTGC |
| | | P7-N714- | 978 | CAAGCAGAAGACGGCATACGAGATTCATG |
| | | READ2os | | AGCGTGACTGGAGTTCAGACGTGTGC |
| | | P7-N715- | 979 | CAAGCAGAAGACGGCATACGAGATCCTGA |
| | | READ2os | | GATGTGACTGGAGTTCAGACGTGTGC |
| Kappa | 5′ primer | P5-S513- | 980 | AATGATACGGCGACCACCGAGATCTACACT |
| chain | (column) | READ1os | | CGACTAGACACTCTTTCCCTACACGACGC |
| | | P5-S515- | 981 | AATGATACGGCGACCACCGAGATCTACACT |
| | | READ1os | | TCTAGCTACACTCTTTCCCTACACGACGC |
| | | P5-S516- | 982 | AATGATACGGCGACCACCGAGATCTACACC |
| | | READ1os | | CTAGAGTACACTCTTTCCCTACACGACGC |
| | | P5-S517- | 983 | AATGATACGGCGACCACCGAGATCTACACG |
| | | READ1os | | CGTAAGAACACTCTTTCCCTACACGACGC |
| | | P5-S518- | 984 | AATGATACGGCGACCACCGAGATCTACACC |
| | | READ1os | | TATTAAGACACTCTTTCCCTACACGACGC |
| | | P5-S520- | 985 | AATGATACGGCGACCACCGAGATCTACACA |
| | | READ1os | | AGGCTATACACTCTTTCCCTACACGACGC |
| | | P5-S521- | 986 | AATGATACGGCGACCACCGAGATCTACACG |
| | | READ1os | | AGCCTTAACACTCTTTCCCTACACGACGC |
| | | P5-S522- | 987 | AATGATACGGCGACCACCGAGATCTACACT |
| | | READ1os | | TATGCGAACACTCTTTCCCTACACGACGC |
| | 3′ primer | P7-N701- | 968 | CAAGCAGAAGACGGCATACGAGATTCGCC |
| | (Row) | READ2os | | TTAGTGACTGGAGTTCAGACGTGTGC |
| | | P7-N702- | 969 | CAAGCAGAAGACGGCATACGAGATCTAGT |
| | | READ2os | | ACGGTGACTGGAGTTCAGACGTGTGC |
| | | P7-N703- | 970 | CAAGCAGAAGACGGCATACGAGATTTCTGC |
| | | READ2os | | CTGTGACTGGAGTTCAGACGTGTGC |
| | | P7-N704- | 971 | CAAGCAGAAGACGGCATACGAGATGCTCA |
| | | READ2os | | GGAGTGACTGGAGTTCAGACGTGTGC |
| | | P7-N705- | 972 | CAAGCAGAAGACGGCATACGAGATAGGAG |
| | | READ2os | | TCCGTGACTGGAGTTCAGACGTGTGC |
| | | P7-N706- | 973 | CAAGCAGAAGACGGCATACGAGATCATGC |
| | | READ2os | | CTAGTGACTGGAGTTCAGACGTGTGC |
| | | P7-N707- | 974 | CAAGCAGAAGACGGCATACGAGATGTAGA |
| | | READ2os | | GAGGTGACTGGAGTTCAGACGTGTGC |
| | | P7-N710- | 975 | CAAGCAGAAGACGGCATACGAGATCAGCC |
| | | READ2os | | TCGGTGACTGGAGTTCAGACGTGTGC |
| | | P7-N711- | 976 | CAAGCAGAAGACGGCATACGAGATTGCCT |
| | | READ2os | | CTTGTGACTGGAGTTCAGACGTGTGC |
| | | P7-N712- | 977 | CAAGCAGAAGACGGCATACGAGATTCCTCT |
| | | READ2os | | ACGTGACTGGAGTTCAGACGTGTGC |
| | | P7-N714- | 978 | CAAGCAGAAGACGGCATACGAGATTCATG |
| | | READ2os | | AGCGTGACTGGAGTTCAGACGTGTGC |
| | | P7-N715- | 979 | CAAGCAGAAGACGGCATACGAGATCCTGA |
| | | READ2os | | GATGTGACTGGAGTTCAGACGTGTGC |
| Lambda | 5′ primer | P5-S502- | 960 | AATGATACGGCGACCACCGAGATCTACACC |
| chain | (column) | READ1os | | TCTCTATACACTCTTTCCCTACACGACGC |
| | | P5-S503- | 961 | AATGATACGGCGACCACCGAGATCTACACT |
| | | READ1os | | ATCCTCTACACTCTTTCCCTACACGACGC |
| | | P5-S505- | 962 | AATGATACGGCGACCACCGAGATCTACACG |
| | | READ1os | | TAAGGAGACACTCTTTCCCTACACGACGC |
| | | P5-S506- | 963 | AATGATACGGCGACCACCGAGATCTACACA |
| | | READ1os | | CTGCATAACACTCTTTCCCTACACGACGC |
| | | P5-S507- | 964 | AATGATACGGCGACCACCGAGATCTACACA |
| | | READ1os | | AGGAGTAACACTCTTTCCCTACACGACGC |
| | | P5-S508- | 965 | AATGATACGGCGACCACCGAGATCTACACC |
| | | READ1os | | TAAGCCTACACTCTTTCCCTACACGACGC |
| | | P5-S510- | 966 | AATGATACGGCGACCACCGAGATCTACACC |
| | | READ1os | | GTCTAATACACTCTTTCCCTACACGACGC |
| | | P5-S511- | 967 | AATGATACGGCGACCACCGAGATCTACACT |
| | | READ1os | | CTCTCCGACACTCTTTCCCTACACGACGC |
| | 3′ primer | P7-N716- | 988 | CAAGCAGAAGACGGCATACGAGATTAGCG |
| | (Row) | READ2os | | AGTGTGACTGGAGTTCAGACGTGTGC |
| | | P7-N718- | 989 | CAAGCAGAAGACGGCATACGAGATGTAGC |
| | | READ2os | | TCCGTGACTGGAGTTCAGACGTGTGC |
| | | P7-N719- | 990 | CAAGCAGAAGACGGCATACGAGATTACTA |
| | | READ2os | | CGCGTGACTGGAGTTCAGACGTGTGC |
| | | P7-N720- | 991 | CAAGCAGAAGACGGCATACGAGATAGGCT |
| | | READ2os | | CCGGTGACTGGAGTTCAGACGTGTGC |
| | | P7-N721- | 992 | CAAGCAGAAGACGGCATACGAGATGCAGC |
| | | READ2os | | GTAGTGACTGGAGTTCAGACGTGTGC |
| | | P7-N722- | 993 | CAAGCAGAAGACGGCATACGAGATCTGCG |
| | | READ2os | | CATGTGACTGGAGTTCAGACGTGTGC |
| | | P7-N723- | 994 | CAAGCAGAAGACGGCATACGAGATGAGCG |
| | | READ2os | | CTAGTGACTGGAGTTCAGACGTGTGC |
| | | P7-N724- | 995 | CAAGCAGAAGACGGCATACGAGATCGCTC |
| | | READ2os | | AGTGTGACTGGAGTTCAGACGTGTGC |
| | | P7-N726- | 996 | CAAGCAGAAGACGGCATACGAGATGTCTT |
| | | READ2os | | AGGGTGACTGGAGTTCAGACGTGTGC |
| | | P7-N727- | 997 | CAAGCAGAAGACGGCATACGAGATACTGA |
| | | READ2os | | TCGGTGACTGGAGTTCAGACGTGTGC |
| | | P7-N728- | 998 | CAAGCAGAAGACGGCATACGAGATTAGCT |
| | | READ2os | | GCAGTGACTGGAGTTCAGACGTGTGC |
| | | P7-N729- | 999 | CAAGCAGAAGACGGCATACGAGATGACGT |
| | | READ2os | | CGAGTGACTGGAGTTCAGACGTGTGC |

K = G/T, M = A/C, R = A/G, S = G/C, W = A/T, Y = C/T, H = A/C /T. A “/” indicates the different options. For example, “K” can be G or T.

TABLE 3A
Mab sorting, cloning and sequence method, and IC50 data

| | | | Cloning and | PRNT IC50 (ng/mL) | | | |
|---|---|---|---|---|---|---|---|
| mAb | | Antigen used | sequencing | RSV A/ | RSV B/ | | |
| name | Sites | for sorting | method | Long | Washington | hMPV A | hMPV B |
| M1C7s | II | hMPV WT | Regular | N.D. | N.D. | 15.0 | 9.2 |
| | | monomer | cloning | | | | |
| M1D2 | II | hMPV WT | Regular | N.D. | N.D. | 11.7 | 12.4 |
| | | trimer | cloning | | | | |
| M2A09 | II | hMPV WT | Barcode NGS | N.D. | N.D. | 13.3 | 45.6 |
| | | trimer | | | | | |
| M2C06 | II | hMPV WT | Barcode NGS | N.D. | N.D. | 20.4 | 50.2 |
| | | trimer | | | | | |
| M3C04 | II | hMPV WT | Barcode NGS | N.D. | N.D. | 3870.0 | 86.2 |
| | | trimer | | | | | |
| M3E03 | II | hMPV WT | Barcode NGS | N.D. | N.D. | 19.9 | 76.2 |
| | | trimer | | | | | |
| M4A02 | II | hMPV WT | Barcode NGS | 23.58 | 36.39 | 186.5 | 92.7 |
| | | trimer | | | | | |
| M6E10 | II | hMPV WT | Regular | N.D. | N.D. | 23.7 | 10.4 |
| | | trimer | cloning | | | | |
| M7D5 | II | hMPV WT | Regular | N.D. | N.D. | 35.4 | 35.9 |
| | | trimer | cloning | | | | |
| M2B6 | III | hMPV WT | Regular | 9 | 9.2 | 44.0 | 23.7 |
| | | monomer | cloning | | | | |
| M3A01 | III | hMPV WT | Barcode NGS | 13.17 | 34.38 | 608.9 | 120.1 |
| | | trimer | | | | | |
| M3A06 | III | hMPV WT | Barcode NGS | 7.972 | 27.41 | 310.5 | 416.4 |
| | | trimer | | | | | |
| M3A08 | III | hMPV WT | Barcode NGS | 6.292 | 29 | 251.5 | 923.1 |
| | | trimer | | | | | |
| M3B02 | III | hMPV WT | Barcode NGS | 4.393 | 15.32 | 1061.0 | 1545.0 |
| | | trimer | | | | | |
| M3E04 | III | hMPV WT | Barcode NGS | 7.719 | 18.38 | 3622.0 | 2248.0 |
| | | trimer | | | | | |
| M3E10 | III | hMPV WT | Barcode NGS | N.D. | N.D. | >10000 | >10000 |
| | | trimer | | | | | |
| M4A05 | III | hMPV WT | Barcode NGS | 8.371 | 29.46 | 56.9 | 42.5 |
| | | trimer | | | | | |
| M2B05 | III | hMPV WT | Barcode NGS | N.D. | N.D. | 7.8 | 53.2 |
| | | trimer | | | | | |
| M2B06 | III′ | hMPV WT | Barcode NGS | N.D. | N.D. | 23.0 | 20.6 |
| | | trimer | | | | | |
| M2C07 | III′ | hMPV WT | Barcode NGS | N.D. | N.D. | 61.6 | 117.5 |
| | | trimer | | | | | |
| M2D03 | III′ | hMPV WT | Barcode NGS | N.D. | N.D. | >10000 | 38.2 |
| | | trimer | | | | | |
| M2D11 | III′ | hMPV WT | Barcode NGS | N.D. | N.D. | 11.7 | 56.0 |
| | | trimer | | | | | |
| M3C11 | III′ | hMPV WT | Barcode NGS | N.D. | N.D. | >10000 | 287.8 |
| | | trimer | | | | | |
| M3D05 | III′ | hMPV WT | Barcode NGS | N.D. | N.D. | 762.8 | 237.5 |
| | | trimer | | | | | |
| M3D10 | III′ | hMPV WT | Barcode NGS | N.D. | N.D. | 56.4 | 49.9 |
| | | trimer | | | | | |
| M4A06 | III′ | hMPV WT | Barcode NGS | N.D. | N.D. | 366.8 | 156.2 |
| | | trimer | | | | | |
| M4A12 | III′ | hMPV WT | Barcode NGS | N.D. | N.D. | >10000 | 74.4 |
| | | trimer | | | | | |
| M4C02 | III′ | hMPV WT | Barcode NGS | N.D. | N.D. | 577.1 | 172.7 |
| | | trimer | | | | | |
| M1A04 | IV | hMPV WT | Barcode NGS | N.D. | N.D. | 87.2 | 128.4 |
| | | trimer + RSV | | | | | |
| | | DS-Cav1 | | | | | |
| M1C11 | IV | hMPV WT | Regular | N.D. | N.D. | 5925.0 | 443.6 |
| | | trimer | cloning | | | | |
| M2A11 | IV | hMPV WT | Barcode NGS | N.D. | N.D. | 2160.0 | 1575.0 |
| | | trimer | | | | | |
| M2B01 | IV | hMPV WT | Barcode NGS | N.D. | N.D. | 1000.0 | 768.2 |
| | | trimer | | | | | |
| M2B12 | IV | hMPV WT | Barcode NGS | N.D. | N.D. | 109.6 | 129.7 |
| | | trimer | | | | | |
| M2C05 | IV | hMPV WT | Barcode NGS | N.D. | N.D. | 15.2 | 51.3 |
| | | trimer | | | | | |
| M2D01 | IV | hMPV WT | Barcode NGS | N.D. | N.D. | 2240.0 | 4411.0 |
| | | trimer | | | | | |
| M2D2 | IV | hMPV WT | Regular | 9.6 | 3.9 | 29.6 | 58.4 |
| | | trimer | cloning | | | | |
| M3C05 | IV | hMPV WT | Barcode NGS | N.D. | N.D. | 18.4 | 7.3 |
| | | trimer | | | | | |
| M3C12 | IV | hMPV WT | Barcode NGS | N.D. | N.D. | 7.0 | 1.0 |
| | | trimer | | | | | |
| M3D09 | IV | hMPV WT | Barcode NGS | N.D. | N.D. | 1998.0 | 280.0 |
| | | trimer | | | | | |
| M3D12 | IV | hMPV WT | Barcode NGS | N.D. | N.D. | 23.1 | 14.5 |
| | | trimer | | | | | |
| M3F03 | IV | hMPV WT | Barcode NGS | N.D. | N.D. | 231.5 | 71.7 |
| | | trimer | | | | | |
| M4B01 | IV | hMPV WT | Barcode NGS | N.D. | N.D. | 37.1 | 62.8 |
| | | trimer | | | | | |
| M4C03 | IV | hMPV WT | Barcode NGS | 5074 | >10000 | 2020.0 | 526.3 |
| | | trimer | | | | | |
| M4C04 | IV | hMPV WT | Barcode NGS | N.D. | N.D. | 77.8 | 48.0 |
| | | trimer | | | | | |
| M4C08 | IV | hMPV WT | Barcode NGS | 5584 | 3274 | 3116.0 | 834.6 |
| | | trimer | | | | | |
| M2A02 | IV′ | hMPV WT | Barcode NGS | N.D. | N.D. | 628.1 | 351.7 |
| | | trimer | | | | | |
| M2A05 | IV′ | hMPV WT | Barcode NGS | N.D. | N.D. | >10000 | 65.7 |
| | | trimer | | | | | |
| M2A06 | IV′ | hMPV WT | Barcode NGS | N.D. | N.D. | 402.0 | 2500.0 |
| | | trimer | | | | | |
| M2B10 | IV′ | hMPV WT | Barcode NGS | N.D. | N.D. | 3505.0 | 2280.0 |
| | | trimer | | | | | |
| M2D07 | IV′ | hMPV WT | Barcode NGS | N.D. | N.D. | 1066.0 | 2500.0 |
| | | trimer | | | | | |
| M2D10 | IV′ | hMPV WT | Barcode NGS | N.D. | N.D. | 758.6 | 1343.0 |
| | | trimer | | | | | |
| M2F01 | IV′ | hMPV WT | Barcode NGS | N.D. | N.D. | >10000 | >10000 |
| | | trimer | | | | | |
| M4B04 | IV′ | hMPV WT | Barcode NGS | N.D. | N.D. | 4592.0 | 1971.0 |
| | | trimer | | | | | |
| M4B05 | IV′ | hMPV WT | Barcode NGS | N.D. | N.D. | 1380.0 | 479.2 |
| | | trimer | | | | | |
| M4B11 | IV′ | hMPV WT | Barcode NGS | N.D. | N.D. | 652.3 | 237.9 |
| | | trimer | | | | | |
| M4F8 | IV′ | hMPV WT | Regular | N.D. | N.D. | 2442.0 | 2211.0 |
| | | trimer | cloning | | | | |
| M2A03 | IV′ | hMPV WT | Barcode NGS | N.D. | N.D. | 1422.0 | 145.1 |
| | | trimer | | | | | |
| M3A07 | IV′ | hMPV WT | Barcode NGS | N.D. | N.D. | 936.0 | 1724.0 |
| | | trimer | | | | | |
| M5F2 | IV′ | hMPV WT | Regular | N.D. | N.D. | >10000 | >10000 |
| | | trimer | cloning | | | | |
| M4B03 | V | hMPV WT | Barcode NGS | N.D. | N.D. | 34.2 | 670.6 |
| | | trimer | | | | | |
| M4B06 | V | hMPV WT | Barcode NGS | N.D. | N.D. | 3.5 | 2.2 |
| | | trimer | | | | | |
| M2C08 | V | hMPV WT | Barcode NGS | N.D. | N.D. | 329.3 | 1554.0 |
| | | trimer | | | | | |
| M3D04 | V | hMPV WT | Barcode NGS | N.D. | N.D. | 200.2 | 326.6 |
| | | trimer | | | | | |
| M1C7 | V | hMPV WT | Regular | 22.5 | 1105 | 8.3 | 8.5 |
| | | trimer | cloning | | | | |
| M2B11 | V | hMPV WT | Barcode NGS | N.D. | N.D. | 62.7 | 8.8 |
| | | trimer | | | | | |
| M2C11 | V | hMPV WT | Barcode NGS | N.D. | N.D. | 72.0 | 58.8 |
| | | trimer | | | | | |
| M2D08 | V | hMPV WT | Barcode NGS | 10.72 | 14.47 | 2400.0 | 2500.0 |
| | | trimer | | | | | |
| M3C06 | V | hMPV WT | Barcode NGS | N.D. | N.D. | >10000 | 61.7 |
| | | trimer | | | | | |
| M4A10 | V | hMPV WT | Barcode NGS | N.D. | N.D. | >10000 | 30.1 |
| | | trimer | | | | | |
| M1A08 | β | hMPV WT | Barcode NGS | N.D. | N.D. | 2274.0 | 719.5 |
| | | trimer + RSV | | | | | |
| | | DS-Cav1 | | | | | |
| M2B07 | β | hMPV WT | Barcode NGS | N.D. | N.D. | 625.0 | 426.0 |
| | | trimer | | | | | |
| M2B09 | β | hMPV WT | Barcode NGS | N.D. | N.D. | 1000-2500 | 2010.0 |
| | | trimer | | | | | |
| M2E04 | β | hMPV WT | Barcode NGS | N.D. | N.D. | 6970.0 | 23.1 |
| | | trimer | | | | | |
| M2E05 | β | hMPV WT | Barcode NGS | N.D. | N.D. | 20.1 | 51.7 |
| | | trimer | | | | | |
| M2E06 | β | hMPV WT | Barcode NGS | N.D. | N.D. | 104.8 | 139.1 |
| | | trimer | | | | | |
| M4A08 | β | hMPV WT | Barcode NGS | N.D. | N.D. | 135.8 | 343.7 |
| | | trimer | | | | | |
| M2E11L | β | hMPV WT | Barcode NGS | N.D. | N.D. | 625.0 | 625.0 |
| | | trimer | | | | | |
| M1A11 | α | hMPV WT | Barcode NGS | N.D. | N.D. | 2278.5 | 355.7 |
| | | trimer + RSV | | | | | |
| | | DS-Cav1 | | | | | |
| M2C10 | α | hMPV WT | Barcode NGS | N.D. | N.D. | 547.2 | 2500.0 |
| | | trimer | | | | | |
| M2E01 | α | hMPV WT | Barcode NGS | N.D. | N.D. | 463.2 | 591.3 |
| | | trimer | | | | | |
| M2E08 | α | hMPV WT | Barcode NGS | N.D. | N.D. | 625.0 | 2500.0 |
| | | trimer | | | | | |
| M3A04 | α | hMPV WT | Barcode NGS | N.D. | N.D. | 1148.0 | 1435.0 |
| | | trimer | | | | | |
| M3A12 | α | hMPV WT | Barcode NGS | N.D. | N.D. | 332.1 | 234.0 |
| | | trimer | | | | | |
| M3C07 | α | hMPV WT | Barcode NGS | N.D. | N.D. | >10000 | >10000 |
| | | trimer | | | | | |
| M3D03 | α | hMPV WT | Barcode NGS | N.D. | N.D. | >10000 | >10000 |
| | | trimer | | | | | |
| M8C10 | α | hMPV WT | Regular | N.D. | N.D. | 370.0 | 660.5 |
| | | trimer | cloning | | | | |
| M3A03 | UC | hMPV WT | Barcode NGS | N.D. | N.D. | >10000 | >10000 |
| | | trimer | | | | | |
| M3A05 | UC | hMPV WT | Barcode NGS | N.D. | N.D. | >10000 | >10000 |
| | | trimer | | | | | |
| M3A09 | UC | hMPV WT | Barcode NGS | N.D. | N.D. | >10000 | >10000 |
| | | trimer | | | | | |
| M3A11 | UC | hMPV WT | Barcode NGS | N.D. | N.D. | >10000 | >10000 |
| | | trimer | | | | | |
| M3B04 | UC | hMPV WT | Barcode NGS | N.D. | N.D. | >10000 | >10000 |
| | | trimer | | | | | |
| M3B06 | UC | hMPV WT | Barcode NGS | N.D. | N.D. | >10000 | >10000 |
| | | trimer | | | | | |
| M3B07 | UC | hMPV WT | Barcode NGS | N.D. | N.D. | >10000 | >10000 |
| | | trimer | | | | | |
| M3B12 | UC | hMPV WT | Barcode NGS | N.D. | N.D. | >10000 | >10000 |
| | | trimer | | | | | |
| M3C01 | UC | hMPV WT | Barcode NGS | N.D. | N.D. | >10000 | >10000 |
| | | trimer | | | | | |
| M3D06 | UC | hMPV WT | Barcode NGS | N.D. | N.D. | >10000 | >10000 |
| | | trimer | | | | | |
| M3D08 | UC | hMPV WT | Barcode NGS | N.D. | N.D. | >10000 | >10000 |
| | | trimer | | | | | |
| M3E02 | UC | hMPV WT | Barcode NGS | N.D. | N.D. | >10000 | >10000 |
| | | trimer | | | | | |
| M3E05 | UC | hMPV WT | Barcode NGS | N.D. | N.D. | >10000 | >10000 |
| | | trimer | | | | | |
| M3E06 | UC | hMPV WT | Barcode NGS | N.D. | N.D. | >10000 | >10000 |
| | | trimer | | | | | |
| M3E08 | UC | hMPV WT | Barcode NGS | N.D. | N.D. | >10000 | >10000 |
| | | trimer | | | | | |
| M1A01 | UC | hMPV WT | Barcode NGS | N.D. | N.D. | 3405.5 | 3997.0 |
| | | trimer + RSV | | | | | |
| | | DS-Cav1 | | | | | |
| M1A12 | UC | hMPV WT | Barcode NGS | N.D. | N.D. | 1508.5 | 5000.0 |
| | | trimer + RSV | | | | | |
| | | DS-Cav1 | | | | | |
| M2B04 | UC | hMPV WT | Barcode NGS | N.D. | N.D. | 1111.0 | 1002.0 |
| | | trimer | | | | | |
| M2C01 | UC | hMPV WT | Barcode NGS | N.D. | N.D. | >10000 | >10000 |
| | | trimer | | | | | |
| M2D02 | UC | hMPV WT | Barcode NGS | N.D. | N.D. | >10000 | 10000.0 |
| | | trimer | | | | | |
| M2D09 | UC | hMPV WT | Barcode NGS | N.D. | N.D. | >10000 | >10000 |
| | | trimer | | | | | |
| M2E07 | UC | hMPV WT | Barcode NGS | N.D. | N.D. | 2500.0 | 2500.0 |
| | | trimer | | | | | |
| M3E01 | UC | hMPV WT | Barcode NGS | N.D. | N.D. | >10000 | >10000 |
| | | trimer | | | | | |
| M3E09 | UC | hMPV WT | Barcode NGS | N.D. | N.D. | >10000 | >10000 |
| | | trimer | | | | | |
| M3F02 | UC | hMPV WT | Barcode NGS | N.D. | N.D. | >10000 | >10000 |
| | | trimer | | | | | |
| M4A11 | UC | hMPV WT | Barcode NGS | N.D. | N.D. | >10000 | >10000 |
| | | trimer | | | | | |
| M4B02 | UC | hMPV WT | Barcode NGS | N.D. | N.D. | >10000 | >10000 |
| | | trimer | | | | | |

N.D.—not detectable; UC—uncharacterized.

TABLE 3B
VH information of the mAbs

| | VH | | | | | | | | | |
|---|---|---|---|---|---|---|---|---|---|---|
| | | | | CDR1 | CDR2 | CDR3 | | | | |
| mAb | | | | se- | se- | se- | CDR | nt | nt | aa |
| name | Germline | | | quence | quence | quence | lengths | sub. | ID% | ID% |
| MIC7s | IGHV1- | IGHD2- | IGHJ3*02 | GGTFT | IIPAV | ATVLD | 8, 8, 12 | 34 | 88.435 | 78.4 |
| | 69*09 | 8*02 | | RLT | DIT | WSNAL | | | | |
| | | | | (SEQ ID | (SEQ ID | DI | | | | |
| | | | | NO: 2) | NO: 3) | (SEQ ID | | | | |
| | | | | | | NO: 4) | | | | |
| MID2 | IGHV4- | | IGHJ6*03 | GGSVN | LNHIG | ARRPP | 9, 7, 18 | 27 | 90.785 | 78.6 |
| | 4*02 | | | SDHW | TF | PLPEY | | | | |
| | | | | (SEQ ID | (SEQ ID | FYYYF | | | | |
| | | | | NO: 5) | NO: 6) | MDV | | | | |
| | | | | | | (SEQ ID | | | | |
| | | | | | | NO: 7) | | | | |
| M2A09 | IGHV1- | IGHD3- | IGHJ4*02 | GGTFS | IIPIV | ATEGL | 8, 8, 15 | 35 | 88.176 | 81.4 |
| | 69*04 | 22*01 | | NYI | GME | ESGYP | | | | |
| | | | | (SEQ ID | (SEQ ID | PYLE | | | | |
| | | | | NO: 8) | NO: 9) | H | | | | |
| | | | | | | (SEQ ID | | | | |
| | | | | | | NO: 10) | | | | |
| M2C06 | IGHV1- | IGHD3- | IGHJ3*02 | GGTFS | IIPLV | VRDLP | 8, 8, 15 | 46 | 84.459 | 74.5 |
| | 69*09 | 9*01 | | NDI | NMT | SAGFP | | | | |
| | | | | (SEQ ID | (SEQ ID | APF | | | | |
| | | | | NO: 11) | NO: 12) | DI | | | | |
| | | | | | | (SEQ ID | | | | |
| | | | | | | NO: 13) | | | | |
| M3C04 | IGHV5- | IGHD3- | IGHJ3*02 | GYNFA | IYPGD | ARPLA | 8, 8, 11 | 11 | 96.246 | 90.8 |
| | 51*01 | 16*02 | | NYW | SDT | VSAFD | | | | |
| | | | | (SEQ ID | (SEQ ID | I | | | | |
| | | | | NO: 14) | NO: 15) | (SEQ ID | | | | |
| | | | | | | NO: 16) | | | | |
| M3E03 | IGHV3- | IGHD3- | IGHJ4*02 | GFPFD | VWYDG | ARGMI | 8, 8, 9 | 25 | 91.468 | 83.7 |
| | 33*01 | 16*01 | | SFA | GDK | TFDY | | | | |
| | | | | (SEQ ID | (SEQ ID | (SEQ ID | | | | |
| | | | | NO: 17) | NO: 18) | NO: 19) | | | | |
| M4A02 | IGHV3- | IGHD1- | IGHJ5*02 | GFMFT | ITGGG | AREDY | 8, 8, 16 | 36 | 87.838 | 77.8 |
| | 21*01 | 14*01 | | SYA | NFV | NSGAN | | | | |
| | | | | (SEQ ID | (SEQ ID | LHWFD | | | | |
| | | | | NO: 20) | NO: 21) | P | | | | |
| | | | | | | (SEQ ID | | | | |
| | | | | | | NO: 22) | | | | |
| M6E10 | IGHV1- | IGHD2- | IGHJ6*02 | GGIFD | IIPPF | ARDRT | 8, 8, 15 | 30 | 89.865 | 81.6 |
| | 69*01 | 21*01 | | TNV | GTA | VIKYA | | | | |
| | | | | (SEQ ID | (SEQ ID | DGMD | | | | |
| | | | | NO: 23) | NO: 24) | V | | | | |
| | | | | | | (SEQ ID | | | | |
| | | | | | | NO: 25) | | | | |
| M7D5 | IGHV1- | IGHD4- | IGHJ6*02 | GGTLS | IIPIF | AQDRT | 8, 8, 15 | 27 | 90.878 | 84.7 |
| | 69*01 | 17*01 | | NYA | GTA | VTKYA | | | | |
| | | | | (SEQ ID | (SEQ ID | HGLD | | | | |
| | | | | NO: 26) | NO: 27) | V | | | | |
| | | | | | | (SEQ ID | | | | |
| | | | | | | NO: 28) | | | | |
| M2B6 | IGHV4- | IGHD4- | IGHJ5*01 | GASIG | IYYSG | ASLTI | 8, 7, 12 | 35 | 87.931 | 77.1 |
| | 59*01 | 23*01 | | GYY | RT | TPGWF | | | | |
| | | | | (SEQ ID | (SEQ ID | DS | | | | |
| | | | | NO: 29) | NO: 30) | (SEQ ID | | | | |
| | | | | | | NO: 31) | | | | |
| M3A01 | IGHV3- | IGHD3- | IGHJ5*01 | AFSIS | ITGGS | ARETQ | 8, 8, 13 | 27 | 90.878 | 84.7 |
| | 21*01 | 10*01 | | GYN | NYI | PSPGS | | | | |
| | | | | (SEQ ID | (SEQ ID | FDS | | | | |
| | | | | NO: 32) | NO: 33) | (SEQ ID | | | | |
| | | | | | | NO: 34) | | | | |
| M3A06 | IGHV3- | IGHD3- | IGHJ3*02 | GFTLT | ISSSS | ARVAR | 8, 8, 12 | 11 | 96.271 | 94.9 |
| | 21*01 | 10*01 | | SYS | SYI | ELDPF | | | | |
| | | | | (SEQ ID | (SEQ ID | DM | | | | |
| | | | | NO: 35) | NO: 36) | (SEQ ID | | | | |
| | | | | | | NO: 37) | | | | |
| M3A08 | IGHV3- | IGHD2- | IGHJ5*01 | GFSIT | ISSSS | ARDAG | 8, 8, 13 | 29 | 90.203 | 87.8 |
| | 21*01 | 8*02 | | SYG | SYI | LVPPW | | | | |
| | | | | (SEQ ID | (SEQ ID | MDS | | | | |
| | | | | NO: 38) | NO: 39) | (SEQ ID | | | | |
| | | | | | | NO: 40) | | | | |
| M3B02 | IGHV3- | IGHD2- | IGHJ2*01 | EFTLS | ISSSS | VRERP | 8, 8, 14 | 24 | 91.892 | 84.7 |
| | 21*01 | 2*02 | | GYA | SYI | TAILP | | | | |
| | | | | (SEQ ID | (SEQ ID | YLDL | | | | |
| | | | | NO: 41) | NO: 42) | (SEQ ID | | | | |
| | | | | | | NO: 43) | | | | |
| M3E04 | IGHV3- | IGHD3- | IGHJ5*02 | GFTFS | ISASS | ARANY | 8, 8, 18 | 5 | 98.305 | 96.9 |
| | 21*01 | 10*01 | | SYS | SYI | GSGNY | | | | |
| | | | | (SEQ ID | (SEQ ID | YNPKW | | | | |
| | | | | NO: 44) | NO: 45) | FDP | | | | |
| | | | | | | (SEQ ID | | | | |
| | | | | | | NO: 46) | | | | |
| M3E10 | IGHV1- | IGHD2- | IGHJ6*02 | GGTIN | IIPIF | ARGTR | 8, 8, 24 | 16 | 94.576 | 89.8 |
| | 69*01 | 15*01 | | SYA | GTA | YIAVM | | | | |
| | | | | (SEQ ID | (SEQ ID | VPSMQ | | | | |
| | | | | NO: 47) | NO: 48) | DYYFF | | | | |
| | | | | | | GMDV | | | | |
| | | | | | | (SEQ ID | | | | |
| | | | | | | NO: 49) | | | | |
| M4A05 | IGHV3- | | IGHJ4*02 | GFSLS | ITASS | ATNGK | 8, 8, 12 | 35 | 88.014 | 82.5 |
| | 21*01 | | | GYG | SYI | LPPYF | | | | |
| | | | | (SEQ ID | (SEQ ID | DH | | | | |
| | | | | NO: 50) | NO: 51) | (SEQ ID | | | | |
| | | | | | | NO: 52) | | | | |
| M2B05 | IGHV4- | IGHD3- | IGHJ5*02 | GGSFG | IDHGG | ARGFG | 8, 7, 14 | 13 | 95.563 | 87.6 |
| | 34*01 | 3*01 | | GYH | NT | VVINN | | | | |
| | | | | (SEQ ID | (SEQ ID | WFDP | | | | |
| | | | | NO: 53) | NO: 54) | (SEQ ID | | | | |
| | | | | | | NO: 55) | | | | |
| M2B06 | IGHV4- | IGHD2- | IGHJ5*02 | GASIT | IFHSG | ARDDQ | 9, 7, 15 | 19 | 93.581 | 88.8 |
| | 4*02 | 2*01 | | SSHW | ST | IVVMP | | | | |
| | | | | (SEQ ID | (SEQ ID | AGFD | | | | |
| | | | | NO: 56) | NO: 57) | R | | | | |
| | | | | | | (SEQ ID | | | | |
| | | | | | | NO: 58) | | | | |
| M2C07 | IGHV4- | IGHD5- | IGHJ4*02 | GDSVT | INYSG | ARANV | 10, 7, 15 | 27 | 90.604 | 81.8 |
| | 31*03 | 12*01 | | SGAY | RT | VGYSG | | | | |
| | | | | S | (SEQ ID | FYFD | | | | |
| | | | | (SEQ ID | NO: 60) | Y | | | | |
| | | | | NO: 59) | | (SEQ ID | | | | |
| | | | | | | NO: 61) | | | | |
| M2D03 | IGHV4- | IGHD6- | IGHJ4*02 | GDSVT | INYSG | ARANV | 10, 7, 15 | 28 | 90.268 | 80.8 |
| | 31*03 | 19*01 | | SGAH | RT | VGYSG | | | | |
| | | | | S | (SEQ ID | YYFD | | | | |
| | | | | (SEQ ID | NO: 63) | Y | | | | |
| | | | | NO: 62) | | (SEQ ID | | | | |
| | | | | | | NO: 64) | | | | |
| M2D11 | IGHV4- | IGHD3- | IGHJ5*02 | GGSFS | VDHGG | ARGFG | 8, 7, 14 | 19 | 93.515 | 85.6 |
| | 34*01 | 3*01 | | GYH | NT | VVINN | | | | |
| | | | | (SEQ ID | (SEQ ID | WFDP | | | | |
| | | | | NO: 65) | NO: 66) | (SEQ ID | | | | |
| | | | | | | NO: 67) | | | | |
| M3C11 | IGHV4- | IGHD3- | IGHJ4*02 | GGSLS | IYYSG | ARGEA | 8, 7, 15 | 8 | 97.26 | 95.9 |
| | 59*01 | 3*01 | | SYC | GT | SGYLN | | | | |
| | | | | (SEQ ID | (SEQ ID | YYFD | | | | |
| | | | | NO: 68) | NO: 69) | Y | | | | |
| | | | | | | (SEQ ID | | | | |
| | | | | | | NO: 70) | | | | |
| M3D05 | IGHV1- | IGHD1- | IGHJ6*02 | GYTFS | INPNS | VRGQQ | 8, 8, 16 | 16 | 94.576 | 89.8 |
| | 2*02 | 1*01 | | GYY | GGT | LTRQY | | | | |
| | | | | (SEQ ID | (SEQ ID | YFGMD | | | | |
| | | | | NO: 71) | NO: 72) | V | | | | |
| | | | | | | (SEQ ID | | | | |
| | | | | | | NO: 73) | | | | |
| M3D10 | IGHV4- | IGHD2- | IGHJ4*02 | GGSVS | IYHNG | ATVSF | 8, 7, 15 | 18 | 93.836 | 89.6 |
| | 59*02 | 21*01 | | TYY | NI | IGYSL | | | | |
| | | | | (SEQ ID | (SEQ ID | YYFD | | | | |
| | | | | NO: 74) | NO: 75) | R | | | | |
| | | | | | | (SEQ ID | | | | |
| | | | | | | NO: 76) | | | | |
| M4A06 | IGHV3- | IGHD3- | IGHJ3*01 | GFSLT | ISASG | ARVGR | 8, 8, 20 | 23 | 92.15 | 85.7 |
| | 48*03 | 10*01 | | TDE | HTI | LGGVG | | | | |
| | | | | (SEQ ID | (SEQ ID | YYYAL | | | | |
| | | | | NO: 77) | NO: 78) | GPFDL | | | | |
| | | | | | | (SEQ ID | | | | |
| | | | | | | NO: 79) | | | | |
| M4A12 | IGHV4- | IGHD4- | IGHJ3*01 | GDSVT | MYYSG | GEATL | 10, 7, 13 | 35 | 88.255 | 81.8 |
| | 39*01 | 17*01 | | RNAY | ST | TTYDA | | | | |
| | | | | Y | (SEQ ID | FDV | | | | |
| | | | | (SEQ ID | NO: 81) | (SEQ ID | | | | |
| | | | | NO: 80) | | NO: 82) | | | | |
| M4C02 | IGHV4- | IGHD4- | IGHJ5*02 | GGSIS | IYYGG | ARVGS | 10, 7, 15 | 25 | 91.611 | 88.9 |
| | 39*02 | 11*01 | | SSSL | SS | PVTTT | | | | |
| | | | | Y | (SEQ ID | VWFD | | | | |
| | | | | (SEQ ID | NO: 84) | P | | | | |
| | | | | NO: 83) | | (SEQ ID | | | | |
| | | | | | | NO: 85) | | | | |
| M1A04 | IGHV3- | IGHD5- | IGHJ6*02 | GFNVR | IYGGG | ARDPG | 8, 7, 18 | 20 | 93.127 | 85.6 |
| | 66*02 | 24*01 | | NYY | TT | TVMST | | | | |
| | | | | (SEQ ID | (SEQ ID | ENLQG | | | | |
| | | | | NO: 86) | NO: 87) | MDV | | | | |
| | | | | | | (SEQ ID | | | | |
| | | | | | | NO: 88) | | | | |
| MIC11 | IGHV1- | IGHD3- | IGHJ3*01 | GGSFS | IIPLS | ARSGA | 8, 8, 22 | 26 | 91.156 | 81.6 |
| | 69*01 | 22*01 | | SYP | GTT | PTYYY | | | | |
| | | | | (SEQ ID | (SEQ ID | DSSGF | | | | |
| | | | | NO: 89) | NO: 90) | HGGAF | | | | |
| | | | | | | HF | | | | |
| | | | | | | (SEQ ID | | | | |
| | | | | | | NO: 91) | | | | |
| M2A11 | IGHV4- | IGHD2- | IGHJ6*02 | GGSIT | IYYSG | ARCMP | 8, 7, 12 | 17 | 94.138 | 88.7 |
| | 59*01 | 8*01 | | SHY | ST | LYHVM | | | | |
| | | | | (SEQ ID | (SEQ ID | HV | | | | |
| | | | | NO: 92) | NO: 93) | (SEQ ID | | | | |
| | | | | | | NO: 94) | | | | |
| M2B01 | IGHV3- | IGHD2- | IGHJ4*02 | GFTFS | ISYDG | ARDFQ | 8, 8, 18 | 13 | 95.608 | 92.9 |
| | 30*04 | 8*01 | | SHA | SNR | PSPMV | | | | |
| | | | | (SEQ ID | (SEQ ID | YGVGY | | | | |
| | | | | NO: 95) | NO: 96) | FDY | | | | |
| | | | | | | (SEQ ID | | | | |
| | | | | | | NO: 97) | | | | |
| M2B12 | IGHV3- | IGHD6- | IGHJ4*02 | GFTFS | ISYHG | AKEAA | 8, 8, 12 | 22 | 92.568 | 88.8 |
| | 30*01 | 19*01 | | SSA | TNK | VSGNF | | | | |
| | | | | (SEQ ID | (SEQ ID | DS | | | | |
| | | | | NO: 98) | NO: 99) | (SEQ ID | | | | |
| | | | | | | NO: 100) | | | | |
| M2C05 | IGHV3- | IGHD2- | IGHJ4*02 | GFTFS | INDRG | TRAYV | 8, 8, 17 | 24 | 91.864 | 83.7 |
| | 23*01 | 8*01 | | NYA | ETT | VLSVY | | | | |
| | | | | (SEQ ID | (SEQ ID | APKGF | | | | |
| | | | | NO: 101) | NO: 102) | DY | | | | |
| | | | | | | (SEQ ID | | | | |
| | | | | | | NO: 103) | | | | |
| M2D01 | IGHV4- | IGHD1- | IGHJ3*01 | RGAIT | ISSSG | ASSSG | 8, 7, 14 | 33 | 88.621 | 80.2 |
| | 59*08 | 26*01 | | DYH | ST | RYWGG | | | | |
| | | | | (SEQ ID | (SEQ ID | ALDL | | | | |
| | | | | NO: 104) | NO: 105) | (SEQ ID | | | | |
| | | | | | | NO: 106) | | | | |
| M2D2 | IGHV1- | IGHD1- | IGHJ5*02 | GYNFD | IDPSG | ARLEA | 8, 8, 10 | 29 | 90.136 | 85.7 |
| | 46*02 | 1*01 | | NYY | GRI | THEG | | | | |
| | | | | (SEQ ID | (SEQ ID | P | | | | |
| | | | | NO: 107) | NO: 108) | (SEQ ID | | | | |
| | | | | | | NO: 109) | | | | |
| M3C05 | IGHV3- | IGHD3- | IGHJ6*02 | GFGFR | INNDG | AREFG | 8, 8, 15 | 13 | 95.608 | 93.9 |
| | 74*01 | 10*01 | | SYW | SSA | VLDYH | | | | |
| | | | | (SEQ ID | (SEQ ID | YGMD | | | | |
| | | | | NO: 110) | NO: 111) | V | | | | |
| | | | | | | (SEQ ID | | | | |
| | | | | | | NO: 112) | | | | |
| M3C12 | IGHV3- | IGHD2- | IGHJ6*02 | GFSFG | TSSDG | ARELG | 8, 8, 15 | 15 | 94.932 | 91.8 |
| | 74*01 | 15*01 | | SYY | STT | ILDYY | | | | |
| | | | | (SEQ ID | (SEQ ID | YGMD | | | | |
| | | | | NO: 113) | NO: 114) | V | | | | |
| | | | | | | (SEQ ID | | | | |
| | | | | | | NO: 115) | | | | |
| M3D09 | IGHV3- | IGHD3- | IGHJ4*02 | GFIFS | ISYDG | ARDPE | 8, 8, 15 | 26 | 91.216 | 80.6 |
| | 30*04 | 16*01 | | NYA | TNK | RLADL | | | | |
| | | | | (SEQ ID | (SEQ ID | GRMD | | | | |
| | | | | NO: 116) | NO: 117) | F | | | | |
| | | | | | | (SEQ ID | | | | |
| | | | | | | NO: 118) | | | | |
| M3D12 | IGHV4- | IGHD6- | IGHJ4*02 | GASVS | IYHNG | ARLAV | 10, 7, 13 | 22 | 92.593 | 87.9 |
| | 30- | 13*01 | | SEYY | DT | AGVTG | | | | |
| | 4*01 | | | Y | (SEQ ID | FHY | | | | |
| | | | | (SEQ ID | NO: 120) | (SEQ ID | | | | |
| | | | | NO: 119) | | NO: 121) | | | | |
| M3F03 | IGHV3- | IGHD5- | IGHJ4*03 | GFTFS | MSHDG | AKDPG | 8, 8, 18 | 8 | 97.297 | 94.9 |
| | 30*18 | 18*01 | | RYA | SNR | GLGYS | | | | |
| | | | | (SEQ ID | (SEQ ID | YGLGY | | | | |
| | | | | NO: 122) | NO: 123) | FDY | | | | |
| | | | | | | (SEQ ID | | | | |
| | | | | | | NO: 124) | | | | |
| M4B01 | IGHV3- | IGHD5- | IGHJ3*02 | GFNFN | ISGSG | AKSQN | 8, 8, 16 | 29 | 90.136 | 86.7 |
| | 23*01 | 24*01 | | TYA | GVK | PQLVF | | | | |
| | | | | (SEQ ID | (SEQ ID | FDSFD | | | | |
| | | | | NO: 125) | NO: 126) | M | | | | |
| | | | | | | (SEQ ID | | | | |
| | | | | | | NO: 127) | | | | |
| M4C03 | IGHV3- | IGHD3- | IGHJ6*02 | GFRFN | IDSRS | AKALC | 8, 8, 22 | 24 | 91.864 | 86.7 |
| | 48*02 | 3*01 | | AFS | NII | VVNYD | | | | |
| | | | | (SEQ ID | (SEQ ID | FWSGL | | | | |
| | | | | NO: 128) | NO: 129) | LGCAM | | | | |
| | | | | | | DV | | | | |
| | | | | | | (SEQ ID | | | | |
| | | | | | | NO: 130) | | | | |
| M4C04 | IGHV2- | IGHD3- | IGHJ5*02 | GFSLN | IDWDG | AQSVI | 10, 7, 17 | 18 | 93.96 | 88.9 |
| | 70*11 | 22*01 | | TYGMS | DK | VPAQS | | | | |
| | | | | (SEQ ID | (SEQ ID | FVSWF | | | | |
| | | | | NO: 131) | NO: 132) | DP | | | | |
| | | | | | | (SEQ ID | | | | |
| | | | | | | NO: 133) | | | | |
| M4C08 | IGHV3- | IGHD3- | IGHJ6*02 | GFKFN | IDSRS | AKALC | 8, 8, 22 | 23 | 92.203 | 87.8 |
| | 48*02 | 3*01 | | AFS | NII | VVNYD | | | | |
| | | | | (SEQ ID | (SEQ ID | FWSGL | | | | |
| | | | | NO: 134) | NO: 135) | LGCAM | | | | |
| | | | | | | DV | | | | |
| | | | | | | (SEQ ID | | | | |
| | | | | | | NO: 136) | | | | |
| M2A02 | IGHV1- | IGHD3- | IGHJ3*02 | GDIFS | IIPLV | ARDRT | 8, 8, 13 | 36 | 87.838 | 80.6 |
| | 69*09 | 9*01 | | SHA | GIR | DWSGG | | | | |
| | | | | (SEQ ID | (SEQ ID | LDI | | | | |
| | | | | NO: 137) | NO: 138) | (SEQ ID | | | | |
| | | | | | | NO: 139) | | | | |
| M2A05 | IGHV2- | IGHD3- | IGHJ4*02 | GFSLS | IYWDD | VRKHY | 10, 7, 9 | 29 | 90.068 | 77 |
| | 5*02 | 3*01 | | ANSVG | DK | DLLQ | | | | |
| | | | | (SEQ ID | (SEQ ID | (SEQ ID | | | | |
| | | | | NO: 140) | NO: 141) | NO: 142) | | | | |
| M2A06 | IGHV1- | IGHD3- | IGHJ5*02 | GYSFS | INPSG | VRSLA | 8, 8, 11 | 21 | 92.857 | 85.7 |
| | 46*03 | 10*01 | | NYY | GST | NGSGS | | | | |
| | | | | (SEQ ID | (SEQ ID | P | | | | |
| | | | | NO: 143) | NO: 144) | (SEQ ID | | | | |
| | | | | | | NO: 145) | | | | |
| M2B10 | IGHV3- | IGHD3- | IGHJ4*02 | GFTFS | ISYDG | AREGL | 8, 8, 10 | 10 | 96.622 | 91.8 |
| | 30*04 | 3*01 | | NYP | RTK | EYLNY | | | | |
| | | | | (SEQ ID | (SEQ ID | (SEQ ID | | | | |
| | | | | NO: 146) | NO: 147) | NO: 148) | | | | |
| M2D07 | IGHV3- | IGHD3- | IGHJ4*02 | GFTFT | ISHDG | ARDFP | 8, 8, 17 | 20 | 93.243 | 87.8 |
| | 30- | 22*01 | | SFS | IIK | GSRGF | | | | |
| | 3*01 | | | (SEQ ID | (SEQ ID | FRYYF | | | | |
| | | | | NO: 149) | NO: 150) | DH | | | | |
| | | | | | | (SEQ ID | | | | |
| | | | | | | NO: 151) | | | | |
| M2D10 | IGHV1- | IGHD3- | IGHJ5*02 | GRAFL | MNPGS | ARGEV | 8, 8, 14 | 35 | 88.176 | 80.6 |
| | 8*01 | 22*01 | | TYD | GNA | SMRWR | | | | |
| | | | | (SEQ ID | (SEQ ID | WLDP | | | | |
| | | | | NO: 152) | NO: 153) | (SEQ ID | | | | |
| | | | | | | NO: 154) | | | | |
| M2F01 | IGHV1- | IGHD6- | IGHJ4*02 | EDTFT | INPSG | ARQGR | 8, 8, 11 | 12 | 95.918 | 94.9 |
| | 46*03 | 13*01 | | NYY | GST | TAACD | | | | |
| | | | | (SEQ ID | (SEQ ID | Y | | | | |
| | | | | NO: 155) | NO: 156) | (SEQ ID | | | | |
| | | | | | | NO: 157) | | | | |
| M4B04 | IGHV3- | IGHD3- | IGHJ4*02 | GFTFR | ILHDG | ARESL | 8, 8, 10 | 22 | 92.568 | 86.7 |
| | 30- | 3*01 | | SYT | TNR | EWMLD | | | | |
| | 3*01 | | | (SEQ ID | (SEQ ID | (SEQ ID | | | | |
| | | | | NO: 158) | NO: 159) | NO: 160) | | | | |
| M4B05 | IGHV1- | IGHD3- | IGHJ3*02 | GYSFI | INPSG | AREQA | 8, 8, 11 | 28 | 90.541 | 83.7 |
| | 46*03 | 10*01 | | SYY | GST | VGAYD | | | | |
| | | | | (SEQ ID | (SEQ ID | I | | | | |
| | | | | NO: 161) | NO: 162) | (SEQ ID | | | | |
| | | | | | | NO: 163) | | | | |
| M4B11 | IGHV1- | IGHD1- | IGHJ1*01 | GYIFT | VNPYS | AKDTS | 8, 8, 14 | 26 | 91.096 | 84.7 |
| | 2*02 | 14*01 | | GYF | GAT | NSGPD | | | | |
| | | | | (SEQ ID | (SEQ ID | LLEH | | | | |
| | | | | NO: 164) | NO: 165) | (SEQ ID | | | | |
| | | | | | | NO: 166) | | | | |
| M4F8 | IGHV1- | IGHD3- | IGHJ4*03 | EYTFT | INPTG | ARDRG | 8, 8, 11 | 31 | 89.527 | 82.7 |
| | 46*01 | 10*01 | | NHY | GST | MRYFD | | | | |
| | | | | (SEQ ID | (SEQ ID | Y | | | | |
| | | | | NO: 167) | NO: 168) | (SEQ ID | | | | |
| | | | | | | NO: 169) | | | | |
| M2A03 | IGHV4- | IGHD3- | IGHJ5*02 | GGSIS | IFYFG | ARHIL | 10, 7, 16 | 20 | 93.311 | 86.9 |
| | 39*01 | 22*01 | | SSSNS | ST | SSSGV | | | | |
| | | | | (SEQ ID | (SEQ ID | SGWLD | | | | |
| | | | | NO: 170) | NO: 171) | P | | | | |
| | | | | | | (SEQ ID | | | | |
| | | | | | | NO: 172) | | | | |
| M3A07 | IGHV1- | IGHD2- | IGHJ3*02 | GYTFT | VHGGN | ATAMP | 8, 8, 10 | 16 | 94.521 | 87.6 |
| | 3*01 | 2*01 | | RHA | ENT | GAFN | | | | |
| | | | | (SEQ ID | (SEQ ID | M | | | | |
| | | | | NO: 173) | NO: 174) | (SEQ ID | | | | |
| | | | | | | NO: 175) | | | | |
| M5F2 | IGHV3- | IGHD2- | IGHJ3*01 | GFSFS | IKPDG | ARDRG | 8, 8, 20 | 24 | 91.864 | 84.7 |
| | 7*03 | 15*01 | | TFW | SEK | YCRGG | | | | |
| | | | | (SEQ ID | (SEQ ID | SCYRR | | | | |
| | | | | NO: 176) | NO: 177) | NAFDL | | | | |
| | | | | | | (SEQ ID | | | | |
| | | | | | | NO: 178) | | | | |
| M4B03 | IGHV1- | IGHD1- | IGHJ4*02 | GYIFS | ITAGS | AREYN | 8, 8, 12 | 32 | 87.838 | 80.6 |
| | 3*01 | 20*01 | | NYR | GNT | WNDAF | | | | |
| | | | | (SEQ ID | (SEQ ID | PY | | | | |
| | | | | NO: 179) | NO: 180) | (SEQ ID | | | | |
| | | | | | | NO: 181) | | | | |
| M4B06 | IGHV3- | IGHD5- | IGHJ3*02 | GFTFS | IGGSV | AKVPS | 8, 8, 15 | 16 | 91.186 | 85.7 |
| | 23*01 | 24*01 | | NYA | GTT | FFVFE | | | | |
| | | | | (SEQ ID | (SEQ ID | DGFD | | | | |
| | | | | NO: 182) | NO: 183) | M | | | | |
| | | | | | | (SEQ ID | | | | |
| | | | | | | NO: 184) | | | | |
| M2C08 | IGHV1- | IGHD3- | IGHJ3*02 | GFTFS | ISADN | TRGPA | 8, 8, 17 | 36 | 91.864 | 81.6 |
| | 3*01 | 22*01 | | LYT | SNT | YYFDS | | | | |
| | | | | (SEQ ID | (SEQ ID | SGFAF | | | | |
| | | | | NO: 185) | NO: 186) | DI | | | | |
| | | | | | | (SEQ ID | | | | |
| | | | | | | NO: 187) | | | | |
| M3D04 | IGHV3- | IGHD3- | IGHJ6*02 | GFTFE | IKSKL | ATDRS | 8, 10, 22 | 21 | 93.377 | 89 |
| | 15*01 | 3*01 | | SAW | DHGTT | DVRFL | | | | |
| | | | | (SEQ ID | (SEQ ID | EWLGN | | | | |
| | | | | NO: 188) | NO: 189) | YQFGM | | | | |
| | | | | | | DV | | | | |
| | | | | | | (SEQ ID | | | | |
| | | | | | | NO: 190) | | | | |
| MIC7 | IGHV3- | IGHD1- | IGHJ4*02 | GFTFS | ITYDG | VKSGS | 8, 8, 14 | 28 | 90.411 | 84.7 |
| | 30- | 26*01 | | SYA | SGK | FSSAF | | | | |
| | 3*01 | | | (SEQ ID | (SEQ ID | WLED | | | | |
| | | | | NO: 191) | NO: 192) | (SEQ ID | | | | |
| | | | | | | NO: 193) | | | | |
| M2B11 | IGHV1- | IGHD3- | IGHJ3*02 | GYTFT | MNPNS | ARNRH | 8, 8, 19 | 20 | 93.197 | 89.8 |
| | 8*01 | 10*01 | | SHD | ATT | YYYAD | | | | |
| | | | | (SEQ ID | (SEQ ID | GPTRD | | | | |
| | | | | NO: 194) | NO: 195) | AFDI | | | | |
| | | | | | | (SEQ ID | | | | |
| | | | | | | NO: 196) | | | | |
| M2C11 | IGHV1- | IGHD3- | IGHJ3*02 | GYTFT | MNPNS | ARGRH | 8, 8, 19 | 25 | 94.595 | 89.8 |
| | 8*01 | 3*01 | | SHD | DTT | NYYTN | | | | |
| | | | | (SEQ ID | (SEQ ID | GPTRD | | | | |
| | | | | NO: 197) | NO: 198) | GFDI | | | | |
| | | | | | | (SEQ ID | | | | |
| | | | | | | NO: 199) | | | | |
| M2D08 | IGHV3- | IGHD3- | IGHJ4*02 | GFVFR | ISDDG | ARGDY | 8, 8, 13 | 20 | 87.797 | 81.6 |
| | 30*09 | 16*01 | | FSG | SQK | GVSYF | | | | |
| | | | | (SEQ ID | (SEQ ID | LAH | | | | |
| | | | | NO: 200) | NO: 201) | (SEQ ID | | | | |
| | | | | | | NO: 202) | | | | |
| M3C06 | IGHV1- | IGHD3- | IGHJ3*01 | GGTLS | IIPVM | ATARG | 8, 8, 17 | 19 | 91.525 | 87.6 |
| | 69*09 | 10*01 | | NYA | NVA | LGSLE | | | | |
| | | | | (SEQ ID | (SEQ ID | GGDAF | | | | |
| | | | | NO: 203) | NO: 204) | DV | | | | |
| | | | | | | (SEQ ID | | | | |
| | | | | | | NO: 205) | | | | |
| M4A10 | IGHV1- | IGHD3- | IGHJ5*01 | GYTFN | INPNS | ARDNF | 8, 8, 20 | 12 | 95.608 | 91.8 |
| | 2*02 | 3*01 | | GHY | GGT | GFWSA | | | | |
| | | | | (SEQ ID | (SEQ ID | SSGRH | | | | |
| | | | | NO: 206) | NO: 207) | NWLDS | | | | |
| | | | | | | (SEQ ID | | | | |
| | | | | | | NO: 208) | | | | |
| M1A08 | IGHV1- | IGHD1- | IGHJ4*02 | GVTLT | INPAR | ARSGS | 8, 8, 18 | 9 | 89.078 | 81.6 |
| | 2*06 | 26*01 | | DYY | GGT | THNIY | | | | |
| | | | | (SEQ ID | (SEQ ID | SGFYA | | | | |
| | | | | NO: 209) | NO: 210) | FDH | | | | |
| | | | | | | (SEQ ID | | | | |
| | | | | | | NO: 211) | | | | |
| M2B07 | IGHV3- | IGHD3- | IGHJ3*02 | GFTFN | VKSDG | ARESR | 8, 8, 20 | 18 | 94.595 | 88.8 |
| | 74*01 | 9*01 | | NYW | SIT | PYDPL | | | | |
| | | | | (SEQ ID | (SEQ ID | TGSHP | | | | |
| | | | | NO: 212) | NO: 213) | DAFDI | | | | |
| | | | | | | (SEQ ID | | | | |
| | | | | | | NO: 214) | | | | |
| M2B09 | IGHV1- | IGHD2- | IGHJ3*01 | GYTFT | ISVHT | ARDRT | 8, 8, 17 | 12 | 87.838 | 77.6 |
| | 18*01 | 15*01 | | SYG | DDT | DISGR | | | | |
| | | | | (SEQ ID | (SEQ ID | VRDAF | | | | |
| | | | | NO: 215) | NO: 216) | DL | | | | |
| | | | | | | (SEQ ID | | | | |
| | | | | | | NO: 217) | | | | |
| M2E04 | IGHV4- | IGHD1- | IGHJ4*02 | RFSIN | ISHAG | AASSN | 9, 7, 12 | 23 | 92.784 | 84.5 |
| | 38- | 1*01 | | SGYY | SI | WHNAL | | | | |
| | 2*02 | | | (SEQ ID | (SEQ ID | EY | | | | |
| | | | | NO: 218) | NO: 219) | (SEQ ID | | | | |
| | | | | | | NO: 220) | | | | |
| M2E05 | IGHV4- | IGHD4- | IGHJ4*02 | RYSIN | VSHAG | AASSN | 9, 7, 12 | 32 | 90.378 | 82.5 |
| | 38- | 11*01 | | SGYY | VT | WHNAL | | | | |
| | 2*02 | | | (SEQ ID | (SEQ ID | EY | | | | |
| | | | | NO: 221) | NO: 222) | (SEQ ID | | | | |
| | | | | | | NO: 223) | | | | |
| M2E06 | IGHV3- | IGHD3- | IGHJ6*02 | AFTFS | INTSG | ARFFY | 8, 8, 9 | 0 | 93.127 | 87.8 |
| | 23*04 | 10*01 | | DYA | GRT | VMDV | | | | |
| | | | | (SEQ ID | (SEQ ID | (SEQ ID | | | | |
| | | | | NO: 224) | NO: 225) | NO: 226) | | | | |
| M4A08 | IGHV3- | IGHD2- | IGHJ4*02 | GFNFR | TNGDG | ARERL | 8, 8, 20 | 10 | 91.554 | 80.6 |
| | 74*01 | 8*02 | | THW | SVT | DYSST | | | | |
| | | | | (SEQ ID | (SEQ ID | RGHFP | | | | |
| | | | | NO: 227) | NO: 228) | GDFDS | | | | |
| | | | | | | (SEQ ID | | | | |
| | | | | | | NO: 229) | | | | |
| M2E11L | IGHV1- | IGHD3- | IGHJ6*03 | GGTFT | IIPFV | ARVRG | 8, 8, 19 | 9 | 93.22 | 86.7 |
| | 69*13 | 10*01 | | NFA | GPA | FGETA | | | | |
| | | | | (SEQ ID | (SEQ ID | YFYHY | | | | |
| | | | | NO: 230) | NO: 231) | YMDV | | | | |
| | | | | | | (SEQ ID | | | | |
| | | | | | | NO: 232) | | | | |
| M1A11 | IGHV4- | IGHD6- | IGHJ5*02 | GDSIS | IYYTG | ARQEG | 10, 7, 14 | 9 | 93.645 | 88.9 |
| | 39*01 | 13*01 | | SSSFY | ST | SSWSN | | | | |
| | | | | (SEQ ID | (SEQ ID | WFDP | | | | |
| | | | | NO: 233) | NO: 234) | (SEQ ID | | | | |
| | | | | | | NO: 235) | | | | |
| M2C10 | IGHV3- | IGHD3- | IGHJ4*02 | GFTFN | ISDDG | ARDPT | 8, 8, 14 | 0 | 95.946 | 90.8 |
| | 30*04 | 16*01 | | NYA | SKK | PLGEY | | | | |
| | | | | (SEQ ID | (SEQ ID | YFDY | | | | |
| | | | | NO: 236) | NO: 237) | (SEQ ID | | | | |
| | | | | | | NO: 238) | | | | |
| M2E01 | IGHV4- | IGHD3- | IGHJ4*02 | GGSIS | IYYSG | ARLVS | 10, 7, 12 | 11 | 96.98 | 93.9 |
| | 39*01 | 16*01 | | SSTYY | ST | PYDQF | | | | |
| | | | | (SEQ ID | (SEQ ID | DY | | | | |
| | | | | NO: 239) | NO: 240) | (SEQ ID | | | | |
| | | | | | | NO: 241) | | | | |
| M2E08 | IGHV3- | IGHD1- | IGHJ6*03 | GFMFS | ISGSG | AKDLG | 8, 8, 14 | 15 | 93.919 | 89.8 |
| | 23*04 | 26*01 | | SYA | GNT | NDYFY | | | | |
| | | | | (SEQ ID | (SEQ ID | YIDV | | | | |
| | | | | NO: 242) | NO: 243) | (SEQ ID | | | | |
| | | | | | | NO: 244) | | | | |
| M3A04 | IGHV3- | IGHD3- | IGHJ4*02 | GLTFS | ISYDG | ARGTA | 8, 8, 20 | 24 | 95.932 | 92.9 |
| | 30- | 3*01 | | TYV | TNE | DYDVW | | | | |
| | 3*01 | | | (SEQ ID | (SEQ ID | SGSAW | | | | |
| | | | | NO: 245) | NO: 246) | GGFDY | | | | |
| | | | | | | (SEQ ID | | | | |
| | | | | | | NO: 247) | | | | |
| M3A12 | IGHV4- | IGHD3- | IGHJ4*02 | GDSIS | SVYSG | ARGND | 10, 7, 13 | 10 | 92.282 | 85.9 |
| | 30- | 3*01 | | SSDYC | DT | FWSGS | | | | |
| | 4*01 | | | (SEQ ID | (SEQ ID | FDY | | | | |
| | | | | NO: 248) | NO: 249) | (SEQ ID | | | | |
| | | | | | | NO: 250) | | | | |
| M3C07 | IGHV4- | IGHD3- | IGHJ3*02 | GGSIS | IYYTG | ARHET | 10, 7, 13 | 0 | 89.298 | 81.8 |
| | 39*01 | 16*01 | | RSTHY | NT | YDYGV | | | | |
| | | | | (SEQ ID | (SEQ ID | FNI | | | | |
| | | | | NO: 251) | NO: 252) | (SEQ ID | | | | |
| | | | | | | NO: 253) | | | | |
| M3D03 | IGHV1- | IGHD3- | IGHJ6*02 | GYTFT | ISAYN | ARDAP | 8, 8, 16 | 6 | 100 | 100 |
| | 18*01 | 10*01 | | SYG | GNT | FGRDD | | | | |
| | | | | (SEQ ID | (SEQ ID | GYGMD | | | | |
| | | | | NO: 254) | NO: 255) | V | | | | |
| | | | | | | (SEQ ID | | | | |
| | | | | | | NO: 256) | | | | |
| M8C10 | IGHV3- | IGHD3- | IGHJ3*01 | GFTFT | ISSDG | ARGYY | 8, 8, 13 | 17 | 96.61 | 93.9 |
| | 30- | 22*01 | | TYT | NNK | SGGDA | | | | |
| | 3*01 | | | (SEQ ID | (SEQ ID | FDV | | | | |
| | | | | NO: 257) | NO: 258) | (SEQ ID | | | | |
| | | | | | | NO: 259) | | | | |
| M3A03 | IGHV1- | IGHD3- | IGHJ4*02 | AYTFT | ISAYR | ARTSY | 8, 8, 15 | 22 | 96.939 | 94.9 |
| | 18*01 | 22*01 | | SYG | GNT | YDSSY | | | | |
| | | | | (SEQ ID | (SEQ ID | YPID | | | | |
| | | | | NO: 260) | NO: 261) | Y | | | | |
| | | | | | | (SEQ ID | | | | |
| | | | | | | NO: 262) | | | | |
| M3A05 | IGHV2- | IGHD3- | IGHJ4*02 | GFSLN | IYWDG | AHREN | 10, 7, 11 | 0 | 96.99 | 95 |
| | 5*02 | 16*01 | | TGGVS | DK | SAYID | | | | |
| | | | | (SEQ ID | (SEQ ID | F | | | | |
| | | | | NO: 263) | NO: 264) | (SEQ ID | | | | |
| | | | | | | NO: 265) | | | | |
| M3A09 | IGHV3- | IGHD6- | IGHJ4*02 | GFTFS | ISYDG | ARDVQ | 8, 8, 12 | 22 | 100 | 100 |
| | 30- | 13*01 | | SYA | SNK | QQLAL | | | | |
| | 3*01 | | | (SEQ ID | (SEQ ID | DY | | | | |
| | | | | NO: 266) | NO: 267) | (SEQ ID | | | | |
| | | | | | | NO: 268) | | | | |
| M3A11 | IGHV3- | IGHD4- | IGHJ5*02 | GFTFS | ISSTG | ARGKT | 8, 8, 13 | 0 | 96.271 | 92.9 |
| | 48*04 | 11*01 | | DYS | STI | TVTIW | | | | |
| | | | | (SEQ ID | (SEQ ID | FDP | | | | |
| | | | | NO: 269) | NO: 270) | (SEQ ID | | | | |
| | | | | | | NO: 271) | | | | |
| M3B04 | IGHV4- | IGHD4- | IGHJ6*03 | GGSIS | IYHSG | ARRPN | 9, 7, 20 | 15 | 94.898 | 90.8 |
| | 4*02 | 17*01 | | SNNW | ST | DYGDY | | | | |
| | | | | (SEQ ID | (SEQ ID | SYYYY | | | | |
| | | | | NO: 272) | NO: 273) | YYMDV | | | | |
| | | | | | | (SEQ ID | | | | |
| | | | | | | NO: 274) | | | | |
| M3B06 | IGHV1- | IGHD3- | IGHJ4*02 | GYNFP | ISAYN | ARVPR | 8, 8, 17 | 42 | 91.864 | 81.6 |
| | 18*01 | 10*01 | | AYG | VNT | PPLSG | | | | |
| | | | | (SEQ ID | (SEQ ID | TYSTA | | | | |
| | | | | NO: 275) | NO: 276) | DF | | | | |
| | | | | | | (SEQ ID | | | | |
| | | | | | | NO: 277) | | | | |
| M3B07 | IGHV4- | IGHD3- | IGHJ6*02 | GGSFS | INHSG | ARGPI | 8, 7, 18 | 45 | 96.587 | 92.8 |
| | 34*01 | 9*01 | | GYY | ST | RYSSY | | | | |
| | | | | (SEQ ID | (SEQ ID | YYYYG | | | | |
| | | | | NO: 278) | NO: 279) | LDV | | | | |
| | | | | | | (SEQ ID | | | | |
| | | | | | | NO: 280) | | | | |
| M3B12 | IGHV3- | IGHD1- | IGHJ6*03 | GFTFS | IRYDG | AKDSG | 8, 8, 15 | 28 | 100 | 100 |
| | 30*02 | 26*01 | | SYG | SNK | SYYYY | | | | |
| | | | | (SEQ ID | (SEQ ID | YYMD | | | | |
| | | | | NO: 281) | NO: 282) | V | | | | |
| | | | | | | (SEQ ID | | | | |
| | | | | | | NO: 283) | | | | |
| M3C01 | IGHV4- | IGHD4- | IGHJ4*02 | GGSIS | VFYSG | ARHSW | 10, 7, 10 | 28 | 97.993 | 94.9 |
| | 39*01 | 23*01 | | SSSY | ST | DRWV | | | | |
| | | | | F | (SEQ ID | Y | | | | |
| | | | | (SEQ ID | NO: 285) | (SEQ ID | | | | |
| | | | | NO: 284) | | NO: 286) | | | | |
| M3D06 | IGHV3- | IGHD3- | IGHJ4*02 | GFPFN | INNDG | ARAAR | 8, 8, 12 | 17 | 94.237 | 89.8 |
| | 74*01 | 10*01 | | FYW | SSP | FGDYV | | | | |
| | | | | (SEQ ID | (SEQ ID | DQ | | | | |
| | | | | NO: 287) | NO: 288) | (SEQ ID | | | | |
| | | | | | | NO: 289) | | | | |
| M3D08 | IGHV4- | IGHD3- | IGHJ5*02 | GVSIS | LYTSG | ARDVR | 8, 7, 18 | 11 | 92.491 | 86.6 |
| | 4*07 | 3*01 | | SHY | TT | LTVFG | | | | |
| | | | | (SEQ ID | (SEQ ID | ALHNW | | | | |
| | | | | NO: 290) | NO: 291) | FDP | | | | |
| | | | | | | (SEQ ID | | | | |
| | | | | | | NO: 292) | | | | |
| M3E02 | IGHV4- | IGHD6- | IGHJ2*01 | GGSIS | IYTSG | ARDAS | 8, 7, 16 | 26 | 100 | 100 |
| | 4*07 | 13*01 | | SYY | ST | GLSWY | | | | |
| | | | | (SEQ ID | (SEQ ID | DWYFD | | | | |
| | | | | NO: 293) | NO: 294) | L | | | | |
| | | | | | | (SEQ ID | | | | |
| | | | | | | NO: 295) | | | | |
| M3E05 | IGHV3- | IGHD6- | IGHJ4*02 | GFSVR | IYSDG | ARDPT | 8, 7, 16 | 12 | 92.491 | 86.6 |
| | 53*01 | 13*01 | | TNY | RT | FYSSS | | | | |
| | | | | (SEQ ID | (SEQ ID | WWYLD | | | | |
| | | | | NO: 296) | NO: 297) | H | | | | |
| | | | | | | (SEQ ID | | | | |
| | | | | | | NO: 298) | | | | |
| M3E06 | IGHV3- | IGHD3- | IGHJ6*03 | GFTFS | IKQDG | ARYYY | 8, 8, 20 | 15 | 100 | 100 |
| | 7*01 | 22*01 | | SYW | SEK | DSSGY | | | | |
| | | | | (SEQ ID | (SEQ ID | YYYYY | | | | |
| | | | | NO: 299) | NO: 300) | YYMDV | | | | |
| | | | | | | (SEQ ID | | | | |
| | | | | | | NO: 301) | | | | |
| M3E08 | IGHV3- | IGHD2- | IGHJ6*02 | GFSFS | VSAGG | AKDKY | 8, 8, 14 | 0 | 94.932 | 91.8 |
| | 23*01 | 2*01 | | SYV | GST | PHYYY | | | | |
| | | | | (SEQ ID | (SEQ ID | GMDV | | | | |
| | | | | NO: 302) | NO: 303) | (SEQ ID | | | | |
| | | | | | | NO: 304) | | | | |
| M1A01 | IGHV1- | IGHD5- | IGHJ5*02 | GGTFD | IIPLV | ASAFQ | 7, 8, 11 | 47 | 85.666 | 77.3 |
| | 69*09 | 24*01 | | NA | DIV | DGWFD | | | | |
| | | | | (SEQ ID | (SEQ ID | P | | | | |
| | | | | NO: 305) | NO: 306) | (SEQ ID | | | | |
| | | | | | | NO: 307) | | | | |
| M1A12 | IGHV1- | IGHD5- | IGHJ4*02 | GDIMS | IIPLF | AREEN | 8, 8, 11 | 17 | 84.797 | 73.5 |
| | 69*09 | 24*01 | | SHS | GMK | LSWLD | | | | |
| | | | | (SEQ ID | (SEQ ID | Y | | | | |
| | | | | NO: 308) | NO: 309) | (SEQ ID | | | | |
| | | | | | | NO: 310) | | | | |
| M2B04 | IGHV1- | IGHD1- | IGHJ5*02 | GYSIT | SSAHT | AMGAY | 8, 8, 14 | 36 | 90.411 | 85.6 |
| | 18*01 | 26*01 | | TYG | GNT | TSSRN | | | | |
| | | | | (SEQ ID | (SEQ ID | WFDP | | | | |
| | | | | NO: 311) | NO: 312) | (SEQ ID | | | | |
| | | | | | | NO: 313) | | | | |
| M2C01 | IGHV3- | IGHD3- | IGHJ4*02 | GFTFS | IKPHT | TTGPR | 8, 10, 9 | 26 | 90.698 | 87 |
| | 15*01 | 16*01 | | NAW | DGAT | LSLM | | | | |
| | | | | (SEQ ID | T | (SEQ ID | | | | |
| | | | | NO: 314) | (SEQ ID | NO: 316) | | | | |
| | | | | | NO: 315) | | | | | |
| M2D02 | IGHV1- | IGHD1- | IGHJ4*02 | GGTLT | IIPFL | ARDHS | 8, 8, 11 | 24 | 94.257 | 88.8 |
| | 69*09 | 26*01 | | RYT | DIA | GNYFD | | | | |
| | | | | (SEQ ID | (SEQ ID | L | | | | |
| | | | | NO: 317) | NO: 318) | (SEQ ID | | | | |
| | | | | | | NO: 319) | | | | |
| M2D09 | IGHV3- | IGHD1- | IGHJ4*02 | GYTFS | ISGSG | ARGLY | 8, 8, 10 | 20 | 96.271 | 93.9 |
| | 23*01 | 1*01 | | SYA | ANT | TTGDY | | | | |
| | | | | (SEQ ID | (SEQ ID | (SEQ | | | | |
| | | | | NO: 320) | NO: 321) | ID | | | | |
| | | | | | | NO: 322) | | | | |
| M2E07 | IGHV3- | IGHD6- | IGHJ6*03 | GFTFS | ITESG | AKDMG | 8, 8, 23 | 28 | 91.216 | 84.7 |
| | 23*04 | 13*01 | | SYA | DDA | LYKSS | | | | |
| | | | | (SEQ ID | (SEQ ID | WYGNF | | | | |
| | | | | NO: 323) | NO: 324) | FYYYY | | | | |
| | | | | | | MDV | | | | |
| | | | | | | (SEQ ID | | | | |
| | | | | | | NO: 325) | | | | |
| M3E01 | IGHV3- | IGHD5- | IGHJ6*02 | GFTVS | IYDGG | ARDRG | 8, 7, 18 | 20 | 95.904 | 92.8 |
| | 53*01 | 12*01 | | SKY | ST | TYSGY | | | | |
| | | | | (SEQ ID | (SEQ ID | DWYSG | | | | |
| | | | | NO: 326) | NO: 327) | MDV | | | | |
| | | | | | | (SEQ ID | | | | |
| | | | | | | NO: 328) | | | | |
| M3E09 | IGHV3- | IGHD1- | IGHJ6*03 | GFRFD | INWIG | ARGVG | 8, 8, 13 | 16 | 94.915 | 91.8 |
| | 20*01 | 26*01 | | DYA | GTT | YYYYY | | | | |
| | | | | (SEQ ID | (SEQ ID | MDV | | | | |
| | | | | NO: 329) | NO: 330) | (SEQ ID | | | | |
| | | | | | | NO: 331) | | | | |
| M3F02 | IGHV4- | IGHD7- | IGHJ4*02 | GGSIS | IYYSG | ARITG | 10, 7, 14 | 36 | 99.662 | 100 |
| | 30- | 27*01 | | SGDYY | ST | EGLPQ | | | | |
| | 4*01 | | | (SEQ ID | (SEQ ID | AFDY | | | | |
| | | | | NO: 332) | NO: 333) | (SEQ ID | | | | |
| | | | | | | NO: 334) | | | | |
| M4A11 | IGHV1- | IGHD2- | IGHJ6*02 | GYTFS | ISTHN | ARMIP | 8, 8, 24 | 25 | 84.014 | 76.5 |
| | 18*04 | 2*02 | | NYG | QNV | VGTTY | | | | |
| | | | | (SEQ ID | (SEQ ID | TPYWT | | | | |
| | | | | NO: 335) | NO: 336) | GWTPG | | | | |
| | | | | | | GMDI | | | | |
| | | | | | | (SEQ ID | | | | |
| | | | | | | NO: 337) | | | | |
| M4B02 | IGHV4- | IGHD2- | IGHJ3*02 | GGSIS | IYYSG | ARLGV | 10, 7, 13 | 13 | 94.295 | 92.9 |
| | 39*01 | 15*01 | | SSSYY | ST | GLYEA | | | | |
| | | | | (SEQ ID | (SEQ ID | FDI | | | | |
| | | | | NO: 338) | NO: 339) | (SEQ ID | | | | |
| | | | | | | NO: 340) | | | | |

nt sub—nucleotide substitution; nt ID%—nt identity percentage; amino acid identity percentage.

TABLE 3C
VL information of the mAbs

| | | | VL | | | | | | |
|---|---|---|---|---|---|---|---|---|---|
| mAb | | | | | | CDR | nt | nt | aa |
| name | Germline | | CDR1 sequence | CDR2 sequence | CDR3 sequence | lengths | sub. | ID% | ID% |
| MIC7s | IGKV2- | IGKJ1* | ESLLNSDGKTY | DLS | MQGLHLPWT | 11, 3, 9 | 19 | 93.645 | 90 |
| | 29*03 | 01 | (SEQ ID NO: 341) | (SEQ ID NO: 342) | (SEQ ID NO: 343) | | | | |
| MID2 | IGKV1- | IGKJ2* | QSIRGH | GAS | QQSYSNPYS | 6, 3, 9 | 17 | 94.014 | 90.5 |
| | 39*01 | 03 | (SEQ ID NO: 344) | (SEQ ID NO: 345) | (SEQ ID NO: 346) | | | | |
| M2A09 | IGKV3- | IGKJ1* | QTVGAH | GAS | QQYGASPPT | 6, 3, 9 | 20 | 93.103 | 88.5 |
| | 20*01 | 01 | (SEQ ID NO: 347) | (SEQ ID NO: 348) | (SEQ ID NO: 349) | | | | |
| M2C06 | IGKV3- | IGKJ1* | QSLNSAS | GAS | QQYAFSPPT | 7, 3, 9 | 23 | 92.069 | 84.4 |
| | 20*01 | 01 | (SEQ ID NO: 350) | (SEQ ID NO: 351) | (SEQ ID NO: 352) | | | | |
| M3C04 | IGKV1- | IGKJ2* | QSISNY | AAS | QQSASTPHT | 6, 3, 9 | 7 | 97.552 | 94.7 |
| | 39*01 | 01 | (SEQ ID NO: 353) | (SEQ ID NO: 354) | (SEQ ID NO: 355) | | | | |
| M3E03 | IGKV3- | IGKJ1* | QNVDSSY | GAS | QQYGTSPTWT | 7, 3, 10 | 16 | 94.425 | 91.7 |
| | 20*01 | 01 | (SEQ ID NO: 356) | (SEQ ID NO: 357) | (SEQ ID NO: 358) | | | | |
| M4A02 | IGLVI- | IGLJ2* | SSNIGAFFD | GDR | QSYDRGLSAWV | 9, 3, 11 | 22 | 92.617 | 87.8 |
| | 40*01 | 01 | (SEQ ID NO: 359) | (SEQ ID NO: 360) | (SEQ ID NO: 361) | | | | |
| M6E10 | IGKV1- | IGKJ4* | QTISPY | GTN | QQSHTTPLT | 6, 3, 9 | 27 | 90.493 | 84.2 |
| | 39*01 | 01 | (SEQ ID NO: 362) | (SEQ ID NO: 363) | (SEQ ID NO: 364) | | | | |
| M7D5 | IGKV1- | IGKJ4* | QSLLTY | SAS | QQSHTTPLT | 6, 3, 9 | 30 | 89.437 | 84.2 |
| | 39*01 | 01 | (SEQ ID NO: 365) | (SEQ ID NO: 366) | (SEQ ID NO: 367) | | | | |
| M2B6 | IGLV1- | IGLJ2 | SSNIGAGYD | GNT | QSYERNLDGAL | 9, 3, 11 | 27 | 90.878 | 87.9 |
| | 40*01 | 01 | (SEQ ID NO: 368) | (SEQ ID NO: 369) | (SEQ ID NO: 370) | | | | |
| M3A01 | IGLV1- | IGLJ2* | SSNIGAGYD | GNY | QSYDTSLSGWI | 9, 3, 11 | 11 | 96.309 | 94.9 |
| | 40*01 | 01 | (SEQ ID NO: 371) | (SEQ ID NO: 372) | (SEQ ID NO: 373) | | | | |
| M3A06 | IGLV1- | IGLJ3* | SSNIGAGYD | ANN | QSYDRSLSGSV | 9, 3, 11 | 7 | 97.659 | 94.9 |
| | 40*01 | 02 | (SEQ ID NO: 374) | (SEQ ID NO: 375) | (SEQ ID NO: 376) | | | | |
| M3A08 | IGLV1- | IGLJ2* | SSNLGTGYD | GNT | QSYDSSLGGLV | 9, 3, 11 | 22 | 92.617 | 86.9 |
| | 40*01 | 01 | (SEQ ID NO: 377) | (SEQ ID NO: 378) | (SEQ ID NO: 379) | | | | |
| M3B02 | IGLV1- | IGLJ3* | SSNIGAGYD | GNN | QSYDRSLSGWV | 9, 3, 11 | 9 | 96.98 | 94.9 |
| | 40*01 | 02 | (SEQ ID NO: 380) | (SEQ ID NO: 381) | (SEQ ID NO: 382) | | | | |
| M3E04 | IGLV1- | IGLJ2* | SSNIGAGYD | GNN | QSYDSSLSGSI | 9, 3, 11 | 9 | 96.99 | 96 |
| | 40*01 | 01 | (SEQ ID NO: 383) | (SEQ ID NO: 384) | (SEQ ID NO: 385) | | | | |
| M3E10 | IGKV1- | IGKJ5* | QGISSY | AAS | QQVTSYPFSIT | 6, 3, 11 | 4 | 98.596 | 96.8 |
| | 9*01 | 01 | (SEQ ID NO: 386) | (SEQ ID NO: 387) | (SEQ ID NO: 388) | | | | |
| M4A05 | IGLV1- | IGLJ2* | GSNIGAGYD | DNN | QSYDRNLEGVV | 9, 3, 11 | 15 | 94.845 | 88.9 |
| | 40*01 | 01 | (SEQ ID NO: 389) | (SEQ ID NO: 390) | (SEQ ID NO: 391) | | | | |
| M2B05 | IGKV1- | IGKJ2* | QTINSY | TVS | QQSYNSPYT | 6, 3, 9 | 18 | 93.662 | 90.5 |
| | 39*01 | 01 | (SEQ ID NO: 392) | (SEQ ID NO: 393) | (SEQ ID NO: 394) | | | | |
| M2B06 | IGKV1- | IGKJ2* | QTISSY | GAS | QQSKSIPYT | 6, 3, 9 | 17 | 94.035 | 89.5 |
| | 39*01 | 01 | (SEQ ID NO: 395) | (SEQ ID NO: 396) | (SEQ ID NO: 397) | | | | |
| M2C07 | IGKV1- | IGKJ4* | QSVGAY | AGS | QQSLGDFLT | 6, 3, 9 | 21 | 92.308 | 83.5 |
| | 39*01 | 01 | (SEQ ID NO: 398) | (SEQ ID NO: 399) | (SEQ ID NO: 400) | | | | |
| M2D03 | IGKV1- | IGKJ4* | QSVGAY | AGS | QQSLGDFLT | 6, 3, 9 | 17 | 93.773 | 87.9 |
| | 39*01 | 01 | (SEQ ID NO: 401) | (SEQ ID NO: 402) | (SEQ ID NO: 403) | | | | |
| M2D11 | IGKV1- | IGKJ2* | QTISSY | TVS | QQSYGAPYT | 6, 3, 9 | 11 | 96.127 | 92.6 |
| | 39*01 | 01 | (SEQ ID NO: 404) | (SEQ ID NO: 405) | (SEQ ID NO: 406) | | | | |
| M3C11 | IGLV3- | IGLJ3* | KLGDKY | QDN | QAWDSGTV | 6, 3, 8 | 8 | 97.153 | 95.7 |
| | 1*01 | 02 | (SEQ ID NO: 407) | (SEQ ID NO: 408) | (SEQ ID NO: 409) | | | | |
| M3D05 | IGLV1- | IGLJ3* | SSNIRSNA | YDG | AAWDDSLNGPV | 8, 3, 11 | 17 | 94.257 | 90.8 |
| | 36*01 | 02 | (SEQ ID NO: 410) | (SEQ ID NO: 411) | (SEQ ID NO: 412) | | | | |
| M3D10 | IGKV1- | IGKJ2* | QNIASF | AAS | QQSYNTPFT | 6, 3, 9 | 15 | 94.718 | 90.5 |
| | 39*01 | 01 | (SEQ ID NO: 413) | (SEQ ID NO: 414) | (SEQ ID NO: 415) | | | | |
| M4A06 | IGKV3- | IGKJ2* | QSITSNY | GAS | QQYGSSLYI | 7, 3, 9 | 7 | 97.552 | 95.8 |
| | 20*01 | 01 | (SEQ ID NO: 416) | (SEQ ID NO: 417) | (SEQ ID NO: 418) | | | | |
| M4A12 | IGLV1- | IGLJ2* | NSNIGKNY | ENN | GAWDSSLNGVL | 8, 3, 11 | 10 | 96.61 | 92.8 |
| | 51*02 | 01 | (SEQ ID NO: 419) | (SEQ ID NO: 420) | (SEQ ID NO: 421) | | | | |
| M4C02 | IGKV1- | IGKJ4* | QTISTY | AAS | QQTYSSLALT | 6, 3, 10 | 11 | 96.113 | 92.6 |
| | 39*01 | 01 | (SEQ ID NO: 422) | (SEQ ID NO: 423) | (SEQ ID NO: 424) | | | | |
| M1A04 | IGKV1- | IGKJ2* | QTIRSY | AAT | QQSFSTLYS | 6, 3, 9 | 8 | 97.173 | 93.6 |
| | 39*01 | 03 | (SEQ ID NO: 425) | (SEQ ID NO: 426) | (SEQ ID NO: 427) | | | | |
| MIC11 | IGKV4- | IGKJ2* | QSVLYSSNSKSY | WAS | QQFYTSPHT | 12, 3, 9 | 6 | 98.026 | 94.1 |
| | 1*01 | 01 | (SEQ ID NO: 428) | (SEQ ID NO: 429) | (SEQ ID NO: 430) | | | | |
| M2A11 | IGLV1- | IGLJ3* | SSDIGAGSD | GNT | QSYDSGLSGYV | 9, 3, 11 | 8 | 97.315 | 96 |
| | 40*01 | 02 | (SEQ ID NO: 431) | (SEQ ID NO: 432) | (SEQ ID NO: 433) | | | | |
| M2B01 | IGKV3- | IGKJ1* | QSVTSN | GAS | QQYNAWPPWT | 6, 3, 10 | 5 | 98.258 | 97.9 |
| | 15*01 | 01 | (SEQ ID NO: 434) | (SEQ ID NO: 435) | (SEQ ID NO: 436) | | | | |
| M2B12 | IGLV4- | IGLJ1* | SGPSNYA | VNSDGSH | QTWGSGIYV | 7, 7, 9 | 17 | 94.276 | 88.9 |
| | 69*01 | 01 | (SEQ ID NO: 437) | (SEQ ID NO: 438) | (SEQ ID NO: 439) | | | | |
| M2C05 | IGKV1- | IGKJ2* | QSVSNW | KAS | QHYNGYPYT | 6, 3, 9 | 15 | 94.662 | 88.3 |
| | 5*03 | 01 | (SEQ ID NO: 440) | (SEQ ID NO: 441) | (SEQ ID NO: 442) | | | | |
| M2D01 | IGKV4- | IGKJ2* | QSLLYTSNNKNS | WAS | QQYYTAPYT | 12, 3, 9 | 17 | 94.371 | 90.1 |
| | 1*01 | 01 | (SEQ ID NO: 443) | (SEQ ID NO: 444) | (SEQ ID NO: 445) | | | | |
| M2D2 | IGLV1- | IGLJ1* | NSNIGSST | SDN | GTWDDGLNGPYV | 8, 3, 12 | 19 | 93.581 | 89.8 |
| | 44*01 | 01 | (SEQ ID NO: 446) | (SEQ ID NO: 447) | (SEQ ID NO: 448) | | | | |
| M3C05 | IGLV1- | IGLJ3* | SSDIGSNS | SSI | ATWDDSLNGPV | 8, 3, 11 | 8 | 97.297 | 93.9 |
| | 44*01 | 02 | (SEQ ID NO: 449) | (SEQ ID NO: 450) | (SEQ ID NO: 451) | | | | |
| M3C12 | IGLV1- | IGLJ2* | SSNIGRNP | KNS | AAWDDSLNGPV | 8, 3, 11 | 9 | 96.959 | 93.9 |
| | 44*01 | 01 | (SEQ ID NO: 452) | (SEQ ID NO: 453) | (SEQ ID NO: 454) | | | | |
| M3D09 | IGKV4- | IGKJ2* | QSVLFSPNNKNY | WAS | QQYNSNPYT | 12, 3, 9 | 11 | 96.358 | 94.1 |
| | 1*01 | 01 | (SEQ ID NO: 455) | (SEQ ID NO: 456) | (SEQ ID NO: 457) | | | | |
| M3D12 | IGKV3- | IGKJ4* | RSVRNY | DAS | QQRSNWGLT | 6, 3, 9 | 9 | 96.809 | 93.6 |
| | 11*01 | 01 | (SEQ ID NO: 458) | (SEQ ID NO: 459) | (SEQ ID NO: 460) | | | | |
| M3F03 | IGKV3 | IGKJ1* | QSVTNN | GAS | QQYNGWPPWT | 6, 3, 10 | 7 | 97.561 | 94.7 |
| | D- | 01 | (SEQ ID NO: 461) | (SEQ ID NO: 462) | (SEQ ID NO: 463) | | | | |
| | 15*01 | | | | | | | | |
| M4B01 | IGLV1- | IGLJ2* | TSNIGNSY | EDN | GTWDNSLTEVV | 8, 3, 11 | 16 | 94.521 | 90.7 |
| | 51*02 | 01 | (SEQ ID NO: 464) | (SEQ ID NO: 465) | (SEQ ID NO: 466) | | | | |
| M4C03 | IGLV1- | IGLJ3* | TSNIGAGFD | ANT | QTYDTTLSGSTV | 9, 3, 12 | 19 | 93.645 | 86.9 |
| | 40*01 | 02 | (SEQ ID NO: 467) | (SEQ ID NO: 468) | (SEQ ID NO: 469) | | | | |
| M4C04 | IGKV3- | IGKJ1* | QSVRSN | GAS | QQYNNWPPGA | 6, 3, 10 | 6 | 97.909 | 95.8 |
| | 15*01 | 01 | (SEQ ID NO: 470) | (SEQ ID NO: 471) | (SEQ ID NO: 472) | | | | |
| M4C08 | IGLV1- | IGLJ3* | TSNIGAGFD | ANT | QTYDRSLSGSTV | 9, 3, 12 | 14 | 95.318 | 89.9 |
| | 40*01 | 02 | (SEQ ID NO: 473) | (SEQ ID NO: 474) | (SEQ ID NO: 475) | | | | |
| M2A02 | IGKV3- | IGKJ4* | QRIGSY | DAS | QQRTDWFT | 6, 3, 8 | 9 | 96.809 | 91.5 |
| | 11*01 | 01 | (SEQ ID NO: 476) | (SEQ ID NO: 477) | (SEQ ID NO: 478) | | | | |
| M2A05 | IGKV3- | IGKJ1* | RSVNSDY | AAS | QQYGSAVWT | 7, 3, 9 | 27 | 90.426 | 82.1 |
| | 20*01 | 01 | (SEQ ID NO: 479) | (SEQ ID NO: 480) | (SEQ ID NO: 481) | | | | |
| M2A06 | IGKV1- | IGKJ1* | QSISSR | DAS | QHYSIYSRWT | 6, 3, 10 | 10 | 96.479 | 90.5 |
| | 5*01 | 01 | (SEQ ID NO: 482) | (SEQ ID NO: 483) | (SEQ ID NO: 484) | | | | |
| M2B10 | IGKV3- | IGKJ3* | QSISSNS | GTS | QQYGSSPLT | 7, 3, 9 | 7 | 97.578 | 93.8 |
| | 20*01 | 01 | (SEQ ID NO: 485) | (SEQ ID NO: 486) | (SEQ ID NO: 487) | | | | |
| M2D07 | IGKV1- | IGKJ2* | QSISND | SAS | QQSYTTPPL | 6, 3, 9 | 18 | 93.728 | 88.4 |
| | 39*01 | 01 | (SEQ ID NO: 488) | (SEQ ID NO: 489) | (SEQ ID NO: 490) | | | | |
| M2D10 | IGKV1- | IGKJ1* | QTISNF | AAL | QQSYITPWT | 6, 3, 9 | 20 | 92.958 | 87.4 |
| | 39*01 | 01 | (SEQ ID NO: 491) | (SEQ ID NO: 492) | (SEQ ID NO: 493) | | | | |
| M2F01 | IGKV1- | IGKJ3* | QGIRSW | AAS | QQSNSFSLFT | 6, 3, 10 | 5 | 98.227 | 96.8 |
| | 12*01 | 01 | (SEQ ID NO: 494) | (SEQ ID NO: 495) | (SEQ ID NO: 496) | | | | |
| M4B04 | IGKV3- | IGKJ3* | QSLSGN | GTS | QQYNNWPPVFT | 6, 3, 11 | 7 | 97.561 | 95.8 |
| | 15*01 | 01 | (SEQ ID NO: 497) | (SEQ ID NO: 498) | (SEQ ID NO: 499) | | | | |
| M4B05 | IGKV3- | IGKJ4* | QLINGNY | DVS | QHYGRSPSPT | 7, 3, 10 | 24 | 91.667 | 82.3 |
| | 20*01 | 01 | (SEQ ID NO: 500) | (SEQ ID NO: 501) | (SEQ ID NO: 502) | | | | |
| M4B11 | IGKV3- | IGKJ2* | QSISNF | DVS | QQRSNWPPGYT | 6, 3, 11 | 13 | 95.47 | 90.5 |
| | 11*01 | 01 | (SEQ ID NO: 503) | (SEQ ID NO: 504) | (SEQ ID NO: 505) | | | | |
| M4F8 | IGLV1- | IGLJ2* | SSNIGATYD | GNN | QSYDSVLSGSVI | 9, 3, 12 | 17 | 94.138 | 93.9 |
| | 40*01 | 01 | (SEQ ID NO: 506) | (SEQ ID NO: 507) | (SEQ ID NO: 508) | | | | |
| M2A03 | IGLV3- | IGLJ1* | KLGSKY | QDT | QAWDSSSFV | 6, 3, 9 | 12 | 95.745 | 92.6 |
| | 1*01 | 01 | (SEQ ID NO: 509) | (SEQ ID NO: 510) | (SEQ ID NO: 511) | | | | |
| M3A07 | IGKV1- | IGKJ2* | QSISSW | KAF | QQYGSFSEFT | 6, 3, 10 | 9 | 96.831 | 93.7 |
| | 5*03 | 01 | (SEQ ID NO: 512) | (SEQ ID NO: 513) | (SEQ ID NO: 514) | | | | |
| M5F2 | IGLV2- | IGLJ3* | SGDVGGYTF | EVT | SSYSSTNNLRV | 9, 3, 11 | 16 | 94.595 | 88.8 |
| | 8*01 | 02 | (SEQ ID NO: 515) | (SEQ ID NO: 516) | (SEQ ID NO: 517) | | | | |
| M4B03 | IGLV3- | IGLJ7* | NIGSKS | DDS | HVSDDNSDHVF | 6, 3, 11 | 11 | 93.403 | 87.5 |
| | 21*02 | 01 | (SEQ ID NO: 518) | (SEQ ID NO: 519) | (SEQ ID NO: 520) | | | | |
| M4B06 | IGLV1- | IGLJ1* | GSNIGNNY | NTD | AAWDDTLNGYV | 8, 3, 11 | 16 | 90.136 | 78.6 |
| | 44*01 | 01 | (SEQ ID NO: 521) | (SEQ ID NO: 522) | (SEQ ID NO: 523) | | | | |
| M2C08 | IGKV3- | IGKJ4* | QSVSRTY | GTS | QHFGSSLT | 7, 3, 8 | 7 | 95.804 | 90.5 |
| | 20*01 | 01 | (SEQ ID NO: 524) | (SEQ ID NO: 525) | (SEQ ID NO: 526) | | | | |
| M3D04 | IGKV3- | IGKJ1* | HSVSSSY | GAS | QQYLTSPPGWT | 7, 3, 11 | 20 | 97.586 | 95.8 |
| | 20*01 | 01 | (SEQ ID NO: 527) | (SEQ ID NO: 528) | (SEQ ID NO: 529) | | | | |
| MIC7 | IGKV3- | IGKJ1* | QSISNF | DAS | QQRSTWT | 6, 3, 7 | 21 | 95.39 | 92.6 |
| | 11*01 | 01 | (SEQ ID NO: 530) | (SEQ ID NO: 531) | (SEQ ID NO: 532) | | | | |
| M2B11 | IGLV3- | IGLJ2* | ELGHKY | QDT | QAWDSGTVL | 6, 3, 9 | 7 | 92.933 | 89.4 |
| | 1*01 | 01 | (SEQ ID NO: 533) | (SEQ ID NO: 534) | (SEQ ID NO: 535) | | | | |
| M2C11 | IGLV3- | IGLJ1* | DLGNQY | QDN | QAWDSRTYV | 6, 3, 9 | 15 | 93.617 | 87.2 |
| | 1*01 | 01 | (SEQ ID NO: 536) | (SEQ ID NO: 537) | (SEQ ID NO: 538) | | | | |
| M2D08 | IGKV3- | IGKJ2* | QNIYNY | DAS | QQRSDWT | 6, 3, 7 | 12 | 96.14 | 92.6 |
| | 11*01 | 01 | (SEQ ID NO: 539) | (SEQ ID NO: 540) | (SEQ ID NO: 541) | | | | |
| M3C06 | IGKV4- | IGKJ5* | LSLLYSSNNKNY | WAS | QQYYSTPIT | 12, 3, 9 | 7 | 96.026 | 93.1 |
| | 1*01 | 01 | (SEQ ID NO: 542) | (SEQ ID NO: 543) | (SEQ ID NO: 544) | | | | |
| M4A10 | IGLV6- | IGLJ2* | TGNIASNY | EDN | QSYDSGHHVV | 8, 3, 10 | 11 | 96.622 | 92.9 |
| | 57*01 | 01 | (SEQ ID NO: 545) | (SEQ ID NO: 546) | (SEQ ID NO: 547) | | | | |
| M1A08 | IGKV4- | IGKJ4* | ESVLYNFNNKNY | WAS | HQFYSTHS | 12, 3, 8 | 8 | 96.346 | 94 |
| | 1*01 | 01 | (SEQ ID NO: 548) | (SEQ ID NO: 549) | (SEQ ID NO: 550) | | | | |
| M2B07 | IGKV4- | IGKJ2* | ESIFYYSNSKSY | WAS | QQYYTNPRT | 12, 3, 9 | 9 | 94.702 | 90.1 |
| | 1*01 | 01 | (SEQ ID NO: 551) | (SEQ ID NO: 552) | (SEQ ID NO: 553) | | | | |
| M2B09 | IGLV3- | IGLJ1* | NIGSKS | DDS | HVWDSYSDPNYV | 6, 3, 12 | 13 | 97.552 | 95.8 |
| | 21*02 | 01 | (SEQ ID NO: 554) | (SEQ ID NO: 555) | (SEQ ID NO: 556) | | | | |
| M2E04 | IGLV1- | IGLJ2* | RSNMGINW | RNS | AVWDDSLDGRL | 8, 3, 11 | 11 | 93.22 | 88.8 |
| | 47*01 | 01 | (SEQ ID NO: 557) | (SEQ ID NO: 558) | (SEQ ID NO: 559) | | | | |
| M2E05 | IGLV1- | IGLJ2* | SSNMGINW | RNS | AVWDDVVSGRL | 8, 3, 11 | 11 | 92.881 | 88.8 |
| | 47*01 | 01 | (SEQ ID NO: 560) | (SEQ ID NO: 561) | (SEQ ID NO: 562) | | | | |
| M2E06 | IGKV1- | IGKJ3* | QGVRND | AAS | LQVYSYPFT | 6, 3, 9 | 0 | 97.544 | 93.7 |
| | 6*01 | 01 | (SEQ ID NO: 563) | (SEQ ID NO: 564) | (SEQ ID NO: 565) | | | | |
| M4A08 | IGKV4- | IGKJ1* | ESVLYTSNSKNY | WAS | QQYFTIPRT | 12, 3, 9 | 13 | 95.066 | 90.1 |
| | 1*01 | 01 | (SEQ ID NO: 566) | (SEQ ID NO: 567) | (SEQ ID NO: 568) | | | | |
| M2E11 | IGLV8- | IGLJ3* | SGSVSTSHF | STI | VLYLGGGIPHWV | 9, 3, 12 | 4 | 95.904 | 89.8 |
| L | 61*01 | 02 | (SEQ ID NO: 569) | (SEQ ID NO: 570) | (SEQ ID NO: 571) | | | | |
| M1A11 | IGKV1- | IGKJ1* | QSISTY | VAS | QQGYSIPWT | 6, 3, 9 | 5 | 97.535 | 95.8 |
| | 39*01 | 01 | (SEQ ID NO: 572) | (SEQ ID NO: 573) | (SEQ ID NO: 574) | | | | |
| M2C10 | IGKV1- | IGKJ4* | QTISGY | TAS | QQSYRMPLT | 6, 3, 9 | 0 | 96.043 | 91.6 |
| | 39*01 | 01 | (SEQ ID NO: 575) | (SEQ ID NO: 576) | (SEQ ID NO: 577) | | | | |
| M2E01 | IGKV1- | IGKJ2* | QTITTY | AAS | QQSYSTPYT | 6, 3, 9 | 2 | 97.183 | 93.7 |
| | 39*01 | 01 | (SEQ ID NO: 578) | (SEQ ID NO: 579) | (SEQ ID NO: 580) | | | | |
| M2E08 | IGLV1- | IGLJ2* | NFNIGAGYD | GNT | QSYDSSLSMV | 9, 3, 10 | 9 | 96.939 | 91.8 |
| | 40*01 | 01 | (SEQ ID NO: 581) | (SEQ ID NO: 582) | (SEQ ID NO: 583) | | | | |
| M3A04 | IGKV1- | IGKJ2* | QSISSY | TVS | QQSYNIPYT | 6, 3, 9 | 11 | 95.423 | 93.7 |
| | 39*01 | 01 | (SEQ ID NO: 584) | (SEQ ID NO: 585) | (SEQ ID NO: 586) | | | | |
| M3A12 | IGKV1- | IGKJ2* | QSIGTY | AAS | QQSYTKPYT | 6, 3, 9 | 8 | 96.071 | 91.6 |
| | 39*01 | 01 | (SEQ ID NO: 587) | (SEQ ID NO: 588) | \(SEQ ID NO: 589) | | | | |
| M3C07 | IGKV3- | IGKJ2* | QTVSSDY | DAS | QQYGSPYT | 7, 3, 8 | 1 | 96.085 | 91.5 |
| | 20*01 | 01 | (SEQ ID NO: 590) | (SEQ ID NO: 591) | (SEQ ID NO: 592) | | | | |
| M3D03 | IGKV1- | IGKJ3* | QSISSY | AAS | QQSYSTPRT | 6, 3, 9 | 1 | 100 | 100 |
| | 39*01 | 01 | (SEQ ID NO: 593) | (SEQ ID NO: 594) | (SEQ ID NO: 595) | | | | |
| M8C10 | IGKV1- | IGKJ2* | QTITIW | DAV | QQYNNYPYT | 6, 3, 9 | 9 | 95.374 | 92.6 |
| | 5*01 | 01 | (SEQ ID NO: 596) | (SEQ ID NO: 597) | (SEQ ID NO: 598) | | | | |
| M3A03 | IGLV3- | IGLJ2* | NIGSKS | DDS | QVWDSSGDRVV | 6, 3, 11 | 18 | 98.601 | 97.9 |
| | 21*02 | 01 | (SEQ ID NO: 599) | (SEQ ID NO: 600) | (SEQ ID NO: 601) | | | | |
| M3A05 | IGLV1- | IGLJ1* | SSNIGNNY | DNN | GTWDSSLSVGFYV | 8, 3, 13 | 0 | 98.311 | 97.9 |
| | 51*01 | 01 | (SEQ ID NO: 602) | (SEQ ID NO: 603) | (SEQ ID NO: 604) | | | | |
| M3A09 | IGKV3- | IGKJ1* | QSVSSY | DAS | QQRSNWPPKWT | 6, 3, 11 | 24 | 100 | 100 |
| | 11*01 | 01 | (SEQ ID NO: 605) | (SEQ ID NO: 606) | (SEQ ID NO: 607) | | | | |
| M3A11 | IGLV3- | IGLJ3* | NIGSKS | DDS | QVWHSSSDWV | 6, 3, 10 | 0 | 99.298 | 98.9 |
| | 21*02 | 02 | (SEQ ID NO: 608) | (SEQ ID NO: 609) | (SEQ ID NO: 610) | | | | |
| M3B04 | IGKV3- | IGKJ5* | QSINRN | DAS | QQRSHWVT | 6, 3, 8 | 4 | 96.809 | 93.6 |
| | 11*01 | 01 | (SEQ ID NO: 611) | (SEQ ID NO: 612) | (SEQ ID NO: 613) | | | | |
| M3B06 | IGKV1- | IGKJ1* | QTINNW | KAS | QQYISYSRT | 6, 3, 9 | 7 | 96.127 | 94.7 |
| | 5*03 | 01 | (SEQ ID NO: 614) | (SEQ ID NO: 615) | (SEQ ID NO: 616) | | | | |
| M3B07 | IGKV3- | IGKJ1* | QSVSSSY | STF | QQYGSSPRT | 7, 3, 9 | 20 | 97.232 | 91.7 |
| | 20*01 | 01 | (SEQ ID NO: 617) | (SEQ ID NO: 618) | (SEQ ID NO: 619) | | | | |
| M3B12 | IGKV3- | IGKJ4* | QSVSSY | DAS | QQRSNWPPLT | 6, 3, 10 | 13 | 99.652 | 100 |
| | 11*01 | 01 | (SEQ ID NO: 620) | (SEQ ID NO: 621) | (SEQ ID NO: 622) | | | | |
| M3C01 | IGKV1- | IGKJ4* | QDISSW | AAS | QQANSFPLT | 6, 3, 9 | 12 | 99.65 | 98.9 |
| | 12*01 | 01 | (SEQ ID NO: 623) | (SEQ ID NO: 624) | (SEQ ID NO: 625) | | | | |
| M3D06 | IGKV1- | IGKJ4* | QGISSA | DAS | QQFNSYPLT | 6, 3, 9 | 8 | 96.831 | 96.8 |
| | 13*02 | 01 | (SEQ ID NO: 626) | (SEQ ID NO: 627) | (SEQ ID NO: 628) | | | | |
| M3D08 | IGKV1- | IGKJ5* | QSIGTH | AAS | QQSYSTPSIT | 6, 3, 10 | 13 | 93.684 | 90.5 |
| | 39*01 | 01 | (SEQ ID NO: 629) | (SEQ ID NO: 630) | (SEQ ID NO: 631) | | | | |
| M3E02 | IGKV3- | IGKJ2* | QSVSSSY | GAS | QQYGSSPT | 7, 3, 8 | 17 | 100 | 100 |
| | 20*01 | 01 | (SEQ ID NO: 632) | (SEQ ID NO: 633) | (SEQ ID NO: 634) | | | | |
| M3E05 | IGLV3- | IGLJ2* | NIGVRN | RDN | QVWDSNTAHVI | 6, 3, 11 | 5 | 91.549 | 85.4 |
| | 9*01 | 01 | (SEQ ID NO: 635) | (SEQ ID NO: 636) | (SEQ ID NO: 637) | | | | |
| M3E06 | IGKV3- | IGKJ2* | QSVSSN | GAS | QQYNNWPPYT | 6, 3, 10 | 7 | 100 | 100 |
| | 15*01 | 01 | (SEQ ID NO: 638) | (SEQ ID NO: 639) | (SEQ ID NO: 640) | | | | |
| M3E08 | IGLV1- | IGLJ1* | SSNIGRNY | RNN | AAWDDSLSGYV | 8, 3, 11 | 0 | 98.639 | 96.9 |
| | 47*01 | 01 | (SEQ ID NO: 641) | (SEQ ID NO: 642) | (SEQ ID NO: 643) | | | | |
| M1A01 | IGKV3- | IGKJ5* | QSLSRY | DAS | HQRHNWIT | 6, 3, 8 | 35 | 97.518 | 94.7 |
| | 11*01 | 01 | (SEQ ID NO: 644) | (SEQ ID NO: 645) | (SEQ ID NO: 646) | | | | |
| M1A12 | IGKV3- | IGKJ2* | LRLSTN | DAS | HQHSNWYT | 6, 3, 8 | 1 | 92.908 | 87.4 |
| | 11*01 | 01 | (SEQ ID NO: 647) | (SEQ ID NO: 648) | (SEQ ID NO: 649) | | | | |
| M2B04 | IGKV3- | IGKJ4* | QNINNF | DAS | QQRANWPLT | 6, 3, 9 | 19 | 95.423 | 91.6 |
| | 11*01 | 01 | (SEQ ID NO: 650) | (SEQ ID NO: 651) | (SEQ ID NO: 652) | | | | |
| M2C01 | IGKV4- | IGKJ1* | QSVLYTSNNRNF | WAS | QQYYSIPPM | 12, 3, 9 | 29 | 96.066 | 94.1 |
| | 1*01 | 01 | (SEQ ID NO: 653) | (SEQ ID NO: 654) | (SEQ ID NO: 655) | | | | |
| M2D02 | IGKV3- | IGKJ1* | QSVTSN | GAS | QQYDHWPRT | 6, 3, 9 | 12 | 97.203 | 91.6 |
| | 15*01 | 01 | (SEQ ID NO: 656) | (SEQ ID NO: 657) | (SEQ ID NO: 658) | | | | |
| M2D09 | IGKV1- | IGKJ5* | QSMSNS | AAS | QQSYSTPIT | 6, 3, 9 | 7 | 95.423 | 92.6 |
| | 39*01 | 01 | (SEQ ID NO: 659) | (SEQ ID NO: 660) | (SEQ ID NO: 661) | | | | |
| M2E07 | IGLV2- | IGLJ2* | SSNIGSFDL | EGD | CSYARSRTLV | 9, 3, 10 | 13 | 94.257 | 85.9 |
| | 23*01 | 01 | (SEQ ID NO: 662) | (SEQ ID NO: 663) | (SEQ ID NO: 664) | | | | |
| M3E01 | IGKV1- | IGKJ1* | QGISSY | AAS | QQLKSYPGT | 6, 3, 9 | 20 | 98.246 | 95.8 |
| | 9*01 | 01 | (SEQ ID NO: 665) | (SEQ ID NO: 666) | (SEQ ID NO: 667) | | | | |
| M3E09 | IGKV3- | IGKJ1* | QSVSSNY | DAS | QQYGSSPET | 7, 3, 9 | 18 | 97.569 | 94.8 |
| | 20*01 | 01 | (SEQ ID NO: 668) | (SEQ ID NO: 669) | (SEQ ID NO: 670) | | | | |
| M3F02 | IGLV3- | IGLJ2* | KLGDKY | QDS | QAWDSSTAV | 6, 3, 9 | 11 | 100 | 100 |
| | 1*01 | 01 | (SEQ ID NO: 671) | (SEQ ID NO: 672) | (SEQ ID NO: 673) | | | | |
| M4A11 | IGLV1- | IGLJ2* | TSNIGRNF | GND | ATWDDSVNGPV | 8, 3, 11 | 12 | 88.176 | 82.7 |
| | 47*01 | 01 | (SEQ ID NO: 674) | (SEQ ID NO: 675) | (SEQ ID NO: 676) | | | | |
| M4B02 | IGKV1- | IGKJ5* | QSISSY | AAS | QQSYSTPNT | 6, 3, 9 | 10 | 99.649 | 98.9 |
| | 39*01 | 01 | (SEQ ID NO: 677) | (SEQ ID NO: 678) | (SEQ ID NO: 679) | | | | |

nt sub—nucleotide substitution; nt ID%—nt identity percentage; amino acid identity percentage.

TABLE 3D
Sequences of antibody VHs and VLs that bind
human metapneumovirus

| | SEQ | |
|---|---|---|
| | ID | |
| Identifier | NO | Amino acid sequence |
| M1A01_VH | 680 | QVQLVQSGAEVKKPGSSVKVSCWASGGTFD |
| | | NAINWLRQAPGQGPEWMGRIIPLVDIVNRA |
| | | PKFQDRVSFTADKSTNTVYMVLNSLTSDDT |
| | | AVYYCASAFQDGWFDPWGQGTLVTVSS |
| M1A01_VK | 681 | EIVLTQSPATLSLSPGERATLSCRASQSLS |
| | | RYLAWYQQKPGQAPRLLIYDASNRATGVPA |
| | | RFSGSGSGTDFTLTISSLEPEDFAVYYCHQ |
| | | RHNWITFGQGTRLEIK |
| M1A04_VH | 682 | EVQLVESGGGLVRPGGSLRLSCAASGFNVR |
| | | NYYMSWVRQAPGKGLEWISVIYGGGTTFYA |
| | | GSVKGRFTISRDDSKSTLFLQMNSLRADDT |
| | | AVYYCARDPGTVMSTENLQGMDVWGQGTTV |
| | | TVSS |
| M1A04_VK | 683 | DIQMTQSPSSLSASVGDRVTITCRASQTIR |
| | | SYLNWYQQKPGKAPNLLIYAATSLQSGVPS |
| | | RFSGSGSGTDFTLTISSLQPEDSATYYCQQ |
| | | SFSTLYSFGQGTRLEIK |
| M1A08_VH | 684 | QVQLVQSGAEVKKPGASVKVSCKASGVTLT |
| | | DYYFHWIRQAPGQALEWMGRINPARGGTHY |
| | | AEKFEDRVTMTTDTSTNTVYMDLSSLRSDD |
| | | TAVYYCARSGSTHNIYSGFYAFDHWGQGTL |
| | | VTVSS |
| M1A08_VK | 685 | DIVMTQSPDSLAVSLGERATINCKSSESVL |
| | | YNFNNKNYLAWYQQKPGQPPKLLIYWASTR |
| | | ESGVPERFSGSGSGTDFTLTISSLQAEDVA |
| | | VYYCHQFYSTHSFGGGTKVEIK |
| MIA11_VH | 686 | QLQLQESGPGLVKPSETLSLTCTVSGDSIS |
| | | SSSFYWGWIRQPPGKGLEWIATIYYTGSTE |
| | | YNPSLKSRISISVDTSMNQFSLRLRSVTAA |
| | | DTAVYYCARQEGSSWSNWFDPWGQGTLVTV |
| | | SS |
| M1All_VK | 687 | DIQMTQSPSSLSASVGDRVTITCRASQSIS |
| | | TYLNWYQQKPGKAPKLLIYVASSLQSGVPS |
| | | RFSGSGSGTDFTLTISSLQPEDFATYYCQQ |
| | | GYSIPWTFGHGTKVEIK |
| M1A12_VH | 688 | QVQLVQSGTEVKKPGSSVKVSCKDPGDIMS |
| | | SHSIHWVRQAPGQGPEWMGRIIPLFGMKSY |
| | | APNFQGRVSISVDTSTNTAYLELSGLKSDD |
| | | TAVYFCAREENLSWLDYWGQGTQVIVSS |
| M1A12_VK | 689 | EIVLTQSPATLSLSPGDRATLSCRASLRLS |
| | | TNIAWYQQKPGQAPRLVIYDASKRATGIPA |
| | | RFSGSGSGTDFTLTISSLEPEDFAVYYCHQ |
| | | HSNWYTFGQGTKLEIK |
| M2A02_VH | 690 | QVQLVQSGAEVKRPGSSVKVSCKASGDIFS |
| | | SHAISWVRQTPGQGLKWMGRIIPLVGIRNY |
| | | AQDLQGRVTISADKSINTVYMELTNLRFDD |
| | | TAVYYCARDRTDWSGGLDIWGQGTRVTVSS |
| M2A02_VK | 691 | EIVLTQSPATLSLSPGERATLSCRASQRIG |
| | | SYLAWYQQKPGQAPRLLIYDASNRATGIPA |
| | | RFSGSGSGTDFTLTISRLEPEDSAIYYCQQ |
| | | RTDWFTFGGGTKVEIK |
| M2A03_VH | 692 | QLQLQESGPGLVKPSETLSLTCSVSGGSIS |
| | | SSSNSWTWIRQPPGKGLEWIATIFYFGSTY |
| | | YNPSLRSRVAISVDTSENQFSLKLTSVTAA |
| | | DTAVYFCARHILSSSGVSGWLDPWGQGTLV |
| | | TVSS |
| M2A03_VL | 693 | SYELTQPPSVSVSPGQTASITCSGEKLGSK |
| | | YAHWYQQKPGRSPVLVIYQDTKRPSGIPER |
| | | FSGSNSGSTATLTISGTQAMDEADYYCQAW |
| | | DSSSFVFGTGTTVTVL |
| M2A05_VH | 694 | QITLKESGPTLVKPTQTLTLTCTVSGFSLS |
| | | ANSVGVAWFRQPPGKAPEWLGVIYWDDDKR |
| | | FSPSLKTRLSITKDASTSQVVLRLTNVSPL |
| | | DTATYYCVRKHYDLLQWGQGAPVTVSS |
| M2A05_VK | 695 | EIVLTQSPGTLSLSPGEGATLSCRASRSVN |
| | | SDYIAWYQHKPGLAPRLLIYAASTRATDIP |
| | | DRFRADVSGTDFTLTIDRLEPDDFAVYYCQ |
| | | QYGSAVWTFGQGTRVEVK |
| M2A06_VH | 696 | QVQLVQSGAEVKKPGASVKISCKASGYSFS |
| | | NYYIQWVRQAPGQGLEWLGIINPSGGSTDY |
| | | AQKFEGRVSMTRDTSTNTVYMEMSSLRSED |
| | | TASYYCVRSLANGSGSPWGQGTLVIVSS |
| M2A06_VK | 697 | DIQMTQSPSTLSASVGDRVTITCRASQSIS |
| | | SRLAWYQQKPGKAPNLLLYDASRSESGVPS |
| | | RFSGSGSGTEFTLTISSLQPDDVATYYCQH |
| | | YSIYSRWTFGQGTKVEIK |
| M2A09_VH | 698 | QVQLVQSGAGVRKPGSSVKVSCKASGGTFS |
| | | NYIISWVRQAPGQGLEYIGRIIPIVGMEKY |
| | | EEKFQGRVKITADESTSTVSMELTSLKSED |
| | | TATYYCATEGLESGYPPYLEHWGRGTLVIV |
| | | SS |
| M2A09_VK | 699 | EIVLTQSPGTLSLSPGERATLSCRTSQTVG |
| | | AHLAWYQQKPGQSPRLLIYGASSRATGIPD |
| | | RFSGSGSGTVFTLTINRLEPEDLAVYYCQQ |
| | | YGASPPTFGQGTKVEIK |
| M2A11_VH | 700 | QVQLQESGPGLVKPSETLSLTCTVSGGSIT |
| | | SHYWSWVRQPPGKGLEWIAYIYYSGSTDYN |
| | | PSLKSRVTISLDTSKNQVSLRLNSVTTADT |
| | | AVYFCARCMPLYHVMHVWGHGTTVTVSS |
| M2A11_VL | 701 | QSVLTQPPSVSGAPGQRVTISCTGSSSDIG |
| | | AGSDVHWYQQLPGTAPKLLIYGNTNRPSGV |
| | | PDRFSGSKSGTSASLAITGLQAEDEADYYC |
| | | QSYDSGLSGYVFGGGTKLTVL |
| M2B01_VH | 702 | QVQLVESGGGVVQPGKSLRLSCAASGFTFS |
| | | SHAMDWVRQVPGKGLEWVAVISYDGSNRYY |
| | | ADSVKGRFTISRDTSKNTLYLQMNSLRPED |
| | | TAVYYCARDFQPSPMVYGVGYFDYWGQGTL |
| | | VTVSP |
| M2B01_VK | 703 | EIVMTQSPATLSVSPGERATLSCRASQSVT |
| | | SNLAWYQQKPGQAPRLLIYGASTRATGIPA |
| | | RFSGSGSGTEFTLTISSLQSEDFAVYYCQQ |
| | | YNAWPPWTFGQGTKVEIR |
| M2B04_VH | 704 | QVQLVQSGPEVKKPGASVKVSCKASGYSIT |
| | | TYGIDWVRQAPGQGLEWIGWSSAHTGNTNY |
| | | AEKFQGRVTMTTDTSTYTTYMELRSLKSDD |
| | | TAVYYCAMGAYTSSRNWFDPWGQGTLVIVS |
| | | S |
| M2B04_VK | 705 | EIVLTQSPATLSLSPGERATLSCRASQNIN |
| | | NFLAWYQQKAGQAPRLLIYDASNRATGIPG |
| | | RFSGSGSGTDFTLTISSLEPEDFAVYYCQQ |
| | | RANWPLTFGGGTKVEVK |
| M2B05_VH | 706 | QVQLQQWGAGLLRPSETLSLTCTVYGGSFG |
| | | GYHWSWIRQSPGKGLEWIGEIDHGGNTNFN |
| | | PSLKSRVTISVDRSKKQFSLKLTSVTAADT |
| | | AVYYCARGFGVVINNWFDPWGQGTLVTVSS |
| M2B05_VK | 707 | DIQMTQSPSSLSASVGDRVTITCRASQTIN |
| | | SYLNWYQQKPGKAPTLLIYTVSSLQSGVPS |
| | | RFSGSGSGTVFSLTISSLQPEDFATYYCQQ |
| | | SYNSPYTFGQGTKLEIK |
| M2B06_VH | 708 | QVQLQESGPRLVKPSGTLSLTCAVSGASIT |
| | | SSHWWTWVRQPPGKGLEWIGRIFHSGSTNY |
| | | NPSLKSRVTISLDKSRNQFSLTLTSVTAAD |
| | | TAVYYCARDDQIVVMPAGFDRWGQGALVIV |
| | | SS |
| M2B06_VK | 709 | DIQMTQSPSSLSASVGDRVTITCRASQTIS |
| | | SYLNWYQQTPGKAPNLLIYGASRLQGGVPS |
| | | RFSGSGSGTDFTLTISSLQPEDFAIYFCQQ |
| | | SKSIPYTFGQGTRLEIK |
| M2B07_VH | 710 | EVQLVESGGGLVRPGGSLRLSCAASGFTFN |
| | | NYWMHWVRQAPGKGLVWVSHVKSDGSITNY |
| | | ADSVKGRFTISRDNAKNTLYLEMSSLRVED |
| | | TAVYYCARESRPYDPLTGSHPDAFDIWGQG |
| | | TMVTVSS |
| M2B07_VK | 711 | DIVMTQSPDSLAVSLGERATINCKSSESIF |
| | | YYSNSKSYLAWYRQKPGQPPKLLIYWASTR |
| | | ESGVPDRFSGSGSGTDFTLTISSLQPEDVA |
| | | VYYCQQYYTNPRTFGQGTRLEIK |
| M2B09_VH | 712 | QVQLVQSGPEVKGPGASVRVSCKANGYTFT |
| | | SYGINWVRQTPGQGLQWMGWISVHTDDTKD |
| | | VQQFQDRVTMTTDTSTSTVFMELRNLRFDD |
| | | TAVYYCARDRTDISGRVRDAFDLWGQGTLV |
| | | TVSS |
| M2B09_VL | 713 | SYVLTQPPSVSVAPGQTAKITCGGNNIGSK |
| | | SVHWYRQKPGQAPVLVVYDDSDRPSGIPER |
| | | FSGSNSGNTATLTISRVEAGDEADYYCHVW |
| | | DSYSDPNYVFGSGTKVTVL |
| M2B10_VH | 714 | QVQLVESGGGVVQPGRSLRLSCAASGFTFS |
| | | NYPMHWVRQAPGKGLAWVALISYDGRTKYY |
| | | ADSVKGRFTISRDNSMNTVYLQMNSLRAED |
| | | TAVYYCAREGLEYLNYWGQGTLVTVSS |
| M2B10_VK | 715 | EIVLTQSPGTLSLSPGERATLSCRASQSIS |
| | | SNSLAWYQQKPGQAPRLLSYGTSNRATGIP |
| | | DRFSGSGSGTDFTLTISRLEPEDFAVYYCQ |
| | | QYGSSPLTFGPGTKVDIK |
| M2B11_VH | 716 | QVQLVQSGTEVKKPGASVKVSCKASGYTFT |
| | | SHDINWLRQATGQGLEWLGWMNPNSATTGY |
| | | AQKFQGRVTLTRDISISTAYMELSSLRSDD |
| | | TAVYYCARNRHYYYADGPTRDAFDIWGQGT |
| | | VVTVSS |
| M2B11_VL | 717 | SYELTQTPTVSVSPGQTASITCSGDELGHK |
| | | YTSWYQQKPGQSPVLVVYQDTKRPSGIPER |
| | | FSGSNSGNTATLTISGTQVMDEADYYCQAW |
| | | DSGTVLLGGGTKLTVL |
| M2B12_VH | 718 | QVQLVESGGGVVQPGRSLRLSCEASGFTFS |
| | | SSAMHWVRQAPGKGLEWVAIISYHGTNKDY |
| | | SDSVRGRFTISRDNSKNTLYLQMNGLRGDD |
| | | TAVYYCAKEAAVSGNFDSWGQGTMVTVSS |
| M2B12_VL | 719 | QLVLTQSPSASASLGASVKLTCTLISGPSN |
| | | YAIAWHRQQPQKGPRYLMKVNSDGSHTKGD |
| | | GIPDRFSGSSSGTERYLIISNLQSEDEADY |
| | | YCQTWGSGIYVFGPGTKVTVL |
| M2C01_VH | 720 | EVQLVESGGDLAKPGGSLRLSCEASGFTFS |
| | | NAWMSWVRQAPGKGLEWVGQIKPHTDGATT |
| | | DYAAPVKGRFTISRDDSKSTLYLRMNILRI |
| | | EDTAMYYCTTGPRLSLMWGQGTRVTVSS |
| M2C01_VK | 721 | DIVMTQSPDSLAVSLGERATINCKSSQSVL |
| | | YTSNNRNFLAWYQQKPGQPPKLLIHWASTR |
| | | ESGVPDRFSGSGSGTDFTLTINSLQAEDVA |
| | | VYYCQQYYSIPPMFGQGTKVEIK |
| M2C05_VH | 722 | EVQLLESGGGLVQPGGSLRLSCAGSGFTFS |
| | | NYAMSWVRQAPGKGLEWVSDINDRGETTHY |
| | | ADSVKGRFTISRDNSKKKLFLQMNDLRAED |
| | | TAIYYCTRAYVVLSVYAPKGFDYWGQGTLV |
| | | SVSS |
| M2C05_VK | 723 | DIQMTQSPSILSASEGDRVTITCRASQSVS |
| | | NWLAWYQQKSGRAPKLLIYKASSLETGVPS |
| | | RFSGSGSGTEFTLTINNLQPDDFATYYCQH |
| | | YNGYPYTFGQGTKLEIK |
| M2C06_VH | 724 | QVQLVQSGAEVKKPGSSVKVSCRPSGGTFS |
| | | NDILSWVRQAPGQRLEWMGRIIPLVNMTNI |
| | | AQKFQGRLTLVADKSTAIVHLELGSLRAED |
| | | TAIYYCVRDLPSAGFPAPFDIWGQGTRVTV |
| | | SA |
| M2C06_VK | 725 | EIVLTQSPGSLSLSPGARATVSCRASQSLN |
| | | SASLVWYQQRVGQAPRLLMFGASNRATGIP |
| | | DRFSGSGSGTDFTLTISRLEPEDFAVYYCQ |
| | | QYAFSPPTFGQGTKVEIK |
| M2C07_VH | 726 | QVQLQESGPGLVAPSQTLSLICDVSGDSVT |
| | | SGAYSWSWIRQHPVKGLEWIAYINYSGRTN |
| | | YNPSLQSRLVISVDRSKNQFSLRLSSVTAA |
| | | DTAVYYCARANVVGYSGFYFDYWGQGTLVA |
| | | VSS |
| M2C07_VK | 727 | DIQMTQSPSSLSASVGDTVTITCRASQSVG |
| | | AYLNWYQQKPGKAPKLLVFAGSSLQTGVPS |
| | | RFSGRGSRTEFTLTITNLQREDFASYYCQQ |
| | | SLGDFLTFGGGTKVEIK |
| M2C08_VH | 728 | QVQLVQSGAEVKKPGASVIVSCRASGFTFS |
| | | LYTMHWVRQAPGQGLEWMGWISADNSNTKY |
| | | SQKYQDRVTITMDTSATTLFMEISSLRSED |
| | | TAVYYCTRGPAYYFDSSGFAFDIWGQGTLV |
| | | TVSS |
| M2C08_VK | 729 | EIVLTQSPGTLSLSPGERATLSCRASQSVS |
| | | RTYLGWYQQRPGLAPRLLIYGTSSRATGIP |
| | | DRFSGSGSGTDFTLTISRLEPEDFATYYCQ |
| | | HFGSSLTFGGGTKVEIK |
| M2C10_VH | 730 | QVQLVESGGGVVQPGRSLRLSCAASGFTFN |
| | | NYAMHWVRQAPGKGLEWVALISDDGSKKYF |
| | | ADALKGRFTIFRDNSKNTLYLQMNSLRAED |
| | | TAVYYCARDPTPLGEYYFDYWGQGTLVTVS |
| | | S |
| M2C10_VK | 731 | DIQMTQSPSSLSASVGDRVTITCRASQTIS |
| | | GYLNWYQQKPGKAPNLLIYTASSLESGVPS |
| | | RFSGSGSETDFTLTISSLQPEDFATYYCQQ |
| | | SYRMPLTFGGGTRVSLK |
| M2C11_VH | 732 | QVQLVQSGAEVRKPGASVKVSCKASGYTFT |
| | | SHDINWVRQAPGQGLEWMGWMNPNSDTTGY |
| | | AQKFQGRVTMARDISMSTAYMELSSLTSED |
| | | TAVYYCARGRHNYYTNGPTRDGFDIWGQGT |
| | | MVTVSS |
| M2C11_VL | 733 | SYELTQPPSVSVSPGQTATITCSGDDLGNQ |
| | | YANWFQQRPGQSPVLVIYQDNKRPSGIPER |
| | | FSGSSSGNTATLTISGTQPLDEADYYCQAW |
| | | DSRTYVFGTGTKVTVL |
| M2D01_VH | 734 | QVQLQESGPGVVKPSETLSLTCTVSRGAIT |
| | | DYHLNWIRQPPGKGLEWIGYISSSGSTNYN |
| | | PSLKSRVTISQVMSGTQFSLKLTSMSAADT |
| | | ALYYCASSSGRYWGGALDLWGQGTQVIVSS |
| M2D01_VK | 735 | DIVMTQSPDSLAVPLGERATINCKSSQSLL |
| | | YTSNNKNSLAWYQQKPGQPPKLLLFWASTR |
| | | EFRVPDRFSGSGSGTDFTLTISSLQAEDVA |
| | | VYYCQQYYTAPYTFGPGTKLEIK |
| M2D02_VH | 736 | QVQLVQSGAEVKKPGSSVKVSCKASGGTLT |
| | | RYTISWVRQAPGQGLEWMGRIIPFLDIANY |
| | | APRFQGRVTMTADKSTSTAYMELSSLTSQD |
| | | TAIYYCARDHSGNYFDLWGQGTLVTVSS |
| M2D02_VK | 737 | EIVMTQSPATLSVSPGERATLSCRASQSVT |
| | | SNLAWYQRKPGQAPRLLIYGASTGATDIPG |
| | | RFSGSGSGTEFTLTISGLQSEDFAVYYCQQ |
| | | YDHWPRTFGQGTKVEIK |
| M2D03_VH | 738 | QVQLQESGPGLVAPSQTLSLICDVSGDSVT |
| | | SGAHSWSWIRQHPVKGLEWIAYINYSGRTN |
| | | YNPSLQSRLVISVDRSKNQFSLRLSSVTAA |
| | | DTAVYYCARANVVGYSGYYFDYWGQGTLVA |
| | | VSS |
| M2D03_VK | 739 | DIQMTQSPSSLSASVGDRVTITCRASQSVG |
| | | AYLNWYQQKPGKAPKLLIFAGSSLQTGVPS |
| | | RFSGRGSRTEFTLTISNLQREDFATYYCQQ |
| | | SLGDFLTFGGGTKVEIK |
| M2D07_VH | 740 | QVQLVESGGAVVQPGRSLRLSCAASGFTFT |
| | | SFSMHWVRQAPGRGLEWVAVISHDGIIKYY |
| | | ADSVKGRFTISRDNSKNTVFLQMNSLRPED |
| | | TAVFYCARDFPGSRGFFRYYFDHWGQGTLV |
| | | TVSS |
| M2D07_VK | 741 | DIQMTQSPSSLSASVGGSVTITCRVDQSIS |
| | | NDLHWYQQKPGKAPKLLISSASSLQSGVPS |
| | | RFSGSGSGTDFTLTITSLQPEDFATYYCQQ |
| | | SYTTPPLFGQGTTLEIK |
| M2D08_VH | 742 | QVQLVESGGGVVQPGRSLRLSCAASGFVFR |
| | | FSGMHWIRQAPGKGLQWVAHISDDGSQKYY |
| | | ADSVTGRFAISRDNSRNTLFLEMTGLRGDD |
| | | TAVYYCARGDYGVSYFLAHWGLGELVIVSS |
| M2D08_VK | 743 | EIVLTQSPATLSLSPGERATLSCRASQNIY |
| | | NYLAWYQQKPGQAPRLVIYDASNRATGIPA |
| | | RFSGSGSETDFTLTISSLEPEDFAVYYCQQ |
| | | RSDWTFGQGTKLEIK |
| M2D09_VH | 744 | EVQLLESGGGLVQPGGSLRLSCAASGYTFS |
| | | SYAMSWVRQAPGKGLEWVSGISGSGANTYY |
| | | ADSVKGRFTISRDNSQNTLYLQMNSLRAED |
| | | TAVYYCARGLYTTGDYWGQGTLVTVSS |
| M2D09_VK | 745 | DIQMTQSPSSLSASVGDRVTITCRASQSMS |
| | | NSLNWYQQKPGKAPKLLIYAASSLQSGVPS |
| | | RFSGSRSGSVFTLTISNLQPEDFATYYCQQ |
| | | SYSTPITFGQGTRLEIK |
| M2D10_VH | 746 | QVQLVQSGAEVKKPGASVRISCKPSGRAFL |
| | | TYDINWVRQAPGQGLEWMAGMNPGSGNAAY |
| | | APKFQGRLTMIRNTSISTDYLELSSLTSED |
| | | TAVYYCARGEVSMRWRWLDPWGQGSLVTVT |
| | | S |
| M2D10_VK | 747 | DIQMTQSPSSLSASVGDRVTITCRASQTIS |
| | | NFLNWYQQKPGRAPKVLIYAALNLQSGVPS |
| | | RFRGSGSGTDFTLTINNLQPEDFAIYYCQQ |
| | | SYITPWTFGQGTKVEIK |
| M2D11_VH | 748 | QVQLQQWGAGLLKPSETLSLTCAVSGGSFS |
| | | GYHWTWIRQPPGKGLEWIGEVDHGGNTNYN |
| | | PSLMSRVTISVDMSTKQFSLRLTSMTAADT |
| | | AVYYCARGFGVVINNWFDPWGQGTLVTVTS |
| M2D11_VK | 749 | DIQMTQSPSSLSASVGDRVTITCRASQTIS |
| | | SYLNWYQQKPGKAPNLLIYTVSTLQSGVPS |
| | | RFSGSGSGTDFTLTISSLQPEDFATYYCQQ |
| | | SYGAPYTFGQGTKLEIK |
| M2E01_VH | 750 | QLQLQESGPGLVKPSETLSLTCTVSGGSIS |
| | | SSTYYWGWIRQPPGKGLEWIGTIYYSGSTD |
| | | YNPSLKSRLTISVDTSKNQFSLRLRSVTAA |
| | | DTAVYYCARLVSPYDQFDYWGQGTLVTVSS |
| M2E01_VK | 751 | DIQMTQSPSSLSASVGDRVTITCRASQTIT |
| | | TYLSWYLQKPGKAPKLLIYAASSWQSGVPS |
| | | RFSGSGSGTDFTLTISSLQPEDFATYYCQQ |
| | | SYSTPYTFGQGTKLEIK |
| M2E04_VH | 752 | QVQLQESGPGLVKPSEILSLTCTVSRFSIN |
| | | SGYYWGWIRQPPGKGLEWIGCISHAGSIYS |
| | | NPSLESRVTLSLDTSKNQFSLKLTSVIAAD |
| | | TATYYCAASSNWHNALEYWGQGTLVVVSS |
| M2E04_VL | 753 | QSVLTQPPSASGTPGQTVTISCSGSRSNMG |
| | | INWVYWYQQLPGTAPKLVIYRNSQRPSGVP |
| | | DRFSGSQSGTSASLAISGLRSEDEAVYYCA |
| | | VWDDSLDGRLFGGGTKLTVL |
| M2E05_VH | 754 | QVQLQESGPGLVKPSEILSLTCTVSRYSIN |
| | | SGYYWGWIRQPPGKGLEWVGCVSHAGVTYY |
| | | NPSLESRVAVSLDTSKNQFSLKMTSVSAAD |
| | | TAMYYCAASSNWHNALEYWGQGTLVAVSS |
| M2E05_VL | 755 | QSVLTQPPSASGTPGQSVTISCSGSSSNMG |
| | | INWVYWYQQLPGTAPKLLIYRNSQRPSGVP |
| | | DRFSGSQSGTSASLAISGLRSEDEGHYYCA |
| | | VWDDVVSGRLFGGGTKLTVL |
| M2E06_VH | 756 | EVQLVESGGDLVQPGGSLRLSCEASAFTFS |
| | | DYAMTWVRQAPGKGLEWVSSINTSGGRTYY |
| | | ADSVKGRFTISRDNSKNTLYLQMKSLRAED |
| | | TAVYFCARFFYVMDVWGQGTTVTVSS |
| M2E06_VK | 757 | AIQMTQSPSSLSASVGDRVTITCRASQGVR |
| | | NDLGWYQQKPGKAPKLLINAASGLQSGVPS |
| | | RFSGSGSDTDFTLTISSLQPEDFATYYCLQ |
| | | VYSYPFTFGPGTKVDIK |
| M2E07_VH | 758 | EVQLVESGGGLVQTGGSLRLACAASGFTFS |
| | | SYAMSWVRQAPGKGLEWVSTITESGDDAYY |
| | | ADSVKGRLTISRDNSKNTLFLHLNGLRSED |
| | | TARYYCAKDMGLYKSSWYGNFFYYYYMDVW |
| | | GEGTTVTVSS |
| M2E07_VL | 759 | QSALTQPASVSGSPGQSITISCTGSSSNIG |
| | | SFDLISWYQQHPGKAPKFIIYEGDKRPSGV |
| | | SYRFSGSKSGSTASLTISGLQAEDEAYYYC |
| | | CSYARSRTLVFGGGTKVTVL |
| M2E08_VH | 760 | EVQLVESGGDLVLPGGSLRLSCEASGFMFS |
| | | SYAMNWVRQAPGKGLERVSGISGSGGNTYY |
| | | ADSVKGRFTISRDNSKNIVYLQMNSLRAED |
| | | TAVYYCAKDLGNDYFYYIDVWGKGTTVMVS |
| | | S |
| M2E08_VL | 761 | QSVLTQPPSVSGAPGQRVTISCTGTNFNIG |
| | | AGYDVQWYQQLPGAAPKLLIHGNTYRPSGV |
| | | PDRFSGSKSGTSASLAITGLQAEDEADYYC |
| | | QSYDSSLSMVFGGGTKLTVL |
| M2E11_VH | 762 | QVQLVQSGAIMKKPGSSVKVSCKASGGTFT |
| | | NFAFSWVRQAPGQGLEWMGGIIPFVGPANY |
| | | APQFQGRVTITADESTTTAYMELNSLRSED |
| | | TAVYYCARVRGFGETAYFYHYYMDVWGKGT |
| | | TVTVSS |
| M2E11_VL | 763 | QTVVTQEPSLSVSPGGTVTLTCGLSSGSVS |
| | | TSHFPSWYQQIPGQPPRTLIYSTITRSSGV |
| | | PDRFSGSILGNKATLTITGAQADDECDYYC |
| | | VLYLGGGIPHWVFGGGTKLTVL |
| M2F01_VH | 764 | QVQLVQSGAEVKKPGASVKVSCKASEDTFT |
| | | NYYMHWVRQAPGQGLEWMGIINPSGGSTDY |
| | | AQKFQGRVTMTRDTSTSTVYMELSSLRSED |
| | | TAMYYCARQGRTAACDYWGQGTLVTVSS |
| M2F01_VK | 765 | DIQMTQSPSSVSASVGDRVTITCRASQGIR |
| | | SWLAWYQQKPGKAPKLLIYAASTLQSGVPS |
| | | RFSGSGSGTDFTLTISSLQPEDFATYYCQQ |
| | | SNSFSLFTFGPGTRVDIK |
| M3A01_VH | 766 | EVQLVESGGGLVKPGGSLRLSCSVSAFSIS |
| | | GYNMHWVRQAPGKGLEWVSSITGGSNYINY |
| | | ADSVKGRFTISRDNAQNSLYLQMKSLRAED |
| | | TAVYYCARETQPSPGSFDSWGQGTLVTVSS |
| M3A01_VL | 767 | QSVLTQPPSVSGAPGQRVTISCTGSSSNIG |
| | | AGYDVNWYQQLPGTAPKLLIYGNYNRPSGV |
| | | PDRFSASKSGTSASLAITGLQAEDEADYWC |
| | | QSYDTSLSGWIFGGGTKLTVL |
| M3A03_VH | 768 | QVQLVQSGAEVKKPGASVKVSCKASAYTFT |
| | | SYGITWVRQAPGQGLEWMGWISAYRGNTKY |
| | | AQKLQGRVTMTTDTSTSTASMELRSLRSDD |
| | | TAVYYCARTSYYDSSYYPIDYWGQGTLVTV |
| | | SS |
| M3A03_VL | 769 | SYVLTQPPSVSVAPGQTARITCGGNNIGSK |
| | | SVQWYQQKPGQAPVLVVYDDSDRPSGIPER |
| | | FSGSNSGNTATLTISRVEAGDEADYYCQVW |
| | | DSSGDRVVFGGGTKLTVL |
| M3A04_VH | 770 | QVQLVESGGGVVQPGRSLRLSCAASGLTFS |
| | | TYVMHWVRQAPGKGLEWVAVISYDGTNEHY |
| | | ADSVKGRFTISRDNSKNTLYLQMNSLRPED |
| | | TAVYYCARGTADYDVWSGSAWGGFDYWGQG |
| | | TLVTVSS |
| M3A04_VK | 771 | DIQMTQSPSSLSASVGDRVTITCRASQSIS |
| | | SYLNWYQQKPGKAPKLLMYTVSSLPSGVPS |
| | | RFSGSGSGTDFTLTISSLQPEDFATYYCQQ |
| | | SYNIPYTFGQGTKLEIK |
| M3A05_VH | 772 | QITLKESGPTLVKPTQTLTLTCTFSGFSLN |
| | | TGGVSVGWIRQPPGKALEWLALIYWDGDKR |
| | | YSPSLKSRLTITKDTSKNQVVFTMTNMDPV |
| | | DTATYYCAHRENSAYIDFWGQGTLVTVSS |
| M3A05_VL | 773 | QSVLTQPPSVSAAPGQKVTISCSGSSSNIG |
| | | NNYVSWYQQLPGAAPKLLIYDNNKRPSGIP |
| | | DRFSGSKSGTSASLGITGLQTGDEADYYCG |
| | | TWDSSLSVGFYVFGTGTKVTVL |
| M3A06_VH | 774 | EVQLVESGGGLVKPGGSLRLSCAASGFTLT |
| | | SYSMNWVRQAPGKGLEWVSSISSSSSYIHY |
| | | ADSVKGRFTISRDNAKNSVYLQMNSLRAED |
| | | TAAYYCARVARELDPFDMWGQGTVVTVAS |
| M3A06_VL | 775 | QSVLTQPPSVSGAPGQRVTISCTGSSSNIG |
| | | AGYDVHWYQQLPGTAPKLLVYANNNRPSGV |
| | | PDRFSGSKSGTSASLAITGLQADDEADYYC |
| | | QSYDRSLSGSVFGGGTKLTVL |
| M3A07_VH | 776 | QVQLVQSGAEVQKPGASVKVSCKASGYTFT |
| | | RHAIHWVRQAPGQRLEWIGWVHGGNENTKY |
| | | SQKLQGRVTITRDTSASTAYMELNSLRYED |
| | | TAVYYCATAMPGAFNMWGQGTVVTVSS |
| M3A07_VK | 777 | DIQMTQSPSTLSASVGDRVTITCRASQSIS |
| | | SWVAWYQQKPGKAPKLLIYKAFSLETGVPS |
| | | RFSGSGSGTEFTLTISSLQPDDCATYYCQQ |
| | | YGSFSEFTFGQGTKLEIK |
| M3A08_VH | 778 | EVQLVESGGGLVKPGGSLRLSCAASGFSIT |
| | | SYGMHWVRQAPGKGLEWVSSISSSSSYISY |
| | | RDSVKGRFTVSRDNAGNSLSLQMNSLRGED |
| | | TAVYHCARDAGLVPPWMDSWGQGTLVIVSS |
| M3A08_VL | 779 | QSVLTQPPSVSGAAGQRVTISCTGSSSNLG |
| | | TGYDVHWYRQQPGTAPKLIMFGNTNRPSGV |
| | | PGRFSGSRSGTSASLAITGLQADDEADYYC |
| | | QSYDSSLGGLVFGGGTKLTVL |
| M3A09_VH | 780 | QVQLVESGGGVVQPGRSLRLSCAASGFTFS |
| | | SYAMHWVRQAPGKGLEWVAVISYDGSNKYY |
| | | ADSVKGRFTISRDNSKNTLYLQMNSLRAED |
| | | TAVYYCARDVQQQLALDYWGQGTLVTVSS |
| M3A09_VK | 781 | EIVLTQSPATLSLSPGERATLSCRASQSVS |
| | | SYLAWYQQKPGQAPRLLIYDASNRATGIPA |
| | | RFSGSGSGTDFTLTISSLEPEDFAVYYCQQ |
| | | RSNWPPKWTFGQGTKVEIK |
| M3A11_VH | 782 | EVQLVESGGGLVQPGGSLRLSCAASGFTFS |
| | | DYSMNWVRQAPGQGLEWISYISSTGSTIYY |
| | | ADSVRGRFTISRDNAKNSLYLQMNTLRAED |
| | | TAVYYCARGKTTVTIWFDPWGQGTLVTVSS |
| M3A11_VL | 783 | SYVLTQPPSVSVAPGQTARITCGGNNIGSK |
| | | SVHWYQQKPGQAPVLVVYDDSDRPSGIPER |
| | | FSGSNSGNTATLTISRVEAGDEADYYCQVW |
| | | HSSSDWVFGGGTRLTVL |
| M3A12_VH | 784 | QVQLQESGPQLVKPSQTLSLTCTVSGDSIS |
| | | SSDYCWSWIRQSPGKGLEWIGHSVYSGDTQ |
| | | YNPSLKSRLSISVDTSKNQFSLRLRSVTAA |
| | | DTAVYYCARGNDFWSGSFDYWGQGALVIVS |
| | | S |
| M3A12_VK | 785 | DIQMTQSPSSLSASVGDRVTITCRASQSIG |
| | | TYLNWYQQKPGKAPKVLIYAASSLQSGVPS |
| | | RFSGRGSGTDFTLTVSSLQPEDFASYYCQQ |
| | | SYTKPYTFGQGTKLEIK |
| M3B02_VH | 786 | EVQLVESGGGLVKPGGSLRISCVASEFTLS |
| | | GYAMNWVRQVPGKGLEWVSSISSSSSYINY |
| | | ADFVKGRFTISRDNAKNSVFLQLNSLRPED |
| | | TALYYCVRERPTAILPYLDLWGRGTLVTVS |
| | | S |
| M3B02_VL | 787 | QSVLTQPPSVSGAPGQRVTISCTGSSSNIG |
| | | AGYDVQWYQQSPGTAPKLLIHGNNNRPSGV |
| | | PDRFSGSKSGTSASLAITGLQAEDEADYYC |
| | | QSYDRSLSGWVFGGGTKLTVL |
| M3B04_VH | 788 | QVQLQESGPGLVKPSETVSLTCTVSGGSIS |
| | | SNNWWSWVRQSPGKGLEWIGEIYHSGSTNF |
| | | NPSLKSRVTISVDKSKNHFSLKLGSLTAAD |
| | | TAVYYCARRPNDYGDYSYYYYYYMDVWGKG |
| | | TTVTVSS |
| M3B04_VK | 789 | EIVLTQSPATLSLSPGESATLSCRASQSIN |
| | | RNLAWYQQKPGQAPRLLIYDASNRATGIPA |
| | | RFSGSGSGTDFTLTISSLEPEDFAVYYCQQ |
| | | RSHWVTFGQGTRLEIK |
| M3B06_VH | 790 | QVQLVQSGPEVRKPGASVKVSCRTSGYNFP |
| | | AYGISWLRQAPGQGFEWMGWISAYNVNTNY |
| | | AQNLQDRVAMTTDTSTSTAFLELRSLTSDD |
| | | TGMYYCARVPRPPLSGTYSTADFWGQGTLV |
| | | TVSS |
| M3B06_VK | 791 | DIQMTQSPSTLSASVGDRVTITCRASQTIN |
| | | NWLAWYQQKPGKAPKLLIYKASSLESGVPS |
| | | RFSGSGSGTEFTLTISSLQPEDFATYYCQQ |
| | | YISYSRTFGQGTKVEVK |
| M3B07_VH | 792 | QVQLQQWGAGLLKPSETLSLTCAVYGGSFS |
| | | GYYWSWIRQSPGKGLEWIGEINHSGSTNYN |
| | | LSFKSRVTISIDTSKNQFSLKVTSVTAADT |
| | | AVYFCARGPIRYSSYYYYYGLDVWGQGTTV |
| | | TVSS |
| M3B07_VK | 793 | EIVLTQSPDTLSLSPGERATLSCRASQSVS |
| | | SSYLAWYQQKPGQAPRLIIYSTFIRVTGFP |
| | | DRFSGSGSGTDFTLTISRLEPEDFAVYYCQ |
| | | QYGSSPRTFGQGTKVEIK |
| M3B12_VH | 794 | QVQLVESGGGVVQPGGSLRLSCAASGFTFS |
| | | SYGMHWVRQAPGKGLEWVAFIRYDGSNKYY |
| | | ADSVKGRFTISRDNSKNTLYLQMNSLRAED |
| | | TAVYYCAKDSGSYYYYYYMDVWGKGTTVTV |
| | | SS |
| M3B12_VK | 795 | EIVLTQSPATLSLSPGERATLSCRASQSVS |
| | | SYLAWYQQKPGQAPRLLIYDASNRATGIPA |
| | | RFSGSGSGTDFTLTISSLEPEDFAVYYCQQ |
| | | RSNWPPLTFGGGTKVEIK |
| M3C01_VH | 796 | QLQLQESGPGLVKPSETLSLTCTVSGGSIS |
| | | SSSYFWGWIRQPPGKGLEWIGSVFYSGSTY |
| | | YNPSLKSRVTISVDTPKNQFSLRLSSVTAA |
| | | DTAVYYCARHSWDRWVYWGQGTLVTVSS |
| M3C01_VK | 797 | DIQMTQSPSSVSASVGDRVTITCRASQDIS |
| | | SWLAWYQQKPGKAPKLLIYAASSLQSGVPS |
| | | RFSGSGSGTDFTLTISSLQPEDFATYYCQQ |
| | | ANSFPLTFGGGTKVEIK |
| M3C04_VH | 798 | EVQLVQSGAEVKKPGESLKISCKGSGYNFA |
| | | NYWIGWVRQMPGKGLEWMGIIYPGDSDTRR |
| | | NPSFQGHVTMSVDQSISTAYLQWSSLKASD |
| | | TAMYYCARPLAVSAFDIWGQGTMVTVSS |
| M3C04_VK | 799 | DIQMTQSPSSLSASVGDKLTITCRASQSIS |
| | | NYLNWYQQKSGKAPKLLIYAASSLQSGVPS |
| | | RFSGSGSGTDFTLTISSLQPEDFATYYCQQ |
| | | SASTPHTFGQGTKVEIV |
| M3C05_VH | 800 | EVQLVESGGGLVQPGGSLRLSCVASGFGFR |
| | | SYWMHWVRQAPGKGLVWVSRINNDGSSASY |
| | | ADSVKGRFTISRDNAKNTLYLQMNSLRAED |
| | | TALYYCAREFGVLDYHYGMDVWGQGTTVTV |
| | | SS |
| M3C05_VL | 801 | QSVLTQPPSASGTPGQRVTISCSGSSSDIG |
| | | SNSVIWYQQLPGTAPKLLIYSSIQRPSGVP |
| | | DRFSGSKSGTSASLAISGLQSEDEADYYCA |
| | | TWDDSLNGPVFGGGTKLTVL |
| M3C06_VH | 802 | QVQLVQSGAEVKKPGSSVKVSCKAYGGTLS |
| | | NYAISWVRQAPGQGLHWMGRIIPVMNVANY |
| | | ADNFQGRVTITADKSTSTAYMELSSLRSED |
| | | AAIYYCATARGLGSLEGGDAFDVWGQGTML |
| | | TVSS |
| M3C06_VK | 803 | DIVMTQSPDSVAVSLGERATISCKSSLSLL |
| | | YSSNNKNYLVWYQQKPGQPPKLLFYWASTR |
| | | ESGVPDRFSASGSGTDFTLTISSLQAEDVA |
| | | VYYCQQYYSTPITFGQGTRLEIR |
| M3C07_VH | 804 | QLQLQESGPGLVKPWETLSLTCIVSGGSIS |
| | | RSTHYWGWIRQSPEMGLEWIGSIYYTGNTY |
| | | YNPSFKSRVTISVDTSRNQLSLRVNSVSAA |
| | | DTARYYCARHETYDYGVFNIWGQGAMVTVS |
| | | A |
| M3C07_VK | 805 | EIVLTQSPGTLSLSPGERAILSCRASQTVS |
| | | SDYLAWYQQRPGQAPRLLIYDASSRATGVP |
| | | DRFRGSGSGTDFTLTISRLEPEDFGVYYCQ |
| | | QYGSPYTFGQGTKLEIK |
| M3C11_VH | 806 | QVQLQESGPGLVKPSETLSLTCTVSGGSLS |
| | | SYCWSWIRQPPGKGLEWIGYIYYSGGTNYN |
| | | PSLKSRVTISVDTSKNQFSLNLSSVTAADT |
| | | AVYYCARGEASGYLNYYFDYWGQGTLVAVS |
| | | S |
| M3C11_VL | 807 | SYELTQPPSVSVSPGQTASITCSGHKLGDK |
| | | YAYWYQQKPGQSPVLVIYQDNKRPSGIPER |
| | | FSGSNSGNTATLTISGTQAMDEADYYCQAW |
| | | DSGTVFGGGTKLTVL |
| M3C12_VH | 808 | EVQLVESGGGLVQPGGSLRLSCAASGFSFG |
| | | SYYMHWVRQAPGKGLVWVSRTSSDGSTTSY |
| | | ADSVKGRFSISRDNAKNTLYLQMDSLRAED |
| | | TAVYYCARELGILDYYYGMDVWGQGTTVTV |
| | | SS |
| M3C12_VL | 809 | QSVLTQPPSASGTPGQRVTISCSGSSSNIG |
| | | RNPVSWYQQLPGTAPKHLIYKNSQRPSGVP |
| | | DRFSGSKSGTSASLAISGLQSEDEADYYCA |
| | | AWDDSLNGPVFGGGTKLTVL |
| M3D03_VH | 810 | QVQLVQSGAEVKKPGASVKVSCKASGYTFT |
| | | SYGISWVRQAPGQGLEWMGWISAYNGNTNY |
| | | AQKLQGRVTMTTDTSTSTAYMELRSLRSDD |
| | | TAVYYCARDAPFGRDDGYGMDVWGQGTTVT |
| | | VSS |
| M3D03_VK | 811 | DIQMTQSPSSLSASVGDRVTITCRASQSIS |
| | | SYLNWYQQKPGKAPKLLIYAASSLQSGVPS |
| | | RFSGSGSGTDFTLTISSLQPEDFATYYCQQ |
| | | SYSTPRTFGPGTKVDIK |
| M3D04_VH | 812 | EVQLVESGGDLVEPGGSLRLSCAASGFTFE |
| | | SAWMSWVRQAPGKGLEWVGRIKSKLDHGTT |
| | | DYAAPVKGRFTLSRDDSKNTLFLQMNNLKT |
| | | EDTGVYYCATDRSDVRFLEWLGNYQFGMDV |
| | | WGQGTTVAVSS |
| M3D04_VK | 813 | EIVLTQSPGTLSLSPGERATLSCRASHSVS |
| | | SSYLAWYQQKPGQAPRLLIFGASSRATGIP |
| | | DRFSGSGSGTDFTLTISRLEPEDFAVYYCQ |
| | | QYLTSPPGWTFGQGTKVEIK |
| M3D05_VH | 814 | QVQLVQSGAEVKQPGASVKVSCKASGYTFS |
| | | GYYIQWVRQAPGQGLEWMGWINPNSGGTNF |
| | | AQKFQGRVTMTSDTSTSAAYMELSSLRSDD |
| | | TAVYYCVRGQQLTRQYYFGMDVWGQGTTVT |
| | | VSS |
| M3D05_VL | 815 | QSVLTQPPSVSGAPGQRVIISCSGSSSNIR |
| | | SNAVNWYQQLPGKAPKLLIFYDGLLPSGVS |
| | | DRFSGSKTGNSASLAISGLQSEDEADYYCA |
| | | AWDDSLNGPVFGGGTKLTVL |
| M3D06_VH | 816 | EVQLVESGGGLVQPGGSLRLSCAASGFPFN |
| | | FYWMHWVRQAPREGLVWVSRINNDGSSPNY |
| | | ADSVWGRFTISRDNAKNTLYLQMHSLRAED |
| | | TAVYYCARAARFGDYVDQWGQGTLVTVSS |
| M3D06_VK | 817 | AIQLTQSPSSLSASMGDRVTITCRASQGIS |
| | | SAFAWYQQKPGKAPKLLIYDASTLESGVPS |
| | | RFSGSGSGTDFTLTISSLQPEDFATYYCQQ |
| | | FNSYPLTFGGGTKVEIK |
| M3D08_VH | 818 | QVQLQESGPGLVKPSETLSLTCSVSGVSIS |
| | | SHYWSWIRQPAGKGLEWIGRLYTSGTTTYN |
| | | PSLNSRVTMSVDTTNNRLSLRLTSVTAADT |
| | | AVYYCARDVRLTVFGALHNWFDPWGQGILV |
| | | TVSS |
| M3D08_VK | 819 | DIQMTQSPSSLSASVGDRVTITCRASQSIG |
| | | THLNWYQQQRGRAPKLLVYAASGLQSGVPS |
| | | RFSGSGSGTYFTLTISSLQPEDFATYYCQQ |
| | | SYSTPSITFGQGSRLDIK |
| M3D09_VH | 820 | QVQLVESGGGVVQSGKSLGLSCIASGFIFS |
| | | NYALHWVRQAPGKGLEWLSVISYDGTNKYY |
| | | ANSVRGRFTVSRDNSKNTLFLHMNSLGVDD |
| | | TAMYYCARDPERLADLGRMDFWGRGTLVAV |
| | | LS |
| M3D09_VK | 821 | DIVMTQSPDSLAVSLGERATINCKSSQSVL |
| | | FSPNNKNYLAWYQQKPGQPPKLLIYWASTR |
| | | EYGVPERFSGSGSGTDFTLTISSLQAEDVA |
| | | VYYCQQYNSNPYTFGQGTKLEIR |
| M3D10_VH | 822 | QVQLQESGPGLVKPSETLSLTCTVSGGSVS |
| | | TYYWTWIRQPPGKGLDWIGYIYHNGNINYN |
| | | PSLKSRVTMSVDTSKNQFSLKLTSVTAADT |
| | | ALYYCATVSFIGYSLYYFDRWGQGTLVTVS |
| | | S |
| M3D10_VK | 823 | DIQMTQSPSSLSASVGDRVTITCRASQNIA |
| | | SFLNWYQQRPGKAPDLLIYAASTLQTGVPS |
| | | RFSGSGSGTDFTLAISSLQPEDFATYYCQQ |
| | | SYNTPFTFGQGTKLEIK |
| M3D12_VH | 824 | QVQLQESGPGLVKPSQTLSLTCTVSGASVS |
| | | SEYYYWSWIRQPPGKGLEWIGYIYHNGDTY |
| | | YNPSLKGRGIISMDTSKNQFSLKLISVTAA |
| | | DTAVYYCARLAVAGVTGFHYWGLGTLVTVS |
| | | S |
| M3D12_VK | 825 | EIVLTQSPDTLSLSPGERATLSCRASRSVR |
| | | NYLAWYQQKPGQAPRLLIYDASNRAPGIPA |
| | | RFSGSGSETDFTLTISSLEPEDFAVYYCQQ |
| | | RSNWGLTFGGGTKVEIK |
| M3E01_VH | 826 | EVQLVESGGGLIQPGGSLRLSCTASGFTVS |
| | | SKYMSWVRQAPGKGLEWVSLIYDGGSTYYA |
| | | DSVKGRFLISRDNAKNTLYLQMHSLRAEDT |
| | | AVYYCARDRGTYSGYDWYSGMDVWGQGTTV |
| | | TVSS |
| M3E01_VK | 827 | DIQLTQSPSFLSASVGDRITITCRASQGIS |
| | | SYLAWYQQKPGKAPKLLIYAASTLQSGVPS |
| | | RFSGSGSGTEFNVTISSLQPEDFATYYCQQ |
| | | LKSYPGTFGQGTKVEIE |
| M3E02_VH | 828 | QVQLQESGPGLVKPSETLSLTCTVSGGSIS |
| | | SYYWSWIRQPAGKGLEWIGRIYTSGSTNYN |
| | | PSLKSRVTMSVDTSKNQFSLKLSSVTAADT |
| | | AVYYCARDASGLSWYDWYFDLWGRGTLVTV |
| | | SS |
| M3E02_VK | 829 | EIVLTQSPGTLSLSPGERATLSCRASQSVS |
| | | SSYLAWYQQKPGQAPRLLIYGASSRATGIP |
| | | DRFSGSGSGTDFTLTISRLEPEDFAVYYCQ |
| | | QYGSSPTFGQGTKLEIK |
| M3E03_VH | 830 | QVQLVESGGGVVQPGRSLRLSCVASGFPFD |
| | | SFAMHWVRQAPGKGLEWVAIVWYDGGDKFY |
| | | SESVKGRFSISRDNSRNTVFLQMNSLRAED |
| | | TAVYYCARGMITFDYWGPGTLVTVSS |
| M3E03_VK | 831 | EIVLTQSPGTLSLSPGERATLSCRASQNVD |
| | | SSYLAWYQQKPGQAPRLLMYGASTRVAGIP |
| | | DRFSGSGSGTDFTLTISRLEPEDFVVYYCQ |
| | | QYGTSPTWTFGQGTKVEIK |
| M3E04_VH | 832 | EVQLVESGGGLVKPGGSLRLSCAGSGFTFS |
| | | SYSMNWVRQAPGKGLEWVSSISASSSYIYY |
| | | ADSLKGRFTISRDNAKNSLYLQMNSLRAED |
| | | TAVYYCARANYGSGNYYNPKWFDPWGQGTL |
| | | VTVSS |
| M3E04_VL | 833 | QSVLTQPPSVSGAPGQRVTISCTGSSSNIG |
| | | AGYDVHWYQQFPGTAPKVLIYGNNNRPSGV |
| | | PDRFSGSKSGTSASLAISGLQAEDEADYYC |
| | | QSYDSSLSGSIFGGGTKLTVL |
| M3E05_VH | 834 | EVQLVESGGSVIQPGGSLRLSCAASGFSVR |
| | | TNYMNWVRQAPGKGLEWVSVIYSDGRTYYS |
| | | DSVKGRFTISRDNSMNNVFLQMNSLRAEDT |
| | | AVYYCARDPTFYSSSWWYLDHWGQGTQVTV |
| | | SS |
| M3E05_VL | 835 | SYELTQALSVSVARGQTAKITCGGNNIGVR |
| | | NVHWYQQKPGQAPVLVIYRDNIRPSGIPER |
| | | FSGSNSGNMAALTISSAQEGDEADYYCQVW |
| | | DSNTAHVIFGGGTKLTVL |
| M3E06_VH | 836 | EVQLVESGGGLVQPGGSLRLSCAASGFTFS |
| | | SYWMSWVRQAPGKGLEWVANIKQDGSEKYY |
| | | VDSVKGRFTISRDNAKNSLYLQMNSLRAED |
| | | TAVYYCARYYYDSSGYYYYYYYYMDVWGKG |
| | | TTVTVSS |
| M3E06_VK | 837 | EIVMTQSPATLSVSPGERATLSCRASQSVS |
| | | SNLAWYQQKPGQAPRLLIYGASTRATGIPA |
| | | RFSGSGSGTEFTLTISSLQSEDFAVYYCQQ |
| | | YNNWPPYTFGQGTKLEIK |
| M3E08_VH | 838 | EVQLLESGGGLVQPGGSLRLSCAASGFSFS |
| | | SYVMSWVRQAPGKGLEWVSTVSAGGGSTHY |
| | | ADSVKGRFTISRDNSKNTLYLQMNSLRAED |
| | | TAVFYCAKDKYPHYYYGMDVWGQGTTVSVS |
| | | S |
| M3E08_VL | 839 | QSVLTQPPSASETPGQRVTISCSGSSSNIG |
| | | RNYVYWYQQLPGTAPKLLIYRNNQRPSGVP |
| | | DRFSGSESGTSASLAISGLRSEDEADYYCA |
| | | AWDDSLSGYVFGTGTKVTVL |
| M3E09_VH | 840 | EVQLVESGGGVVRPGGSLRLSCAASGFRED |
| | | DYAMNWVRQAPGKGLEWVSGINWIGGTTKY |
| | | ADSVKGRFTISRDNTKNSLYLQMNSLRAED |
| | | TALYYCARGVGYYYYYMDVWGRGTTVTVSS |
| M3E09_VK | 841 | EIVLTQSPGTLSLSPGERATLSCRATQSVS |
| | | SNYLAWYQQKPGQAPRLLIYDASSRAIGIP |
| | | DRFSGSGSGTDFTLTISRLESEDFAVYYCQ |
| | | QYGSSPETFGQGTKVEIK |
| M3E10_VH | 842 | QVQLVQSGAEVKKPGSSVKVSCKASGGTIN |
| | | SYAISWVRQAPGQGPEWLGGIIPIFGTANY |
| | | AQNFPGRVTITADASTSTVYMELSSLRSDD |
| | | TAIYYCARGTRYIAVMVPSMQDYYFFGMDV |
| | | WGQGTTVTVSS |
| M3E10_VK | 843 | DIQLTQSPSFLSASVGDRVTITCRASQGIS |
| | | SYLAWYQQKPGQAPKLLIYAASTLQSGVPS |
| | | RFSGSGSGTEFTLTISSLQPEDFATYYCQQ |
| | | VTSYPFSITFGQGTRLEIK |
| M3F02_VH | 844 | QVQLQESGPGLVKPSQTLSLTCTVSGGSIS |
| | | SGDYYWSWIRQPPGKGLEWIGYIYYSGSTY |
| | | YNPSLKSRVTISVDTSKNQFSLKLSSVTAA |
| | | DTAVYYCARITGEGLPQAFDYWGQGTLVTV |
| | | SS |
| M3F02_VL | 845 | SYELTQPPSVSVSPGQTASITCSGDKLGDK |
| | | YACWYQQKPGQSPVLVIYQDSKRPSGIPER |
| | | FSGSNSGNTATLTISGTQAMDEADYYCQAW |
| | | DSSTAVFGGGTKLTVL |
| M3F03_VH | 846 | QVQLVESGGGVVQPGGSLRLSCAASGFTFS |
| | | RYAMHWVRQAPGKGLEWVAVMSHDGSNRYY |
| | | ADSVKGRFTISRDNSKNTLYLQMNSLRAED |
| | | TAVYYCAKDPGGLGYSYGLGYFDYWGQGTL |
| | | VTVSS |
| M3F03_VK | 847 | EIVMTQSPATLSVSPGERATLSCRASQSVT |
| | | NNLAWYQQKPGQAPRLLIYGASTRATGIPA |
| | | RISGSGSGTEFTLTINSLQSEDFAVYYCQQ |
| | | YNGWPPWTFGQGTKVEIK |
| M4A02_VH | 848 | EVQLVESGGGLVEPGGSLRLSCAASGFMFT |
| | | SYAMHWVRQAPGRGLEWVSSITGGGNFVEY |
| | | AVSVQGRFTVSRDNNQNSLYLQMNSLRVED |
| | | TAMYYCAREDYNSGANLHWFDPWGQGTLVT |
| | | VSS |
| M4A02_VL | 849 | QSVLTQPPSVSGAPGQRVTISCTGSSSNIG |
| | | AFFDVHWYSHLPGTAPKLLIYGDRNRPSGV |
| | | PERFSGSKSGASASLTITGLRAEDEADYYC |
| | | QSYDRGLSAWVFGGGTRVTVL |
| M4A05_VH | 850 | EVQLVESGGGLVKPGGSLRLSCAAFGFSLS |
| | | GYGMNWVRQAPGKGLQWVSSITASSSYITY |
| | | RDSLRGRFTISRDNAQNSLYLDMTALRVED |
| | | TAVYYCATNGKLPPYFDHWGQGVLVVVSS |
| M4A05_VL | 851 | QSVLTQPPSVSGAPGQRVTISCTGSGSNIG |
| | | AGYDVHWYQQLPGTAPKLLIYDNNHRASGV |
| | | PDRVSASWSGTSASLAITGLQAEDEADYYC |
| | | QSYDRNLEGVVFGGGTKLTVL |
| M4A06_VH | 852 | EVQLVESGGGLVRPGGSLRLSCAASGFSLT |
| | | TDEVNWVRQAPGKGPEWVSFISASGHTIYY |
| | | ADSVKGRFTISRDNGRNSVYLQMNSLRAED |
| | | TAVYYCARVGRLGGVGYYYALGPFDLWGQG |
| | | TMVTVSS |
| M4A06_VK | 853 | EIVLTQSPGTLSLSPGERATLSCRASQSIT |
| | | SNYLAWYQQKPGQAPRLLIYGASSRATGIP |
| | | DRFSGSGSGTDFTLTISRLQPEDFAVYYCQ |
| | | QYGSSLYIFGQGTKLEIK |
| M4A08_VH | 854 | EVQLVESGGALVRPGGSLRVSCEASGFNFR |
| | | THWMHWVRQAPGKGLVWVARTNGDGSVTNY |
| | | ADSVKGRFTFFRDNAKNTLFLQMTSLRVDD |
| | | TAVYYCARERLDYSSTRGHFPGDFDSWGQG |
| | | TLVTVSS |
| M4A08_VK | 855 | DIVMTQSPDSLAVSLGERATINCKSSESVL |
| | | YTSNSKNYLAWYQQKPGHPPKVLIYWASTR |
| | | ESGVPDRFSGSGSGTDFSLSISSLQAEDVA |
| | | VYYCQQYFTIPRTFGQGTKVEIK |
| M4A10_VH | 856 | QVQLVQSGAEVKKPGASVKVSCKASGYTFN |
| | | GHYLHWVRQAPGQGLEWMGWINPNSGGTDY |
| | | AQRFQGRVTMTRGTSISTAYMELSRLTSDD |
| | | TAVYFCARDNFGFWSASSGRHNWLDSWGQG |
| | | TLVIVSS |
| M4A10_VL | 857 | NFMLTQPHSVSESPGKTVTISCTRSTGNIA |
| | | SNYVQWYQQRPGSSPTTVIYEDNQRPSAVP |
| | | DRFSGSIDSSSNSASLTVSGLRTEDEADYY |
| | | CQSYDSGHHVVFGGGTKLTVL |
| M4A11_VH | 858 | QVQLVQSGSEVKRPGASVRVSCTASGYTFS |
| | | NYGVSWMRQAPGHGLEWMGWISTHNQNVRY |
| | | SQNFQGRVTVTTDTSTSTAYMDLRGLKPDD |
| | | TAVYFCARMIPVGTTYTPYWTGWTPGGMDI |
| | | WGQGTTVIVSS |
| M4A11_VL | 859 | QSVLTQPPSASGTPGQRVIMSCSGTTSNIG |
| | | RNFVYWYQQLPKEAPTLLIYGNDQRPAGVP |
| | | DRFSGSTSGTSASLAISGLRSGDEADYYCA |
| | | TWDDSVNGPVFGGGTKLTVL |
| M4A12_VH | 860 | QLQLQESGSGPGLVKPSETLSLICNASGDS |
| | | VTRNAYYWGWLRQPPGKGLEWIGIMYYSGS |
| | | TFYNPSLKSRLTISVDTSKNQFSLKLRSVT |
| | | AADTATYYCGEATLTTYDAFDVWGHGTMVT |
| | | VSS |
| M4A12_VL | 861 | QSVLTQPPSVSAAPGQRVTISCSGGNSNIG |
| | | KNYVSWYQQVPGTAPKLLIYENNKRPSGIP |
| | | DRFSGSKSGTSATLGITGLQTGDEADYYCG |
| | | AWDSSLNGVLFGGGTRLTVL |
| M4B01_VH | 862 | EVQLLESGGGLVQPGGSLRLSCAASGFNFN |
| | | TYAMSWVRQAPGKGLKWISTISGSGGVKYY |
| | | ADSVKGRFTISRDNSGSILYLQMNRLRADD |
| | | TAVYYCAKSQNPQLVFFDSFDMWGQGSMVT |
| | | VSS |
| M4B01_VL | 863 | QSVLTQAPSASAAPGQKVTISCSGSTSNIG |
| | | NSYVSWYQQVPGTAPKLLIYEDNQRPSGIP |
| | | DRFSGSKSGTSATLGITGLQTGDEADYYCG |
| | | TWDNSLTEVVFGGGTKLTVL |
| M4B02_VH | 864 | QLQLQESGPSETLSLTCTVSGGSISSSSYY |
| | | WGWIRQPPGKGLEWIGSIYYSGSTYYNPSL |
| | | KSRVTISVDTSKNQFPLKLSSVTATDTAVY |
| | | YCARLGVGLYEAFDIWGQGTMVTVSS |
| M4B02_VK | 865 | DIQMTQSPSSLSASVGDRVTITCRASQSIS |
| | | SYLNWYQQKPGKAPKLLIYAASSLQSGVPS |
| | | RFSGSGSGTDFTLTISSLQPEDFAIYYCQQ |
| | | SYSTPNTFGQGTRLEIK |
| M4B03_VH | 866 | QVQLVQSGAEVKKPGASVKVSCKASGYIFS |
| | | NYRMHWVRQAPGQRLEWMGWITAGSGNTRY |
| | | SEQFQGRVAFTSDTDATTVSMELSGLRFED |
| | | TAVYFCAREYNWNDAFPYWGQGTLVIVSS |
| M4B03_VL | 867 | SYVLTQPPSVSVAPGQTARIPCGGNNIGSK |
| | | SVHWYQQKSGRAPVLVVYDDSDRPPGIPDR |
| | | FSGSNSGNTATLTINKIEAGDEADYYCHVS |
| | | DDNSDHVFFGGGTQLTVL |
| M4B04_VH | 868 | QVQLVESGGGVVQPGKSLRLSCAASGFTFR |
| | | SYTMFWVRQAPGKGLEWVAIILHDGTNRYY |
| | | TDSVKGRFTISRDNTKNTVYLQMNSLRPED |
| | | TAMYYCARESLEWMLDWGQGTLVTVSS |
| M4B04_VK | 869 | EIVMTQSPATLSVSPGERATLSCRASQSLS |
| | | GNLAWFQQKPGQAPRLLIYGTSTRATGIPA |
| | | RFSGSGSGTEFTLTISSLQSEDFAVYYCQQ |
| | | YNNWPPVFTFGPGTKVDIK |
| M4B05_VH | 870 | QVQLVQSGPEVRKPGASVIISCKASGYSFI |
| | | SYYIHWVRQAPGQGLEWMGIINPSGGSTEY |
| | | AQKYQGRLTITRDTSTTTVSMTLSSLRSDD |
| | | TAVYFCAREQAVGAYDIWGQGTMVTVSS |
| M4B05_VK | 871 | EIVLTQSPGTLSLSPGERATLSCRAGQLIN |
| | | GNYLAWYQKKPGQAPSLLIYDVSRRAAGVP |
| | | DRFTGSGSGTDFTLTISRLEPDDFAVYYCQ |
| | | HYGRSPSPTFGGGTKVEIK |
| M4B06_VH | 872 | EVQLLESGGGFVQPGGSLRLSCVVTGFTFS |
| | | NYAMCWVRQAPGKGPEWVSGIGGSVGTTYY |
| | | ADSVKGRFTISRDNSKNTLYLQMSGLRAED |
| | | TARYYCAKVPSFFVFEDGFDMWGQGTVVTV |
| | | SS |
| M4B06_VL | 873 | QSVLTQPPSASGNPGQRVTISCYGSGSNIG |
| | | NNYVHWFRQLPGTAPRLLIYNTDILVPGVP |
| | | DRFSGSESGTSASLAISGLQSGDEADYFCA |
| | | AWDDTLNGYVFGPGTTVTVL |
| M4B11_VH | 874 | QVQLVQSGTEVQTPGASVKVSCKTSGYIFT |
| | | GYFMHWVRQAPGQGFEWMGWVNPYSGATTY |
| | | AQKFQGRVTMTRDTSISTVYMELSRLTSDD |
| | | TAIYYCAKDTSNSGPDLLEHWGQGTLVIVS |
| | | S |
| M4B11_VK | 875 | EIVLTQSPATLSLSPGERATLSCRASQSIS |
| | | NFLVWYQRKPGQAPRLLIFDVSKRATGIPT |
| | | RFSGSGSGTDFTLTISSLEPEDFAVYYCQQ |
| | | RSNWPPGYTFGQGTKLEIK |
| M4C02_VH | 876 | QLQLQESGPRLVKPSETLSLTCSVSGGSIS |
| | | SSSLYWGWIRQSPGKGLEWIGTIYYGGSSD |
| | | YNPSLKSRLTMSVDTSKNHFSLRLSSVTAA |
| | | DTAVYYCARVGSPVTTTVWFDPWGQGTLVT |
| | | VSS |
| M4C02_VK | 877 | DIQMTQSPSSLAASIGDRVTITCRASQTIS |
| | | TYLNWYQQKPGKAPKLLIYAASTLQSGVPS |
| | | RFSGSGSGTDFTLTISSLQPEDFATYYCQQ |
| | | TYSSLALTFGGGTKVEIR |
| M4C03_VH | 878 | EVQLVESGGTLVQPGGSLRLSCAASGFRFN |
| | | AFSMNWVRQAPGKGLEWVSYIDSRSNIIYY |
| | | ADSVKGRFTISRDNAKTSLYLRMSSLRDED |
| | | TAVYYCAKALCVVNYDFWSGLLGCAMDVWG |
| | | QGTTVTVSS |
| M4C03_VL | 879 | QSVLTQPPSVSGAPGQRVTISCTGTTSNIG |
| | | AGFDVHWYQQVPGTAPKLLIYANTDRPSGV |
| | | PDRFSASKSGTSASLAITGLQVEDEAHYYC |
| | | QTYDTTLSGSTVFGGGTKLTVL |
| M4C04_VH | 880 | RVTLRESGPALLKPTQTLTLTCTFSGFSLN |
| | | TYGMSVNWIRQPPGKALEWLSRIDWDGDKY |
| | | FNTSLKTRLTISKDTSKNQVVLTMTNMEPV |
| | | DTATYYCAQSVIVPAQSFVSWFDPWGPGTL |
| | | VTVSS |
| M4C04_VK | 881 | EIVMTQSPATLSVSPGERATLSCRASQSVR |
| | | SNLAWYQQKPGQAPRLLIYGASSRATGIPA |
| | | RFSGSGSGTEFTLTISSLQSEDLAIYYCQQ |
| | | YNNWPPGAFGQGTKVEIK |
| M4C08_VH | 882 | EVQLVESGGTLVQPGGSLRLSCAASGFKFN |
| | | AFSMNWVRQAPGKGLEWVSYIDSRSNIIYY |
| | | ADSVKGRFTISRDNAKNSLYLRMSSLRDED |
| | | TAVYYCAKALCVVNYDFWSGLLGCAMDVWG |
| | | QGTTVTVSS |
| M4C08_VL | 883 | QSVLTQPPSVSGAPGQRVTISCTGNTSNIG |
| | | AGFDVHWYQQVPGTAPKLLIYANTDRPSGV |
| | | PDRFSASKSGTSASLAITGLQAEDEADYYC |
| | | QTYDRSLSGSTVFGGGTKLTVL |
| MIC11_VH | 884 | QVQLVQSGAEMKKPGSSVKVSCKTSGGSFS |
| | | SYPVSWVRQAPGQGPEWMGGIIPLSGTTHY |
| | | AQMFQGRVSITADDSTRTVFMELTSLRSGD |
| | | TAVYYCARSGAPTYYYDSSGFHGGAFHFWG |
| | | QGTVVTVSS |
| MIC11_VL | 885 | DIVMTQSPDSLAVSLGERATINCKSSQSVL |
| | | YSSNSKSYLAWYQQKPGQPPKLLIYWASSR |
| | | ESGVPDRFSGSGSGTDFTLTISSLQAEDVA |
| | | VYYCQQFYTSPHTFGQGTKLEIKRT |
| MIC7_VH | 886 | QVQLVESGGGLVQPGRSLRLSCAASGFTFS |
| | | SYAMHWVRQAPGKGLEWVAIITYDGSGKYY |
| | | ADSLEGRITISRDDPKSMLYLHMRSLTAED |
| | | TAVYYCVKSGSFSSAFWLEDWGQGTLVTVS |
| | | S |
| MIC7_VK | 887 | EIVLTQSPATLSLSPGERATLSCRASQSIS |
| | | NFVAWYQQKPGQAPRLLIYDASNGATGTPA |
| | | RFSGSGSGTDFTLTISSLEPEDFAVYYCQQ |
| | | RSTWTFGQGTKVEIKRT |
| MIC7s_VH | 888 | QVQLVQSGPEVRKPGSSVRVSCKASGGTFT |
| | | RLTITWVRQAPGQGLEWMGRIIPAVDITNY |
| | | APKFQGRVTITADKSTGAVYMDLISLTSDD |
| | | TAIYFCATVLDWSNALDIWGQGTMVTVSS |
| MIC7s_VK | 889 | DIVMTQTPLSLSVTPGQPASISCKSSESLL |
| | | NSDGKTYLFWYLQKPGQSPQVLIYDLSSRF |
| | | SGVPERFSGSGSGTDFTLKISRVEPEDVGI |
| | | YYCMQGLHLPWTFGRGTRLEIKRT |
| MID2_VH | 890 | QVQLQESGPGLVKPSGTLSLTCTVSGGSVN |
| | | SDHWWTWVRQSPETGLEWIGELNHIGTFNY |
| | | NPSFKSRATISIDTSKNQFSLTLTSVAAAD |
| | | TAVYYCARRPPPLPEYFYYYFMDVWGNGTT |
| | | VTVSS |
| MID2_VK | 891 | DIQMTQSPSSLSASVGDRVTITCRTSQSIR |
| | | GHINWYQQKPGKGPKLLIYGASSLQSGVPS |
| | | RFSGSGSGTDFTLTISSLQAEDFATYYCQQ |
| | | SYSNPYSFGRGTKLEIKRT |
| M2B6_VH | 892 | QVQLQESGPGLVKPSETLSLTCSVSGASIG |
| | | GYYWGWIRQPPGKGLEWLGYIYYSGRTVYS |
| | | PSLRGRLALSLDMSRQQLSLRLRSVAAADT |
| | | AVYYCASLTITPGWFDSWGPGTLVTVSS |
| M2B6_VL | 893 | QSVLTQPPSVSGAPGQRVTISCTGSSSNIG |
| | | AGYDVHWYQQFPGSAPKLLIYGNTKRPSGV |
| | | PDRFSGSTSGSSASLAITGLQTEDEAEYYC |
| | | QSYERNLDGALFGGGTKLSVL |
| M2D2_VH | 894 | QVQLVQSGAEVKKPGASVKVSCKASGYNFD |
| | | NYYMSWVRQAPGQGLEWLGIIDPSGGRISY |
| | | APKFQGRVTLTRDTSTSTVYMELRGLRFED |
| | | TALYYCARLEATHEGPWGQGTLVTVSS |
| M2D2_VL | 895 | QSVLTQPPSASGTPGQRVTISCSGSNSNIG |
| | | SSTVNWYQQLPGTAPKLLIYSDNHRPSGVP |
| | | DRFSGSRSGTSASLVISGLHSEDEADYYCG |
| | | TWDDGLNGPYVFGTGTKVTVL |
| M4F8_VH | 896 | QVQLVQSGPEVKKPGASVKISCKASEYTFT |
| | | NHYLHWVRQAPGQGLEWLGIINPTGGSTYY |
| | | AGDFQGRVTLTRDASTTTVYMELSSVKSED |
| | | TAIYYCARDRGMRYFDYWGQGTLVTVSS |
| M4F8_VL | 897 | QSVLTQPPSVSGAPGQRVSISCTGISSNIG |
| | | ATYDVHWYQQLPGTAPKLLIYGNNNRPSGV |
| | | PDRFSGSKSGTSASLAITGLQAEDEAVYYC |
| | | QSYDSVLSGSVIFGGGTILTVL |
| M5F2_VH | 898 | EVQLVESGGGLVQLGGSLRLSCAGSGFSFS |
| | | TFWMSWVRQAPGKGLEWLANIKPDGSEKFY |
| | | VDSVKGRITLSRDNANNSLYLQMRNLRDDD |
| | | TAVYYCARDRGYCRGGSCYRRNAFDLWGQG |
| | | TMVTVSS |
| M5F2_VL | 899 | QSALTQPPSASGSPGQSVTISCTGTSGDVG |
| | | GYTFVSWYQQYPGKAPKLIIYEVTKRPSGV |
| | | PDRFSGSKSGNTASLTVSGLQTEDEADYFC |
| | | SSYSSTNNLRVFGGGTKLTVL |
| M6E10_VH | 900 | QVQLVQSGAEVKKPGSSVKVSCNASGGIFD |
| | | TNVINWVRQAPGQGLEWMGGIIPPFGTALY |
| | | AHQFQGRLTLTVDESTNTAYMDLSSLRSDD |
| | | TAVYFCARDRTVIKYADGMDVWGQGTTVTV |
| | | SS |
| M6E10_VK | 901 | DIQMTQSPSSLSASVGDKVAITCRASQTIS |
| | | PYLNWYQQKPGKAPNLLIYGTNKLQSGVPL |
| | | RFSGSGSGTDFTLTINGLQPEDFATYFCQQ |
| | | SHTTPLTFGGGTKVEIKRT |
| M7D5_VH | 902 | QVQLVQSGAEVKKPGSSVKVSCKASGGTLS |
| | | NYAISWVRQAPGQGLEWMGGIIPIFGTAMY |
| | | AHNFQDRVTFTADESTNTVYMAVSRLTSDD |
| | | TAVYYCAQDRTVTKYAHGLDVWGQGTTVTV |
| | | SS |
| M7D5_VK | 903 | DIQMTQSPSSLSASVGDRITITCPASQSLL |
| | | TYLNWYQQKPGKAPKLLIYSASELPSGVPS |
| | | RFSGSGSGTEFTLTVTGLQPDDFATYYCQQ |
| | | SHTTPLTFGGGTKVEIKRT |
| M8C10_VH | 904 | QVQLVESGGGVVQPGRSLRLSCAASGFTFT |
| | | TYTMHWVRQAPGKGLEWVALISSDGNNKYY |
| | | ADSVKGRFTISRDNSKNTLYLQMSSLRAED |
| | | TAVYYCARGYYSGGDAFDVWGQGTMVTVSS |
| M8C10_VK | 905 | DIQMTQSPSTLSASVGDRVTITCRASQTIT |
| | | IWLAWYQQKPGKAPKLLIYDAVSLESGVPS |
| | | RFRGSGSGTDFTLTISSLQPDDFATYYCQQ |
| | | YNNYPYTFGQGTKLEIKRT |

### Example 3. Characterization of an Ultra-Potent PreF-Specific hMPV F Neutralizing mAb M4B06

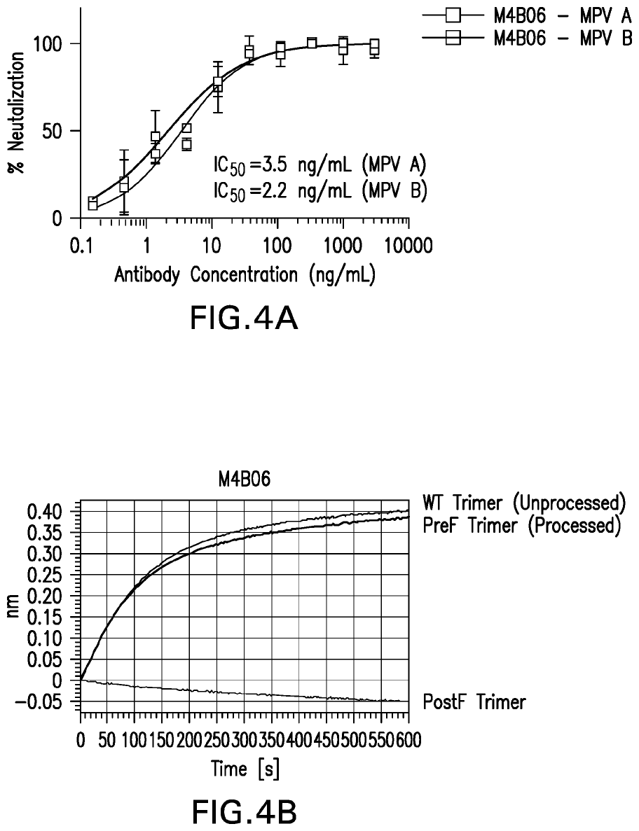

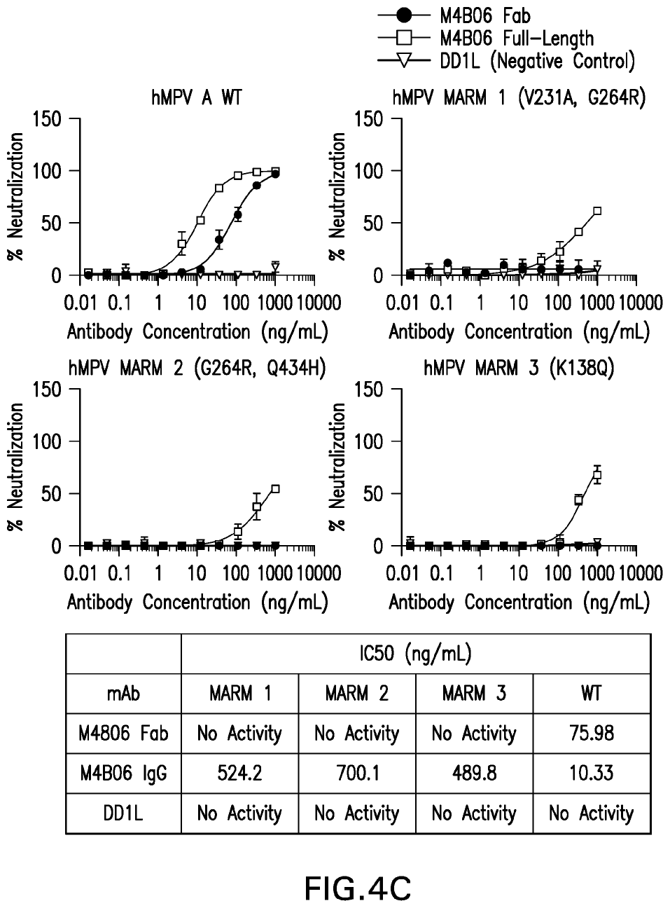

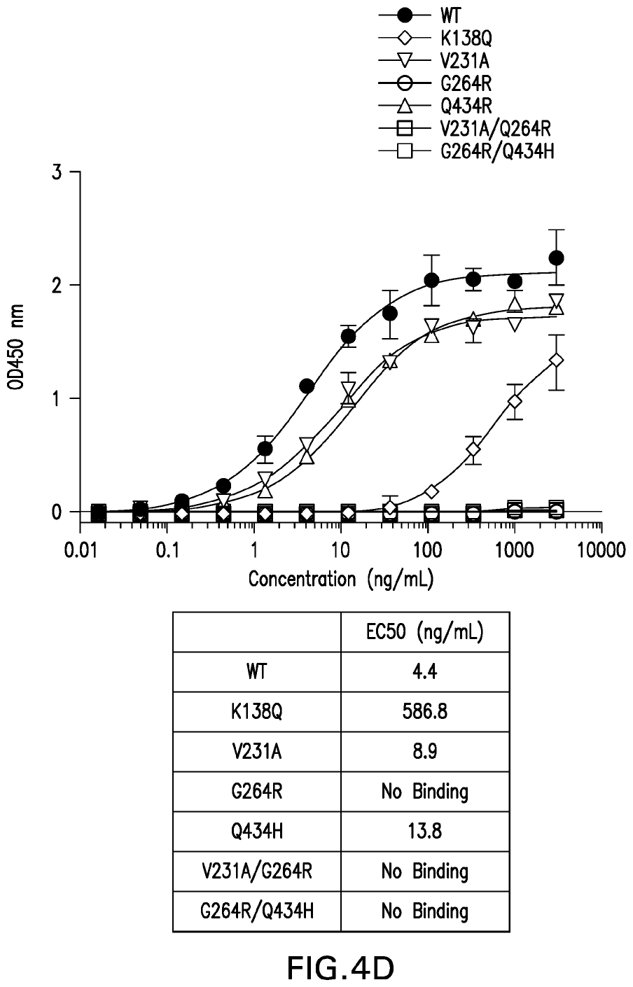

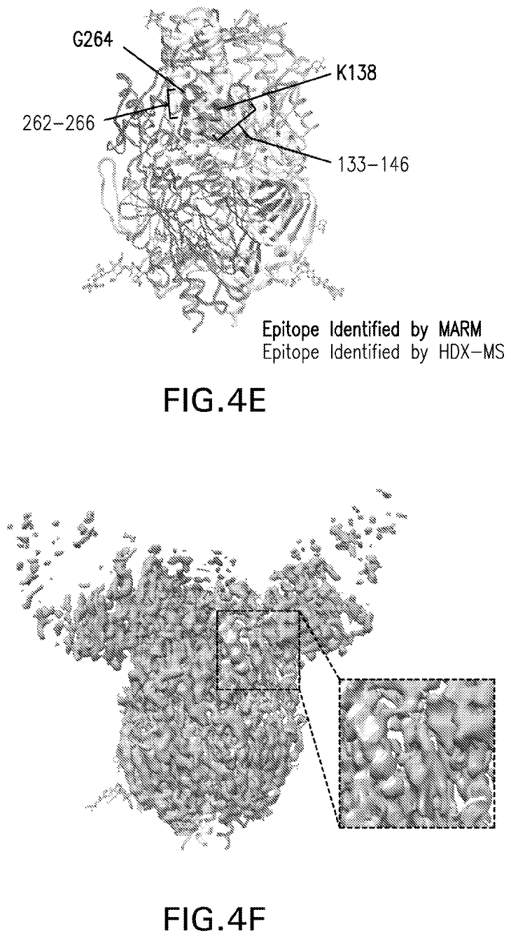

Among isolated mAbs, clone number M4B06 showed ultra-high hMPV neutralization potency on both tested hMPV A and B subtypes with IC50 at 3.5 ng/mL and 2.2 ng/mL respectively (FIG. 4A). BLI binding assay showed that M4B06 only bound WT and stabilized PreF protein but did not bind PostF protein, indicating that M4B06 targeted a PreF-specific epitope (FIG. 4B).

To further understand the epitope of M4B06, monoclonal antibody resistant mutants (MARMs) of an hMPV A strain were generated under the selection pressure of M4B06. After three rounds of culture with increasing concentration of M4B06 Fab, it was observed that the hMPV virus was able to replicate under the Fab concentration of 40 μg/mL, which is more than 500 folds of the IC50 of the wild-type virus. Sequencing of single purified plaques of MARMs revealed three types of MARMs: V231A/G264R, G264R/Q434H, and K138Q. The neutralization potency of M4B06 IgG to these three types of MARMs reduced the potency of the immunoglobulin 52-fold, 70-fold, and 49-fold respectively. TheM4B06 Fab failed to neutralize any type of MARM of the hMPV A strain at all, confirming that the mutations generated in these MARMs impaired the neutralizing activity of M4B06 (FIG. 4C).

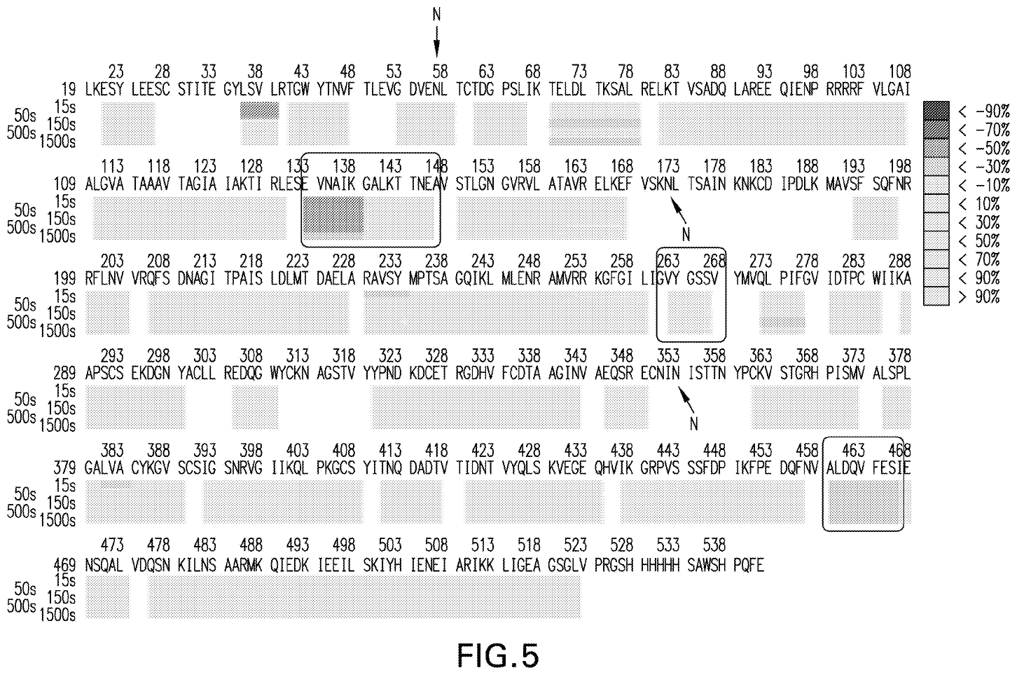

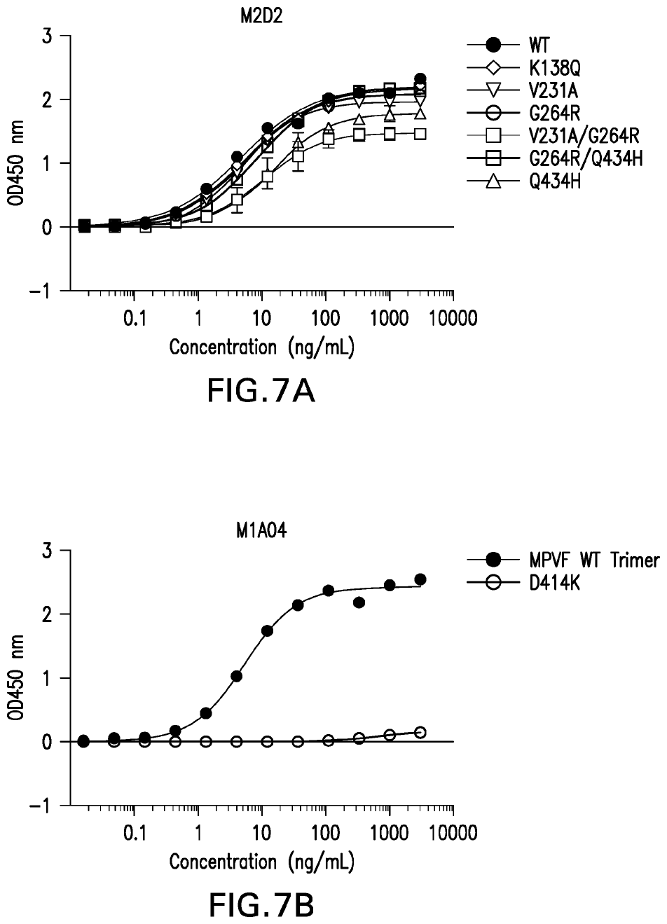

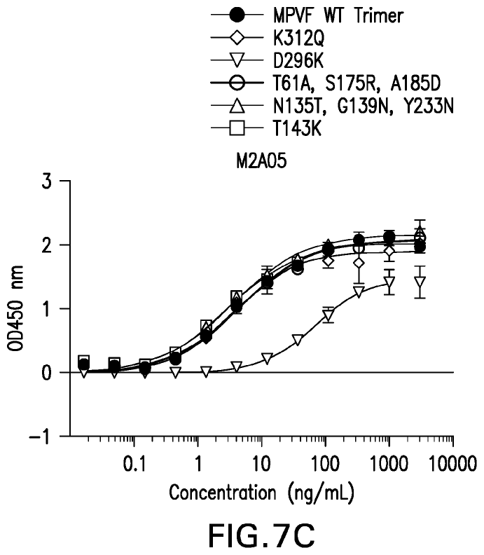

Additionally, investigators generated hMPV F variants carrying single and double MARM-derived mutations to determine whether the mutations/variations interrupted M4B06 binding. Data from the ELISA binding assay showed that these mutations did not impact the binding to a control antibody M2D2, which targets a different epitope [53], such that it may be that the mutant proteins were folded similarly as the WT protein (FIG. 7A). However, K138Q reduced binding for at least 100 fold and single or double mutants carrying G264R completely abolished M4B06 binding, suggesting that K138 and G264 are critical for M4B06 binding and likely located within the epitope of M4B06 (FIG. 4D). K138 and G264 are both solvent-exposed and in proximity to each other, located at a region equivalent to antigenic site V previously defined in RSV F (FIG. 4E). In a parallel epitope mapping study by hydrogen-deuterium exchange mass spectrometry (HDX-MS), peptides 131-146, 137-146, 260-266 showed significant reduced exchange rate between hMPV F-M4B06 complex and hMPV F alone. It may be that these peptides contained the epitope residues of M4B06, which is consistent with the results from MARM study (FIG. 4E, and FIG. 5). FIG. 5 is a heat map plot showing the difference in deuteration levels of the processed hMPV PreF trimer protein alone compared to the antigen in the presence of the M4B06 monovalent Fab at five time points (15, 50, 150, 500, and 1500 sec). Slower deuterium exchange indicated regions containing the binding sites. White areas are ‘gaps’ for which there was no sequence coverage, and thus no HDX-MS information was obtained.

Moreover, the identified epitope of M4B06 was further confirmed by a CryoEM map of M4B06-hMPV F complex at a resolution of 3.75 Å, which clearly showed that three M4B06 Fabs sit on the epitope area at a nearly perpendicular angle (FIG. 4F). Taken together, investigators identified a highly potent neutralizing mAb M4B06 targeting a hMPV PreF-specific epitope that is similar to the previously defined site V of RSV F.

### Example 4. Epitope Mapping of hMPV F-Specific mAbs

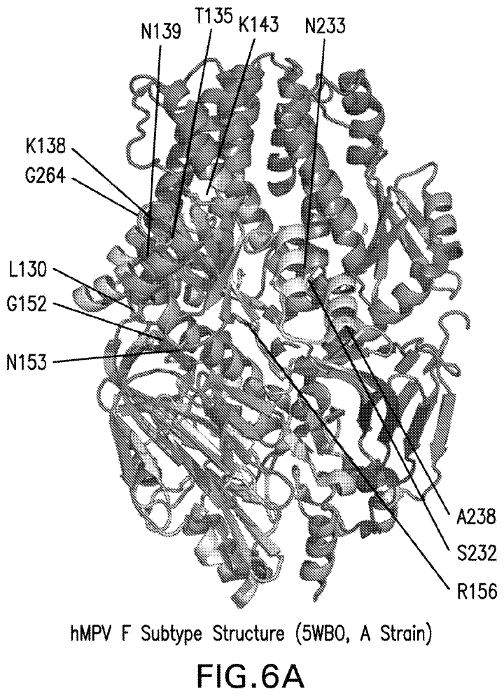

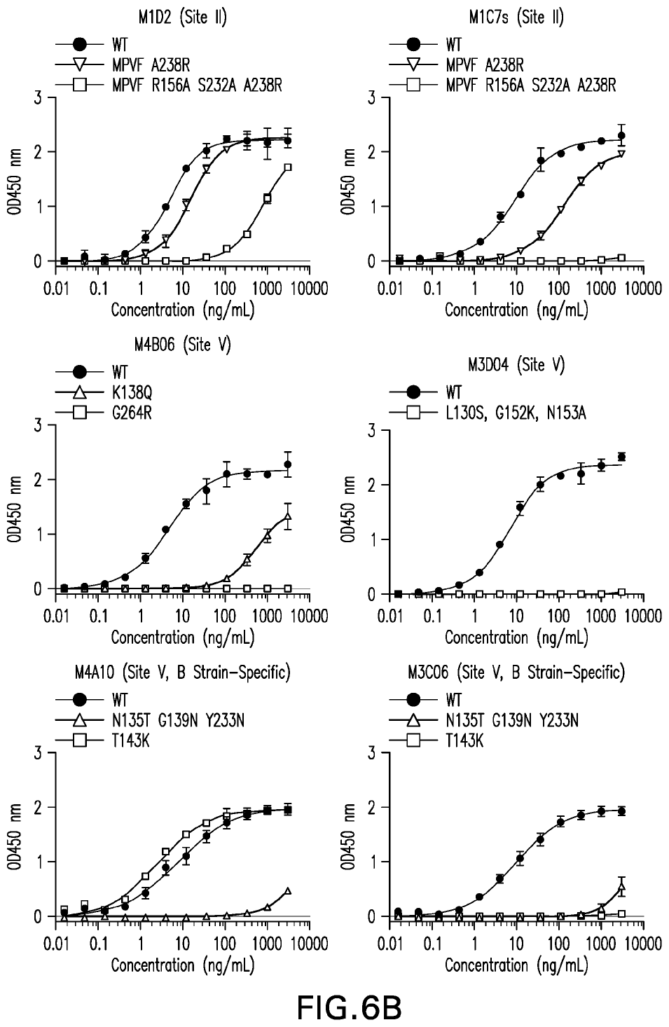

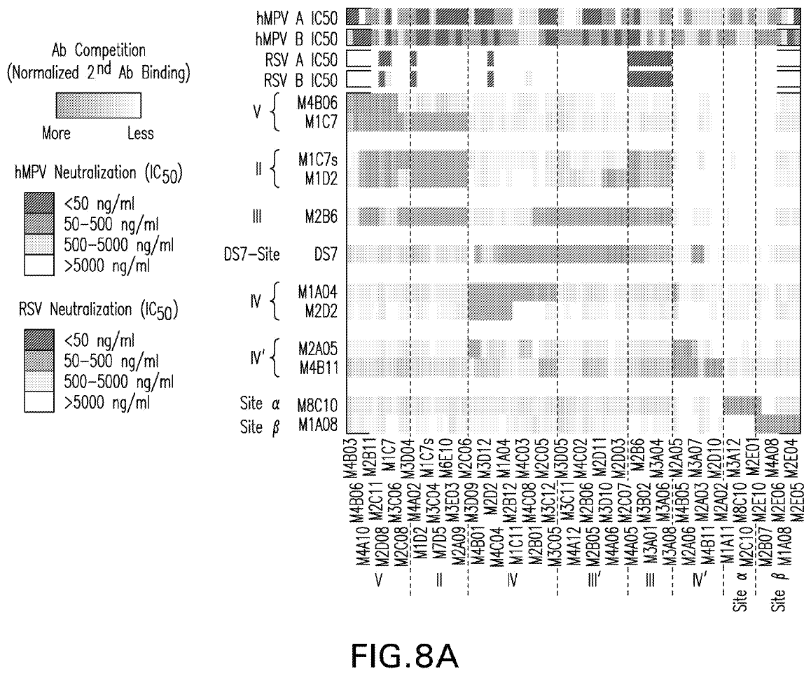

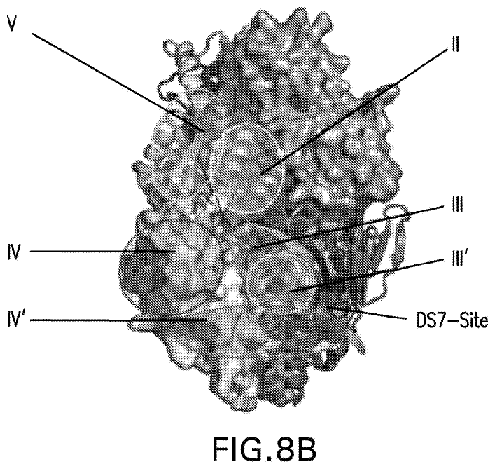

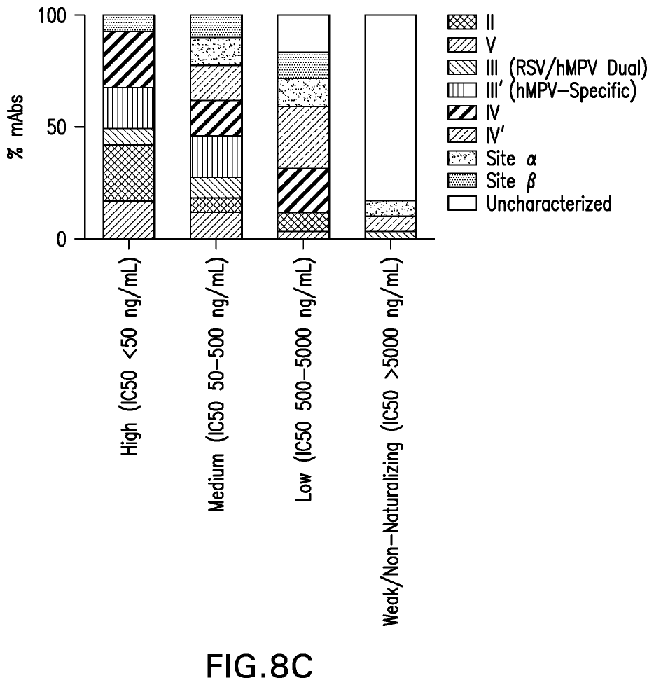

To map the targeted antigenic sites of all isolated hMPV F-specific mAbs, investigators designed a panel of hMPV F knock-out mutants and tested ELISA binding with isolated hMPV-F specific mAbs. A panel of mAbs (M1D2, M1C7s, M3D04, M4A10, M3C06, M4B06) were observed to reduce binding to PreF mutants on a variety of residues located at regions equivalent to antigenic site II and V defined on RSV F (FIG. 6A and FIG. 6B). In addition, M1A04 have been mapped to site IV (FIG. 7B). Antibodies M1D2, M1C7s, M1A04, together with aforementioned M4B06 (site V) and previously reported mAbs DS7 (DS7-site; Wen, X., et al., 2012. Nat Struct Mol Biol 19, 461-463), M2B6 (site III, cross-neutralizes RSV/hMPV), M2D2 (site IV, cross-neutralized RSV/hMPV), M1C7 (site V, cross-neutralized RSV/hMPV), were selected as reference mAbs to group isolated mAbs by BLI-based epitope binning. See also Xiao, X., et al., 2019 mAbs, 11:8, 1415-1427 citing M2B6, M2D2, M1C7. Based on the preliminary binning results, M4B11, M2A05, M8C10, and M1A08 were included as additional reference mAbs for epitope binning to characterize those mAbs that did not show significant competition with the reference mAbs described above (FIG. 8A).

Based on epitope binning, hMPV F-specific mAbs were categorized into eight groups (FIG. 8A). Four groups of mAbs showed typical competition profiles similar to the mAbs of previously defined sites V, II, III, IV of RSV F, thus these mAbs were mapped to sites V, II, III, IV, respectively. Binding analysis to different variants of PreF proteins revealed that mAbs use multiple binding modes to interact with antigenic sites II and V, as mutations within these sites impacted their binding differently (FIG. 6A and FIG. 6B). The other four groups of mAbs showed novel competition profiles that have not been reported. (1) The first group of mAbs significantly competed with site III mAb M2B6 and DS7 and had less competition with site II mAbs M1C7s and M1D2, suggesting they targeted a region between classical site III and DS-7 site. Unlike classical site III mAbs that cross-neutralize both hMPV and RSV, this group of mAbs only neutralized hMPV. The investigators named this antigenic site as site III′ (FIG. 8A, FIG. 8B); (2) The second group of mAbs mostly competed with two mAbs within its own group. A representative mAb in this group, M2A05, only neutralized hMPV B subtype.

To further map its epitope, the investigators analysed the M2A05 antibody binding to a panel of hMPV-B F variants that converted subtype B-specific residues to their counterparts in hMPV-A F. Data show that a single D296K mutation of F antigen significantly reduced binding of M2A05 (FIG. 7C), suggesting that D296 is within the epitope of M2A05. This antigenic site, which sits below classical site IV on the hMPV F structure, was named as site IV′ (FIG. 8A and FIG. 8B). (3) The third group of mAbs competed with antibody M8C10, which is an mAb that targets an epitope at internal trimerization interface (named as site α). These data may indicate that these mAbs all likely target internal epitopes of hMPV F. (4) The last group, named as site β, did not compete with any six groups mapped on hMPV PreF surface. Most of site β mAbs only competed with mAb M1A08 within its group, except M2E11L that competed with both antibody M8C10 and antibody M1A08. The investigators were not able to determine the detailed epitopes of site β by knock-out mutants or obtained its crystal structure, thus the epitope of site β remains to be further explored in detail. Interestingly, investigators did not find evidence showing any mAb targeted site Ø. In the mAb group with high neutralizing potency (IC₅₀<50 ng/mL), only less than 20% mAbs targeted site V (FIG. 8C), and 67% of mAbs targeted site II, IV, and III′, which are located on the side of hMPV F antigen (FIG. 8B). This is significantly different from findings of RSV F, which suggested immune-dominance of sites 0 and V [48].

### Example 5. Correlations Among Antigenic Sites, Neutralization Potency, and Binding-Specificity to Different hMPV F Conformations

To investigate the binding-specificity of isolated mAbs to different conformations of hMPV F antigens, investigators analysed the binding affinity of isolated mAbs to three forms of hMPV F antigens by BLI. (1) the uncleaved hMPV PreF trimer [34], which was used for B cell sorting and epitope binning (unprocessed PreF, see FIG. 2A); (2) the prefusion stabilized hMPV F trimer 115BV, which showed efficient cleavage between F1 and F2 after co-expression with furin [28] (processed PreF, see FIG. 2B); and (3) an hMPV PostF, as confirmed by EM negative staining (FIG. 9). See also Table 4. Due to the high aviditiy between antigen trimer and bivalent antibodies, the dissociation of many antibodies was too slow to determine the Kd. Therefore, we used the absolute binding response (nm) as an indicator of apparent binding affinity.

TABLE 4
Data from BLI-based assays for anti-hMPV antibodies and different antigens

| | | Octet Binding (actual response in nm) | | | Processed PreF |
|---|---|---|---|---|---|
| | | | Processed | | trimer/ |
| | | Unprocessed | hMPV | | Unprocessed |
| | | hMPV PreF | PreF | MPV | PreF trimer |
| mAbs | Sites | trimer | trimer | PostF | binding Ratio |
| M4B03 | V | 0.3242 | 0.1078 | −0.061 | 0.33 |
| M4B06 | V | 0.4008 | 0.3839 | −0.0518 | 0.96 |
| M2C08 | V | 0.415 | 0.1571 | −0.0185 | 0.38 |
| M3D04 | V | 0.4255 | 0.2359 | 0.0251 | 0.55 |
| M1C7 | V | 0.3121 | 0.3593 | 0.0541 | 1.15 |
| M2B11 | V | 0.3798 | 0.4977 | −0.0289 | 1.31 |
| M2C11 | V | 0.3488 | 0.3616 | −0.03 | 1.04 |
| M2D08 | V | 0.4161 | 0.1823 | −0.0414 | 0.44 |
| M3C06 | V | 0.2082 | 0.1946 | −0.0338 | 0.93 |
| M4A10 | V | 0.3252 | 0.2298 | −0.0388 | 0.71 |
| M1C7s | II | 0.303 | 0.3637 | 0.1649 | 1.20 |
| M1D2 | II | 0.4184 | 0.4595 | 0.5537 | 1.10 |
| M2A09 | II | 0.3311 | 0.4419 | 0.0797 | 1.33 |
| M2C06 | II | 0.4078 | 0.4722 | 0.4362 | 1.16 |
| M3C04 | II | 0.3791 | 0.3097 | 0.409 | 0.82 |
| M3E03 | II | 0.4045 | 0.4268 | 0.4198 | 1.06 |
| M4A02 | II | 0.4194 | 0.4194 | 0.0488 | 1.00 |
| M6E10 | II | 0.3366 | 0.3933 | 0.0923 | 1.17 |
| M7D5 | II | 0.2961 | 0.3471 | 0.0826 | 1.17 |
| M2B6 | III | 0.3586 | 0.3515 | 0.1991 | 0.98 |
| M3A01 | III | 0.251 | 0.2606 | −0.062 | 1.04 |
| M3A06 | III | 0.4511 | 0.1838 | −0.047 | 0.41 |
| M3A08 | III | 0.422 | 0.2486 | −0.0217 | 0.59 |
| M3B02 | III | 0.3247 | 0.1015 | 0.0061 | 0.31 |
| M3E04 | III | 0.3003 | 0.0401 | 0.0262 | 0.13 |
| M3E10 | III | 0.3506 | 0.17 | 0.0082 | 0.48 |
| M4A05 | III | 0.4082 | 0.3998 | 0.0001 | 0.98 |
| M2B05 | III′ | 0.4473 | 0.5418 | 0.486 | 1.21 |
| M2B06 | III′ | 0.3889 | 0.4014 | 0.4356 | 1.03 |
| M2C07 | III′ | 0.418 | 0.4398 | 0.4541 | 1.05 |
| M2D03 | III′ | 0.4246 | 0.4659 | 0.5028 | 1.10 |
| M2D11 | III′ | 0.41 | 0.4359 | 0.4299 | 1.06 |
| M3C11 | III′ | 0.3165 | 0.0794 | 0.4326 | 0.25 |
| M3D05 | III′ | 0.3375 | 0.0831 | 0.5389 | 0.25 |
| M3D10 | III′ | 0.3798 | 0.3965 | 0.4731 | 1.04 |
| M4A06 | III′ | 0.3599 | 0.2809 | 0.449 | 0.78 |
| M4A12 | III′ | 0.3754 | 0.0773 | 0.4315 | 0.21 |
| M4C02 | III′ | 0.3351 | 0.3211 | 0.475 | 0.96 |
| M1A04 | IV | 0.4531 | 0.5573 | 0.6179 | 1.23 |
| M1C11 | IV | 0.3511 | 0.0597 | 0.5428 | 0.17 |
| M2A11 | IV | 0.2847 | 0.0658 | 0.293 | 0.23 |
| M2B01 | IV | 0.3148 | 0.0607 | 0.4652 | 0.19 |
| M2B12 | IV | 0.3775 | 0.4375 | 0.445 | 1.16 |
| M2C05 | IV | 0.3985 | 0.4636 | 0.2644 | 1.16 |
| M2D01 | IV | 0.3301 | 0.0622 | 0.4926 | 0.19 |
| M2D2 | IV | 0.3618 | 0.3513 | 0.4538 | 0.97 |
| M3C05 | IV | 0.4069 | 0.4839 | 0.3099 | 1.19 |
| M3C12 | IV | 0.3962 | 0.4559 | 0.4963 | 1.15 |
| M3D09 | IV | 0.2724 | 0.2157 | 0.4106 | 0.79 |
| M3D12 | IV | 0.4648 | 0.5163 | 0.496 | 1.11 |
| M3F03 | IV | 0.3924 | 0.1066 | 0.563 | 0.27 |
| M4B01 | IV | 0.4613 | 0.5106 | 0.5561 | 1.11 |
| M4C03 | IV | 0.2628 | 0.0323 | 0.5061 | 0.12 |
| M4C04 | IV | 0.3418 | 0.3026 | 0.4417 | 0.89 |
| M4C08 | IV | 0.2224 | 0.0268 | 0.467 | 0.12 |
| M2A02 | IV′ | 0.2613 | 0.0795 | −0.0402 | 0.30 |
| M2A05 | IV′ | 0.3779 | 0.4468 | 0.4493 | 1.18 |
| M2A06 | IV′ | 0.2371 | 0.052 | −0.0352 | 0.22 |
| M2B10 | IV′ | 0.2087 | 0.0278 | −0.0646 | 0.13 |
| M2D07 | IV′ | 0.3236 | 0.0757 | 0.0143 | 0.23 |
| M2D10 | IV′ | 0.3311 | 0.1294 | 0.0464 | 0.39 |
| M2F01 | IV′ | 0.3351 | 0.0736 | −0.0326 | 0.22 |
| M4B04 | IV′ | 0.1983 | 0.0232 | −0.0362 | 0.12 |
| M4B05 | IV′ | 0.2806 | 0.0529 | −0.0395 | 0.19 |
| M4B11 | IV′ | 0.2726 | 0.0489 | 0.4263 | 0.18 |
| M4F8 | IV′ | 0.2708 | 0.0584 | 0.0408 | 0.22 |
| M2A03 | IV′ | 0.3692 | 0.1088 | 0.4771 | 0.29 |
| M3A07 | IV′ | 0.3241 | 0.0536 | −0.022 | 0.17 |
| M5F2 | IV′ | 0.0652 | 0.0092 | 0.0162 | 0.14 |
| M1A08 | β | 0.3389 | 0.125 | 0.0512 | 0.37 |
| M2B07 | β | 0.3668 | 0.1363 | 0.0181 | 0.37 |
| M2B09 | β | 0.3023 | 0.0092 | −0.0435 | 0.03 |
| M2E04 | β | 0.3912 | 0.1994 | 0.0702 | 0.51 |
| M2E05 | β | 0.4264 | 0.2284 | 0.0401 | 0.54 |
| M2E06 | β | 0.4169 | 0.1918 | 0.0123 | 0.46 |
| M4A08 | β | 0.2906 | 0.0728 | 0.0302 | 0.25 |
| M2E11L | β | 0.3318 | 0.1621 | 0.0538 | 0.49 |
| M1A11 | α | 0.342 | 0.0515 | 0.0143 | 0.15 |
| M2C10 | α | 0.3819 | 0.0939 | −0.0261 | 0.25 |
| M2E01 | α | 0.408 | 0.126 | −0.016 | 0.31 |
| M2E08 | α | 0.3488 | 0.0781 | −0.0233 | 0.22 |
| M3A04 | α | 0.3733 | 0.0846 | −0.0261 | 0.23 |
| M3A12 | α | 0.3096 | 0.0766 | 0.0469 | 0.25 |
| M3C07 | α | 0.291 | 0.0745 | −0.0296 | 0.26 |
| M3D03 | α | 0.2419 | 0.0286 | −0.054 | 0.12 |
| M8C10 | α | 0.333 | 0.1099 | 0.0733 | 0.33 |
| M3A03 | Uncharacterized | 0.1815 | 0.0409 | 0.5677 | 0.23 |
| M3A05 | Uncharacterized | 0.0787 | 0.0071 | 0.2286 | 0.09 |
| M3A09 | Uncharacterized | 0.104 | 0.0151 | 0.1577 | 0.15 |
| M3A11 | Uncharacterized | 0.0491 | 0.0247 | 0.4138 | 0.50 |
| M3B04 | Uncharacterized | 0.1769 | 0.0297 | 0.446 | 0.17 |
| M3B06 | Uncharacterized | 0.0542 | 0.0061 | 0.2927 | 0.11 |
| M3B07 | Uncharacterized | 0.0691 | 0.0201 | 0.2915 | 0.29 |
| M3B12 | Uncharacterized | 0.1569 | 0.0102 | 0.4172 | 0.07 |
| M3C01 | Uncharacterized | 0.0597 | 0.0251 | 0.3269 | 0.42 |
| M3D06 | Uncharacterized | 0.1123 | 0.0331 | 0.6997 | 0.29 |
| M3D08 | Uncharacterized | 0.0649 | 0.0113 | 0.3075 | 0.17 |
| M3E02 | Uncharacterized | 0.008 | −0.0033 | 0.1817 | −0.41 |
| M3E05 | Uncharacterized | 0.126 | 0.018 | 0.5092 | 0.14 |
| M3E06 | Uncharacterized | 0.0556 | 0.0101 | 0.3289 | 0.18 |
| M3E08 | Uncharacterized | 0.0728 | 0.0213 | 0.2771 | 0.29 |
| M1A01 | Uncharacterized | 0.3257 | 0.0585 | 0.0169 | 0.18 |
| M1A12 | Uncharacterized | 0.2941 | 0.0714 | 0.0462 | 0.24 |
| M2B04 | Uncharacterized | 0.3278 | 0.1247 | 0.0152 | 0.38 |
| M2C01 | Uncharacterized | 0.107 | 0.0121 | −0.0726 | 0.11 |
| M2D02 | Uncharacterized | 0.2164 | 0.0468 | 0.5406 | 0.22 |
| M2D09 | Uncharacterized | 0.0397 | 0.011 | 0.5632 | 0.28 |
| M2E07 | Uncharacterized | 0.2164 | 0.0476 | 0.0137 | 0.22 |
| M3E01 | Uncharacterized | 0.1777 | 0.0125 | 0.6248 | 0.07 |
| M3E09 | Uncharacterized | 0.2256 | 0.0948 | −0.0506 | 0.42 |
| M3F02 | Uncharacterized | 0.225 | 0.0284 | 0.5175 | 0.13 |
| M4A11 | Uncharacterized | 0.209 | 0.0135 | 0.2736 | 0.06 |
| M4B02 | Uncharacterized | 0.0048 | −0.0056 | 0.0312 | −1.17 |

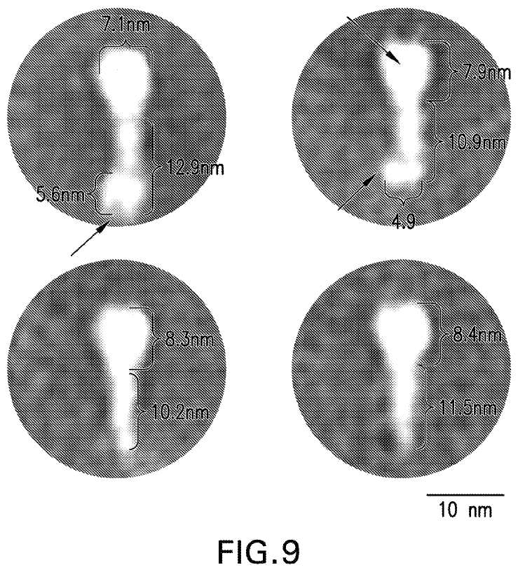

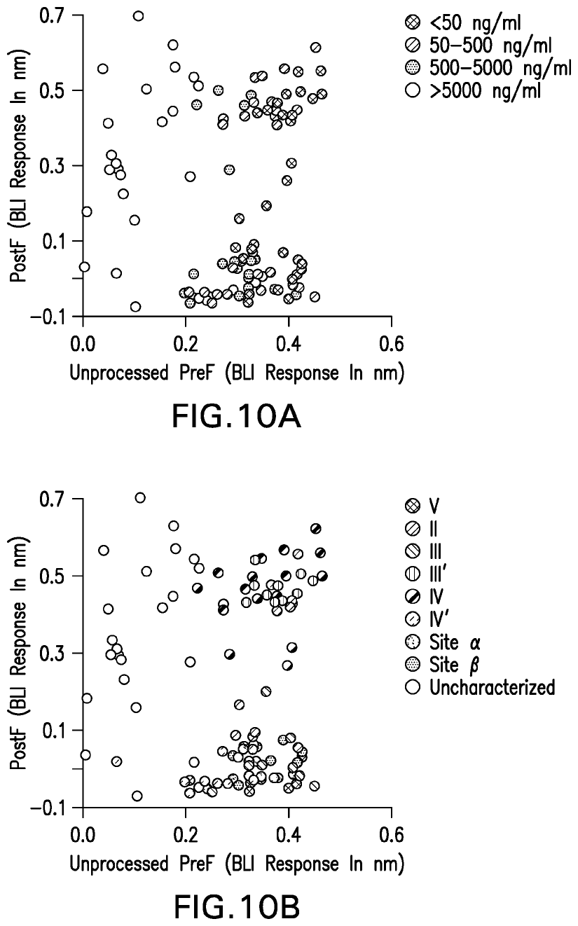

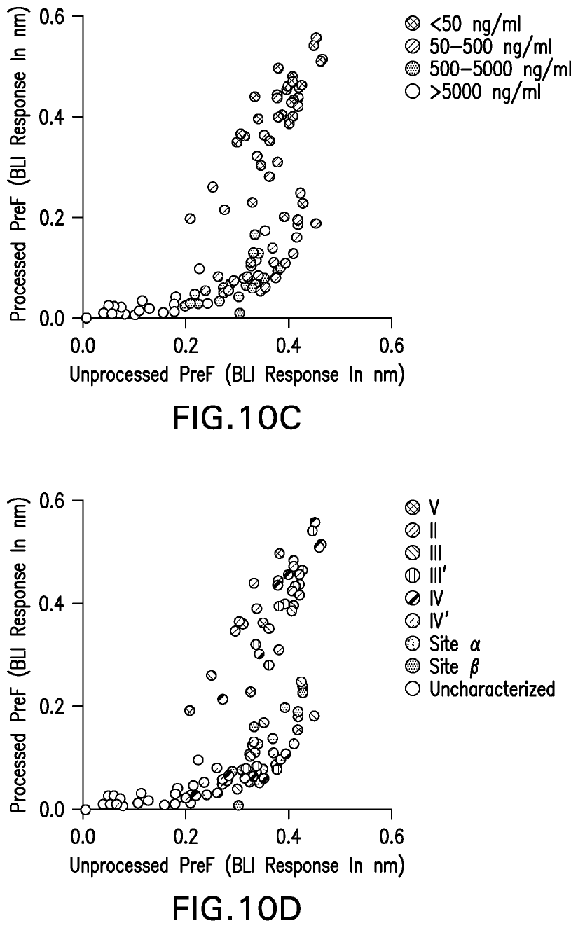

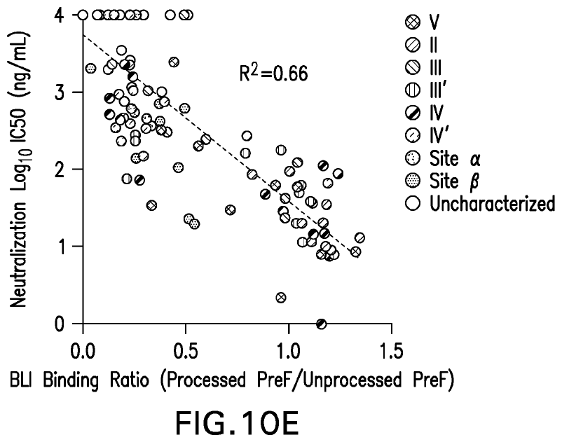

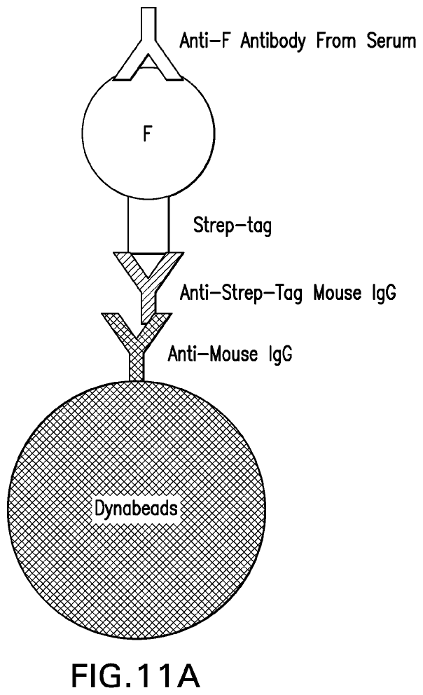

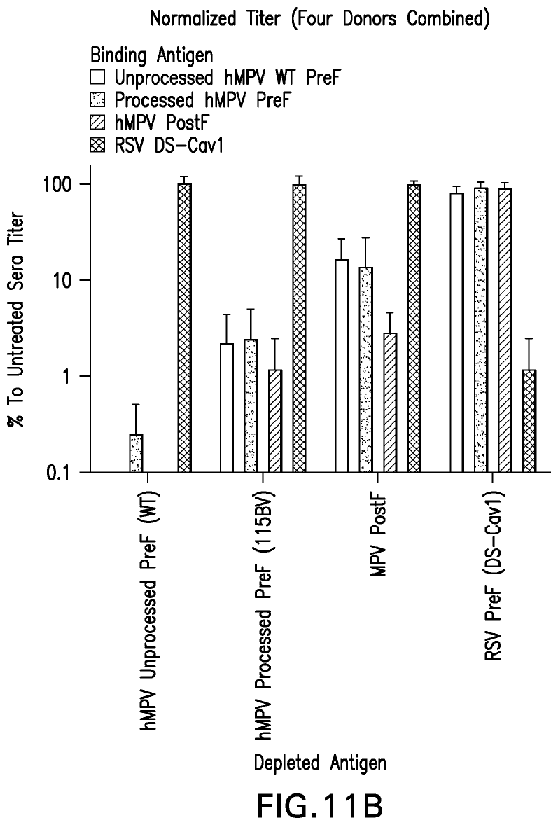

Comparing binding of isolated mAbs to unprocessed PreF with PostF showed that there were three groups of mAbs: PreF-specific, PostF-specific, and Pre/Post-dual mAbs (FIG. 10A). The groups of PreF-specific and Pre/Post-dual mAbs appear to have similar numbers of potent neutralizing mAbs (FIG. 10A). For example, in the 28 mAbs with neutralizing IC₅₀<50 ng/ml and 33 mAbs with neutralizing IC₅₀ between 50-500 ng/ml, 14 and 15 of mAbs were PreF-specific (defined as PostF binding response <0.3), respectively, suggesting that potent neutralizing mAbs could be elicited by both PreF-specific epitopes as well as epitopes presented on both PreF and PostF. Consistent with this data, investigators also determined that PostF was able to deplete most of binding reactivity to PreF in these human donors' serum samples (FIG. 11A and FIG. 11B and Table 5).

The PreF/PostF binding-specificity of mAbs was also well-correlated with their antigenic sites: site III′ and site IV mAbs were mostly Pre/Post-dual binders, site II mAbs had similar number of PreF-specific and Pre/Post dual mAbs, and the rest of other sites including site V, site III, site IV′, site α, and site β, were PreF-specific (FIG. 10B).

The binding of isolated mAbs to unprocessed PreF and as compared to processed PreF was analyzed using assays. Interestingly, there are two apparent groups of mAbs—a group of mAbs showed similar binding affinity to both conformations of PreF; whereas the other group had significantly reduced binding to processed PreF than unprocessed PreF (FIG. 10C and FIG. 10D). These data suggest that there are two types of epitopes—one type of epitopes, enriched in site II, are presented on both processed and unprocessed PreF, (FIG. 10D); and the other group of epitopes, enriched in site IV′, site α, and site β, are presented better in the unprocessed PreF, (FIG. hOD). Site II, III, III′, and IV appear to include epitopes belong to both groups (FIG. 10D). Interestingly, the mAbs of the latter group were observed to have preferential binding to unprocessed PreF showed less neutralization potency than the mAbs of the former group (FIG. 10C). The neutralization potency of mAbs were negatively correlated with the ratio of processed PreF/unprocessed PreF binding (FIG. hOE), highlighting the potential differences of immunogenicity between unprocessed and processed hMPV F antigens.

TABLE 5
Human serum absorption assay data for unprocessed hMPV PreF,
processed hMPV PreF, hMPV PostF, and RSV PreF trimers.

| gELISA binding | | ELISA titer (fold dilution) | | | |
|---|---|---|---|---|---|
| antigens | Absorb antigen | #84974 | #86559 | #21083 | #85359 |
| Unprocessed hMPV WT | Unprocessed hMPV WT | <1 | <1 | <1 | <1 |
| PreF trimer | PreF trimer | | | | |
| Processed stabilized | | <1 | 377 | <1 | <1 |
| hMPV PreF trimer | | | | | |
| (115BV) | | | | | |
| hMPV PostF trimer | | <1 | <1 | <1 | <1 |
| RSV PreF (DS-Cav1) | | 3423 | 5508 | 3975 | 6039 |
| Unprocessed hMPV WT | Processed stabilized | <1 | 144 | 656 | <1 |
| PreF trimer | hMPV PreF trimer | | | | |
| Processed stabilized | (115BV) | <1 | <1 | 521 | <1 |
| hMPV PreF trimer | | | | | |
| (115BV) | | | | | |
| hMPV PostF trimer | | <1 | 41 | 250 | <1 |
| RSV PreF (DS-Cav1) | | 4504 | 2750 | 4771 | 6141 |
| Unprocessed hMPV WT | MPV PostF (dPFv1) | 673 | 26378 | 582 | 22 |
| PreF trimer | | | | | |
| Processed stabilized | | 0 | 15595 | 311 | <1 |
| hMPV PreF trimer | | | | | |
| (115BV) | | | | | |
| hMPV PostF trimer | | 56 | 1891 | 58 | 75 |
| RSV PreF (DS-Cav1) | | 3306 | 4458 | 5293 | 5755 |
| Unprocessed hMPV WT | RSV PreF (DS-Cav1) | 5129 | 55865 | 5520 | 2428 |
| PreF trimer | | | | | |
| Processed stabilized | | 4103 | 36424 | 5350 | 1862 |
| hMPV PreF trimer | | | | | |
| (115BV) | | | | | |
| hMPV PostF trimer | | 5224 | 29467 | 4344 | 2957 |
| RSV PreF (DS-Cav1) | | <1 | <1 | <1 | 239 |
| Unprocessed hMPV WT | PBS buffer | 4989 | 56976 | 7154 | 3966 |
| PreF trimer | | | | | |
| Processed stabilized | | 4555 | 29594 | 5377 | 2753 |
| hMPV PreF trimer | | | | | |
| (115BV) | | | | | |
| hMPV PostF trimer | | 5107 | 25086 | 5279 | 4227 |
| RSV PreF (DS-Cav1) | | 3281 | 4204 | 5359 | 5006 |

### Example 6. Characterization of the Thermostability of Unprocessed and Processed hMPV F Antigens

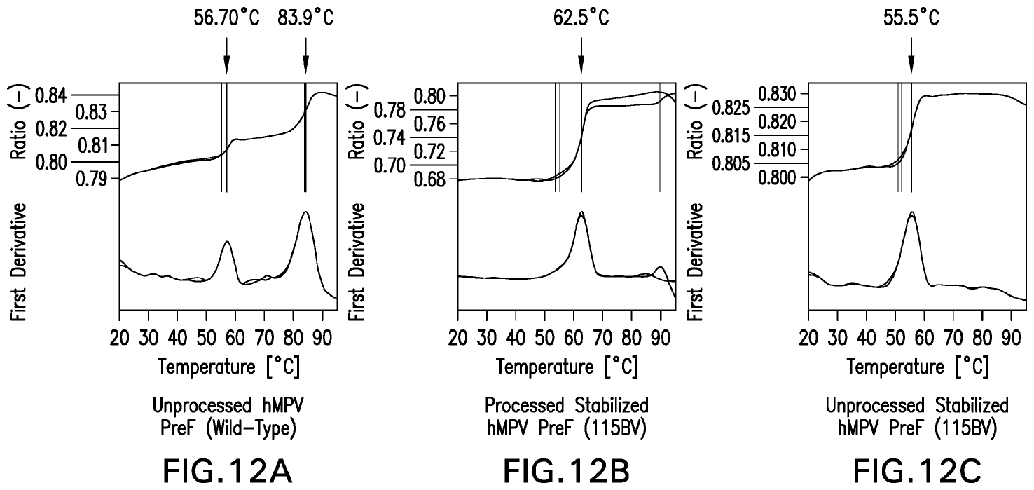

To further characterize the differences between unprocessed and processed hMPV PreF antigens, investigators analysed the thermostability of the antigens by differential scanning fluorimetry. The unprocessed hMPV PreF construct, which does not include A185P stabilizing mutation (FIG. 2A), showed two unfolding transitions with a first transition at 56.7° C. and an additional transition at 83.9° C. (FIG. 12A). In contrast, the melting temperature of furin processed stabilized hMPV PreF 115BV that contains the A185P mutation as well as the RR furin cleavage site at position 82 (FIG. 12A) is with 62.5° C. significantly higher (FIG. 12B), suggesting increased thermostability of the latter protein. To determine whether the improved thermostability is attributed to the introduction of A185P stabilizing mutation or furin-mediated proteolytic cleavage, we further generated unprocessed stabilized hMPV PreF 115BV protein in the absence of co-expressed furin (FIG. 2A). The melting temperature of this A185P stabilized construct that had not been processed by furin is with 55.5° C. (FIG. 12C), similar to unprocessed hMPV PreF protein without the A185P mutation, indicating that the furin-mediated proteolytic cleavage significantly improved the thermostability of hMPV PreF protein. Interestingly, the second high temperature transition could only be observed for the hMPV F construct that does not include the A185P stabilizing mutation. Without being limited by any specific theory or mechanism, it is believed that the addition of the stabilizing A185P mutation shifts this high temperature transition to a temperature above 95° C., which falls outside the observed temperature range of this experiment.

### Discussion

Despite the highly similar structural folding and some conserved epitopes between RSV and hMPV F proteins, whether the antigenicity of hMPV F is similar to RSV F remains unclear to date. In this study, investigators isolated and characterized over one hundred mAbs from multiple healthy adult donors, providing a comprehensive antigenic map of hMPV-F specific mAbs elicited from natural infection. Investigators generated 113 hMPV-F specific mAbs isolated from memory B cells of multiple healthy human donors. The investigators analysed the antibodies' germline usage, epitopes, neutralization potencies, and binding specificities to different forms of F antigen: unprocessed (or uncleaved) prefusion (PreF), processed (or cleaved) PreF, and postfusion (PostF). An mAb with ultra-high neutralization potency on both hMPV A and B viruses has been characterized and mapped to antigenic site V. Data showed that unlike RSV-F specific mAbs, antibodies responses to hMPV F are less dominant against the apex of the antigen, possibly due to the additional glycosylation. The majority of the potent neutralizing mAbs recognize epitopes on the side of hMPV F. Furthermore, novel neutralizing epitopes that differ from previously defined antigenic sites on RSV F were identified, and multiple binding modes of site V and II mAbs have been discovered. Interestingly, mAbs that bind preferentially to the unprocessed PreF showed poor neutralization potency, highlighting the potential differences of immunogenicity between proteolytically unprocessed and processed F antigens. These results elucidate the immune recognition of hMPV infection.

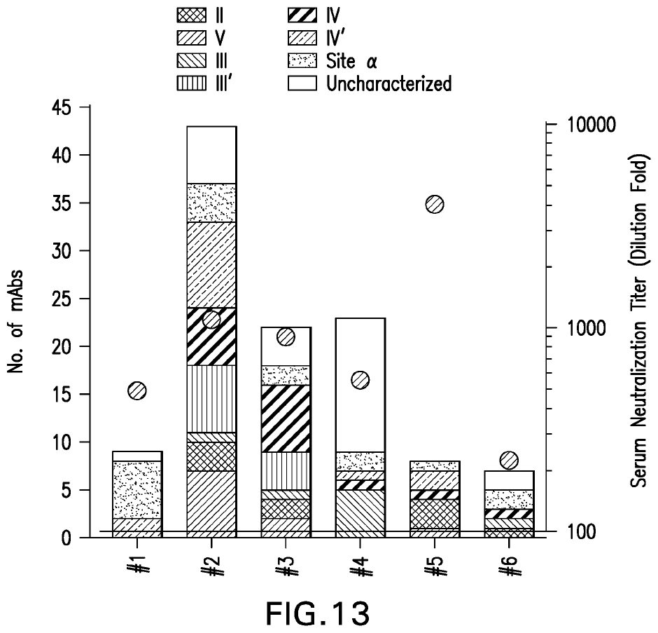

Most of isolated mAbs had significant somatic hypermutations compared with the germline sequences (FIG. 3C), suggesting that the donors, which were pre-screened with hMPV F neutralizing titers, were likely subjected to repeated hMPV infections in the past. Epitope binning and screening with site-knock out mutants suggested that there are eight distinct antigenic sites of hMPV PreF, including four sites that were not previously reported (see FIGS. 8A-C). Data showed that the numbers of isolated mAbs of each site varies by donors (FIG. 13), possibly due to the difference of their immune background. Nevertheless, this did not interfere with the ability for investigators to characterize the common features of mAbs related to their targeted antigenic sites. A panel of mAbs, including M4B06, exhibited high neutralization potency in the in vitro neutralization assay with IC₅₀<50 ng/ml, highlighting their potential applications as prophylactic or therapeutic interventions against hMPV infection. These mAbs were mapped to site II, IV, V, III, and a newly-identified site III′ (Table 3A, Table 3B and Table 3C). Within pre-defined antigenic site II and V, multiple binding modes have also been identified (FIGS. 6A-C). In addition, four new antigenic sites were identified (FIGS. 8A-C). Sites III′ and IV′, which are adjacent to previously defined sites III and IV, induced distinct groups of antibodies.

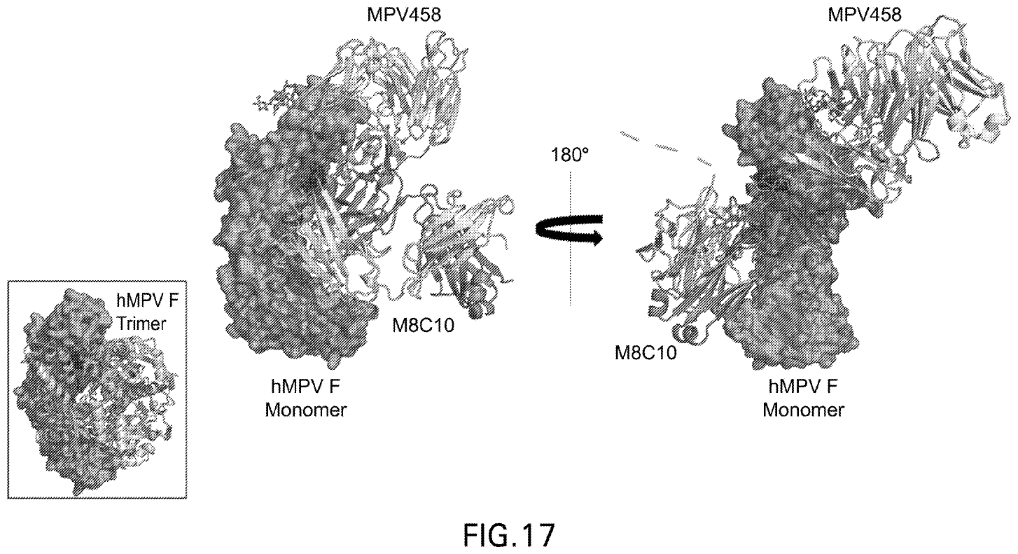

Antibodies against two other novel sites, one located in the trimer interface and another one has yet to be characterized, also comprised a significant portion of the total antibodies that were identified. Another mAb MPV458 has been reported to target the 66-87 helix that is located near the trimer interface of MPV F head region [ref. doi.org/10.1371/journal.ppat.1008942]. The epitope of MPV458 does not overlay with the epitope of M8C10, which is completely buried within the MPV F trimer. See FIG. 17. This data elucidates the breadth of antibody recognition within the trimerization interface. Natural RSV infection elicited antibodies preferably targeting prefusion-specific apex sites such as site Ø and V [48]. In contrast, analysis of human donors' serum samples in our study suggested that PostF was able to deplete most of binding reactivity to PreF (FIG. 11A and FIG. 11B). Moreover, investigators determined the comparable numbers of hMPV PreF specific memory B cells versus PreF/PostF-dual memory B cells and didn't observe the immuno-dominancy of site Ø and V (FIG. 8C), consistent with a previous report that the apex of hMPV F is heavily shielded by the glycans and less accessible for antibody recognition [28]. The hMPV PreF and PostF antigens were reported to elicit comparable neutralizing titers in mice [28], likely driven by the neutralizing mAbs (nAbs) targeting site II, IV, and III′, which were PreF/PostF dual binders as characterized in this study. In summary, the data herein highlight diverse antibody responses against hMPV F during natural infection and reveal novel features which are distinct from studies of antibodies against RSV F.

Since nascent hMPV F protein requires proteolytic activation by transmembrane proteases such as TMPRSS2 at the cell surface or in the virus like particle [26], investigators hypothesized that both forms of PreF antigen may be accessible to humoral immunity. By examining the binding specificity of isolated mAbs to both proteolytically unprocessed and processed PreF forms, a large group of mAbs were identified that had apparent binding preference to unprocessed PreF than processed PreF, highlighting the potential differences of immunogenicity between the two PreF form, despite their highly similar structures in monomer forms.

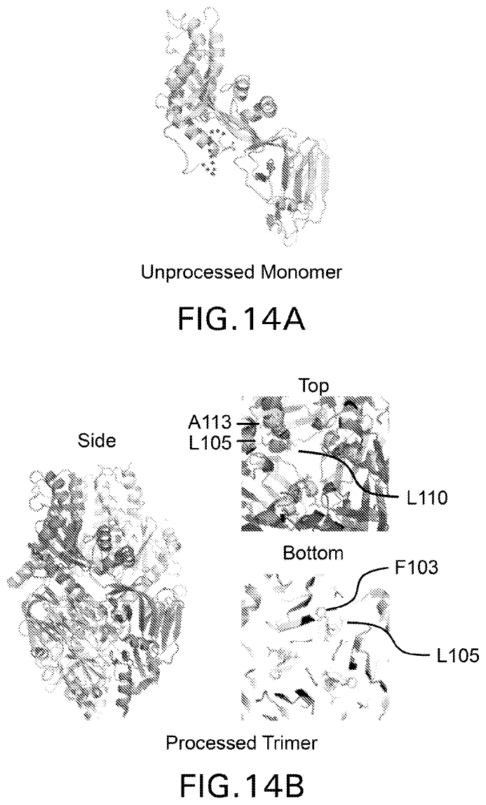

Without wishing to be bound by theory, one possible explanation to this observation is that the proteolytic cleavage generates a hydrophobic end in the N-terminus of F₁ segments, forming a hydrophobic core and stabilizing the trimerization of F protein (FIG. 14A and FIG. 14B). Indeed, DSF results indicate that the proteolytic process may have improved the thermostability of hMPV PreF trimer (FIGS. 12A-C). The unprocessed PreF trimer might present a more dynamic conformation, therefore exposing some epitopes which are less accessible on the processed and more stabilized PreF trimer. For example, a group of unprocessed PreF-preferred mAbs competed with M8C10, an mAb shown to recognize an internal epitope site α within the trimerization interface. Similar mAbs have been reported for other viruses such as influenza [54]. One caveat of our study is that only unprocessed hMPV PreF was used as the bait for antibody isolation, thus investigators would not be able to identify mAbs targeting epitopes that are only present on the processed hMPV PreF. To address this concern, cross-reactivity of different hMPV F conformations were analysed using a serum absorption assay. Data showed that unprocessed PreF depleted most cross-reactivity to processed PreF (FIG. 11A and FIG. 11B and Table 3), accordingly the mAbs binding exclusively to processed PreF do not represent a significant portion of serum hMPV F-specific antibodies.

Data show that the processed PreF was also able to deplete cross-reactivity to unprocessed PreF in the serum (FIG. 11A and FIG. 11B and Table 3), likely because the mAbs with unprocessed PreF-preferred binding still retained weak binding to processed PreF antigen when the antigen was in excess (FIG. 10C and FIG. 10D). Interestingly, data showed that there was a negative correlation between the in vitro neutralization potency and the binding preference to the unprocessed F than processed F, suggesting that processed PreF trimer may serve as an effective immunogen, along with the unprocessed PreF, for eliciting higher neutralizing antibody response. However, it is worth noting that there may be other potential antiviral mechanisms independent of virus neutralization. For example, the previously reported FluA-20 antibody did not show in vitro neutralization activity but was able to inhibit in vitro cell-cell fusion and provide in vivo protection in mice [54]. Therefore, further investigation is needed to better understand the protective roles of such unprocessed PreF-preferred mAbs.

### Example 7. Analysis of the MPV-F:M8C10 Fab Protein Complex

In this Example, the detailed structure of M8C10 in complex with MPV-F was analysed and investigators identified a new epitope within the trimer interface. Although antibodies against RSV have been previously seen to partially open the apical end of the F trimer, to our knowledge, a neutralizing antibody capable of disrupting the trimeric fusion protein has only been seen with influenza hemagglutinin.

### Crystallization and Structure Solution

MPV-F:M8C10 Fab protein complex was screened by sparse matrix using a sitting drop format. The crystals were obtained with a crystallization buffer of 20% PEG 3350 and 200 mM sodium iodide. The crystals were vitrified using a freezing solution containing 20% PEG 3350, 10% PEG 400, and 100 mM sodium iodide. Data were collected at the Advanced Photon Source at Argonne National Labs on beamline, 17-ID.

Data were processed with XDS using an autoprocessing protocol. The phases were determined by molecular replacement using a model of MPV-F (PDB code: 4DAG) and a homology model of the Fab built using the automated antibody homology modeler in MOE. Structures were built iteratively using Coot and refinement with Buster and Phenix. Model geometries were validated using Procheck and Molprobity. Molecular figures were generated using a Pymol molecular visualization system.

### Identification and Characterization of M8C10

Antibody M8C10 was identified from a campaign to isolate MPV-F-reactive antibodies from human donor serum as described in the earlier Examples. Based on a Bio-Layer Interferometry antibody competition assay, investigators were able to identify antibodies that did not compete with previously characterized binding sites of MPV-F, M2B6 for site III, M2D2 for a site IV, and M1C7 for a site V. DS-7 binds a separate site that has been characterized structurally which will be referred to as the DS7-site. In characterizing the antibodies, M8C10 was identified as not competing with these reference antibodies (Table 6) using a bio-layer interferometry competition study.

TABLE 6
Bio-Layer Interferometry competition studies of pairs of antibodies

| | | Site | Site | | DS7- |
|---|---|---|---|---|---|
| | Site α | III | IV | Site V | site |
| | M8C10 | M2B6 | M2D2 | M1C7 | DS7 |
| M8C10 | −0.07 | 0.97 | 0.86 | 0.97 | 0.87 |
| M2B6 | 0.76 | −0.03 | 0.91 | 0.87 | 0.24 |
| M2D2 | 0.75 | 0.77 | 0.00 | 0.80 | 0.65 |
| M1C7 | 0.81 | 0.75 | 0.94 | −0.01 | 0.71 |
| DS7 | 0.73 | 0.90 | 1.00 | 0.80 | −0.07 |

* Values were calculated as: Ab binding/Ag binding smaller number means more competition

To further confirm the M8C10 binds to MPV-F, the binding was tested by Surface Plasmon Resonance. MPV-F was captured on the chip and M8C10 Fab was introduced at increasing analyte concentrations. Based on kinetic fitting, the dissociation constant was calculated to be 1.2 nM.

M8C10 was tested for its ability to inhibit MPV infection in a virus neutralization assay. Two strains of MPV were tested from the A1 and B2 subgroups. M8C10 inhibited MPV strain Al with an IC50 of 370.7 nM and stain B2 with an IC50 of 660.5 nM. While many antibodies can bind MPV-F without being about to neutralize the virus, the M8C10 antibody appears to have sub-micromolar activity against the virus.

### Structure of M8C10

It was observed that the M8C10 antibody did not compete with one of the reference antibodies, we sought to determine the crystal structure of the complex between MPV-F and a Fab fragment of M8C10. Each component was individually purified, and the antigen-antibody complex was formed by mixing with a molar excess of the Fab. The complex was purified from the unbound Fab by size exclusion chromatography. and concentrated for crystallization. A dataset was collected that diffracted to 3.0 Å (Table 7).

TABLE 7
Crystallographic statistics

| Wavelength | 1.00 | |
|---|---|---|
| Resolution range | 43.41-2.995 | (3.102-2.995) |
| Space group | I 2 2 2 | |
| Unit cell | 98.9 117.67 181.22 90 90 90 | |
| Total reflections | 130211 | (14527) |
| Unique reflections | 21574 | (2142) |
| Multiplicity | 6.0 | (6.8) |
| Completeness (%) | 99.50 | (99.86) |
| Mean I/sigma(I) | 7.93 | (2.43) |
| Wilson B-factor | 67.83 | |
| R-merge | 0.165 | (1.017) |
| R-meas | 0.1815 | (1.103) |
| R-pim | 0.07459 | (0.4233) |
| CC½ | 0.992 | (0.823) |
| CC* | 0.998 | (0.95) |
| Reflections used in refinement | 21582 | (2142) |
| Reflections used for R-free | 1132 | (132) |
| R-work | 0.2435 | (0.3560) |
| R-free | 0.2803 | (0.3933) |
| CC(work) | 0.928 | (0.778) |
| CC(free) | 0.893 | (0.652) |

| | |
|---|---|
| Number of non-hydrogen atoms | 6142 |
| macromolecules | 6116 |
| solvent | 26 |
| Protein residues | 817 |
| RMS(bonds) | 0.004 |
| RMS(angles) | 0.77 |
| Ramachandran favored (%) | 94.65 |
| Ramachandran allowed (%) | 5.35 |
| Ramachandran outliers (%) | 0 |
| Rotamer outliers (%) | 0.15 |
| Clashscore | 9.36 |
| Average B-factor | 72.37 |
| macromolecules | 72.45 |
| solvent | 53.96 |

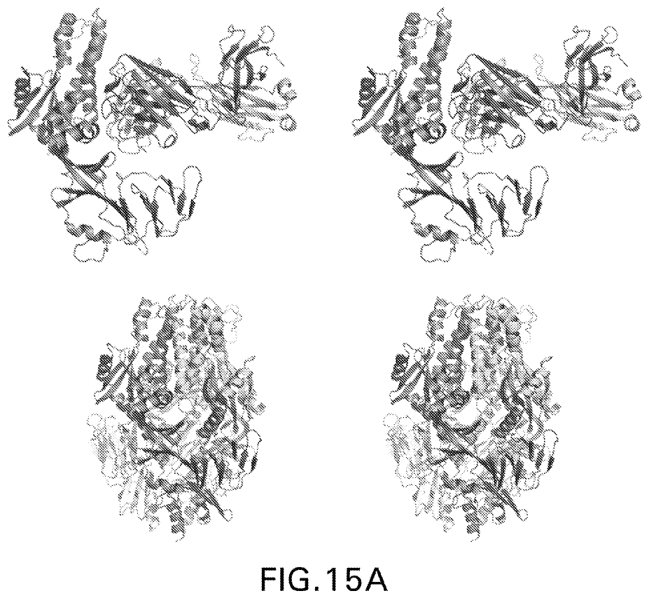

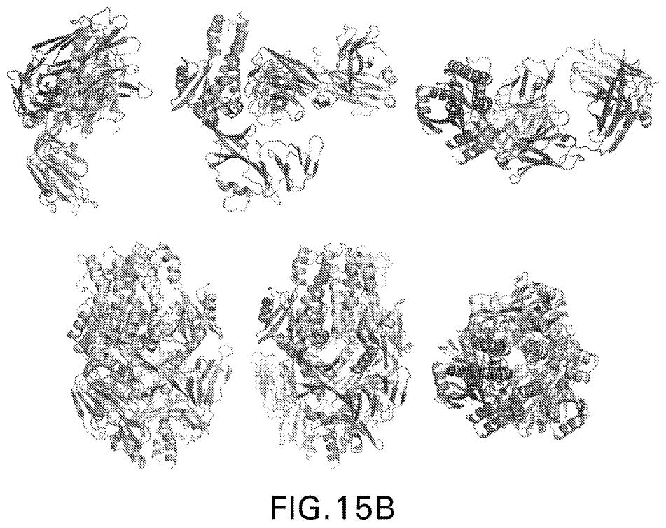

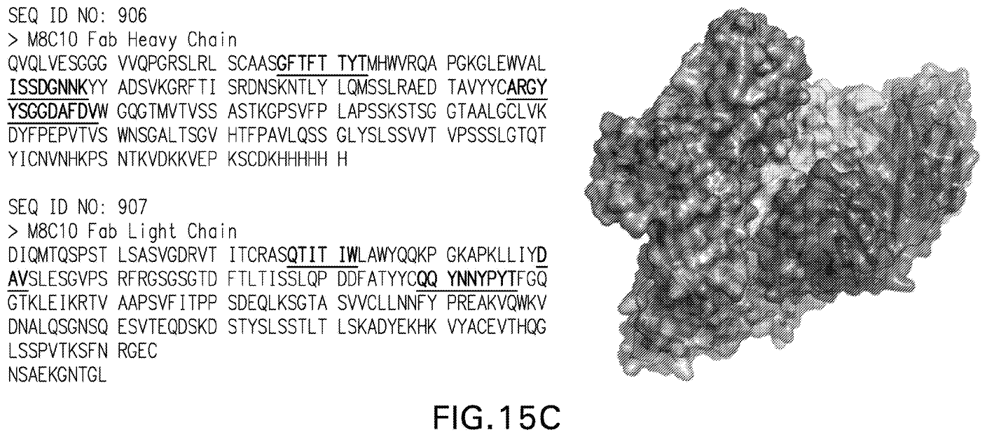

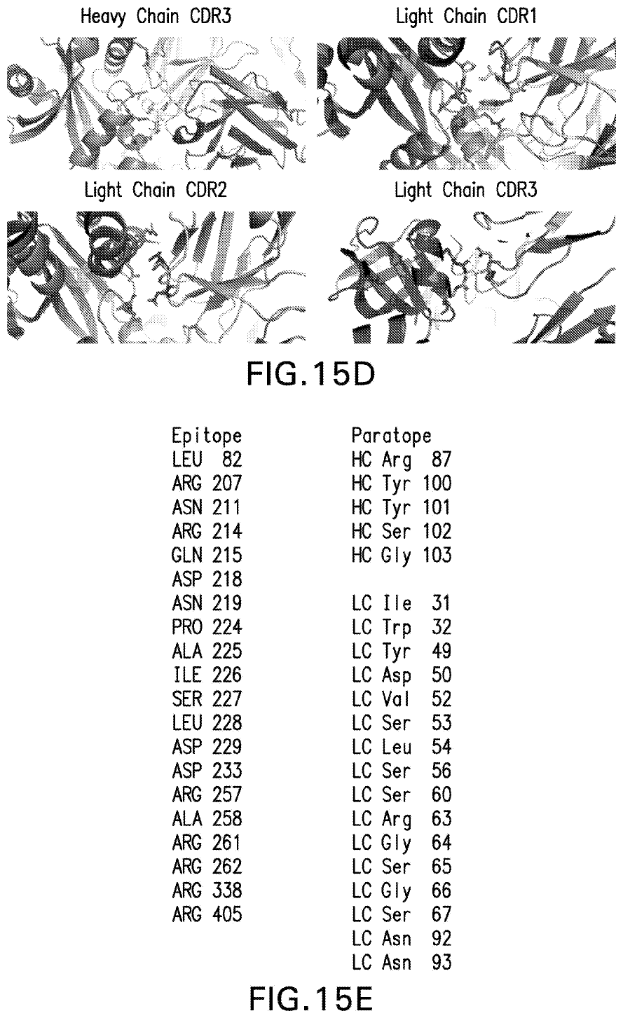

The structure reveals a single copy of the MPV-F:M8C10 Fab complex in the asymmetric unit. The interface of the complex buries a total of 1901 sq. A. All antibody structures against RSV or MPV thus far have demonstrated a binding site that is on the exterior of the surface of the F protein trimer. The structure demonstrates that M8C10 binds to the surface that mediates the trimer interface of F (FIG. 15A and FIG. 15B).

Data shows that the M8C10 Fab binds to MPV-F through interactions mediated by four of the six complementarity determining regions (CDRs). The heavy chain CDR1 and CDR2 do not interact with MPV-F in the structure (FIG. 15C). The hydroxyl oxygen of Tyr 100 in heavy chain CDR3 mediates an interaction with Arg 207 of MPV-F, Tyr 101 makes van der Waals interactions with Leu 228 on MPV-F, and the backbone amide nitrogen makes a salt bridge with Asp 233 on MPV-F. Interactions from the light chain CDR1 loop are primarily made by Ile 31 and Trp 32 which pack against Pro 224, Ala 225, Ile 226, Arg 261, and Arg 262 of MPV-F. From the light chain CDR2, Asp 50 makes a hydrogen bond with Ser 227 on MPV-F and Val 52 makes van der Waals interactions. The light chain CDR3 makes numerous charged interactions with Arg 262 and Arg 338 on MPV-F to the side chain and backbone carbonyl of Asn 92 (FIG. 15D). In addition to the CDRs, the framework regions of the light chain make numerous contacts with MPV-F (FIG. 15E). The list of interacting residues are shown in FIG. 15E.

### MPV-F Epitope Mutations

Based on the MPV-F epitope residues identified in the structure, mutants were designed to both confirm the structural results and identity the residues that are critical for M8C10 binding. Table 8 lists the mutations generated.

TABLE 8
List of MPF-F residues that were mutated

| Original | Mutant |
|---|---|
| Arg 198 | Ala |
| Asn 202 | Ala |
| Asp 209 | Tyr |
| Asn 210 | Ala |
| Ala 216 | Tyr |
| Ser 218 | Tyr |
| Leu 219 | Tyr |
| Asp 220 | Tyr |
| Asp 224 | Ala |
| Ala 249 | Tyr |
| Arg 253 | Ala |
| Arg 329 | Ala |

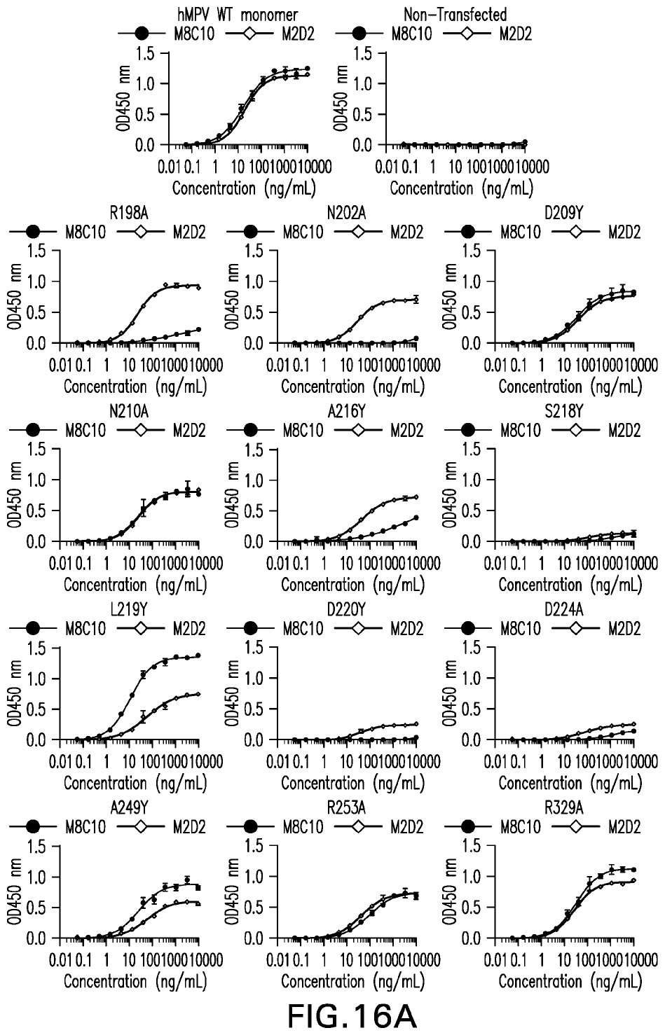

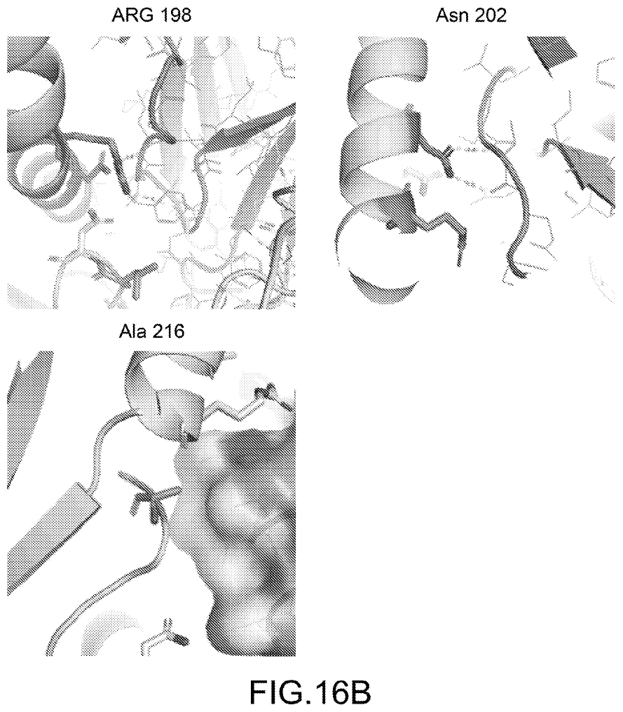

These MPV-F constructs were expressed by transient transfection and the supernatants were tested for M8C10 binding by ELISA. M2D2, an antibody that binds to site IV and should not be affected by the mutations, was used to normalize the signal as a control for expression. (FIG. 16A). Three mutations appeared to make the most profound impact on M8C10 binding, R198A, N202A, A216Y. As shown in FIG. 16B, the Arg 198 residue makes numerous interactions to light chain CDR2 and the residues around it. The Asn 202 residue makes a pair of hydrogen bonds to the back-bone of Leu 54 of the light chain and mutation to alanine would disrupt both bonds. The side chain of Ala 216 makes van der Waals interactions and the introduction of a large side chain would sterically interfere with binding.

Other mutations may also affect M8C10 binding, for example, residues A216Y, L219Y, and A249Y appear to have reduced M8C10 binding but are still able to bind. S218Y, D220Y, and D224A appeared to have reduced expression but could also have reduced M8C10 binding. D210Y, N210A, R253A, and R329A did not appreciably interfere with M8C10 binding.

This disclosure is not limited in its application to the details of construction and the arrangement of components set forth in the following description or illustrated in the drawings. The compositions and methods disclosed herein can have other embodiments and can be practiced or carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### REFERENCES

1. Afonso C L, Amarasinghe G K, Banyai K, Bao Y, Basler C F, Bavari S, et al. Taxonomy of the order Mononegavirales: update 2016. Arch Virol. 2016; 161(8):2351-60. doi: 10.1007/s00705-016-2880-1. PubMed PMID: 27216929; PubMed Central PMCID: PMCPMC4947412.
2. van den Hoogen B G, de Jong J C, Groen J, Kuiken T, de Groot R, Fouchier R A, et al. A newly discovered human pneumovirus isolated from young children with respiratory tract disease. Nat Med. 2001; 7(6):719-24. doi: 10.1038/89098. PubMed PMID: 11385510.
3. Edwards K M, Zhu Y, Griffin M R, Weinberg G A, Hall C B, Szilagyi P G, et al. Burden of human metapneumovirus infection in young children. N Engl J Med. 2013; 368(7):633-43. doi: 10.1056/NEJMoa1204630. PubMed PMID: 23406028; PubMed Central PMCID: PMCPMC3662802.
4. Schuster J E, Williams J V. Human metapneumovirus. Pediatr Rev. 2013; 34(12):558-65. doi: 10.1542/pir.34-12-558. PubMed PMID: 24295817; PubMed Central PMCID: PMCPMC4531267.
5. Jain S, Self W H, Wunderink R G, Fakhran S, Balk R, Bramley A M, et al. Community-Acquired Pneumonia Requiring Hospitalization among U.S. Adults. N Engl J Med. 2015; 373(5):415-27. doi: 10.1056/NEJMoa1500245. PubMed PMID: 26172429; PubMed Central PMCID: PMCPMC4728150.
6. Jain S, Williams D J, Arnold S R, Ampofo K, Bramley A M, Reed C, et al. Community-acquired pneumonia requiring hospitalization among U.S. children. N Engl J Med. 2015; 372(9):835-45. doi: 10.1056/NEJMoa1405870. PubMed PMID: 25714161; PubMed Central PMCID: PMCPMC4697461.
7. van den Hoogen B G, Bestebroer T M, Osterhaus A D, Fouchier R A. Analysis of the genomic sequence of a human metapneumovirus. Virology. 2002; 295(1):119-32. doi: 10.1006/viro.2001.1355. PubMed PMID: 12033771.
8. van den Hoogen B G, van Doornum G J, Fockens J C, Cornelissen J J, Beyer W E, de Groot R, et al. Prevalence and clinical symptoms of human metapneumovirus infection in hospitalized patients. J Infect Dis. 2003; 188(10):1571-7. doi: 10.1086/379200. PubMed PMID: 14624384.
9. Feuillet F, Lina B, Rosa-Calatrava M, Boivin G. Ten years of human metapneumovirus research. J Clin Virol. 2012; 53(2):97-105. doi: 10.1016/j.jcv.2011.10.002. PubMed PMID: 22074934.
10. Boivin G, De Serres G, Cote S, Gilca R, Abed Y, Rochette L, et al. Human metapneumovirus infections in hospitalized children. Emerg Infect Dis. 2003; 9(6):634-40. doi: 10.3201/eid0906.030017. PubMed PMID: 12781001; PubMed Central PMCID: PMCPMC3000156.
11. Deffrasnes C, Hamelin M E, Boivin G. Human metapneumovirus. Semin Respir Crit Care Med. 2007; 28(2):213-21. doi: 10.1055/s-2007-976493. PubMed PMID: 17458775.
12. Esper F, Boucher D, Weibel C, Martinello R A, Kahn J S. Human metapneumovirus infection in the United States: clinical manifestations associated with a newly emerging respiratory infection in children. Pediatrics. 2003; 111(6 Pt 1):1407-10. PubMed PMID: 12777560.
13. Esper F, Martinello R A, Boucher D, Weibel C, Ferguson D, Landry M L, et al. A 1-year experience with human metapneumovirus in children aged <5 years. J Infect Dis. 2004; 189(8):1388-96. doi: 10.1086/382482. PubMed PMID: 15073675.
14. Kahn J S. Epidemiology of human metapneumovirus. Clin Microbiol Rev. 2006; 19(3):546-57. doi: 10.1 128/CMR.00014-06. PubMed PMID: 16847085; PubMed Central PMCID: PMCPMC1539100.
15. Bost P, Giladi A, Liu Y, Bendjelal Y, Xu G, David E, et al. Host-Viral Infection Maps Reveal Signatures of Severe COVID-19 Patients. Cell. 2020. doi: 10.1016/j.cell.2020.05.006. PubMed PMID: 32479746.
16. Koehler P, Comely O A, Bottiger B W, Dusse F, Eichenauer D A, Fuchs F, et al. COVID-19 associated pulmonary aspergillosis. Mycoses. 2020; 63(6):528-34. doi: 10.1111/myc.13096. PubMed PMID: 32339350.
17. Touzard-Romo F, Tape C, Lonks J R. Co-infection with SARS-CoV-2 and Human Metapneumovirus. R I Med J (2013). 2020; 103(2):75-6. PubMed PMID: 32192233.
18. Cox R G, Livesay S B, Johnson M, Ohi M D, Williams J V. The human metapneumovirus fusion protein mediates entry via an interaction with RGD-binding integrins. J Virol. 2012; 86(22):12148-60. doi: 10.1128/JVI.01133-12. PubMed PMID: 22933271; PubMed Central PMCID: PMCPMC3486500.
19. Melero J A, Mas V. The Pneumovirinae fusion (F) protein: A common target for vaccines and antivirals. Virus Res. 2015; 209:128-35. doi: 10.1016/j.virusres.2015.02.024. PubMed PMID: 25738581.
20. Biacchesi S, Skiadopoulos M H, Yang L, Lamirande E W, Tran K C, Murphy B R, et al. Recombinant human Metapneumovirus lacking the small hydrophobic S H and/or attachment G glycoprotein: deletion of G yields a promising vaccine candidate. J Virol. 2004; 78(23):12877-87. doi: 10.1128/JVI.78.23.12877-12887.2004. PubMed PMID: 15542640; PubMed Central PMCID: PMCPMC525014.
21. van den Hoogen B G, Herfst S, Sprong L, Cane P A, Forleo-Neto E, de Swart R L, et al. Antigenic and genetic variability of human metapneumoviruses. Emerg Infect Dis. 2004; 10(4):658-66. doi: 10.3201/eid1004.030393. PubMed PMID: 15200856; PubMed Central PMCID: PMCPMC3323073.
22. Skiadopoulos M H, Biacchesi S, Buchholz U J, Amaro-Carambot E, Surman S R, Collins P L, et al. Individual contributions of the human metapneumovirus F, G, and S H surface glycoproteins to the induction of neutralizing antibodies and protective immunity. Virology. 2006; 345(2):492-501. doi: 10.1016/j.virol.2005.10.016. PubMed PMID: 16300813.
23. Ryder A B, Tollefson S J, Podsiad A B, Johnson J E, Williams J V. Soluble recombinant human metapneumovirus G protein is immunogenic but not protective. Vaccine. 2010; 28(25):4145-52. doi: 10.1016/j.vaccine.2010.04.007. PubMed PMID: 20417260; PubMed Central PMCID: PMCPMC2894472.
24. Wen S C, Williams J V. New Approaches for Immunization and Therapy against Human Metapneumovirus. Clin Vaccine Immunol. 2015; 22(8):858-66. doi: 10.1 128/CVI.00230-15. PubMed PMID: 26063237; PubMed Central PMCID: PMCPMC4519717.
25. Collins P L, Mottet G. Post-translational processing and oligomerization of the fusion glycoprotein of human respiratory syncytial virus. J Gen Virol. 1991; 72 (Pt 12):3095-101. doi: 10.1099/0022-1317-72-12-3095. PubMed PMID: 1765771.
26. Shirogane Y, Takeda M, Iwasaki M, Ishiguro N, Takeuchi H, Nakatsu Y, et al. Efficient multiplication of human metapneumovirus in Vero cells expressing the transmembrane serine protease TMPRSS2. J Virol. 2008; 82(17):8942-6. doi: 10.1128/JVI.00676-08. PubMed PMID: 18562527; PubMed Central PMCID: PMCPMC2519639.
27. Krarup A, Truan D, Furmanova-Hollenstein P, Bogaert L, Bouchier P, Bisschop I J, et al. A highly stable prefusion RSV F vaccine derived from structural analysis of the fusion mechanism. Nat Commun. 2015; 6:8143. doi: 10.1038/ncomms9143. PubMed PMID: 26333350; PubMed Central PMCID: PMCPMC4569726.
28. Battles M B, Mas V, Olmedillas E, Cano O, Vazquez M, Rodriguez L, et al. Structure and immunogenicity of pre-fusion-stabilized human metapneumovirus F glycoprotein. Nat Commun. 2017; 8(1):1528. doi: 10.1038/s41467-017-01708-9. PubMed PMID: 29142300; PubMed Central PMCID: PMCPMC5688127.
29. McLellan J S, Yang Y, Graham B S, Kwong P D. Structure of respiratory syncytial virus fusion glycoprotein in the postfusion conformation reveals preservation of neutralizing epitopes. J Virol. 2011; 85(15):7788-96. doi: 10.1128/JVI.00555-11. PubMed PMID: 21613394; PubMed Central PMCID: PMCPMC3147929.
30. Mas V, Rodriguez L, Olmedillas E, Cano 0, Palomo C, Terron M C, et al. Engineering, Structure and Immunogenicity of the Human Metapneumovirus F Protein in the Postfusion Conformation. PLoS Pathog. 2016; 12(9):e1005859. doi: 10.1371/joumal.ppat.1005859. PubMed PMID: 27611367; PubMed Central PMCID: PMCPMC5017722.
31. McLellan J S, Chen M, Joyce M G, Sastry M, Stewart-Jones G B, Yang Y, et al. Structure-based design of a fusion glycoprotein vaccine for respiratory syncytial virus. Science. 2013; 342(6158):592-8. doi: 10.1126/science.1243283. PubMed PMID: 24179220; PubMed Central PMCID: PMCPMC4461862.
32. Joyce M G, Zhang B, Ou L, Chen M, Chuang G Y, Druz A, et al. Iterative structure-based improvement of a fusion-glycoprotein vaccine against RSV. Nat Struct Mol Biol. 2016; 23(9):811-20. doi: 10.1038/nsmb.3267. PubMed PMID: 27478931; PubMed Central PMCID: PMCPMC5016229.
33. McLellan J S, Chen M, Leung S, Graepel K W, Du X, Yang Y, et al. Structure of RSV fusion glycoprotein trimer bound to a prefusion-specific neutralizing antibody. Science. 2013; 340(6136):1113-7. doi: 10.1126/science.1234914. PubMed PMID: 23618766; PubMed Central PMCID: PMCPMC4459498.
34. Wen X, Krause J C, Leser G P, Cox R G, Lamb R A, Williams J V, et al. Structure of the human metapneumovirus fusion protein with neutralizing antibody identifies a pneumovirus antigenic site. Nat Struct Mol Biol. 2012; 19(4):461-3. doi: 10.1038/nsmb.2250. PubMed PMID: 22388735; PubMed Central PMCID: PMCPMC3546531.
35. Rossey I, McLellan J S, Saelens X, Schepens B. Clinical Potential of Prefusion RSV F-specific Antibodies. Trends Microbiol. 2018; 26(3):209-19. doi: 10.1016/j.tim.2017.09.009. PubMed PMID: 29054341.
36. Gilman M S, Castellanos C A, Chen M, Ngwuta J O, Goodwin E, Mom S M, et al. Rapid profiling of RSV antibody repertoires from the memory B cells of naturally infected adult donors. Sci Immunol. 2016; 1(6). doi: 10.1126/sciimmunol.aaj 1879. PubMed PMID: 28111638; PubMed Central PMCID: PMCPMC5244814.
37. Zhu Q, McLellan J S, Kallewaard N L, Ulbrandt N D, Palaszynski S, Zhang J, et al. A highly potent extended half-life antibody as a potential RSV vaccine surrogate for all infants. Sci Transl Med. 2017; 9(388). doi: 10.1126/scitranslmed.aaj1928. PubMed PMID: 28469033.
38. Gilman M S, Mom S M, Mas V, Chen M, Patel N K, Kramer K, et al. Characterization of a Prefusion-Specific Antibody That Recognizes a Quaternary, Cleavage-Dependent Epitope on the RSV Fusion Glycoprotein. PLoS Pathog. 2015; 11(7):e1005035. doi: 10.1371/joumal.ppat.1005035. PubMed PMID: 26161532; PubMed Central PMCID: PMCPMC4498696.
39. Mousa J J, Kose N, Matta P, Gilchuk P, Crowe J E, Jr. A novel pre-fusion conformation-specific neutralizing epitope on the respiratory syncytial virus fusion protein. Nat Microbiol. 2017; 2:16271. doi: 10.1038/nmicrobiol.2016.271. PubMed PMID: 28134924; PubMed Central PMCID: PMCPMC5463187.
40. Goodwin E, Gilman MSA, Wrapp D, Chen M, Ngwuta J O, Mom S M, et al. Infants Infected with Respiratory Syncytial Virus Generate Potent Neutralizing Antibodies that Lack Somatic Hypermutation. Immunity. 2018; 48(2):339-49 e5. doi: 10.1016/j.immuni.2018.01.005. PubMed PMID: 29396163; PubMed Central PMCID: PMCPMC6005179.
41. Mousa J J, Binshtein E, Human S, Fong R H, Alvarado G, Doranz B J, et al. Human antibody recognition of antigenic site I V on Pneumovirus fusion proteins. PLoS Pathog. 2018; 14(2):e1006837. doi: 10.1371/journal.ppat.1006837. PubMed PMID: 29470533; PubMed Central PMCID: PMCPMC5823459.
42. McLellan J S, Chen M, Chang J S, Yang Y, Kim A, Graham B S, et al. Structure of a major antigenic site on the respiratory syncytial virus fusion glycoprotein in complex with neutralizing antibody 101F. J Virol. 2010; 84(23):12236-44. doi: 10.1128/JVI.01579-10. PubMed PMID: 20881049; PubMed Central PMCID: PMCPMC2976384.
43. Chen Z, Zhang L, Tang A, Callahan C, Pristatsky P, Swoyer R, et al. Discovery and Characterization of Phage Display-Derived Human Monoclonal Antibodies against RSV F Glycoprotein. PLoS One. 2016; 11(6):e0156798. doi: 10.1371/joumal.pone.0156798. PubMed PMID: 27258388; PubMed Central PMCID: PMCPMC4892554.
44. Johnson S, Oliver C, Prince G A, Hemming V G, Pfarr D S, Wang S C, et al. Development of a humanized monoclonal antibody (MEDI-493) with potent in vitro and in vivo activity against respiratory syncytial virus. J Infect Dis. 1997; 176(5):1215-24. PubMed PMID: 9359721.
45. Corti D, Bianchi S, Vanzetta F, Minola A, Perez L, Agatic G, et al. Cross-neutralization of four paramyxoviruses by a human monoclonal antibody. Nature. 2013; 501(7467):439-43. doi: 10.1038/naturel2442. PubMed PMID: 23955151.
46. Tian D, Battles M B, Mom S M, Chen M, Modjarrad K, Kumar A, et al. Structural basis of respiratory syncytial virus subtype-dependent neutralization by an antibody targeting the fusion glycoprotein. Nat Commun. 2017; 8(1):1877. doi: 10.1038/s41467-017-01858-w. PubMed PMID: 29187732; PubMed Central PMCID: PMCPMC5707411.
47. Wen X, Mousa J J, Bates J T, Lamb R A, Crowe J E, Jr., Jardetzky T S. Structural basis for antibody cross-neutralization of respiratory syncytial virus and human metapneumovirus. Nat Microbiol. 2017; 2:16272. doi: 10.1038/nmicrobiol.2016.272. PubMed PMID: 28134915; PubMed Central PMCID: PMCPMC5308794.
48. Ngwuta J O, Chen M, Modjarrad K, Joyce M G, Kanekiyo M, Kumar A, et al. Prefusion F-specific antibodies determine the magnitude of RSV neutralizing activity in human sera. Sci Transl Med. 2015; 7(309):309ra162. doi: 10.1 126/scitranslmed.aac4241. PubMed PMID: 26468324; PubMed Central PMCID: PMCPMC4672383.
49. Magro M, Mas V, Chappell K, Vazquez M, Cano O, Luque D, et al. Neutralizing antibodies against the preactive form of respiratory syncytial virus fusion protein offer unique possibilities for clinical intervention. Proc Natl Acad Sci USA. 2012; 109(8):3089-94. doi: 10.1073/pnas.1115941109. PubMed PMID: 22323598; PubMed Central PMCID: PMCPMC3286924.
50. Ulbrandt N D, Ji H, Patel N K, Riggs J M, Brewah Y A, Ready S, et al. Isolation and characterization of monoclonal antibodies which neutralize human metapneumovirus in vitro and in vivo. J Virol. 2006; 80(16):7799-806. doi: 10.1128/JVI.00318-06. PubMed PMID: 16873237; PubMed Central PMCID: PMCPMC1563801.
51. Williams J V, Chen Z, Cseke G, Wright D W, Keefer C J, Tollefson S J, et al. A recombinant human monoclonal antibody to human metapneumovirus fusion protein that neutralizes virus in vitro and is effective therapeutically in vivo. J Virol. 2007; 81(15):8315-24. doi: 10.1128/JVI.00106-07. PubMed PMID: 17522220; PubMed Central PMCID: PMCPMC1951312.
52. Bar-Peled Y, Diaz D, Pena-Briseno A, Murray J, Huang J, Tripp R A, et al. A potent neutralizing site III-specific human antibody neutralizes human metapneumovirus in vivo. J Virol. 2019. doi: 10.1128/JVI.00342-19. PubMed PMID: 31292250.
53. Xiao X, Tang A, Cox K S, Wen Z, Callahan C, Sullivan N L, et al. Characterization of potent RSV neutralizing antibodies isolated from human memory B cells and identification of diverse RSV/hMPV cross-neutralizing epitopes. MAbs. 2019:1-13. doi: 10.1080/19420862.2019.1654304. PubMed PMID: 31402751.
54. Bangaru S, Lang S, Schotsaert M, Vanderven H A, Zhu X, Kose N, et al. A Site of Vulnerability on the Influenza Virus Hemagglutinin Head Domain Trimer Interface. Cell. 2019; 177(5):1136-52 e18. doi: 10.1016/j.cell.2019.04.011. PubMed PMID: 31100268; PubMed Central PMCID: PMCPMC6629437.
55. Zhang L, Durr E, Galli J D, Cosmi S, Cejas P J, Luo B, et al. Design and characterization of a fusion glycoprotein vaccine for Respiratory Syncytial Virus with improved stability. Vaccine. 2018. doi: 10.1016/j.vaccine.2018.10.032. PubMed PMID: 30340881.
56. Cox K S, Tang A, Chen Z, Horton M S, Yan H, Wang X M, et al. Rapid isolation of dengue-neutralizing antibodies from single cell-sorted human antigen-specific memory B-cell cultures. MAbs. 2016; 8(1):129-40. doi: 10.1080/19420862.2015.1109757. PubMed PMID: 26491897; PubMed Central PMCID: PMCPMC4966506.
57. Masella A P, Bartram A K, Truszkowski J M, Brown D G, Neufeld J D. PANDAseq: paired-end assembler for illumina sequences. BMC Bioinformatics. 2012; 13:31. doi: 10.1186/1471-2105-13-31. PubMed PMID: 22333067; PubMed Central PMCID: PMCPMC3471323.
58. Giudicelli V, Chaume D, Lefranc M P. IMGT/GENE-D B: a comprehensive database for human and mouse immunoglobulin and T cell receptor genes. Nucleic Acids Res. 2005; 33(Database issue):D256-61. doi: 10.1093/nar/gki010. PubMed PMID: 15608191; PubMed Central PMCID: PMCPMC539964.
59. Ye J, Ma N, Madden T L, Ostell J M. IgBLAST: an immunoglobulin variable domain sequence analysis tool. Nucleic Acids Res. 2013; 41(Web Server issue):W34-40. doi: 10.1093/nar/gkt382. PubMed PMID: 23671333; PubMed Central PMCID: PMCPMC3692102.
60. Camacho C, Coulouris G, Avagyan V, Ma N, Papadopoulos J, Bealer K, et al. BLAST+: architecture and applications. BMC Bioinformatics. 2009; 10:421. doi: 10.1186/1471-2105-10-421. PubMed PMID: 20003500; PubMed Central PMCID: PMCPMC2803857.
61. Gupta N T, Vander Heiden J A, Uduman M, Gadala-Maria D, Yaari G, Kleinstein S H. Change-O: a toolkit for analyzing large-scale B cell immunoglobulin repertoire sequencing data. Bioinformatics. 2015; 31(20):3356-8. doi: 10.1093/bioinformatics/btv359. PubMed PMID: 26069265; PubMed Central PMCID: PMCPMC4793929.
62. Wen Z, Citron M, Bett A J, Espeseth A S, Vora K A, Zhang L, et al. Development and application of a higher throughput RSV plaque assay by immunofluorescent imaging. J Virol Methods. 2019; 263:88-95. doi: 10.1016/j.jviromet.2018.10.022. PubMed PMID: 30381239.
63. Zheng S Q, Palovcak E, Armache J P, Verba K A, Cheng Y, Agard D A. MotionCor2: anisotropic correction of beam-induced motion for improved cryo-electron microscopy. Nat Methods. 2017; 14(4):331-2. doi: 10.1038/nmeth.4193. PubMed PMID: 28250466; PubMed Central PMCID: PMCPMC5494038.
64. Zhang K. Gctf: Real-time CTF determination and correction. J Struct Biol. 2016; 193(1):1-12. doi: 10.1016/j.jsb.2015.11.003. PubMed PMID: 26592709; PubMed Central PMCID: PMCPMC4711343.
65. Scheres S H. RELION: implementation of a Bayesian approach to cryo-E M structure determination. J Struct Biol. 2012; 180(3):519-30. doi: 10.1016/j.jsb.2012.09.006. PubMed PMID: 23000701; PubMed Central PMCID: PMCPMC3690530.
66. Pettersen E F, Goddard T D, Huang C C, Couch G S, Greenblatt D M, Meng E C, et al. UCSF Chimera—a visualization system for exploratory research and analysis. J Comput Chem. 2004; 25(13):1605-12. doi: 10.1002/jcc.20084. PubMed PMID: 15264254.
67. Suloway C, Pulokas J, Fellmann D, Cheng A, Guerra F, Quispe J, et al. Automated molecular microscopy: the new Leginon system. J Struct Biol. 2005; 151(1):41-60. doi: 10.1016/j.jsb.2005.03.010. PubMed PMID: 15890530.
68. Lander G C, Stagg S M, Voss N R, Cheng A, Fellmann D, Pulokas J, et al. Appion: an integrated, database-driven pipeline to facilitate E M image processing. J Struct Biol. 2009; 166(1):95-102. doi: 10.1016/j.jsb.2009.01.002. PubMed PMID: 19263523; PubMed Central PMCID: PMCPMC2775544.
69. Rohou A, Grigorieff N. CTFFIND4: Fast and accurate defocus estimation from electron micrographs. J Struct Biol. 2015; 192(2):216-21. doi: 10.1016/j.jsb.2015.08.008. PubMed PMID: 26278980; PubMed Central PMCID: PMCPMC6760662.
70. Voss N R, Yoshioka C K, Radermacher M, Potter C S, Carragher B. DoG Picker and TiltPicker: software tools to facilitate particle selection in single particle electron microscopy. J Struct Biol. 2009; 166(2):205-13. doi: 10.1016/j.jsb.2009.01.004. PubMed PMID: 19374019; PubMed Central PMCID: PMCPMC2768396.
71. Sorzano C O, Bilbao-Castro J R, Shkolnisky Y, Alcorlo M, Melero R, Caffarena-Fernandez G, et al. A clustering approach to multireference alignment of single-particle projections in electron microscopy. J Struct Biol. 2010; 171(2):197-206. doi: 10.1016/j.jsb.2010.03.011. PubMed PMID: 20362059; PubMed Central PMCID: PMCPMC2893300.
72. Kabat E A, Wu T T. Identical V region amino acid sequences and segments of sequences in antibodies of different specificities. Relative contributions of V H and V L genes, minigenes, and complementarity-determining regions to binding of antibody-combining sites. J Immunol. 1991; 147(5):1709-19. PubMed PMID: 1908882.

1. An antibody or antigen binding fragment thereof which specifically binds human metapneumovirus (hMPV), comprising:
three heavy chain complementarity determining regions (CDRs), wherein the CDR1, CDR2, and CDR3 regions comprise the amino acid sequences of SEQ ID NOs: 257, 258, and 259, respectively; and
three light chain CDRs, wherein the CDR1, CDR2, and CDR3 regions comprise the amino acid sequences of SEQ ID NOs: 596, 597, and 598, respectively.

2-124. (canceled)

125. The antibody or antigen binding fragment thereof of claim 1, which comprises heavy chain variable region comprising the amino acid sequence of SEQ ID NO: 904 and comprises a light chain variable region comprising the amino acid sequence of SEQ ID NO: 905.

126-140. (canceled)

141. A nucleic acid comprising a nucleotide sequence that encodes the antibody or antigen binding fragment thereof of claim 1.

142. An expression vector comprising the nucleic acid of claim 141.

143. A cell transformed with an expression vector of claim 142.

144. A pharmaceutical composition for preventing, treating or detecting a virus comprising:
the antibody or antigen binding fragment of claim 1; and
a pharmaceutically acceptable carrier.

145-146. (canceled)

147. A kit for preventing, treating, or detecting hMPV comprising:
the antibody or antigen binding fragment thereof of claim 1; and instructions for use.

148. A method of producing an antibody or antigen binding fragment thereof comprising:
culturing a host cell comprising a polynucleotide encoding the amino acid sequences of any one of the antibody or antigen binding fragments thereof of claim 1 under conditions favorable to expression of the polynucleotide; and optionally, recovering the antibody or antigen binding fragment thereof from the host cell and/or culture medium.

149-155. (canceled)

156. An isolated hMPV F polypeptide comprising residues 1-490 of SEQ ID NO: 911, or a variant thereof, wherein the isolated hMPV F polypeptide does not include residues 491-539 of SEQ ID NO: 1, wherein the variant comprises an amino acid sequence having 95-99% identity to residues 1-490 of SEQ ID NO: 911, optionally wherein the isolated hMPV F polypeptide consists of SEQ ID NO: 911.

157-159. (canceled)

160. The isolated hMPV F polypeptide of claim 156 comprising residues 1-490 of SEQ ID NO: 911, or a variant thereof, and a trimerization domain,
wherein the isolated hMPV F polypeptide does not include residues 491-539 of SEQ ID NO: 1;
optionally wherein the trimerization domain is a GCN4 domain, a T4 foldon domain, a human type XV collagen domain, or a human type XVIII collagen domain; or
optionally wherein the GCN4 domain has the amino acid sequence of SEQ ID NO: 1022, the T4 foldon domain has the amino acid sequence of SEQ ID NO: 1012, the human type XV collagen domain has the amino acid sequence of SEQ ID NO: 1023, and the human type XVIII collagen domain has the amino acid sequence of SEQ ID NO: 1024.

161-162. (canceled)

163. The isolated hMPV F polypeptide of claim 160, wherein a protease cleavage sequence at residues 99-102 is replaced with a furin-cleavage sequence, wherein the furin cleavage sequence is SEQ ID NO: 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, or 1066.

164-166. (canceled)

167. The isolated hMPV F polypeptide of claim 160, wherein the polypeptide has a mutation at residue 185, optionally wherein the mutation is A185P.

168. (canceled)

169. An isolated hMPV F polypeptide comprising residues 1-521 of SEQ ID NO: 910, residues 1-518 of SEQ ID NO: 912, or residues 1-521 of SEQ ID NO: 913, or variants thereof, optionally wherein the variant comprises an amino acid sequence having 95-99% identity to residues 1-521 of SEQ ID NO: 910, 95-99% identity to residues 1-518 of SEQ ID NO: 912, or 95-99% identity to residues 1-521 of SEQ ID NO: 913,
wherein the variant of the isolated hMPV F polypeptide comprising residues 1-521 of SEQ ID NO: 910 does not vary at residues 486-517,
wherein the variant of the isolated hMPV F polypeptide comprising residues 1-518 of SEQ ID NO: 912 does not vary at residues 102-105 and 490-516, and
wherein the variant of the isolated hMPV F polypeptide comprising residues 1-521 of SEQ ID NO: 913 does not vary at residues 99-102, 185, and 485-521,
optionally wherein the isolated hMPV F polypeptide comprises the amino acid sequence of SEQ ID NO: 910, SEQ ID NO: 912, or SEQ ID NO: 913, or
optionally wherein the isolated hMPV F polypeptide consists of residues 1-521 of SEQ ID NO: 910, residues 1-518 of SEQ ID NO: 912, or residues 1-521 of SEQ ID NO: 913.

170-171. (canceled)

172. The isolated hMPV F polypeptide of claim 156, wherein the isolated hMPV F polypeptide comprises a thrombin cleavage sequence and at least one affinity tag sequence,
optionally wherein the at least one affinity tag sequence is a 6×His sequence (SEQ ID NO: 1014) or a strep tag II sequence.

177. An isolated nucleic acid encoding the isolated hMPV F polypeptide of claim 156:
optionally wherein the isolated nucleic acid is codon-optimized.

178. (canceled)

179. The isolated nucleic acid of claim 177 comprising a nucleotide sequence of SEQ ID NO: 1006, or a variant thereof, wherein the variant comprises a nucleotide sequence encoding the amino acid sequence of SEQ ID NO: 911, and wherein the isolated nucleic acid does not include residues 1453-1600 of SEQ ID NO: 1003;
optionally wherein the variant comprises SEQ ID NO: 1007.

180. (canceled)

181. The isolated nucleic acid of claim 177, comprising a nucleotide sequence of SEQ ID NO: 1004 or a variant thereof, SEQ ID NO: 1008 or a variant thereof, or SEQ ID NO: 1010 or a variant thereof,
wherein the variant of the isolated nucleic acid comprising SEQ ID NO: 1004 encodes the amino acid sequence of SEQ ID NO: 910,
wherein the variant of the isolated nucleic acid comprising SEQ ID NO: 1008 encodes the amino acid sequence of SEQ ID NO: 912, and
wherein the variant of the isolated nucleic acid comprising SEQ ID NO: 1010 encodes the amino acid sequence of SEQ ID NO: 913;
optionally wherein the variant of the isolated nucleic acid comprising SEQ ID NO: 1004 comprises SEQ ID NO: 1005,
wherein the variant of the isolated nucleic acid comprising SEQ ID NO: 1008 comprises SEQ ID NO: 1009, and
wherein the variant of the isolated nucleic acid comprising SEQ ID NO: 1010 comprises SEQ ID NO: 1011: or
optionally wherein the variant comprises a nucleic acid sequence having 95-99% identity to the nucleic acid sequence of SEQ ID NO: 1004, SEQ ID NO: 1008, or SEQ ID NO: 1010.

182-183. (canceled)

184. An immunogenic composition comprising the isolated antigenic peptide of claim 156 and a pharmaceutically acceptable carrier;
optionally wherein the immunogenic composition is multivalent;
optionally wherein the immunogenic composition further comprises an adjuvant; or
optionally wherein the immunogenic composition does not comprise an adjuvant.

185-191. (canceled)

192. A method of inducing an immune response in a subject, the method comprising administering to the subject the immunogenic composition of claim 184 in an amount effective to produce an antigen-specific immune response in the subject,
optionally wherein the antigen-specific immune response comprises a T cell response or a B cell response,
optionally wherein the subject is administered a single dose of the immunogenic composition,
optionally wherein the subject is administered a booster dose of the immunogenic composition,
optionally wherein the immunogenic composition is administered to the subject by intradermal injection or intramuscular injection, or
optionally wherein the subject is infected with hMPV, or wherein the subject is at risk of infection from hMPV.

193-203. (canceled)
